SEQUENCE LISTING (492 sequences)

SEQ ID NO: 1 - DNA, 20 bp, Homo sapiens
taaataggcataattttcta

SEQ ID NO: 2 - DNA, 20 bp, Homo sapiens
ttgctttctgctgcttttgc

SEQ ID NO: 3 - DNA, 20 bp, Homo sapiens
gtagctttcaggattcctac

SEQ ID NO: 4 - DNA, 20 bp, Homo sapiens
ggagcttgttctgtcctcag

SEQ ID NO: 5 - DNA, 20 bp, Homo sapiens
gtttaacgttatcattttcc

SEQ ID NO: 6 - DNA, 20 bp, Homo sapiens
gctcatagagacacattcag

SEQ ID NO: 7 - DNA, 20 bp, Homo sapiens
tttctgatgaggaagatgaa

SEQ ID NO: 8 - DNA, 20 bp, Homo sapiens
gatcttagataagaataatc

SEQ ID NO: 9 - DNA, 20 bp, Homo sapiens
ttacttctaaataatattga

SEQ ID NO: 10 - DNA, 20 bp, Homo sapiens
tggaccatggatgcactctg

SEQ ID NO: 11 - DNA, 20 bp, Homo sapiens
gctacatccattccaaggaa

SEQ ID NO: 12 - DNA, 20 bp, Homo sapiens
ttttctcttagatgtctggt

SEQ ID NO: 13 - DNA, 20 bp, Homo sapiens
tgtagaattttaatgtagaa

SEQ ID NO: 14 - DNA, 20 bp, Homo sapiens
tcttacccctgtacccagaa

SEQ ID NO: 15 - DNA, 20 bp, Homo sapiens
cttgcatgatttagctgaat

SEQ ID NO: 16 - DNA, 20 bp, Homo sapiens
cagaagtcattcagccacta

SEQ ID NO: 17 - DNA, 20 bp, Homo sapiens
gtgtgtgtgttatgtgggaa

SEQ ID NO: 18 - DNA, 20 bp, Homo sapiens
ttattcattcagtttagtta

SEQ ID NO: 19 - DNA, 20 bp, Homo sapiens
gggatttggcagattttaat

SEQ ID NO: 20 - DNA, 20 bp, Homo sapiens
ctgccaaatccccccaaaca

SEQ ID NO: 21 - DNA, 20 bp, Homo sapiens
cacaaggttcaagaatcaca

SEQ ID NO: 22 - DNA, 20 bp, Homo sapiens
catcattttttattgtagaa

SEQ ID NO: 23 - DNA, 21 bp, Homo sapiens
gaatgtgtctctatgagccag

SEQ ID NO: 24 - DNA, 21 bp, Homo sapiens
taagatctaattctattagct

SEQ ID NO: 25 - DNA, 20 bp, Homo sapiens
tctcatgaacctttacctct

SEQ ID NO: 26 - DNA, 20 bp, Homo sapiens
tattagcttgaactctgtgc

SEQ ID NO: 27 - DNA, 20 bp, Homo sapiens
cattaaggaagacagatatg

SEQ ID NO: 28 - DNA, 20 bp, Homo sapiens
caggagtgactttgttccat

SEQ ID NO: 29 - DNA, 20 bp, Homo sapiens
gctaccggttacctgaaggg

SEQ ID NO: 30 - DNA, 20 bp, Homo sapiens
tgccacaagaatgatcattc

SEQ ID NO: 31 - DNA, 20 bp, Homo sapiens
tcccatgtgactccccgcct

SEQ ID NO: 32 - DNA, 20 bp, Homo sapiens
tattgttgctttttgagagg

SEQ ID NO: 33 - DNA, 20 bp, Homo sapiens
gtgaatttctattcctgttt

SEQ ID NO: 34 - DNA, 20 bp, Homo sapiens
gcatactttttttaatggaa

SEQ ID NO: 35 - DNA, 20 bp, Homo sapiens
ctcaacacacagaaacaaat

SEQ ID NO: 36 - DNA, 20 bp, Homo sapiens
gaatagataataaggaaata

SEQ ID NO: 37 - DNA, 20 bp, Homo sapiens
tttcttttttcttctactat

SEQ ID NO: 38 - DNA, 20 bp, Homo sapiens
cctgtgtttcaaggggatct

SEQ ID NO: 39 - DNA, 20 bp, Homo sapiens
cccttgaaacacaggaattg

SEQ ID NO: 40 - DNA, 20 bp, Homo sapiens
taagcaatggatttcagtga

SEQ ID NO: 41 - DNA, 20 bp, Homo sapiens
caatggatttcagtgataaa

SEQ ID NO: 42 - DNA, 20 bp, Homo sapiens
tattttccaggtactccact

SEQ ID NO: 43 - DNA, 20 bp, Homo sapiens
tacaaacagggtaatgagac

SEQ ID NO: 44 - DNA, 20 bp, Homo sapiens
tcctgtgtattcatagtata

SEQ ID NO: 45 - DNA, 20 bp, Homo sapiens
gataatcctattgtttgtta

SEQ ID NO: 46 - DNA, 20 bp, Homo sapiens
cactctctccctagtaactc

SEQ ID NO: 47 - DNA, 20 bp, Homo sapiens
gaaacttcactggtctcctt

SEQ ID NO: 48 - DNA, 20 bp, Homo sapiens
ctggcggattacctgagatc

SEQ ID NO: 49 - DNA, 20 bp, Homo sapiens
ctataattatgtactttact

SEQ ID NO: 50 - DNA, 20 bp, Homo sapiens
catgttggccgggctggtct

SEQ ID NO: 51 - RNA, 20 nt, Artificial Sequence
  Guide sequence for CRISPR/Cas12f1 system
uagaaaauuaugccuauuua

SEQ ID NO: 52 - RNA, 20 nt, Artificial Sequence
  Guide sequence for CRISPR/Cas12f1 system
gcaaaagcagcagaaagcaa

SEQ ID NO: 53 - RNA, 20 nt, Artificial Sequence
  Guide sequence for CRISPR/Cas12f1 system
guaggaauccugaaagcuac

SEQ ID NO: 54 - RNA, 20 nt, Artificial Sequence
  Guide sequence for CRISPR/Cas12f1 system
cugaggacagaacaagcucc

SEQ ID NO: 55 - RNA, 20 nt, Artificial Sequence
  Guide sequence for CRISPR/Cas12f1 system
ggaaaaugauaacguuaaac

SEQ ID NO: 56 - RNA, 20 nt, Artificial Sequence
  Guide sequence for CRISPR/Cas12f1 system
cugaaugugucucuaugagc

SEQ ID NO: 57 - RNA, 20 nt, Artificial Sequence
  Guide sequence for CRISPR/Cas12f1 system
uucaucuuccucaucagaaa

SEQ ID NO: 58 - RNA, 20 nt, Artificial Sequence
  Guide sequence for CRISPR/Cas12f1 system
gauuauucuuaucuaagauc

SEQ ID NO: 59 - RNA, 20 nt, Artificial Sequence
  Guide sequence for CRISPR/Cas12f1 system
ucaauauuauuuagaaguaa

SEQ ID NO: 60 - RNA, 20 nt, Artificial Sequence
  Guide sequence for CRISPR/Cas12f1 system
cagagugcauccauggucca

SEQ ID NO: 61 - RNA, 20 nt, Artificial Sequence
  Guide sequence for CRISPR/Cas12f1 system
uuccuuggaauggauguagc

SEQ ID NO: 62 - RNA, 20 nt, Artificial Sequence
  Guide sequence for CRISPR/Cas12f1 system
accagacaucuaagagaaaa

SEQ ID NO: 63 - RNA, 20 nt, Artificial Sequence
  Guide sequence for CRISPR/Cas12f1 system
uucuacauuaaaauucuaca

SEQ ID NO: 64 - RNA, 20 nt, Artificial Sequence
  Guide sequence for CRISPR/Cas12f1 system
uucuggguacagggguaaga

SEQ ID NO: 65 - RNA, 20 nt, Artificial Sequence
  Guide sequence for CRISPR/Cas12f1 system
auucagcuaaaucaugcaag

SEQ ID NO: 66 - RNA, 20 nt, Artificial Sequence
  Guide sequence for CRISPR/Cas12f1 system
uaguggcugaaugacuucug

SEQ ID NO: 67 - RNA, 20 nt, Artificial Sequence
  Guide sequence for CRISPR/Cas12f1 system
uucccacauaacacacacac

SEQ ID NO: 68 - RNA, 20 nt, Artificial Sequence
  Guide sequence for CRISPR/Cas12f1 system
uaacuaaacugaaugaauaa

SEQ ID NO: 69 - RNA, 20 nt, Artificial Sequence
  Guide sequence for CRISPR/Cas12f1 system
auuaaaaucugccaaauccc

SEQ ID NO: 70 - RNA, 20 nt, Artificial Sequence
  Guide sequence for CRISPR/Cas12f1 system
uguuuggggggauuuggcag

SEQ ID NO: 71 - RNA, 20 nt, Artificial Sequence
  Guide sequence for CRISPR/Cas12f1 system
ugugauucuugaaccuugug

SEQ ID NO: 72 - RNA, 20 nt, Artificial Sequence
  Guide sequence for CRISPR/Cas12f1 system
uucuacaauaaaaaaugaug

SEQ ID NO: 73 - RNA, 21 nt, Artificial Sequence
  Guide sequence for CRISPR/Cas12f1 system
cuggcucauagagacacauuc

SEQ ID NO: 74 - RNA, 21 nt, Artificial Sequence
  Guide sequence for CRISPR/Cas12f1 system
agcuaauagaauuagaucuua

SEQ ID NO: 75 - RNA, 20 nt, Artificial Sequence
  Guide sequence for CRISPR/Cas12f1 system
agagguaaagguucaugaga

SEQ ID NO: 76 - RNA, 20 nt, Artificial Sequence
  Guide sequence for CRISPR/Cas12f1 system
gcacagaguucaagcuaaua

SEQ ID NO: 77 - RNA, 20 nt, Artificial Sequence
  Guide sequence for CRISPR/Cas12f1 system
cauaucugucuuccuuaaug

SEQ ID NO: 78 - RNA, 20 nt, Artificial Sequence
  Guide sequence for CRISPR/Cas12f1 system
auggaacaaagucacuccug

SEQ ID NO: 79 - RNA, 20 nt, Artificial Sequence
  Guide sequence for CRISPR/Cas12f1 system
cccuucagguaaccgguagc

SEQ ID NO: 80 - RNA, 20 nt, Artificial Sequence
  Guide sequence for CRISPR/Cas12f1 system
gaaugaucauucuuguggca

SEQ ID NO: 81 - RNA, 20 nt, Artificial Sequence
  Guide sequence for CRISPR/Cas12f1 system
aggcggggagucacauggga

SEQ ID NO: 82 - RNA, 20 nt, Artificial Sequence
  Guide sequence for CRISPR/Cas12f1 system
ccucucaaaaagcaacaaua

SEQ ID NO: 83 - RNA, 20 nt, Artificial Sequence
  Guide sequence for CRISPR/Cas12f1 system
aaacaggaauagaaauucac

SEQ ID NO: 84 - RNA, 20 nt, Artificial Sequence
  Guide sequence for CRISPR/Cas12f1 system
uuccauuaaaaaaaguaugc

SEQ ID NO: 85 - RNA, 20 nt, Artificial Sequence
  Guide sequence for CRISPR/Cas12f1 system
auuuguuucuguguguugag

SEQ ID NO: 86 - RNA, 20 nt, Artificial Sequence
  Guide sequence for CRISPR/Cas12f1 system
uauuuccuuauuaucuauuc

SEQ ID NO: 87 - RNA, 20 nt, Artificial Sequence
  Guide sequence for CRISPR/Cas12f1 system
auaguagaagaaaaaagaaa

SEQ ID NO: 88 - RNA, 20 nt, Artificial Sequence
  Guide sequence for CRISPR/Cas12f1 system
agauccccuugaaacacagg

SEQ ID NO: 89 - RNA, 20 nt, Artificial Sequence
  Guide sequence for CRISPR/Cas12f1 system
caauuccuguguuucaaggg

SEQ ID NO: 90 - RNA, 20 nt, Artificial Sequence
  Guide sequence for CRISPR/Cas12f1 system
ucacugaaauccauugcuua

SEQ ID NO: 91 - RNA, 20 nt, Artificial Sequence
  Guide sequence for CRISPR/Cas12f1 system
uuuaucacugaaauccauug

SEQ ID NO: 92 - RNA, 20 nt, Artificial Sequence
  Guide sequence for CRISPR/Cas12f1 system
aguggaguaccuggaaaaua

SEQ ID NO: 93 - RNA, 20 nt, Artificial Sequence
  Guide sequence for CRISPR/Cas12f1 system
gucucauuacccuguuugua

SEQ ID NO: 94 - RNA, 20 nt, Artificial Sequence
  Guide sequence for CRISPR/Cas12f1 system
uauacuaugaauacacagga

SEQ ID NO: 95 - RNA, 20 nt, Artificial Sequence
  Guide sequence for CRISPR/Cas12f1 system
uaacaaacaauaggauuauc

SEQ ID NO: 96 - RNA, 20 nt, Artificial Sequence
  Guide sequence for CRISPR/Cas12f1 system
gaguuacuagggagagagug

SEQ ID NO: 97 - RNA, 20 nt, Artificial Sequence
  Guide sequence for CRISPR/Cas12f1 system
aaggagaccagugaaguuuc

SEQ ID NO: 98 - RNA, 20 nt, Artificial Sequence
  Guide sequence for CRISPR/Cas12f1 system
gaucucagguaauccgccag

SEQ ID NO: 99 - RNA, 20 nt, Artificial Sequence
  Guide sequence for CRISPR/Cas12f1 system
aguaaaguacauaauuauag

SEQ ID NO: 100 - RNA, 20 nt, Artificial Sequence
  Guide sequence for CRISPR/Cas12f1 system
agaccagcccggccaacaug

SEQ ID NO: 101 - RNA, 37 nt, Artificial Sequence
  Scaffold sequence of wildtype crRNA for CRISPR/Cas12f1 system
guugcagaacccgaauagacgaaugaaggaaugcaac

SEQ ID NO: 102 - RNA, 35 nt, Artificial Sequence
  Scaffold sequence of engineered crRNA for CRISPR/Cas12f1 system
ugcagaacccgaauagacgaaugaaggaaugcaac

SEQ ID NO: 103 - RNA, 33 nt, Artificial Sequence
  Scaffold sequence of engineered crRNA for CRISPR/Cas12f1 system
cagaacccgaauagacgaaugaaggaaugcaac

SEQ ID NO: 104 - RNA, 25 nt, Artificial Sequence
  Scaffold sequence of engineered crRNA for CRISPR/Cas12f1 system
gaauagacgaaugaaggaaugcaac

SEQ ID NO: 105 - RNA, 19 nt, Artificial Sequence
  Scaffold sequence of engineered crRNA for CRISPR/Cas12f1 system
acgaaugaaggaaugcaac

SEQ ID NO: 106 - RNA, 10 nt, Artificial Sequence
  Scaffold sequence of engineered crRNA for CRISPR/Cas12f1 system
ggaaugcaac

SEQ ID NO: 107 - RNA, 37 nt, Artificial Sequence
  Scaffold sequence of engineered crRNA for CRISPR/Cas12f1 system
guugcagaacccgaauagagcaaugaaggaaugcaac

SEQ ID NO: 108 - RNA, 25 nt, Artificial Sequence
  Scaffold sequence of engineered crRNA for CRISPR/Cas12f1 system
gaauagagcaaugaaggaaugcaac

SEQ ID NO: 109 - RNA, 19 nt, Artificial Sequence
  Scaffold sequence of engineered crRNA for CRISPR/Cas12f1 system
agcaaugaaggaaugcaac

SEQ ID NO: 110 - RNA, 161 nt, Artificial Sequence
  Scaffold sequence of wildtype tracrRNA for CRISPR/Cas12f1 system
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaa

SEQ ID NO: 111 - RNA, 141 nt, Artificial Sequence
  Scaffold sequence of engineered tracrRNA for CRISPR/Cas12f1 system
accgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaa

SEQ ID NO: 112 - RNA, 140 nt, Artificial Sequence
  Scaffold sequence of engineered tracrRNA for CRISPR/Cas12f1 system
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuu

SEQ ID NO: 113 - RNA, 120 nt, Artificial Sequence
  Scaffold sequence of engineered tracrRNA for CRISPR/Cas12f1 system
accgcuucaccaaaagcugucccuuaggggauuagaacuugagugaaggugggcugcuugcaucagccuaaugucgagaagugcuuucuucggaaaguaacccucgaaacaaauucauuu

SEQ ID NO: 114 - RNA, 113 nt, Artificial Sequence
  Scaffold sequence of engineered tracrRNA for CRISPR/Cas12f1 system
accgcuucaccaaaagcugucccuuaggggauuagaacuugagugaaggugggcugcuugcaucagccuaaugucgagaagugcuuucuucggaaaguaacccucgaaacaaa

SEQ ID NO: 115 - RNA, 95 nt, Artificial Sequence
  Scaffold sequence of engineered tracrRNA for CRISPR/Cas12f1 system
accgcuucaccuuaggagugaaggugggcugcuugcaucagccuaaugucgagaagugcuuucuucggaaaguaacccucgaaacaaauucauuu

SEQ ID NO: 116 - RNA, 86 nt, Artificial Sequence
  Scaffold sequence of engineered tracrRNA for CRISPR/Cas12f1 system
accgcuucacuuagagugaaggugggcugcuugcaucagccuaaugucgagaagugcuuucuucggaaaguaacccucgaaacaaa

SEQ ID NO: 117 - RNA, 161 nt, Artificial Sequence
  Scaffold sequence of engineered tracrRNA for CRISPR/Cas12f1 system
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucagugcuccucuccaauucugcacaa

SEQ ID NO: 118 - RNA, 141 nt, Artificial Sequence
  Scaffold sequence of engineered tracrRNA for CRISPR/Cas12f1 system
accgcuucaccaaaagcugucccuuaggggauuagaacuugagugaaggugggcugcuugcaucagccuaaugucgagaagugcuuucuucggaaaguaacccucgaaacaaauucagugcuccucuccaauucugcacaa

SEQ ID NO: 119 - RNA, 120 nt, Artificial Sequence
  Scaffold sequence of engineered tracrRNA for CRISPR/Cas12f1 system
accgcuucaccaauuaguugagugaaggugggcugcuugcaucagccuaaugucgagaagugcuuucuucggaaaguaacccucgaaacaaauucagugcuccucuccaauucugcacaa

SEQ ID NO: 120 - RNA, 107 nt, Artificial Sequence
  Scaffold sequence of engineered tracrRNA for CRISPR/Cas12f1 system
accgcuucaccaauuaguugagugaaggugggcugcuugcaucagccuaaugucgagaagugcuuucuucggaaaguaacccucgaaacaaauucagugcuccucuc

SEQ ID NO: 121 - RNA, 101 nt, Artificial Sequence
  Scaffold sequence of engineered tracrRNA for CRISPR/Cas12f1 system
accgcuucaccaauuaguugagugaaggugggcugcuugcaucagccuaaugucgagaagugcuuucuucggaaaguaacccucgaaacaaauucagugcu

SEQ ID NO: 122 - RNA, 202 nt, Artificial Sequence
  Scaffold region for CRISPR/Cas12f1 system
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaac

SEQ ID NO: 123 - RNA, 202 nt, Artificial Sequence
  Scaffold region for CRISPR/Cas12f1 system
cuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucagugcuccucuccaauucugcacaagaaaguugcagaacccgaauagacgaaugaaggaaugcaac

SEQ ID NO: 124 - RNA, 100 nt, Artificial Sequence
  Scaffold region for CRISPR/Cas12f1 system
accgcuucacuuagagugaaggugggcugcuugcaucagccuaaugucgagaagugcuuucuucggaaaguaacccucgaaacaaagaaaggaaugcaac

SEQ ID NO: 125 - RNA, 127 nt, Artificial Sequence
  Scaffold region for CRISPR/Cas12f1 system
accgcuucaccaaaagcugucccuuaggggauuagaacuugagugaaggugggcugcuugcaucagccuaaugucgagaagugcuuucuucggaaaguaacccucgaaacaaagaaaggaaugcaac

SEQ ID NO: 126 - RNA, 182 nt, Artificial Sequence
  Scaffold region for CRISPR/Cas12f1 system
accgcuucaccaaaagcugucccuuaggggauuagaacuugagugaaggugggcugcuugcaucagccuaaugucgagaagugcuuucuucggaaaguaacccucgaaacaaauucagugcuccucuccaauucugcacaagaaaguugcagaacccgaauagagcaaugaaggaaugcaac

SEQ ID NO: 127 - RNA, 10 nt, Artificial Sequence
  U-rich tail sequence
uuucuauuuu

SEQ ID NO: 128 - RNA, 10 nt, Artificial Sequence
  U-rich tail sequence
uuauguuuuu

SEQ ID NO: 129 - protein, 529 aa, Unknown
  Cas12f1 protein derived from Uncultured archaeon
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 130 - protein, 7 aa, Artificial Sequence
  Nuclear Localization Sequence
PKKKRKV

SEQ ID NO: 131 - protein, 16 aa, Artificial Sequence
  Nuclear Localization Sequence
KRPAATKKAGQAKKKK

SEQ ID NO: 132 - protein, 9 aa, Artificial Sequence
  Nuclear Localization Sequence
PAAKRVKLD

SEQ ID NO: 133 - protein, 11 aa, Artificial Sequence
  Nuclear Localization Sequence
RQRRNELKRSP

SEQ ID NO: 134 - protein, 38 aa, Artificial Sequence
  Nuclear Localization Sequence
NQSSNFGPMKGGNFGGRSSGPYGGGGQYFAKPRNQGGY

SEQ ID NO: 135 - protein, 42 aa, Artificial Sequence
  Nuclear Localization Sequence
RMRIZFKNKGKDTAELRRRRVEVSVELRKAKKDEQILKRRNV

SEQ ID NO: 136 - protein, 8 aa, Artificial Sequence
  Nuclear Localization Sequence
VSRKRPRP

SEQ ID NO: 137 - protein, 8 aa, Artificial Sequence
  Nuclear Localization Sequence
PPKKARED

SEQ ID NO: 138 - protein, 8 aa, Artificial Sequence
  Nuclear Localization Sequence
PQPKKKPL

SEQ ID NO: 139 - protein, 12 aa, Artificial Sequence
  Nuclear Localization Sequence
SALIKKKKKMAP

SEQ ID NO: 140 - protein, 5 aa, Artificial Sequence
  Nuclear Localization Sequence
DRLRR

SEQ ID NO: 141 - protein, 7 aa, Artificial Sequence
  Nuclear Localization Sequence
PKQKKRK

SEQ ID NO: 142 - protein, 10 aa, Artificial Sequence
  Nuclear Localization Sequence
RKLKKKIKKL

SEQ ID NO: 143 - protein, 10 aa, Artificial Sequence
  Nuclear Localization Sequence
REKKKFLKRR

SEQ ID NO: 144 - protein, 20 aa, Artificial Sequence
  Nuclear Localization Sequence
KRKGDEVDGVDEVAKKKSKK

SEQ ID NO: 145 - protein, 17 aa, Artificial Sequence
  Nuclear Localization Sequence
RKCLQAGMNLEARKTKK

SEQ ID NO: 146 - DNA, 1580 bp, Artificial Sequence
  Human codon optimized nucleic acid encoding Cas12f1 protein
atggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaagga

SEQ ID NO: 147 - DNA, 26 bp, Artificial Sequence
  Primer sequence
tgaactgactgctaagtacagggaca

SEQ ID NO: 148 - DNA, 20 bp, Artificial Sequence
  Primer sequence
aagcgttaagccgagatcgt

SEQ ID NO: 149 - DNA, 26 bp, Artificial Sequence
  Primer sequence
tgtggcagccattattcctgtctcta

SEQ ID NO: 150 - DNA, 26 bp, Artificial Sequence
  Primer sequence
tctgccttgactagctggttaggtaa

SEQ ID NO: 151 - DNA, 56 bp, Artificial Sequence
  Primer sequence
cactctttccctacacgacgctcttccgatctgaactgactgctaagtacagggac

SEQ ID NO: 152 - DNA, 61 bp, Artificial Sequence
  Primer sequence
gtgactggagttcagacgtgtgctcttccgatctgcaggcaaactttaattaaaatctgcc

SEQ ID NO: 153 - DNA, 54 bp, Artificial Sequence
  Primer sequence
cactctttccctacacgacgctcttccgatctggaacacctggaggtaagtttg

SEQ ID NO: 154 - DNA, 57 bp, Artificial Sequence
  Primer sequence
gtgactggagttcagacgtgtgctcttccgatctcattcagaagtcattcagccact

SEQ ID NO: 155 - DNA, 54 bp, Artificial Sequence
  Primer sequence
cactctttccctacacgacgctcttccgatctttgttcccacataacacacaca

SEQ ID NO: 156 - DNA, 60 bp, Artificial Sequence
  Primer sequence
gtgactggagttcagacgtgtgctcttccgatctttctgggtacaggggtaagagaaagg

SEQ ID NO: 157 - DNA, 53 bp, Artificial Sequence
  Primer sequence
cactctttccctacacgacgctcttccgatcttgtaatcaaaggagggaagca

SEQ ID NO: 158 - DNA, 58 bp, Artificial Sequence
  Primer sequence
gtgactggagttcagacgtgtgctcttccgatctgatgagaatttacagagtgcatcc

SEQ ID NO: 159 - DNA, 58 bp, Artificial Sequence
  Primer sequence
cactctttccctacacgacgctcttccgatctacccagaacaaactattctcccatgg

SEQ ID NO: 160 - DNA, 60 bp, Artificial Sequence
  Primer sequence
gtgactggagttcagacgtgtgctcttccgatctgggtctggtccatattcacagattgt

SEQ ID NO: 161 - DNA, 53 bp, Artificial Sequence
  Primer sequence
cactctttccctacacgacgctcttccgatcttggaccagaccctttgtagtt

SEQ ID NO: 162 - DNA, 56 bp, Artificial Sequence
  Primer sequence
gtgactggagttcagacgtgtgctcttccgatcttgtctctatgagccagcaaaag

SEQ ID NO: 163 - DNA, 60 bp, Artificial Sequence
  Primer sequence
cactctttccctacacgacgctcttccgatcttcatcttcctcatcagaaatagaggctt

SEQ ID NO: 164 - DNA, 60 bp, Artificial Sequence
  Primer sequence
gtgactggagttcagacgtgtgctcttccgatctgcaaaagcagcagaaagcaaactgat

SEQ ID NO: 165 - DNA, 58 bp, Artificial Sequence
  Primer sequence
cactctttccctacacgacgctcttccgatcttgctggctcatagagacacattcagt

SEQ ID NO: 166 - DNA, 58 bp, Artificial Sequence
  Primer sequence
gtgactggagttcagacgtgtgctcttccgatcttgctggagaggataggacagagga

SEQ ID NO: 167 - DNA, 53 bp, Artificial Sequence
  Primer sequence
cactctttccctacacgacgctcttccgatctttccattctccatgtcctctg

SEQ ID NO: 168 - DNA, 60 bp, Artificial Sequence
  Primer sequence
gtgactggagttcagacgtgtgctcttccgatctgggtgacagaatgaaactccatctca

SEQ ID NO: 169 - DNA, 52 bp, Artificial Sequence
  Primer sequence
cactctttccctacacgacgctcttccgatctgtggcagccattattcctgt

SEQ ID NO: 170 - DNA, 57 bp, Artificial Sequence
  Primer sequence
gtgactggagttcagacgtgtgctcttccgatctgaacactgtggtcagaaaactca

SEQ ID NO: 171 - DNA, 58 bp, Artificial Sequence
  Primer sequence
cactctttccctacacgacgctcttccgatctatgtatgttcttttgtacgtgctctt

SEQ ID NO: 172 - DNA, 64 bp, Artificial Sequence
  Primer sequence
gtgactggagttcagacgtgtgctcttccgatctaagacagatatgtaaacacatataatttga

SEQ ID NO: 173 - DNA, 58 bp, Artificial Sequence
  Primer sequence
cactctttccctacacgacgctcttccgatctgtcactcctgtctacactagttctgc

SEQ ID NO: 174 - DNA, 60 bp, Artificial Sequence
  Primer sequence
gtgactggagttcagacgtgtgctcttccgatctaccatatttgacaccacatgcactgt

SEQ ID NO: 175 - DNA, 59 bp, Artificial Sequence
  Primer sequence
cactctttccctacacgacgctcttccgatctttccttaatggaatataagtcttttga

SEQ ID NO: 176 - DNA, 62 bp, Artificial Sequence
  Primer sequence
gtgactggagttcagacgtgtgctcttccgatctccttattatctattccattcttcacaca

SEQ ID NO: 177 - DNA, 55 bp, Artificial Sequence
  Primer sequence
cactctttccctacacgacgctcttccgatctggttcatgagactagaggtcacg

SEQ ID NO: 178 - DNA, 54 bp, Artificial Sequence
  Primer sequence
gtgactggagttcagacgtgtgctcttccgatctctgggcaacacagtgagact

SEQ ID NO: 179 - DNA, 52 bp, Artificial Sequence
  Primer sequence
cactctttccctacacgacgctcttccgatctggttcaagcgattcttctgc

SEQ ID NO: 180 - DNA, 55 bp, Artificial Sequence
  Primer sequence
gtgactggagttcagacgtgtgctcttccgatctcctgaaacttcactggtctcc

SEQ ID NO: 181 - DNA, 52 bp, Artificial Sequence
  Primer sequence
cactctttccctacacgacgctcttccgatctagcctcaacttcccaaagtg

SEQ ID NO: 182 - DNA, 54 bp, Artificial Sequence
  Primer sequence
gtgactggagttcagacgtgtgctcttccgatcttctttttctcccacctaccc

SEQ ID NO: 183 - DNA, 60 bp, Artificial Sequence
  Primer sequence
cactctttccctacacgacgctcttccgatcttttagagttactagggagagagtgatga

SEQ ID NO: 184 - DNA, 54 bp, Artificial Sequence
  Primer sequence
gtgactggagttcagacgtgtgctcttccgatctacacaggagggaaaacagga

SEQ ID NO: 185 - DNA, 54 bp, Artificial Sequence
  Primer sequence
cactctttccctacacgacgctcttccgatcttcctgtgaaaaaggatgaaagg

SEQ ID NO: 186 - DNA, 61 bp, Artificial Sequence
  Primer sequence
gtgactggagttcagacgtgtgctcttccgatcttgaagaataatacaaacagggtaatga

SEQ ID NO: 187 - DNA, 52 bp, Artificial Sequence
  Primer sequence
cactctttccctacacgacgctcttccgatctctcctgttttccctcctgtg

SEQ ID NO: 188 - DNA, 55 bp, Artificial Sequence
  Primer sequence
gtgactggagttcagacgtgtgctcttccgatcttttgagtggagtacctggaaa

SEQ ID NO: 189 - DNA, 57 bp, Artificial Sequence
  Primer sequence
cactctttccctacacgacgctcttccgatcttgtcctctttttagtctcattaccc

SEQ ID NO: 190 - DNA, 55 bp, Artificial Sequence
  Primer sequence
gtgactggagttcagacgtgtgctcttccgatctggaaaaaggctggcgatataa

SEQ ID NO: 191 - DNA, 52 bp, Artificial Sequence
  Primer sequence
cactctttccctacacgacgctcttccgatctatatcgccagcctttttcct

SEQ ID NO: 192 - DNA, 56 bp, Artificial Sequence
  Primer sequence
gtgactggagttcagacgtgtgctcttccgatctgccttgactagctggttaggta

SEQ ID NO: 193 - DNA, 22 bp, Artificial Sequence
  Primer sequence
cttggctcactgcaagctccac

SEQ ID NO: 194 - DNA, 30 bp, Artificial Sequence
  Primer sequence
acagggtaggattcatgtttagaatgatca

SEQ ID NO: 195 - DNA, 20 bp, Homo sapiens
tagaaaattatgcctattta

SEQ ID NO: 196 - DNA, 20 bp, Homo sapiens
gcaaaagcagcagaaagcaa

SEQ ID NO: 197 - DNA, 20 bp, Homo sapiens
gtaggaatcctgaaagctac

SEQ ID NO: 198 - DNA, 20 bp, Homo sapiens
ctgaggacagaacaagctcc

SEQ ID NO: 199 - DNA, 20 bp, Homo sapiens
ggaaaatgataacgttaaac

SEQ ID NO: 200 - DNA, 20 bp, Homo sapiens
ctgaatgtgtctctatgagc

SEQ ID NO: 201 - DNA, 20 bp, Homo sapiens
ttcatcttcctcatcagaaa

SEQ ID NO: 202 - DNA, 20 bp, Homo sapiens
gattattcttatctaagatc

SEQ ID NO: 203 - DNA, 20 bp, Homo sapiens
tcaatattatttagaagtaa

SEQ ID NO: 204 - DNA, 20 bp, Homo sapiens
cagagtgcatccatggtcca

SEQ ID NO: 205 - DNA, 20 bp, Homo sapiens
ttccttggaatggatgtagc

SEQ ID NO: 206 - DNA, 20 bp, Homo sapiens
accagacatctaagagaaaa

SEQ ID NO: 207 - DNA, 20 bp, Homo sapiens
ttctacattaaaattctaca

SEQ ID NO: 208 - DNA, 20 bp, Homo sapiens
ttctgggtacaggggtaaga

SEQ ID NO: 209 - DNA, 20 bp, Homo sapiens
attcagctaaatcatgcaag

SEQ ID NO: 210 - DNA, 20 bp, Homo sapiens
tagtggctgaatgacttctg

SEQ ID NO: 211 - DNA, 20 bp, Homo sapiens
ttcccacataacacacacac

SEQ ID NO: 212 - DNA, 20 bp, Homo sapiens
taactaaactgaatgaataa

SEQ ID NO: 213 - DNA, 20 bp, Homo sapiens
attaaaatctgccaaatccc

SEQ ID NO: 214 - DNA, 20 bp, Homo sapiens
tgtttggggggatttggcag

SEQ ID NO: 215 - DNA, 20 bp, Homo sapiens
tgtgattcttgaaccttgtg

SEQ ID NO: 216 - DNA, 20 bp, Homo sapiens
ttctacaataaaaaatgatg

SEQ ID NO: 217 - DNA, 21 bp, Homo sapiens
ctggctcatagagacacattc

SEQ ID NO: 218 - DNA, 21 bp, Homo sapiens
agctaatagaattagatctta

SEQ ID NO: 219 - DNA, 20 bp, Homo sapiens
agaggtaaaggttcatgaga

SEQ ID NO: 220 - DNA, 20 bp, Homo sapiens
gcacagagttcaagctaata

SEQ ID NO: 221 - DNA, 20 bp, Homo sapiens
catatctgtcttccttaatg

SEQ ID NO: 222 - DNA, 20 bp, Homo sapiens
atggaacaaagtcactcctg

SEQ ID NO: 223 - DNA, 20 bp, Homo sapiens
cccttcaggtaaccggtagc

SEQ ID NO: 224 - DNA, 20 bp, Homo sapiens
gaatgatcattcttgtggca

SEQ ID NO: 225 - DNA, 20 bp, Homo sapiens
aggcggggagtcacatggga

SEQ ID NO: 226 - DNA, 20 bp, Homo sapiens
cctctcaaaaagcaacaata

SEQ ID NO: 227 - DNA, 20 bp, Homo sapiens
aaacaggaatagaaattcac

SEQ ID NO: 228 - DNA, 20 bp, Homo sapiens
ttccattaaaaaaagtatgc

SEQ ID NO: 229 - DNA, 20 bp, Homo sapiens
atttgtttctgtgtgttgag

SEQ ID NO: 230 - DNA, 20 bp, Homo sapiens
tatttccttattatctattc

SEQ ID NO: 231 - DNA, 20 bp, Homo sapiens
atagtagaagaaaaaagaaa

SEQ ID NO: 232 - DNA, 20 bp, Homo sapiens
agatccccttgaaacacagg

SEQ ID NO: 233 - DNA, 20 bp, Homo sapiens
caattcctgtgtttcaaggg

SEQ ID NO: 234 - DNA, 20 bp, Homo sapiens
tcactgaaatccattgctta

SEQ ID NO: 235 - DNA, 20 bp, Homo sapiens
tttatcactgaaatccattg

SEQ ID NO: 236 - DNA, 20 bp, Homo sapiens
agtggagtacctggaaaata

SEQ ID NO: 237 - DNA, 20 bp, Homo sapiens
gtctcattaccctgtttgta

SEQ ID NO: 238 - DNA, 20 bp, Homo sapiens
tatactatgaatacacagga

SEQ ID NO: 239 - DNA, 20 bp, Homo sapiens
taacaaacaataggattatc

SEQ ID NO: 240 - DNA, 20 bp, Homo sapiens
gagttactagggagagagtg

SEQ ID NO: 241 - DNA, 20 bp, Homo sapiens
aaggagaccagtgaagtttc

SEQ ID NO: 242 - DNA, 20 bp, Homo sapiens
gatctcaggtaatccgccag

SEQ ID NO: 243 - DNA, 20 bp, Homo sapiens
agtaaagtacataattatag

SEQ ID NO: 244 - DNA, 20 bp, Homo sapiens
agaccagcccggccaacatg

SEQ ID NO: 245 - RNA, 19 nt, Artificial Sequence
  Guide sequence for CRISPR/Cas12f1 system
cagagugcauccauggucc

SEQ ID NO: 246 - RNA, 21 nt, Artificial Sequence
  Guide sequence for CRISPR/Cas12f1 system
cagagugcauccaugguccag

SEQ ID NO: 247 - RNA, 22 nt, Artificial Sequence
  Guide sequence for CRISPR/Cas12f1 system
cagagugcauccaugguccagg

SEQ ID NO: 248 - RNA, 23 nt, Artificial Sequence
  Guide sequence for CRISPR/Cas12f1 system
cagagugcauccaugguccagga

SEQ ID NO: 249 - RNA, 24 nt, Artificial Sequence
  Guide sequence for CRISPR/Cas12f1 system
cagagugcauccaugguccaggaa

SEQ ID NO: 250 - RNA, 25 nt, Artificial Sequence
  Guide sequence for CRISPR/Cas12f1 system
cagagugcauccaugguccaggaag

SEQ ID NO: 251 - RNA, 19 nt, Artificial Sequence
  Guide sequence for CRISPR/Cas12f1 system
uucuggguacagggguaag

SEQ ID NO: 252 - RNA, 21 nt, Artificial Sequence
  Guide sequence for CRISPR/Cas12f1 system
uucuggguacagggguaagag

SEQ ID NO: 253 - RNA, 22 nt, Artificial Sequence
  Guide sequence for CRISPR/Cas12f1 system
uucuggguacagggguaagaga

SEQ ID NO: 254 - RNA, 23 nt, Artificial Sequence
  Guide sequence for CRISPR/Cas12f1 system
uucuggguacagggguaagagaa

SEQ ID NO: 255 - RNA, 24 nt, Artificial Sequence
  Guide sequence for CRISPR/Cas12f1 system
uucuggguacagggguaagagaaa

SEQ ID NO: 256 - RNA, 25 nt, Artificial Sequence
  Guide sequence for CRISPR/Cas12f1 system
uucuggguacagggguaagagaaag

SEQ ID NO: 257 - RNA, 19 nt, Artificial Sequence
  Guide sequence for CRISPR/Cas12f1 system
cccuucagguaaccgguag

SEQ ID NO: 258 - RNA, 25 nt, Artificial Sequence
  Guide sequence for CRISPR/Cas12f1 system
cccuucagguaaccgguagcuuuug

SEQ ID NO: 259 - RNA, 19 nt, Artificial Sequence
  Guide sequence for CRISPR/Cas12f1 system
aggcggggagucacauggg

SEQ ID NO: 260 - RNA, 21 nt, Artificial Sequence
  Guide sequence for CRISPR/Cas12f1 system
aggcggggagucacaugggag

SEQ ID NO: 261 - RNA, 23 nt, Artificial Sequence
  Guide sequence for CRISPR/Cas12f1 system
aggcggggagucacaugggaguc

SEQ ID NO: 262 - RNA, 24 nt, Artificial Sequence
  Guide sequence for CRISPR/Cas12f1 system
aggcggggagucacaugggaguca

SEQ ID NO: 263 - RNA, 25 nt, Artificial Sequence
  Guide sequence for CRISPR/Cas12f1 system
aggcggggagucacaugggagucac

SEQ ID NO: 264 - RNA, 21 nt, Artificial Sequence
  Guide sequence for CRISPR/Cas12f1 system
aguggaguaccuggaaaauaa

SEQ ID NO: 265 - RNA, 22 nt, Artificial Sequence
  Guide sequence for CRISPR/Cas12f1 system
aguggaguaccuggaaaauaag

SEQ ID NO: 266 - RNA, 23 nt, Artificial Sequence
  Guide sequence for CRISPR/Cas12f1 system
aguggaguaccuggaaaauaagc

SEQ ID NO: 267 - RNA, 24 nt, Artificial Sequence
  Guide sequence for CRISPR/Cas12f1 system
aguggaguaccuggaaaauaagcc

SEQ ID NO: 268 - RNA, 25 nt, Artificial Sequence
  Guide sequence for CRISPR/Cas12f1 system
aguggaguaccuggaaaauaagcca

SEQ ID NO: 269 - RNA, 21 nt, Artificial Sequence
  Guide sequence for CRISPR/Cas12f1 system
gaguuacuagggagagaguga

SEQ ID NO: 270 - RNA, 23 nt, Artificial Sequence
  Guide sequence for CRISPR/Cas12f1 system
gaguuacuagggagagagugaug

SEQ ID NO: 271 - RNA, 24 nt, Artificial Sequence
  Guide sequence for CRISPR/Cas12f1 system
gaguuacuagggagagagugauga

SEQ ID NO: 272 - RNA, 25 nt, Artificial Sequence
  Guide sequence for CRISPR/Cas12f1 system
gaguuacuagggagagagugaugaa

SEQ ID NO: 273 - DNA, 20 bp, Homo sapiens
atccctattggcagtgtctt

SEQ ID NO: 274 - DNA, 100 bp, Artificial Sequence
  Donor sequence for introducing a mutation using CRISPR/Cas9 system
gagccaccgcacctggccccagttgtaattgtgagtatctcatagatatccctattggcagtgtcttagttttattttttattatctttattgtggcagc

SEQ ID NO: 275 - DNA, 26 bp, Artificial Sequence
  Primer sequence
ccattttcaagattgcagctctccaa

SEQ ID NO: 276 - DNA, 26 bp, Artificial Sequence
  Primer sequence
tccaggtgttccaagttacttgttct

SEQ ID NO: 277 - DNA, 26 bp, Artificial Sequence
  Primer sequence
tgactgctaagtacagggacatcttg

SEQ ID NO: 278 - DNA, 26 bp, Artificial Sequence
  Primer sequence
atctgtaatcccagcactttaggagg

SEQ ID NO: 279 - DNA, 26 bp, Artificial Sequence
  Primer sequence
aggaaatggccattttcaagattgca

SEQ ID NO: 280 - DNA, 25 bp, Artificial Sequence
  Primer sequence
agactccacttgttcttttaaggag

SEQ ID NO: 281 - DNA, 26 bp, Artificial Sequence
  Primer sequence
tagagatggggtttcaccttgttagc

SEQ ID NO: 282 - DNA, 25 bp, Artificial Sequence
  Primer sequence
agactccacttgttcttttaaggag

SEQ ID NO: 283 - DNA, 32 bp, Homo sapiens
cctggccccagttgtaattgtgaatatctcat

SEQ ID NO: 284 - DNA, 32 bp, Artificial Sequence
  Mutation sequence
cctggccccagttgtaattgtgagtatctcat

SEQ ID NO: 285 - DNA, 32 bp, Artificial Sequence
  Mutation sequence
cctggccccaattgtaattgtgaatatctcat

SEQ ID NO: 286 - DNA, 69 bp, Homo sapiens
ctcctaaagtgctgggattacagatgtgagccaccgcacctggccccagttgtaattgtgaatatctca

SEQ ID NO: 287 - DNA, 69 bp, Artificial Sequence
  Mutation sequence
ctcctaaagtgctgggattacagatgtgagccaccgcacctggccccagttgtaattgtgagtatctca

SEQ ID NO: 288 - protein, 557 aa, Artificial Sequence
  TnpB
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 289 - protein, 555 aa, Artificial Sequence
  Cas12f1 + CasX 26aa
MEKRINKIRKKLSADNATKPVSRSGPMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 290 - protein, 557 aa, Artificial Sequence
  Cas12f1 + random 28aa
MAGGPGAGSAAPVSSTSSLPLAALNMRVMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 291 - protein, 555 aa, Artificial Sequence
  Cas12f1 + random 26aa
MAGGPGAGSAAPVSSTSSLPLAALNMMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 292 - RNA, 140 nt, Artificial Sequence; identical to SEQ ID NO: 112
  tracrRNA scaffold of Cas12f1, MF

SEQ ID NO: 293 - RNA, 32 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucugcacaa

SEQ ID NO: 294 - RNA, 31 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucugcaca

SEQ ID NO: 295 - RNA, 30 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucugcac

SEQ ID NO: 296 - RNA, 29 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucugca

SEQ ID NO: 297 - RNA, 28 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucugc

SEQ ID NO: 298 - RNA, 27 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucug

SEQ ID NO: 299 - RNA, 26 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauucu

SEQ ID NO: 300 - RNA, 25 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauuc

SEQ ID NO: 301 - RNA, 24 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaauu

SEQ ID NO: 302 - RNA, 23 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaau

SEQ ID NO: 303 - RNA, 22 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuccaa

SEQ ID NO: 304 - RNA, 21 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucucca

SEQ ID NO: 305 - RNA, 20 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucucc

SEQ ID NO: 306 - RNA, 19 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucuc

SEQ ID NO: 307 - RNA, 18 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccucu

SEQ ID NO: 308 - RNA, 17 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccuc

SEQ ID NO: 309 - RNA, 16 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnccu

SEQ ID NO: 310 - RNA, 15 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncncc

SEQ ID NO: 311 - RNA, 14 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncnc

SEQ ID NO: 312 - RNA, 13 nt, Artificial Sequence
  1st Fragment of engineered tracrRNA scaffold
  MISC_FEATURE (9)..(11): n is a, g, c, or u
  MISC_FEATURE (13): n is a, g, c, or u
caaauucannncn

SEQ ID NO: 313 - RNA, 39 nt, Artificial Sequence
  2nd Fragment of engineered tracrRNA scaffold
augucgagaagugcuuucuucggaaaguaacccucgaaa

SEQ ID NO: 314 - RNA, 19 nt, Artificial Sequence
  3rd Fragment of engineered tracrRNA scaffold
ggcugcuugcaucagccua

SEQ ID NO: 315 - RNA, 50 nt, Artificial Sequence
  4th Fragment of engineered tracrRNA scaffold
ccgcuucaccaaaagcugucccuuaggggauuagaacuugagugaaggug

SEQ ID NO: 316 - RNA, 48 nt, Artificial Sequence
  4th Fragment of engineered tracrRNA scaffold
ccgcuucaccaaaagcuguccuuagggauuagaacuugagugaaggug

SEQ ID NO: 317 - RNA, 46 nt, Artificial Sequence
  4th Fragment of engineered tracrRNA scaffold
ccgcuucaccaaaagcugucuuaggauuagaacuugagugaaggug

SEQ ID NO: 318 - RNA, 44 nt, Artificial Sequence
  4th Fragment of engineered tracrRNA scaffold
ccgcuucaccaaaagcuguuuagauuagaacuugagugaaggug

SEQ ID NO: 319 - RNA, 42 nt, Artificial Sequence
  4th Fragment of engineered tracrRNA scaffold
ccgcuucaccaaaagcuguuaguuagaacuugagugaaggug

SEQ ID NO: 320 - RNA, 41 nt, Artificial Sequence
  4th Fragment of engineered tracrRNA scaffold
ccgcuucaccaaaagcuguuaguagaacuugagugaaggug

SEQ ID NO: 321 - RNA, 39 nt, Artificial Sequence
  4th Fragment of engineered tracrRNA scaffold
ccgcuucaccaaaagcuuuagagaacuugagugaaggug

SEQ ID NO: 322 - RNA, 37 nt, Artificial Sequence
  4th Fragment of engineered tracrRNA scaffold
ccgcuucaccaaaagcuuaggaacuugagugaaggug

SEQ ID NO: 323 - RNA, 35 nt, Artificial Sequence
  4th Fragment of engineered tracrRNA scaffold
ccgcuucaccaaaaguuagaacuugagugaaggug

SEQ ID NO: 324 - RNA, 33 nt, Artificial Sequence
  4th Fragment of engineered tracrRNA scaffold
ccgcuucaccaaaauuagacuugagugaaggug

SEQ ID NO: 325 - RNA, 31 nt, Artificial Sequence
  4th Fragment of engineered tracrRNA scaffold
ccgcuucaccaaauuagcuugagugaaggug

SEQ ID NO: 326 - RNA, 29 nt, Artificial Sequence
  4th Fragment of engineered tracrRNA scaffold
ccgcuucaccaauuaguugagugaaggug

SEQ ID NO: 327 - RNA, 27 nt, Artificial Sequence
  4th Fragment of engineered tracrRNA scaffold
ccgcuucaccauuagugagugaaggug

SEQ ID NO: 328 - RNA, 25 nt, Artificial Sequence
  4th Fragment of engineered tracrRNA scaffold
ccgcuucaccuuaggagugaaggug

SEQ ID NO: 329 - RNA, 23 nt, Artificial Sequence
  4th Fragment of engineered tracrRNA scaffold
ccgcuucacuuagagugaaggug

SEQ ID NO: 330 - RNA, 22 nt, Artificial Sequence
  4th Fragment of engineered tracrRNA scaffold
ccgcuucacuuaggugaaggug

SEQ ID NO: 331 - RNA, 20 nt, Artificial Sequence
  4th Fragment of engineered tracrRNA scaffold
ccgcuucauuagugaaggug

SEQ ID NO: 332 - RNA, 18 nt, Artificial Sequence
  4th Fragment of engineered tracrRNA scaffold
ccgcuucuuaggaaggug

SEQ ID NO: 333 - RNA, 16 nt, Artificial Sequence
  4th Fragment of engineered tracrRNA scaffold
ccgcuuuuagaaggug

SEQ ID NO: 334 - RNA, 14 nt, Artificial Sequence
  4th Fragment of engineered tracrRNA scaffold
ccgcuuuagaggug

SEQ ID NO: 335 - RNA, 12 nt, Artificial Sequence
  4th Fragment of engineered tracrRNA scaffold
ccgcuuagggug

SEQ ID NO: 336 - RNA, 10 nt, Artificial Sequence
  4th Fragment of engineered tracrRNA scaffold
ccguuaggug

SEQ ID NO: 337 - RNA, 21 nt, Artificial Sequence
  5th Fragment of engineered tracrRNA scaffold
cuucacugauaaaguggagaa

SEQ ID NO: 338 - RNA, 10 nt, Artificial Sequence
  5th Fragment of engineered tracrRNA scaffold
aaguggagaa

SEQ ID NO: 339 - RNA, 11 nt, Artificial Sequence
  5th Fragment of engineered tracrRNA scaffold
aaaguggagaa

SEQ ID NO: 340 - RNA, 12 nt, Artificial Sequence
  5th Fragment of engineered tracrRNA scaffold
uaaaguggagaa

SEQ ID NO: 341 - RNA, 13 nt, Artificial Sequence
  5th Fragment of engineered tracrRNA scaffold
auaaaguggagaa

SEQ ID NO: 342 - RNA, 14 nt, Artificial Sequence
  5th Fragment of engineered tracrRNA scaffold
gauaaaguggagaa

SEQ ID NO: 343 - RNA, 15 nt, Artificial Sequence
  5th Fragment of engineered tracrRNA scaffold
ugauaaaguggagaa

SEQ ID NO: 344 - RNA, 16 nt, Artificial Sequence
  5th Fragment of engineered tracrRNA scaffold
cugauaaaguggagaa

SEQ ID NO: 345 - RNA, 17 nt, Artificial Sequence
  5th Fragment of engineered tracrRNA scaffold
acugauaaaguggagaa

SEQ ID NO: 346 - RNA, 18 nt, Artificial Sequence
  5th Fragment of engineered tracrRNA scaffold
cacugauaaaguggagaa

SEQ ID NO: 347 - RNA, 19 nt, Artificial Sequence
  5th Fragment of engineered tracrRNA scaffold
ucacugauaaaguggagaa

SEQ ID NO: 348 - RNA, 20 nt, Artificial Sequence
  5th Fragment of engineered tracrRNA scaffold
uucacugauaaaguggagaa

SEQ ID NO: 349 - RNA, 10 nt, Artificial Sequence
  1st region of engineered tracrRNA scaffold, MF
aaguggagaa

SEQ ID NO: 350 - RNA, 11 nt, Artificial Sequence
  1st region of engineered tracrRNA scaffold, MF
aaaguggagaa

SEQ ID NO: 351 - RNA, 12 nt, Artificial Sequence
  1st region of engineered tracrRNA scaffold, MF
uaaaguggagaa

SEQ ID NO: 352 - RNA, 13 nt, Artificial Sequence
  1st region of engineered tracrRNA scaffold, MF
auaaaguggagaa

SEQ ID NO: 353 - RNA, 14 nt, Artificial Sequence
  1st region of engineered tracrRNA scaffold, MF
gauaaaguggagaa

SEQ ID NO: 354 - RNA, 15 nt, Artificial Sequence
  1st region of engineered tracrRNA scaffold, MF
ugauaaaguggagaa

SEQ ID NO: 355 - RNA, 16 nt, Artificial Sequence
  1st region of engineered tracrRNA scaffold, MF
cugauaaaguggagaa

SEQ ID NO: 356 - RNA, 17 nt, Artificial Sequence
  1st region of engineered tracrRNA scaffold, MF
acugauaaaguggagaa

SEQ ID NO: 357 - RNA, 18 nt, Artificial Sequence
  1st region of engineered tracrRNA scaffold, MF
cacugauaaaguggagaa

SEQ ID NO: 358 - RNA, 19 nt, Artificial Sequence
  1st region of engineered tracrRNA scaffold, MF
ucacugauaaaguggagaa

SEQ ID NO: 359 - RNA, 20 nt, Artificial Sequence
  1st region of engineered tracrRNA scaffold, MF
uucacugauaaaguggagaa

SEQ ID NO: 360 - RNA, 21 nt, Artificial Sequence
  1st region of engineered tracrRNA scaffold, MF
cuucacugauaaaguggagaa

SEQ ID NO: 361 - RNA, 10 nt, Artificial Sequence
  2nd region of engineered tracrRNA scaffold, MF
ccuuaggugg

SEQ ID NO: 362 - RNA, 11 nt, Artificial Sequence
  2nd region of engineered tracrRNA scaffold, MF
ccguuaggugg

SEQ ID NO: 363 - RNA, 13 nt, Artificial Sequence
  2nd region of engineered tracrRNA scaffold, MF
ccgcuuagggugg

SEQ ID NO: 364 - RNA, 15 nt, Artificial Sequence
  2nd region of engineered tracrRNA scaffold, MF
ccgcuuuagaggugg

SEQ ID NO: 365 - RNA, 17 nt, Artificial Sequence
  2nd region of engineered tracrRNA scaffold, MF
ccgcuuuuagaaggugg

SEQ ID NO: 366 - RNA, 19 nt, Artificial Sequence
  2nd region of engineered tracrRNA scaffold, MF
ccgcuucuuaggaaggugg

SEQ ID NO: 367 - RNA, 21 nt, Artificial Sequence
  2nd region of engineered tracrRNA scaffold, MF
ccgcuucauuagugaaggugg

SEQ ID NO: 368 - RNA, 23 nt, Artificial Sequence
  2nd region of engineered tracrRNA scaffold, MF
ccgcuucacuuaggugaaggugg

SEQ ID NO: 369 - RNA, 24 nt, Artificial Sequence
  2nd region of engineered tracrRNA scaffold, MF
ccgcuucacuuagagugaaggugg

SEQ ID NO: 370 - RNA, 26 nt, Artificial Sequence
  2nd region of engineered tracrRNA scaffold, MF
ccgcuucaccuuaggagugaaggugg

SEQ ID NO: 371 - RNA, 28 nt, Artificial Sequence
  2nd region of engineered tracrRNA scaffold, MF
ccgcuucaccauuagugagugaaggugg

SEQ ID NO: 372 - RNA, 30 nt, Artificial Sequence
  2nd region of engineered tracrRNA scaffold, MF
ccgcuucaccaauuaguugagugaaggugg

SEQ ID NO: 373 - RNA, 32 nt, Artificial Sequence
  2nd region of engineered tracrRNA scaffold, MF
ccgcuucaccaaauuagcuugagugaaggugg

SEQ ID NO: 374 - RNA, 34 nt, Artificial Sequence
  2nd region of engineered tracrRNA scaffold, MF
ccgcuucaccaaaauuagacuugagugaaggugg

SEQ ID NO: 375 - RNA, 36 nt, Artificial Sequence
  2nd region of engineered tracrRNA scaffold, MF
ccgcuucaccaaaaguuagaacuugagugaaggugg

SEQ ID NO: 376 - RNA, 38 nt, Artificial Sequence
  2nd region of engineered tracrRNA scaffold, MF
ccgcuucaccaaaagcuuaggaacuugagugaaggugg

SEQ ID NO: 377 - RNA, 40 nt, Artificial Sequence
  2nd region of engineered tracrRNA scaffold, MF
ccgcuucaccaaaagcuuuagagaacuugagugaaggugg

SEQ ID NO: 378 - RNA, 42 nt, Artificial Sequence
  2nd region of engineered tracrRNA scaffold, MF
ccgcuucaccaaaagcuguuaguagaacuugagugaaggugg

SEQ ID NO: 379 - RNA, 43 nt, Artificial Sequence
  2nd region of engineered tracrRNA scaffold, MF
ccgcuucaccaaaagcuguuaguuagaacuugagugaaggugg

SEQ ID NO: 380 - RNA, 45 nt, Artificial Sequence
  2nd region of engineered tracrRNA scaffold, MF
ccgcuucaccaaaagcuguuuagauuagaacuugagugaaggugg

SEQ ID NO: 381 - RNA, 47 nt, Artificial Sequence
  2nd region of engineered tracrRNA scaffold, MF
ccgcuucaccaaaagcugucuuaggauuagaacuugagugaaggugg

SEQ ID NO: 382 - RNA, 51 nt, Artificial Sequence
  2nd region of engineered tracrRNA scaffold, MF
ccgcuucaccaaaagcugucccuuaggggauuagaacuugagugaaggugg

SEQ ID NO: 383 - RNA, 55 nt, Artificial Sequence
  3rd region of engineered tracrRNA scaffold, MF
gcugcuugcaucagccuaaugucgagaagugcuuucuucggaaaguaacccucga

SEQ ID NO: 384 - RNA, 53 nt, Artificial Sequence
  3rd region of engineered tracrRNA scaffold, MF
gcugcuugcaucagccuaaugucgagaagugcuuuuucgaaaguaacccucga

SEQ ID NO: 385 - RNA, 51 nt, Artificial Sequence
  3rd region of engineered tracrRNA scaffold, MF
gcugcuugcaucagccuaaugucgagaagugcuuuucgaaguaacccucga

SEQ ID NO: 386 - RNA, 51 nt, Artificial Sequence
  3rd region of engineered tracrRNA scaffold, MF
gcugcuugcaucagccuaaugucgagaagugcucuucggaguaacccucga

SEQ ID NO: 387 - RNA, 49 nt, Artificial Sequence
  3rd region of engineered tracrRNA scaffold, MF
gcugcuugcaucagccuaaugucgagaagugcuuucgaguaacccucga

SEQ ID NO: 388 - RNA, 47 nt, Artificial Sequence
  3rd region of engineered tracrRNA scaffold, MF
gcugcuugcaucagccuaaugucgagaagugcuucgguaacccucga

SEQ ID NO: 389 - RNA, 45 nt, Artificial Sequence
  3rd region of engineered tracrRNA scaffold, MF
gcugcuugcaucagccuaaugucgagaaguguucguaacccucga

SEQ ID NO: 390 - RNA, 43 nt, Artificial Sequence
  3rd region of engineered tracrRNA scaffold, MF
gcugcuugcaucagccuaaugucgagaaguuucgaacccucga

SEQ ID NO: 391 - RNA, 41 nt, Artificial Sequence
  3rd region of engineered tracrRNA scaffold, MF
gcugcuugcaucagccuaaugucgagaaguucgacccucga

SEQ ID NO: 392 - RNA, 40 nt, Artificial Sequence
  3rd region of engineered tracrRNA scaffold, MF
gcugcuugcaucagccuaaugucgagaaguucgcccucga

SEQ ID NO: 393 - RNA, 39 nt, Artificial Sequence
  3rd region of engineered tracrRNA scaffold, MF
gcugcuugcaucagccuaaugucgagaauucgcccucga

SEQ ID NO: 394 - RNA, 38 nt, Artificial Sequence
  3rd region of engineered tracrRNA scaffold, MF
gcugcuugcaucagccuaaugucgagaauucgccucga

SEQ ID NO: 395 - RNA, 37 nt, Artificial Sequence
  3rd region of engineered tracrRNA scaffold, MF
gcugcuugcaucagccuaaugucgagauucgccucga

SEQ ID NO: 396 - RNA, 35 nt, Artificial Sequence
  3rd region of engineered tracrRNA scaffold, MF
gcugcuugcaucagccuaaugucgaguucgcucga

SEQ ID NO: 397 - RNA, 55 nt, Artificial Sequence; identical to SEQ ID NO: 383
  3rd region of engineered tracrRNA scaffold, MF

SEQ ID NO: 398 - RNA, 6 nt, Artificial Sequence
  4th region of engineered tracrRNA scaffold, MF
aacaaa

SEQ ID NO: 399 - RNA, 7 nt, Artificial Sequence
  4th region of engineered tracrRNA scaffold, MF
aacaaau

SEQ ID NO: 400 - RNA, 8 nt, Artificial Sequence
  4th region of engineered tracrRNA scaffold, MF
aacaaauu

SEQ ID NO: 401 - RNA, 9 nt, Artificial Sequence
  4th region of engineered tracrRNA scaffold, MF
aacaaauuc

SEQ ID NO: 402 - RNA, 10 nt, Artificial Sequence
  4th region of engineered tracrRNA scaffold, MF
aacaaauuca

SEQ ID NO: 403 - RNA, 11 nt, Artificial Sequence
  4th region of engineered tracrRNA scaffold, MF
aacaaauucau

SEQ ID NO: 404 - RNA, 12 nt, Artificial Sequence
  4th region of engineered tracrRNA scaffold, MF
aacaaauucauu

SEQ ID NO: 405 - RNA, 13 nt, Artificial Sequence
  4th region of engineered tracrRNA scaffold, MF
aacaaauucauuu

SEQ ID NO: 406 - RNA, 17 nt, Artificial Sequence
  crRNA scaffold of Cas12f1, MF
gaaugaaggaaugcaac

SEQ ID NO: 407 - RNA, 30 nt, Artificial Sequence
  6th fragment of engineered crRNA scaffold, WT
  MISC_FEATURE (19): n is a, g, c, or u
  MISC_FEATURE (21)..(23): n is a, g, c, or u
guugcagaacccgaauagnbnnnugaagga

SEQ ID NO: 408 - RNA, 30 nt, Artificial Sequence
  6th fragment of engineered crRNA scaffold, WT
  MISC_FEATURE (19): n is a, g, c, or u
  MISC_FEATURE (21)..(23): n is a, g, c, or u
guugcagaacccgaauagngnnnugaagga

SEQ ID NO: 409 - RNA, 29 nt, Artificial Sequence
  6th fragment of engineered crRNA scaffold, WT
  MISC_FEATURE (18): n is a, g, c, or u
  MISC_FEATURE (20)..(22): n is a, g, c, or u
uugcagaacccgaauagngnnnugaagga

SEQ ID NO: 410 - RNA, 28 nt, Artificial Sequence
  6th fragment of engineered crRNA scaffold, WT
  MISC_FEATURE (17): n is a, g, c, or u
  MISC_FEATURE (19)..(21): n is a, g, c, or u
ugcagaacccgaauagngnnnugaagga

SEQ ID NO: 411 - RNA, 27 nt, Artificial Sequence
  6th fragment of engineered crRNA scaffold, WT
  MISC_FEATURE (16): n is a, g, c, or u
  MISC_FEATURE (18)..(20): n is a, g, c, or u
gcagaacccgaauagngnnnugaagga

SEQ ID NO: 412 - RNA, 26 nt, Artificial Sequence
  6th fragment of engineered crRNA scaffold, WT
  MISC_FEATURE (15): n is a, g, c, or u
  MISC_FEATURE (17)..(19): n is a, g, c, or u
cagaacccgaauagngnnnugaagga

SEQ ID NO: 413 - RNA, 25 nt, Artificial Sequence
  6th fragment of engineered crRNA scaffold, WT
  MISC_FEATURE (14): n is a, g, c, or u
  MISC_FEATURE (16)..(18): n is a, g, c, or u
agaacccgaauagngnnnugaagga

SEQ ID NO: 414 - RNA, 24 nt, Artificial Sequence
  6th fragment of engineered crRNA scaffold, WT
  MISC_FEATURE (13): n is a, g, c, or u
  MISC_FEATURE (15)..(17): n is a, g, c, or u
gaacccgaauagngnnnugaagga

SEQ ID NO: 415 - RNA, 23 nt, Artificial Sequence
  6th fragment of engineered crRNA scaffold, WT
  MISC_FEATURE (12): n is a, g, c, or u
  MISC_FEATURE (14)..(16): n is a, g, c, or u
aacccgaauagngnnnugaagga

SEQ ID NO: 416 - RNA, 22 nt, Artificial Sequence
  6th fragment of engineered crRNA scaffold, WT
  MISC_FEATURE (11): n is a, g, c, or u
  MISC_FEATURE (13)..(15): n is a, g, c, or u
acccgaauagngnnnugaagga

SEQ ID NO: 417 - RNA, 21 nt, Artificial Sequence
  6th fragment of engineered crRNA scaffold, WT
  MISC_FEATURE (10): n is a, g, c, or u
  MISC_FEATURE (12)..(14): n is a, g, c, or u
cccgaauagngnnnugaagga

SEQ ID NO: 418 - RNA, 20 nt, Artificial Sequence
  6th fragment of engineered crRNA scaffold, WT
  MISC_FEATURE (9): n is a, g, c, or u
  MISC_FEATURE (11)..(13): n is a, g, c, or u
ccgaauagngnnnugaagga

SEQ ID NO: 419 - RNA, 19 nt, Artificial Sequence
  6th fragment of engineered crRNA scaffold, WT
  MISC_FEATURE (8): n is a, g, c, or u
  MISC_FEATURE (10)..(12): n is a, g, c, or u
cgaauagngnnnugaagga

SEQ ID NO: 420 - RNA, 18 nt, Artificial Sequence
  6th fragment of engineered crRNA scaffold, WT
  MISC_FEATURE (7): n is a, g, c, or u
  MISC_FEATURE (9)..(11): n is a, g, c, or u
gaauagngnnnugaagga

SEQ ID NO: 421 - RNA, 17 nt, Artificial Sequence
  6th fragment of engineered crRNA scaffold, WT
  MISC_FEATURE (6): n is a, g, c, or u
  MISC_FEATURE (8)..(10): n is a, g, c, or u
aauagngnnnugaagga

SEQ ID NO: 422 - RNA, 16 nt, Artificial Sequence
  6th fragment of engineered crRNA scaffold, WT
  MISC_FEATURE (5): n is a, g, c, or u
  MISC_FEATURE (7)..(9): n is a, g, c, or u
auagngnnnugaagga

SEQ ID NO: 423 - RNA, 15 nt, Artificial Sequence
  6th fragment of engineered crRNA scaffold, WT
  MISC_FEATURE (4): n is a, g, c, or u
  MISC_FEATURE (6)..(8): n is a, g, c, or u
uagngnnnugaagga

SEQ ID NO: 424 - RNA, 14 nt, Artificial Sequence
  6th fragment of engineered crRNA scaffold, WT
  MISC_FEATURE (3): n is a, g, c, or u
  MISC_FEATURE (5)..(7): n is a, g, c, or u
agngnnnugaagga

SEQ ID NO: 425 - RNA, 12 nt, Artificial Sequence
  6th fragment of engineered crRNA scaffold, WT
  MISC_FEATURE (1): n is a, g, c, or u
  MISC_FEATURE (3)..(5): n is a, g, c, or u
ngnnnugaagga

SEQ ID NO: 426 - RNA, 10 nt, Artificial Sequence
  5th region of engineered crRNA scaffold, MF
gaaugaagga

SEQ ID NO: 427 - RNA, 11 nt, Artificial Sequence
  5th region of engineered crRNA scaffold, MF
cgaaugaagga

SEQ ID NO: 428 - RNA, 12 nt, Artificial Sequence
  5th region of engineered crRNA scaffold, MF
acgaaugaagga

SEQ ID NO: 429 - RNA, 13 nt, Artificial Sequence
  5th region of engineered crRNA scaffold, MF
gacgaaugaagga

SEQ ID NO: 430 - RNA, 14 nt, Artificial Sequence
  5th region of engineered crRNA scaffold, MF
agacgaaugaagga

SEQ ID NO: 431 - RNA, 15 nt, Artificial Sequence
  5th region of engineered crRNA scaffold, MF
uagacgaaugaagga

SEQ ID NO: 432 - RNA, 16 nt, Artificial Sequence
  5th region of engineered crRNA scaffold, MF
auagacgaaugaagga

SEQ ID NO: 433 - RNA, 17 nt, Artificial Sequence
  5th region of engineered crRNA scaffold, MF
aauagacgaaugaagga

SEQ ID NO: 434 - RNA, 18 nt, Artificial Sequence
  5th region of engineered crRNA scaffold, MF
gaauagacgaaugaagga

SEQ ID NO: 435 - RNA, 19 nt, Artificial Sequence
  5th region of engineered crRNA scaffold, MF
cgaauagacgaaugaagga

SEQ ID NO: 436 - RNA, 20 nt, Artificial Sequence
  5th region of engineered crRNA scaffold, MF
ccgaauagacgaaugaagga

SEQ ID NO: 437 - RNA, 21 nt, Artificial Sequence
  5th region of engineered crRNA scaffold, MF
cccgaauagacgaaugaagga

SEQ ID NO: 438 - RNA, 22 nt, Artificial Sequence
  5th region of engineered crRNA scaffold, MF
acccgaauagacgaaugaagga

SEQ ID NO: 439 - RNA, 23 nt, Artificial Sequence
  5th region of engineered crRNA scaffold, MF
aacccgaauagacgaaugaagga

SEQ ID NO: 440 - RNA, 24 nt, Artificial Sequence
  5th region of engineered crRNA scaffold, MF
gaacccgaauagacgaaugaagga

SEQ ID NO: 441 - RNA, 25 nt, Artificial Sequence
  5th region of engineered crRNA scaffold, MF
agaacccgaauagacgaaugaagga

SEQ ID NO: 442 - RNA, 26 nt, Artificial Sequence
  5th region of engineered crRNA scaffold, MF
cagaacccgaauagacgaaugaagga

SEQ ID NO: 443 - RNA, 27 nt, Artificial Sequence
  5th region of engineered crRNA scaffold, MF
gcagaacccgaauagacgaaugaagga

SEQ ID NO: 444 - RNA, 28 nt, Artificial Sequence
  5th region of engineered crRNA scaffold, MF
ugcagaacccgaauagacgaaugaagga

SEQ ID NO: 445 - RNA, 29 nt, Artificial Sequence
  5th region of engineered crRNA scaffold, MF
uugcagaacccgaauagacgaaugaagga

SEQ ID NO: 446 - RNA, 47 nt, Artificial Sequence
  shRNA sequence targeting ATM1
ggagccagauaguuuguauuucaagagaauacaaacuaucuggcucc

SEQ ID NO: 447 - RNA, 47 nt, Artificial Sequence
  shRNA sequence targeting ATM1
gcaagcagcugaaacaaauuucaagagaauuuguuucagcugcuugc

SEQ ID NO: 448 - RNA, 47 nt, Artificial Sequence
  shRNA sequence targeting ATM1
ggagcugauuguagcaacauucaagagauguugcuacaaucagcucc

SEQ ID NO: 449 - RNA, 47 nt, Artificial Sequence
  shRNA sequence targeting ATM1
gcacagaagugccuccaauuucaagagaauuggaggcacuucugugc

SEQ ID NO: 450 - RNA, 47 nt, Artificial Sequence
  shRNA sequence targeting ATM1
ggacauaguuucugggagauucaagagaucucccagaaacuaugucc

SEQ ID NO: 451 - RNA, 47 nt, Artificial Sequence
  shRNA sequence targeting XRCC4
ggaugacacuggcacauuauucaagagauaaugugccagugucaucc

SEQ ID NO: 452 - RNA, 47 nt, Artificial Sequence
  shRNA sequence targeting XRCC4
ggagaguacugaugaggaauucaagagauuccucaucaguacucucc

SEQ ID NO: 453 - RNA, 47 nt, Artificial Sequence
  shRNA sequence targeting XRCC4
gaauccaccuuguuucugauucaagagaucagaaacaagguggauuc

SEQ ID NO: 454 - RNA, 47 nt, Artificial Sequence
  shRNA sequence targeting XRCC4
guacaaguaucuugggagauucaagagaucucccaagauacuuguac

SEQ ID NO: 455 - RNA, 47 nt, Artificial Sequence
  shRNA sequence targeting XRCC4
gaaugcagcucaagaacgauucaagagaucguucuugagcugcauuc

SEQ ID NO: 456 - RNA, 47 nt, Artificial Sequence
  shRNA sequence targeting XLF-1
gcaugagucuggcauuacauucaagagauguaaugccagacucaugc

SEQ ID NO: 457 - RNA, 47 nt, Artificial Sequence
  shRNA sequence targeting XLF-1
gaaagcccuuugucaugaauucaagagauucaugacaaagggcuuuc

SEQ ID NO: 458 - RNA, 47 nt, Artificial Sequence
  shRNA sequence targeting XLF-1
gaacagugcuucccugcaauucaagagauugcagggaagcacuguuc

SEQ ID NO: 459 - RNA, 47 nt, Artificial Sequence
  shRNA sequence targeting XLF-1
ggaaagaccuagagauccauucaagagauggaucucuaggucuuucc

SEQ ID NO: 460 - RNA, 47 nt, Artificial Sequence
  shRNA sequence targeting XLF-1
guauggcagucaccacacauucaagagauguguggugacugccauac

SEQ ID NO: 461 - RNA, 47 nt, Artificial Sequence
  shRNA sequence targeting XRCC6
gcagcauugugcagauacauucaagagauguaucugcacaaugcugc

SEQ ID NO: 462 - RNA, 47 nt, Artificial Sequence
  shRNA sequence targeting XRCC6
gcaggaacaucccuccuuauucaagagauaaggagggauguuccugc

SEQ ID NO: 463 - RNA, 47 nt, Artificial Sequence
  shRNA sequence targeting XRCC6
gcagugcucugcucaucaauucaagagauugaugagcagagcacugc

SEQ ID NO: 464 - RNA, 47 nt, Artificial Sequence
  shRNA sequence targeting XRCC6
ggaucaugcuguucaccaauucaagagauuggugaacagcaugaucc

SEQ ID NO: 465 - RNA, 47 nt, Artificial Sequence
  shRNA sequence targeting XRCC6
ggaucugacuacucacucauucaagagaugagugaguagucagaucc

SEQ ID NO: 466 - RNA, 47 nt, Artificial Sequence
  shRNA sequence targeting LIG4
gcacaaagauggagauguauucaagagauacaucuccaucuuugugc

SEQ ID NO: 467 - RNA, 47 nt, Artificial Sequence
  shRNA sequence targeting LIG4
gcagacacguacuguguaauucaagagauuacacaguacgugucugc

SEQ ID NO: 468 - RNA, 47 nt, Artificial Sequence
  shRNA sequence targeting LIG4
ggagcagacuccugaagaauucaagagauucuucaggagucugcucc

SEQ ID NO: 469 - RNA, 47 nt, Artificial Sequence
  shRNA sequence targeting LIG4
ggaggauucugaucugcaauucaagagauugcagaucagaauccucc

SEQ ID NO: 470 - RNA, 47 nt, Artificial Sequence
  shRNA sequence targeting LIG4
gcaugauccuucuguaggauucaagagauccuacagaaggaucaugc

SEQ ID NO: 471 - RNA, 47 nt, Artificial Sequence
  shRNA sequence targeting DCLRE1C
ggagacuccuacccagauauucaagagauaucuggguaggagucucc

SEQ ID NO: 472 - RNA, 47 nt, Artificial Sequence
  shRNA sequence targeting DCLRE1C
ggacaaagcugacuacagauucaagagaucuguagucagcuuugucc

SEQ ID NO: 473 - RNA, 47 nt, Artificial Sequence
  shRNA sequence targeting DCLRE1C
gcagagcucucguuucacauucaagagaugugaaacgagagcucugc

SEQ ID NO: 474 - RNA, 47 nt, Artificial Sequence
  shRNA sequence targeting DCLRE1C
ggacucugauggagaaucauucaagagaugauucuccaucagagucc

SEQ ID NO: 475 - RNA, 47 nt, Artificial Sequence
  shRNA sequence targeting DCLRE1C
gcagaauucuucccagucauucaagagaugacugggaagaauucugc

SEQ ID NO: 476 - DNA, 8192 bp, Artificial Sequence
  661W/LCA10 cell line doner sequence
tctagacctaacatacatactacttctgctcggttataaagtaatctaattccttaaggagccctatttctttgtagagatgaaaatatttccatgcccagaacttattacaccatgtctgtcattgctttgagaatattattgctgcttctctctttccttggacaacatgtaatgtatatctttagaatgtgaattcatatttaactttgtagttttatgttcttcactattttacatacagctgagagattaaagtatttatatcatatattaatttaaaataataacagctgtaatacttcttaatatatcctaaaaccgctttaaagttcttatgttccttggaatctatctcctgaacatacattggcaaaaataacatattctagtaatttgaaatgatactttttgctttcaatattaggattttttttttgagctgaaatgaactatataaaacatttgtattttcaaaagattcagtgtgaagtgtctagctgctgcccctgccctgctaccctgataacagaatatcctccaggtagctcttctgcaacagaagcagtcacgtgcatctggactgtcacgtgagagctggctctctcagatgaggaagttttgagacttacaaatgttcttgtttttattattgtggcttttaaaacatattggacttttctgaagttttgccagtacagagagtatgaaatatacgagctggaggtttctgccatcctccttctttcttgtttcttcaaccacagtttttagggttcgtgatttttttcttttttatagaaaataagaaaagaggatattttaacggaaacaagacataattcatgtcttttaaccagccttcactgcaaaatgaaaccagatgcctctaaggtagaatagtatatgctcatttacatgaggggaagctgtttgaattagacagaaagttcactacactcaggagtgaattagaactataagagcctcatctccccttcagtggtttggttctttgttgctcccatgatctcactgtactttcttatcatggagcatttcactgggaagaactggaaagagtcccggctccttgttggtgttcatcaaaccacctcctgccattcatgaatgccttttcagagctatcggagaatattgcgttaccctcttcccaggactctaccgaatacctcagattattaaacttagaaacagaaaaaggttgttttggttcagctacttgaaggtttgggactatgacctcattacctttggactgtggtaactcgtatacatcatagtaagagggcgggtaatagggaccttgtcacttcatagccaattataatgaaaagaaaaagagggcaccaagacaaaggtcccacggtcctctttgaaagcacaactaaagtgatcttagagttcacaccagcaccagtcttagctggtgccacttccggattgcaccgtgccggagaagactgtagcatgtgtctttgggggaatgttctatattaaactatagcaaacatgaaataagtcaattaaatccttattgttactatttaggtattttaatcagtcgataacaagaaccaaaccttgcgtttgtgtcacttgaagaaagcagacatagacagcatacattaattctgtcacttaaactgccagatgcttagtggattttaatcttctatgacttagagaaggtatccaaagatatcttctgcttactatactttctcaaattcattgtacttcctttttctctgataacttgtaaaaggactcacttaaatccttaattaaaactttccaaataattgttttctacgagaaagaataacaggaaatattacacatttttcaaatggctttcaactattttgctttcaattgaattttctttttaaaaagtgactaaggctttgccagaaaaaaatatgcaaagttaacttggtatatcctttttttttttttttttttttatttttcaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggaggtaagtttgtgtgattcttgaaccttgtgaaattagccatttttcttcaatatttttgtgtttggggggatttggcagattttaattaaagtttgcctgcatttatataaatttaacagagatataattatccatattattcattcagtttagttataaatattttgttcccacataacacacacacacacacacaatatattatctatttatagtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctataccttttactgaggacagaacaagctcctgattttttcatcttcctcatcagaaatagaggcttatggattttggattattcttatctaagatcctttcacaggagtagaataagatctaattctattagctcaaaagcttttgctggctcatagagacacattcagtaaatgaaaacgttgttctgagtagctttcaggattcctactaaattatgagtcatgtttatcaatattatttagaagtaatcataatcagtttgctttctgctgcttttgccaaagagaggtgattatgttactttttatagaaaattatgcctatttagtgtggtgataatttatttttttccattctccatgtcctctgtcctatcctctccagcattagaaagtcctaggcaagagacatcttgtggataatgtatcaatgagtgatgtttaacgttatcattttcccaaagagtatttttcatctttcctaaagattttttttttttttgagatggagtttcattctgtcacccaggctgagtgcagtggcacgatctcggcttaacgcttactgcatcctctgcctcccagattcaagcagttctcctgcctcagcctctgagtagctgggattacaggtgtgcaccaccacagcagctaatttttttttttttttttttttttttgaggcagagtctcgctctgtcacccaggctggagtgcagtggcgccatcttggctcactgcaagctccacctcccgggttcaggccgttctcctgcctcagcctcctgagtagctggtaccacaggcacccaccatcatgcccggctaattttttgtatttttagtagagatggggtttcaccttgttagccaggatggtgtcgatctcctgaactcgtgatccacccgcctcggcctcctaaagtgctgggattacagatgtgagccaccgcacctggccccagttgtaattgtgagtatctcatacctatccctattggcagtgtcttagttttattttttattatctttattgtggcagccattattcctgtctctatctccagtcttacatcctccttactgccacaagaatgatcattctaaacatgaatcctaccctgtgactcccatgtgactccccgccttaaaaactgtcaaaagctaccggttacctgaagggtaaaagtcaagtcccctacttacctcatgtcatctaaagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagataataaggaaatacaaaaactggatgggtaataaagcaaaagaaaaacttgaaatttgatagtagaagaaaaaagaaatagatgtagattgaggtagaatcaagaagaggattctttttttgttgtttttttttttgaaacagagtctcactgtgttgcccaggctggagtgcagtggagtgatcttggcttactgcaacctctgcctcccaggttcaagcgattcttctgcttcagtctcccgagtagctggaattacaggtgcccaccagcacggccggctaatttagtagagacagggttttgccatgttggccgggctggtctcaaactttggatctcaggtaatccgccagcctcaacttcccaaagtgctgggattacaggcatgagccactgtgcccagcctgttttttttttttaaaggagaccagtgaagtttcaggaggagggaaagaaaatttagagttactagggagagagtgatgaagataagagatgaaagtggtaataagggaaatagcaaaatatcagggtaggtgggagaaaaagagatttgtaacaaacaataggattatcctgtgaaaaaggatgaaaggaagaaaaaaatggatagaaagatatttaaaacaccctcagcctcctgttttccctcctgtgtattcatagtatataaaactataattatgtactttacttaaaaaatatattattattaccttatcgtgcttatttaatcatagcatgtcctctttttagtctcattaccctgtttgtattattcttcataacacttaatacctgacattgtattatatattggcttattttccaggtactccactcaaatataagttctaggatataatttatttatcactgaaatccattgcttagagtacctggcatgtagtaaataggcattctgttttttcaaataaaaaataaaggaacttaagatatatatttatgttatatcgccagcctttttcctcacagctctattctgttgtacagaattacctactttacaattcctgtgtttcaaggggatctcaaatttaacgtgtccacaatgaactcctgatttctgtttctctcctagtcattcttatttcaatatatgttcagttacctaaccagctagtcaaggcagatactttagagttattctgtagtcattctttttccctaccatttttgttttccaaatgtaatttatgtgtgtcttcttcatcctcgcagctctaacccttgtccaaaccagcatcatcactcatctggagttccacaatgtctttctggctagtttccctgatttctctattgacccctttattctccacagtgcagccagaatgattgtttaaaacttcctccttaaaatctttaaattgttttcttttatacgttaagttaaattccagttccttgtcttggcatgccatgccctgcctggtgtggcccctgatggtctctccaacttcatgttttactactattgactcttatttttgcttactctgcttgggtgctccagtcctccaaatcatttcctgctccaatcatttcaatcattttttcctctcagatcttatagtattccaaatgctttcttcctttggagcatctgggtttactaataaatacttcgtacctcacagttcagcttaaatatcaattatttggtggttaagacatccttcaaccgctctatctaaatgttcctttctattattcactggctcagtactctgtttttattttctttctaaatgtcaacttttttttttttgagtcagggtctcactgttgcccaggctcgagtgcagttgcacaatcatagctcattgcagccttgccctcctgggatcaagtaattctcccacctcagcctccaaaatagctgggattacaggtatgcatcaccatgctcagctaattttttgtgtttttttgtagagatgaggtctcactttgttgcccaggctggtctcaaactcctggactcaagtgattctcccacctcagcctcccaaagtgctggggttacaggtgtgagccactgcacctggtcgatactgacttttttttttttgagatggagttttgctctgttgcccaggctagagcgcagtggtgtgatctcagctcactgcaacctccacctcccaggttaaagggattcttctgcctcagtctcctgagtagctgggattacaggcaagtgccatcatgactggctaatttttgtatttttagcactatgtttagtactgtgttggccaggcttgtctcgaactcctgacctcaagtgatccacccacctcagcctcccaaagtgctgggattacaggtgtgagccaccgtaatcggccaacattgacatttttagtagactttttgtttgtttacttgcttattatctgctgccttccacactctggcgaaatcctgccacccacccacacacacataggcactgaatgggcagaactctgaaggccagaattttatatttcttttcactataaacatcatcatctgtcactgatggcacactaggatgctcagcaactgtgtgcatgaaggaagtaagcactagtttgtgaaggctgcaaaactcttgagtattctaagagttttggccaaaatgaatgtacagctttagtggcagaagctaatactcagaaattgaggccgtatattggataacacaggatttggatgattattttaaaataatattttacattgtatatatgtgtgtgtgtgtgtgtgtgtgtgtgtgtatgtgtgtgtgtgtgtatatatatatgtatgtatgtgtattagtccgttctcatgctgctatgaagaaatacctgagactgggtaatttataaaggaaagaggtttaattgactcacagttccacagagctggggaggcctcagaaaacttaacagttatggcagaaggggaagcaaacacatttttcttcacatggtggccggaattagaagaatgtgagccgagcaaaggggaaagccccttataaaaccatcagacatcgtgagaacttactattatgagaatagcgtgggggaaaccacccccacgattcaattacctcccaccaaatccctcccatgacatatgaggattatgggaactatgattcaagatgagatttgggtagggacacagccaaaccatatcagtatgtatatgtatacaagtattatatatatatgtatgtgtttgtatgcatacatgtattatatatggaggaaattctaattttgtaaaaaactggattgtgagttttaagataacttcgtatagcatacattatacgaagttataattaaggagcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggatgaggatcgtttcgcatgattgaac

SEQ ID NO: 477 - DNA, 127 bp, Artificial Sequence
  DNA sequence of guide RNA scaffold sequence (V4.0)
accgcttcaccaaaagctgtcccttaggggattagaacttgagtgaaggtgggctgcttgcatcagcctaatgtcgagaagtgctttcttcggaaagtaaccctcgaaacaaagaaaggaatgcaac

SEQ ID NO: 478 - RNA, 98 nt, Artificial Sequence
  guide RNA scaffold sequence (V4.1)
accgcuucaccgagugaaggugggcugcuugcaucagccuaaugucgagaagugcuuucuucggaaaguaacccucgaaacaaagaaaggaaugcaac

SEQ ID NO: 479 - DNA, 1719 bp, Artificial Sequence
  TnpB Encoding DNA
atgggggagaaaagttcccgccgccgacggaatggaaaaagcggtgcgtggactgctgctataacaagctgtgttgggggtaagatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccaaaaggccggcggccacgaaaaaggccggccaggcaaaaaagaaaaag

SEQ ID NO: 480 - DNA, 1665 bp, Artificial Sequence
  Cas12f1 + CasX 28aa Encoding DNA
atggaaaagagaatcaacaagatcaggaagaagctgagcgccgacaacgccaccaagcctgtgtcaggagtggcccccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 481 - DNA, 1671 bp, Artificial Sequence
  Cas12f1 + random 28aa Encoding DNA
atggctggcggaccaggcgcaggtagtgctgcgccagtttcttcaacttcctccctgcccctggctgcgcttaacatgcgcgtgatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 482 - DNA, 1665 bp, Artificial Sequence
  Cas12f1 + random 26aa Encoding DNA
atggctggcggaccaggcgcaggtagtgctgcgccagtttcttcaacttcctccctgcccctggctgcgcttaacatgatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 483 - DNA, 26 bp, Artificial Sequence
  CEP290 qPCR F#1
tggaccagaccctttgtagttatctt

SEQ ID NO: 484 - DNA, 24 bp, Artificial Sequence
  CEP290 qPCR R#1
tgagccagcaaaagcttttgagct

SEQ ID NO: 485 - DNA, 26 bp, Artificial Sequence
  CEP290 64 qPCR F#01
cttacatcctccttactgccacaaga

SEQ ID NO: 486 - DNA, 26 bp, Artificial Sequence
  CEP290 64 qPCR R#01
actgtggtcagaaaactcagctagtt

SEQ ID NO: 487 - DNA, 26 bp, Artificial Sequence
  inner F
tgtggcagccattattcctgtctcta

SEQ ID NO: 488 - DNA, 26 bp, Artificial Sequence
  inner R
gcaacacagtgagactctgtttcaaa

SEQ ID NO: 489 - DNA, 30 bp, Artificial Sequence
  right arm F
ggaactatgattcaagatgagatttgggta

SEQ ID NO: 490 - DNA, 24 bp, Artificial Sequence
  right arm R
ttggacacttcaactttgagctcc

SEQ ID NO: 491 - DNA, 26 bp, Artificial Sequence
  left arm F
tcttctgtggctatcatgagagcgtc

SEQ ID NO: 492 - DNA, 26 bp, Artificial Sequence
  left arm R
tgggtacaggggtaagagaaagggat

(19) United States
(12) Patent Application Publication
(10) Pub. No.: US 2025/0345460 A1
(43) Pub. Date: Nov. 13, 2025

(54) COMPOSITION AND METHOD FOR TREATMENT OF LCA10 USING RNA-GUIDED NUCLEASE

(71) Applicant: GENKORE INC., Daejeon (KR)

(72) Inventors: Yong-Sam KIM, Daejeon (KR); Do Yon KIM, Daejeon (KR); Seyeon PARK, Daejeon (KR)

(73) Assignee: GENKORE INC., Daejeon (KR)

(21) Appl. No.: 18/290,467

(22) PCT Filed: May 13, 2022
(86) PCT No.: PCT/KR2022/006940

§ 371 Date: Nov. 14, 2023

(30) Foreign Application Priority Data
May 14, 2021 (KR) 10-2021-0063023

(51) Int. Cl. A61K 48/00; A61P 27/02; C12N 9/22; C12N 15/11; C12N 15/113; C12N 15/86

(52) U.S. Cl. CPC A61K 48/005; A61P 27/02; C12N 9/226; C12N 15/111; C12N 15/113; C12N 15/86; C12N 2310/20; C12N 2310/531; C12N 2750/14143

(57) ABSTRACT

The present invention relates to LCA10 treatment using a CRISPR/Cas12f1(Cas14a1) system. In particular, the present invention relates to a composition comprising a CRISPR/Cas12f1(Cas14a1) system for LCA10 treatment, and to a treatment method using same. In addition, the present invention relates to a composition comprising a CRISPR/Cas12f1(Cas14a1) system for artificially manipulating the CEP290 gene, and to a method of editing the CEP290 gene.

### TECHNICAL FIELD

The present disclosure relates to treatment of LCA10 disease using a CRISPR/Cas12f1 (Cas14a1) system. More specifically, the present disclosure relates to a composition for treatment of LCA10 disease, comprising a CRISPR/Cas12f1 (Cas14a1) system, and a treatment method using the same. In addition, the present disclosure relates to a composition for artificially manipulating the human CEP290 gene, the composition comprising a CRISPR/Cas12f1 (Cas14a1) system, and a method of manipulating the CEP290 gene using the same.

### BACKGROUND ART

Leber congenital amaurosis type 10 (LCA10) is an autosomal recessive disease caused by a biallelic loss-of-function mutation in the CEP290 gene. The most common mutation in the CEP290 gene, which causes LCA10, is an adenine to guanine point mutation located within intron 26 of the CEP290 gene (called c.2991+1655A>G), and this mutation creates a new splice donor site so that a region for a 128 base pair cryptic exon is included in the messenger RNA (mRNA), thereby generating a premature stop codon. Currently, there is no approved treatment for LCA10. Gene replacement therapy may be a potential treatment. However, due to a large size (about 7.5 kb) of the normal CEP290 gene, there is a limitation in packaging of adeno-associated virus (AAV), which makes it difficult to develop a therapeutic agent. In order to overcome such limitation, treatment using a CRISPR/Cas9 system is receiving attention and is being developed.

### DISCLOSURE

### Technical Problem

An object of the present disclosure is to provide a CRISPR/Cas12f1 (Cas14a1) system for treatment of LCA10 disease.

Another object of the present disclosure is to provide a composition for treatment of LCA10 disease, comprising a CRISPR/Cas12f1 (Cas14a1) system.

Yet another object of the present disclosure is to provide a method for treating LCA10 disease using a CRISPR/Cas12f1 (Cas14a1) system.

Still yet another object of the present disclosure is to provide a CRISPR/Cas12f1 (Cas14a1) system for artificially manipulating or modifying the CEP290 gene, and a method using the same.

### Technical Solution

The present disclosure provides a composition for genetic manipulation comprising a CRISPR/Cas12f1 system.

The CRISPR/Cas12f1 system may be a composition comprising:
a) at least one guide RNA or a nucleic acid encoding the same; and
b) a Cas12f1 protein or a nucleic acid encoding the same,
wherein the guide RNA has a scaffold region and a guide region,
the scaffold region is a region capable of interacting with the Cas12f1 protein and comprises a crRNA scaffold sequence and a tracrRNA scaffold sequence, with the scaffold region being located at the 5′ end of the guide region, and
the guide region has a guide sequence that forms a complementary bond with a target sequence present in the CEP290 gene, and the guide sequence is at least one sequence selected from SEQ ID NOs: 51 to 100, wherein the complementary bond contains 0 to 5 mismatched bonds.

The crRNA scaffold sequence may be any one sequence selected from SEQ ID NOs: 101 to 109.

The tracrRNA scaffold sequence may be any one sequence selected from SEQ ID NOs: 110 to 121.

The scaffold region may further comprise a linker. Here, the scaffold region may be any one sequence selected from SEQ ID NOs: 122 to 126.

The guide sequence may be any one sequence selected from the group consisting of SEQ ID NOs: 60, 64, 66, 67, 79, 81, 92, 94, 96, and 100.

The composition may comprise two or more guide RNAs or nucleic acids encoding the same. Here, the two or more guide RNAs may comprise a first guide RNA and a second guide RNA. Here, the first guide RNA may comprise any one guide sequence selected from SEQ ID NOs: 51 to 74, and the second guide RNA may comprise any one guide sequence selected from SEQ ID NOs: 75 to 100. Here, the first guide RNA may comprise any one guide sequence selected from the group consisting of SEQ ID NOs: 60, 64, 66, and 67, and the second guide RNA may comprise any one guide sequence selected from the group consisting of SEQ ID NOs: 79, 81, 92, 94, 96, and 100.

The guide RNA may optionally further comprise a U-rich tail sequence at the 3′ end. Here, the U-rich tail sequence may be 5′-(UₐN)_{d}Uₑ-3′, 5′-UₐVUₐVUₑ-3′, or 5′-UₐVUₐVUₐVUₑ-3′. Here, N may be A, C, G, or U. Each V may independently be A, C, or G. a may be an integer of 0 to 4. d may be an integer of 0 to 3. e may be an integer of 1 to 10.

The composition may comprise a nucleic acid encoding at least one guide RNA and a nucleic acid encoding the Cas12f1 protein in one vector. Here, the vector may further comprise a promoter for a nucleic acid encoding the one guide RNA and a promoter for a nucleic acid encoding the Cas12f1 protein. Here, in a case where the composition comprises at least two guide RNAs, the vector may comprise promoters for nucleic acids encoding the respective guide RNAs. Here, the promoter may be a U6 promoter, an H1 promoter, a 7SK promoter, a CMV promoter, an LTR promoter, an Ad MLP promoter, an HSV promoter, an SV40 promoter, a CBA promoter, or an RSV promoter. Here, the vector may be a plasmid, an mRNA (transcript), a PCR amplicon, or a viral vector. Here, the viral vector may be at least one viral vector selected from the group consisting of a retroviral (retrovirus) vector, a lentiviral (lentivirus) vector, an adenoviral (adenovirus vector), an adeno-associated viral vector (adeno-associated virus (AAV) vector), a vaccinia viral (vaccinia virus) vector, a poxviral (poxvirus) vector, and a herpes simplex viral (herpes simplex virus) vector.

The composition may be in a mixed form of a nucleic acid and a protein, that is, in a form of a CRISPR/Cas12f1 complex in which the guide RNA and the Cas12f1 protein are bound to each other.

### Advantageous Effects of Disclosure

The present disclosure relates to treatment of LCA10 disease using the CRISPR/Cas12f1 (Cas14a1) system. Through the techniques disclosed by the present specification, it is possible to provide a composition for treatment of LCA10 disease, comprising the CRISPR/Cas12f1 (Cas14a1) system, and a treatment method using the same. In addition, it is possible to provide a composition for artificially manipulating or modifying the CEP290 gene, the composition comprising the CRISPR/Cas12f1 (Cas14a1) system, and a manipulation or modification method using the same.

### BRIEF DESCRIPTION OF DRAWINGS

FIG. 1 illustrates a schematic diagram explaining a cryptic exon created by IVS26 mutation that is a mutation in the CEP290 gene which causes LCA10.

FIG. 2 illustrates a schematic diagram in which a target region for a CRISPR/Cas12f1 system is set and shown. The target region was set by classifying the upstream 1654 bp portion as Forward region (F region) and the downstream 2000 bp portion as Reverse region (R region) based on IVS26 mutation.

FIG. 3 illustrates results obtained by identifying deletion of mutant fragments caused by a CRISPR/Cas12f1 system using a combination of guide RNAs (two guide RNAs) that target the F region and the R region, respectively.

FIG. 4 illustrates results obtained by identifying deletion of fragments caused by a CRISPR/Cas12f1 system using a combination of guide RNAs (a guide RNA having F14 as a guide sequence and a guide RNA having R22 as a guide sequence), which target the F region and the R region, respectively, in HEK-293T cells.

FIG. 5 illustrates results obtained by identifying deletion of fragments caused by a CRISPR/Cas12f1 system using a combination of guide RNAs (a guide RNA having F14 as a guide sequence and a guide RNA having R22 as a guide sequence), which target the F region and the R region, respectively, in ARPE19-HPV cells.

FIG. 6 illustrates a schematic diagram of an experimental design for introducing a point mutation (c.2991+1655A>G) into intron 26 of the CEP290 locus using a CRISPR/Cas9 system and a single-stranded oligonucleotide (ssODN).

FIG. 7 illustrates results obtained by identifying, with PCR using the primer set in Table 20, whether a point mutation (c.2991+1655A>G) has been introduced in ARPE19/HPV16-LCA10 cells generated by transfecting ARPE19 cells with a CRISPR/Cas9 system expression vector and ssODN.

FIG. 8 illustrates results obtained by identifying, through sequencing analysis, the introduction of a point mutation (c.2991+1655A>G) in the ARPE19/HPV16-LCA10 cells identified in FIG. 7. Here, the wild-type sequence (original sequence) that does not contain the point mutation (c.2991+1655A>G) is 5′-CCTGGCCCCAGTTGTAATTGTGAATATCTCAT-3′ (SEQ ID NO: 283), and the sequence, into which the point mutation (c.2991+1655A>G) has been introduced, is 5′-CCTGGCCCCAGTTGTAATTGTGAGTATCTCAT-3′ (SEQ ID NO: 284). Another point mutation was identified in some of the results, and the sequence containing the other point mutation is 5′-CCTGGCCCCAATTGTAATTGTGAATATCTCAT-3′ (SEQ ID NO: 285). Here, the underlined sequence in the above sequence is a point mutation.

FIG. 9 illustrates results obtained by extracting RNA from each of a clone of produced AC7 cells (hereinafter referred to as LCA10-AC7 cells) and cells of ARPE19/HPV16 (WT), and comparing wild-type (WT) mRNA expression levels of the CEP290 gene using qRT-PCR.

FIG. 10 illustrates results obtained by extracting RNA from each of a clone of produced LCA10-AC7 cells and cells of ARPE19/HPV16 (WT), and comparing expression levels of cryptic exon X, which has been generated by mutation (c.2991+1655A>G) of the CEP290 gene, using qRT-PCR.

FIG. 11 illustrates RT-PCR results, identifying that cryptic exon X has been generated by a point mutation (c.2991+1655A>G) in intron 26 of the CEP290 locus in LCA10-AC7 cells.

FIG. 12 illustrates sequencing results, which identify a point mutation (c.2991+1655A>G) in intron 26 of the CEP290 locus in genomic DNA extracted from LCA10-BC8 cells and LCA10-BD5 cells, and results obtained by extracting RNA from LCA10-BC8 cells and LCA10-BD5 cells and performing analysis. Here, the wild-type sequence (original sequence) that does not contain the point mutation (c.2991+1655A>G) is 5′-CTCCTAAAGTGCTGGGATTACAGATGTGAGCCACCGCACCTGGCCCCAG TTGTAATTGTGAATATCTCA-3′ (SEQ ID NO: 286), and the sequence, into which the point mutation (c.2991+1655A>G) has been introduced, is 5′-CTCCTAAAGTGCTGGGATTACAGATGTGAGCCACCGCACCTGGCCCCAG TTGTAATTGTGAGTATCTCA-3′ (SEQ ID NO: 287). Here, the underlined sequence in the above sequence is a point mutation. Here, the results obtained by extracting RNA and performing analysis show a pattern in which several sequences are mixed between exon 26 and exon 27, which indicates that a mutant mRNA is contained therein.

FIG. 13 illustrates results obtained by identifying, with RT-PCR, that cryptic exon X has been generated by a point mutation (c.2991+1655A>G) in intron 26 of the CEP290 locus in LCA10-AC7 cells, LCA10-BC8 cells, and LCA10-BD5 cells.

FIG. 14 illustrates results obtained by identifying an expression level of cryptic exon X in LCA10-BC8 cells and LCA10-BD5 cells.

FIG. 15 illustrates results obtained by identifying that expression of X-27 of the CEP290 gene is decreased by a guide RNA having F14 as a guide sequence and a guide RNA having R22 as a guide sequence, using produced LCA10-BC8 cells. Here, the comparator group is EDIT101.

FIG. 16 illustrates results obtained by identifying that expression of 26-X-27 of the CEP290 gene is decreased by a guide RNA having F14 as a guide sequence and a guide RNA having R22 as a guide sequence, using produced LCA10-BC8 cells. Here, the comparator group is EDIT101.

FIG. 17 schematically illustrates a method for producing 661W/LCA10 cell line.

FIG. 18 illustrates RT-PCR results, identifying expression of 26-X-27 (in which X means cryptic exon X) of 661W/LCA10 cell line, according to Example 6-2.

FIG. 19 illustrates RT-PCR results, identifying expression of X-27 (in which X means cryptic exon X) of 661W/LCA10 cell line, according to Example 6-2.

FIG. 20 illustrates a graph, showing an expression level of X-27 in each clone of 661W/LCA10 cell line, according to Example 6-2. Here, each label is named according to the following nomenclature: A to H, numbers of 1 to 12 (A to H: characters corresponding to rows of a 96-well plate; numbers of 1 to 12: numbers corresponding to columns in respective rows, for example, E08: clone at row E, column 8 in a 96-well plate).

FIG. 21 illustrates RT-PCR results, identifying deletion of a partial region of the CEP290 gene, caused by a LCA10 Cas12f1 therapeutic agent, in 661W/LCA10-E08 cell line, according to Example 7. Here, F14+R18 and F14+R22 refer to respective guide region combinations, and EDIT101 is a comparator group.

FIG. 22 illustrates q-PCR results, identifying deletion of a partial region of the CEP290 gene, according to Example 7. Here, F14+R18 and F14+R22 refer to respective guide region combinations, and EDIT101 is a control.

FIG. 23 illustrates results obtained by performing target screening of F-regions for a Cas12f1 variant, according to Example 8-1. Each label means a target sequence shown in [Table 1], and each row in the table below the graph represents an indel value obtained as a result of repeated experiments.

FIG. 24 illustrates results obtained by performing target screening of R-regions for a Cas12f1 variant, according to Example 8-1. Each label means a target sequence shown in [Table 2], and each row in the table below the graph represents an indel value obtained as a result of repeated experiments.

FIG. 25 illustrates indel efficiency for target sequences depending on guide RNA scaffolds for a Cas12f1 variant, according to Example 8-2. Each of F14, F18, and R22 refers to a target sequence shown in [Table 1] and [Table 2], ver4.0 refers to a guide RNA scaffold sequence of SEQ ID NO: 477, and ver4.1 refers to a guide RNA scaffold sequence of SEQ ID NO: 478.

FIG. 26 schematically illustrates a one-vector system that expresses a Cas12f1 variant and a guide RNA, according to Example 8-2. Specifically, this figure represents a one-vector system capable of expressing a Cas12f1 variant of SEQ ID NO: 288 and a guide RNA comprising a guide RNA scaffold sequence of SEQ ID NO: 477.

FIG. 27 schematically illustrates a one-vector system that expresses a Cas12f1 variant and a guide RNA, according to Example 8-2. Specifically, this figure represents a one-vector system capable of expressing a Cas12f1 variant of SEQ ID NO: 288 and a guide RNA comprising a guide RNA scaffold sequence of SEQ ID NO: 478.

FIG. 28 illustrates indel efficiency for target sequences depending on lengths of a guide sequence for a Cas12f1 variant, according to Example 8-3. F14-19 mer to F14-25 mer represent respective guide sequences shown in Tables 27 and 28, and ver4.0 means that a guide RNA scaffold sequence of SEQ ID NO: 277 was used. Each row in the table below the graph represents an indel value obtained as a result of repeated experiments.

FIG. 29 illustrates indel efficiency for target sequences depending on lengths of a guide sequence for a Cas12f1 variant, according to Example 8-3. R18-20 mer to R18-25 mer represent respective guide sequences shown in Tables 27 and 28, and ver4.0 means that a guide RNA scaffold sequence of SEQ ID NO: 277 was used. Each row in the table below the graph represents an indel value obtained as a result of repeated experiments.

FIG. 30 illustrates indel efficiency for target sequences depending on lengths of a guide sequence for a Cas12f1 variant, according to Example 8-3. R22-20 mer to R22-25 mer represent respective guide sequences shown in Tables 27 and 28, and ver4.0 means that a guide RNA scaffold sequence of SEQ ID NO: 277 was used. Each row in the table below the graph represents an indel value obtained as a result of repeated experiments.

FIG. 31 illustrates inhibitory effects on expression of X-27, caused by LCA10 Cas12f1 variant therapeutic agents, for ARPE19/LCA10-BC08 cell line, according to Example 9. Here, ARPE19/LCA10-BC08 (no treat) refers to a negative control that is not treated with the therapeutic agent; F14+R18 and F14+F22 mean that treatment was performed with the LCA10 Cas12f1 variant therapeutic agents in which respective target sequences shown in [Table 1] and [Table 2] are combined; and EDIT101 is a comparator group.

FIG. 32 illustrates inhibitory effects on expression of X-27, caused by LCA10 Cas12f1 variant therapeutic agents, for 661W/LCA10-E08 cell line, according to Example 9. Here, 661W/LCA10-E08 (no treat) refers to a negative control that is not treated with the therapeutic agent; F14+R18 and F14+F22 mean that treatment was performed with the LCA10 Cas12f1 variant therapeutic agents in which respective target sequences shown in [Table 1] and [Table 2] are combined; and EDIT101 is a comparator group.

FIG. 33 illustrates effects of deleting the intron 26 region of CEP290 in a case where a LCA10 Cas12f1 therapeutic agent and a factor for decreasing activity of NHEJ are used in combination, according to Example 10. Here, Cas12f F14/R22 represents a LCA10 Cas12f1 therapeutic agent which comprises a Cas12f1 protein and guide RNAs targeting F14 and R22, respectively; and TnpB F14/R22 represents a LCA10 Cas12f1 variant therapeutic agent which comprises a Cas12f1 protein variant of SEQ ID NO: 288 and guide RNAs targeting F14 and R22, respectively. In addition, Empty vector is a negative control; shscramble is a positive control; shDCLRE1C-3 represents a result for a case where one nucleic acid encoding shRNA of SEQ ID NO: 473 is contained; and shDCLRE1C-3+shDCLRE1C-3 represents a result for a case where two nucleic acids, each of which encodes shRNA of SEQ ID NO: 473, are contained.

### MODE OF DISCLOSURE

### Definition of Terms

Definitions of terms used in this specification are as follows.

### Nucleic Acid, Nucleotide, Nucleoside, and Base

“Nucleic acid” is a biomolecule (or biopolymer) composed of nucleotide units and is also called a polynucleotide. The nucleic acid comprises both DNA and RNA. “Nucleotide” is a unit composed of phosphoric acid, a pentose sugar, and a base (or nucleobase). In RNA (ribonucleic acid), the pentose sugar is ribose, and in DNA (deoxyribonucleic acid), the pentose sugar is deoxyribose. The nucleotide has one selected from adenine (A), guanine (G), cytosine (C), thymine (T) and uracil (U) as a nucleobase. Here, adenine, guanine, and cytosine exist both in RNA and DNA, thymine exists only in DNA, and uracil exists only in RNA. The nucleotide may also be said to be composed of phosphoric acid and a nucleoside. Here, “nucleoside” consists of a pentose sugar and a nucleobase. The nucleoside is classified into adenosine, thymidine, cytidine, guanosine, and uridine according to the type of nucleobase. Each nucleoside is abbreviated as U (uridine), A (adenosine), T (thymidine), C (cytidine) and G (guanosine). In addition, the nucleotide is abbreviated as U (uridine monophosphate), A (adenosine monophosphate), T (thymidine monophosphate), C (cytidine monophosphate) and G (guanosine monophosphate) according to the type of nucleoside. In addition, the terms include all meanings recognized by those skilled in the art, and may be appropriately interpreted according to the context.

In the present specification, bases, nucleosides, nucleotides, nucleic acids, RNA, and DNA are abbreviated as A, T, G, C, and U depending on the type of base. The above abbreviation may be appropriately interpreted depending on the context. For example, the sequence 5′-UUUUU-3′ may be a sequence of five consecutive bases (uracil), a sequence of five consecutive nucleosides (uridine) and/or a sequence of five consecutive nucleotides (uridine monophosphate). In addition, in the case of a nucleic acid, RNA, and DNA, nucleotides constituting the nucleic acid, RNA, and DNA are abbreviated as uridine, adenosine, thymidine, cytidine and guanosine according to the type of nucleoside. The above abbreviation may be appropriately interpreted depending on the context. For example, RNA comprising a sequence of four consecutive uridines may be interpreted as RNA comprising four consecutive uridine monophosphate nucleotides.

### Target Sequence, Target Strand, Non-Target Strand

“Target sequence”, which is a sequence present in a target nucleic acid or target gene, refers to a sequence recognized by a guide RNA of a CRISPR/Cas12f1 system (or CRISPR/Cas14a1 system) or a target sequence to be modified by a CRISPR/Cas12f1 system (or CRISPR/Cas14a1 system). Specifically, the target sequence refers to a sequence having complementarity to a guide sequence included in the guide RNA or a sequence complementarily binding to the guide sequence.

“Target strand” refers to a strand comprising a target sequence. When the target nucleic acid or target gene is single-stranded, the strand may be a target strand. Alternatively, when the target nucleic acid or target gene is double-stranded, one of the double-strand may be a target strand, and the other strand may be a strand complementary to the target strand. Here, the strand complementary to the target strand is referred to as a “non-target strand.”

The non-target strand comprises a protospacer adjacent motif (PAM) sequence and a protospacer sequence. The PAM sequence is a sequence recognized by a Cas12f1 (or Cas14a1) protein of a CRISPR/Cas12f1 system (or CRISPR/Cas14a1 system). The protospacer sequence, which is located at the 5′ end or the 3′ end of the PAM sequence, is a sequence having complementarity to a target sequence or a sequence that forms a complementary bond with a target sequence. Correlation between the protospacer sequence and the target sequence is similar to correlation between the target sequence and the guide sequence. Due to these characteristics, in general, a protospacer sequence may be used to design a guide sequence. That is, when designing a guide sequence complementarily binding to a target sequence, the guide sequence may be designed as a nucleotide sequence having the same nucleotide sequence as the protospacer sequence. Here, the guide sequence is designed by replacing T with U in the nucleotide sequence of the protospacer sequence.

### Vector

“Vector”, unless otherwise specified, refers collectively to any material capable of transporting a genetic material into a cell. For example, a vector may be a DNA molecule comprising a genetic material of interest, for example, a nucleic acid encoding an effector protein (Cas protein) of a CRISPR/Cas system, and/or a nucleic acid encoding a guide RNA; however, the vector is not limited thereto. The term includes all meanings recognized by those skilled in the art, and may be appropriately interpreted according to the context.

### Operably Linked

The term “operably linked” means that a particular component is arranged with another component in a functional relationship, that is, a particular component is linked to another component so that the particular component can perform its intended function. For example, in a case where a promoter sequence is operably linked to a sequence encoding protein A, the promoter is linked to the sequence encoding the protein A so that the promoter transcribes and/or expresses the sequence encoding the protein A in a cell. In addition, the term includes all meanings recognized by those skilled in the art, and may be appropriately interpreted according to the context.

### Engineered

The term “engineered” is used to distinguish it from a material, a molecule, or the like whose configuration already exists in nature, and this means that the material, the molecule, or the like has undergone artificial modification. For example, the “engineered guide RNA” refers to a guide RNA obtained by applying artificial modification to the configuration of a naturally occurring guide RNA. In addition, the term includes all meanings recognized by those skilled in the art, and may be appropriately interpreted according to the context.

### Nuclear Localization Sequence or Signal (NLS)

In a case where a substance outside the cell's nucleus is transported into the nucleus by nuclear transport, “nuclear localization sequence or signal (NLS)” refers to a peptide of a certain length or a sequence thereof, wherein the peptide is attached to a protein to be transported and acts as a type of “tag.” As used herein, the term “NLS” includes all meanings recognized by those skilled in the art, and may be appropriately interpreted according to the context.

Unless defined otherwise, all technical and scientific terms used herein have the same meanings as commonly understood by those skilled in the art to which the present disclosure belongs. Although methods and materials similar or equivalent to those described herein may be used in practice or experimentation of the present disclosure, suitable methods and materials are described below. All publications, patents, and other references mentioned herein are incorporated by reference in their entirety. Additionally, the materials, methods, and examples are illustrative only and not intended to limit the present disclosure.

Hereinafter, the present disclosure will be described.

### Overview—Treatment of LCA10 Disease Using CRISPR/Cas9

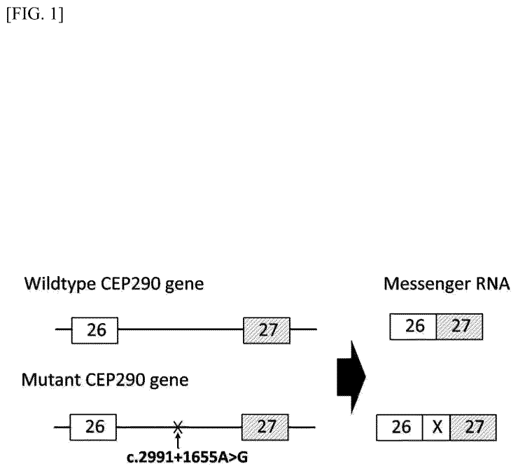

Leber congenital amaurosis type 10 (LCA10) is an autosomal recessive disease caused by a biallelic loss-of-function mutation in the CEP290 gene. Most patients are early infants, with clinically severe cone-rod dystrophy and decreased or lost vision. The protein encoded by the normal CEP290 gene is located in the photoreceptor-connecting cilium and is a necessary factor for phototransduction and outer segment regeneration. The most common mutation in the CEP290 gene, which causes LCA10, is IVS26 mutation. The IVS26 mutation is an adenine to guanine point mutation (c.2991+1655A>G) located within intron 26 between exon 26 and exon 27 of the CEP290 gene, and this mutation creates a new splice donor site so that a region for a 128 base pair cryptic exon is included in the messenger RNA (mRNA), thereby additionally generating cryptic exon X between exon 26 and exon 27 (FIG. 1). This aberrant splicing is more prominent in human photoreceptor cells than in other cell types. Currently, there is no approved treatment for LCA10. Although gene therapy is a potential therapeutic method, a large size (approximately 7.5 kb) of the normal CEP290 gene exceeds packaging capacity of adeno-associated virus (AAV). In order to overcome such limitation, treatment using a CRISPR/Cas9 system is receiving attention and is being developed. In particular, EDIT-101, a therapeutic agent using a CEP290-specific guide RNA and Staphylococcus aureus Cas9 (SaCas9), is being developed by Editas. EDIT-101 uses two guide RNAs with high specificity to the CEP290 locus which allow splicing defect in CEP290 to be removed through gene editing that deletes a mutated part. In this way, a pair of guide RNAs is used to induce inversion or elimination of IVS26 mutation, which leads to restoration of normal splicing and functional CEP290 expression (Morgan L. Maeder et al., Nature Medicine, 25, 229-233 (2019)).

Meanwhile, the present inventors have increased efficiency of the CRISPR/Cas12f1 system, which is a new CRISPR/Cas system, through previous studies, and named this system the CRISPR/Cas12f1-ge system. The CRISPR/Cas12f1 system is a new CRISPR/Cas system that was first reported in a previous study (Harrington et al., Science, 362, 839-842 (2018)), and it has been reported that despite the advantage of a significantly small size, the system has no or extremely low double-stranded DNA cleavage activity which limits its application to gene editing technology. In order to overcome such limitation, the present inventors researched, developed, and completed an engineered guide RNA that enhances double-stranded DNA (dsDNA) cleavage activity so that it can be used for gene editing (Korean Patent Application Nos. 10-2021-0051552, 10-2021-0050093, and 10-2021-0044152). The CRISPR/Cas12f1 system has a significantly smaller Cas protein size than the CRISPR/Cas9 system, which makes it possible to solve the difficulty of loading the CRISPR/Cas9 system on adeno-associated virus (AAV) due to the size of most previously studied Cas proteins and the difficulty of applying the CRISPR/Cas9 system as a gene therapy. In addition, the CRISPR/Cas12f1 system has a feature of inducing dsDNA cleavage outside a protospacer sequence. This feature means that even after the first trial of NHEJ-mediated indel mutation, the dsDNA cleavage-NHEJ process can be executed through additional trials until the protospacer sequence is significantly modified. These multiple cleavage and repair processes may provide more opportunities for reliable cleavage of a target sequence (and a protospacer sequence), and the CRISPR/Cas12f1 system with this feature may have excellent clinical utility in the field of gene therapy.

Based on previous approaches for treating LCA10 disease, the present inventors have introduced a new CRISPR/Cas12f1 system for treatment of LCA10 disease. Introduction of the CRISPR/Cas12f1 system has advantages over the existing CRISPR/Cas9 system, such as ease of loading it on adeno-associated virus (AAV) and reliable gene editing caused by multiple cleavage and repair processes. Accordingly, the present inventors have developed a therapeutic agent and a therapeutic method for LCA10 disease, using the CRISPR/Cas12f1 system having theses advantages.

Hereinafter, the therapeutic agent and the therapeutic method for LCA10 disease, using the CRISPR/Cas12f1 system, will be described in detail.

### <CRISPR/Cas12f1 System for Treatment of LCA10 Disease>

In an aspect of the present disclosure, there is provided a CRISPR/Cas12f1 system for treatment of LCA10 disease. As described above, the LCA10 disease is a disease caused by IVS26 mutation of the CEP290 gene. In order to treat this LCA10 disease, a therapeutic strategy may be used which induces removal or inversion of all or part of the 128 base pair cryptic exon generated by the causative IVS26 mutation so that the normal CEP290 gene is expressed.

For this therapeutic strategy, in an aspect of the present disclosure, a CRISPR/Cas12f1 system is used. The CRISPR/Cas12f1 system disclosed herein includes both a wild-type CRISPR/Cas12f1 system and an artificially modified CRISPR/Cas12f1 system. Here, the artificially modified CRISPR/Cas12f1 system refers to a CRISPR/Cas12f1 system obtained by artificially modifying at least one of the elements, which constitute the CRISPR/Cas12f1 system, that is, a guide RNA and a Cas12f1 protein. For example, the artificially modified CRISPR/Cas12f1 system may comprise an artificially modified guide RNA and a Cas12f1 protein. The artificially modified CRISPR/Cas12f1 system includes CRISPR/Cas12f1-ge system. Thus, the CRISPR/Cas12f1 systems disclosed herein include both a wild-type CRISPR/Cas12f1 system and an artificially modified CRISPR/Cas12f1 system (for example, CRISPR/Cas12f1-ge system).

The CRISPR/Cas12f1 system is used to induce removal or inversion of all or part of the 128 base pair cryptic exon. The CRISPR/Cas12f1 system more effectively removes or inverts all or part of the causative cryptic exon through reliable gene editing caused by multiple cleavage and repair processes, thereby resulting in an increased therapeutic effect. In addition, the CRISPR/Cas12f1 system has a significantly smaller size than the existing CRISPR/Cas9 system, and thus can be more effectively used in application as a therapeutic agent, such as securing additional space (capacity) when inserted into a viral vector such as AAV.

More specifically, the CRISPR/Cas12f1 system for treating LCA10 disease comprises a guide RNA targeting the CEP290 gene and a Cas12f1 protein.

The guide RNA targets the intron 26 region in the CEP290 gene. Here, the intron 26 region contains IVS26 mutation, that is, a point mutation in which adenine located in intron 26 is altered to guanine (referred to as c.2991+1655A>G).

Hereinafter, each component and target of the CRISPR/Cas12f1 system for treatment of LCA10 disease are described in detail.

### 1. Target Gene

LCA10 disease is known to be caused by a point mutation (c.2991+1655A>G) created in the intron 26 region of the CEP290 gene. This point mutation, that is, IVS26 mutation (c.2991+1655A>G) creates a new splice donor site in the intron 26 region, and the resulting aberrant splicing leads to formation of a 128 base pair cryptic exon within an mRNA during transcription of the CEP290 gene, thereby resulting in expression of abnormal CEP290 protein or inhibited expression of normal CEP290 protein. Therefore, for treatment of LCA10 disease, the CEP290 gene was selected as a target, that is, a target gene, of the CRISPR/Cas12f1 system. In particular, the CEP290 gene contains IVS26 mutation (c.2991+1655A>G). As used herein, the “CEP290 gene” refers to CEP290 gene containing IVS26 mutation (c.2991+1655A>G). Here, the CEP290 gene containing IVS26 mutation (c.2991+1655A>G) is also referred to as “abnormal CEP290 gene”, “CEP290 gene variant,” or “CEP290 gene (IVS26)”, and the terms are used interchangeably herein. In addition, the CEP290 gene, which does not contain IVS26 mutation (c.2991+1655A>G), or the CEP gene, which normally expresses CEP290 protein, is referred to as “wild-type CEP290 gene,” “normal CEP290 gene,” or “functional CEP290 gene,” and the terms are used interchangeably herein.

### 1-1) Target Region

For treatment of LCA10 disease, the CRISPR/Cas12f1 system targets the CEP290 gene. More specifically, the CRISPR/Cas12f1 system targets a region in the CEP290 gene. The region in the CEP290 gene is a target region for the CRISPR/Cas12f1 system, and the target region comprises a target sequence that binds complementarily to a guide RNA constituting the CRISPR/Cas12f1 system.

The region, that is, the target region, in the CEP290 gene is the intron 26 region in the CEP290 gene. Here, the intron 26 region contains IVS26 mutation (c.2991+1655A>G). Here, the intron 26 region may be divided into two regions based on the IVS26 mutation (c.2991+1655A>G). That is, the intron 26 region may be divided into an upstream region and a downstream region based on the IVS26 mutation (c.2991+1655A>G). Here, the upstream region may be a region between the 3′ end of exon 26 of the CEP290 gene and the IVS26 mutation (c.2991+1655A>G). Here, the downstream region may be a region between the IVS26 mutation (c.2991+1655A>G) and the 5′ end of exon 27 of the CEP290 gene.

In an embodiment, the target region may be a partial region of the intron 26 region containing the IVS26 mutation (c.2991+1655A>G).

In another embodiment, the target region may be an upstream region based on the IVS26 mutation (c.2991+1655A>G) in the intron 26 region. Alternatively, the target region may be a region between the 3′ end of exon 26 of the CEP290 gene and the IVS26 mutation (c.2991+1655A>G).

In yet another embodiment, the target region may be a downstream region based on the IVS26 mutation (c.2991+1655A>G) in the intron 26 region. Alternatively, the target region may be a region between the IVS26 mutation (c.2991+1655A>G) and the 5′ end of exon 27 of the CEP290 gene.

The target region is double-stranded DNA and is composed of a target strand and a non-target strand. Here, the target strand comprises a target sequence and is a strand to which the guide RNA constituting the CRISPR/Cas12f1 system binds. The non-target strand is a strand complementary to the target strand and comprises a protospacer adjacent motif (PAM) sequence and a protospacer sequence. Here, the protospacer sequence is a sequence having complementarity to the target sequence or a sequence that forms a complementary bond with the target sequence. The target sequence, the target strand, the non-target strand, and the protospacer sequence, as described above, are as previously described in the section “Definition of terms.”

### i) Target Sequence

The target sequence is a partial sequence present in the target region and is a sequence that binds complementarily to the guide RNA constituting the CRISPR/Cas12f1 system. In addition, the target sequence is as previously described in the section “Definition of terms,”, and has the same characteristics and relationships as the target sequence, the target strand, the non-target strand, and the protospacer sequence as described in the above section.

The target sequence may be a sequence of 15 to 40 nucleotides. For example, the target sequence may be a sequence of 15 to 20, 15 to 25, 15 to 30, 15 to 35, or 15 to 40 nucleotides. Alternatively, the target sequence may be a sequence of 20 to 25, 20 to 30, 20 to 35, or 20 to 40 nucleotides. Alternatively, the target sequence may be a sequence of 25 to 30, 25 to 35, or 25 to 40 nucleotides. Alternatively, the target sequence may be a sequence of 30 to 35 or 30 to 40 nucleotides. Alternatively, the target sequence may be a sequence of 35 to 40 nucleotides. In another example, the target sequence may be a sequence of 15, 16, 17, 18, 19, 20, 21, 22, 23, 24, 25, 26, 27, 28, 29, 30, 31, 32, 33, 34, 35, 36, 37, 38, 39, or 40 nucleotides.

In an embodiment, the target sequence may be a sequence of 15 to 40 nucleotides present in a partial region of the intron 26 region containing the IVS26 mutation (c.2991+1655A>G).

In another embodiment, the target sequence may be a sequence of 15 to 40 nucleotides present in an upstream region based on the IVS26 mutation (c.2991+1655A>G) in the intron 26 region. Alternatively, the target sequence may be a sequence of 15 to 40 nucleotides present in a region between the 3′ end of exon 26 of the CEP290 gene and the IVS26 mutation (c.2991+1655A>G). In an embodiment, the target sequences are summarized in Table 1. In Table 1, based on the c.2991+1655A>G gene mutation, the upstream 1654 bp portion was classified as a forward region (F region), and the target sequences are summarized based on protospacer sequences present in the F region. In order to easily distinguish the respective sequences, the sequences present in the F region were classified as F and numbered, and are summarized in such a manner in Table 1.

TABLE 1
Target sequences

| Name | Target sequence | SEQ ID NO |
|---|---|---|
| Target-F01 | 5′-TAAATAGGCATAATTTTCTA-3′ | 1 |
| Target-F02 | 5′-TTGCTTTCTGCTGCTTTTGC-3′ | 2 |
| Target-F03 | 5′-GTAGCTTTCAGGATTCCTAC-3′ | 3 |
| Target-F04 | 5′-GGAGCTTGTTCTGTCCTCAG-3′ | 4 |
| Target-F05 | 5′-GTTTAACGTTATCATTTTCC-3′ | 5 |
| Target-F06 | 5′-GCTCATAGAGACACATTCAG-3′ | 6 |
| Target-F07 | 5′-TTTCTGATGAGGAAGATGAA-3′ | 7 |
| Target-F08 | 5′-GATCTTAGATAAGAATAATC-3′ | 8 |
| Target-F09 | 5′-TTACTTCTAAATAATATTGA-3′ | 9 |
| Target-F10 | 5′-TGGACCATGGATGCACTCTG-3 | 10 |
| Target-F11 | 5′-GCTACATCCATTCCAAGGAA-3′ | 11 |
| Target-F12 | 5′-TTTTCTCTTAGATGTCTGGT-3′ | 12 |
| Target-F13 | 5′-TGTAGAATTTTAATGTAGAA-3′ | 13 |
| Target-F14 | 5′-TCTTACCCCTGTACCCAGAA-3′ | 14 |
| Target-F15 | 5′-CTTGCATGATTTAGCTGAAT-3′ | 15 |
| Target-F16 | 5′-CAGAAGTCATTCAGCCACTA-3 | 16 |
| Target-F17 | 5′-GTGTGTGTGTTATGTGGGAA-3′ | 17 |
| Target-F18 | 5′-TTATTCATTCAGTTTAGTTA-3′ | 18 |
| Target-F19 | 5′-GGGATTTGGCAGATTTTAAT-3′ | 19 |
| Target-F20 | 5′-CTGCCAAATCCCCCCAAACA-3′ | 20 |
| Target-F21 | 5′-CACAAGGTTCAAGAATCACA-3′ | 21 |
| Target-F22 | 5′-CATCATTTTTTATTGTAGAA-3′ | 22 |
| Target-F23 | 5′-GAATGTGTCTCTATGAGCCAG-3′ | 23 |
| Target-F24 | 5′-TAAGATCTAATTCTATTAGCT-3′ | 24 |

In another embodiment, the target sequence may be a sequence of 15 to 40 nucleotides present in a downstream region based on the IVS26 mutation (c.2991+1655A>G) in the intron 26 region. Alternatively, the target sequence may be a sequence of 15 to 40 nucleotides present in a region between the IVS26 mutation (c.2991+1655A>G) and the 5′ end of exon 27 of the CEP290 gene. In an embodiment, the target sequences are summarized in Table 2. In Table 2, based on the c.2991+1655A>G gene mutation, the downstream 2000 bp portion was classified as a reverse region (R region), and the target sequences are summarized based on protospacer sequences present in the R region. In order to easily distinguish the respective sequences, the sequences present in the R region were classified as R and numbered, and are summarized in such a manner in Table 2.

TABLE 2
Target sequences

| Name | Target sequence (20 nt) | SEQ ID NO |
|---|---|---|
| Target-R01 | 5′-TCTCATGAACCTTTACCTCT-3′ | 25 |
| Target-R02 | 5′-TATTAGCTTGAACTCTGTGC-3′ | 26 |
| Target-R03 | 5′-CATTAAGGAAGACAGATATG-3′ | 27 |
| Target-R04 | 5′-CAGGAGTGACTTTGTTCCAT-3′ | 28 |
| Target-R05 | 5′-GCTACCGGTTACCTGAAGGG-3′ | 29 |
| Target-R06 | 5′-TGCCACAAGAATGATCATTC-3′ | 30 |
| Target-R07 | 5′-TCCCATGTGACTCCCCGCCT-3′ | 31 |
| Target-R08 | 5′-TATTGTTGCTTTTTGAGAGG-3′ | 32 |
| Target-R09 | 5′-GTGAATTTCTATTCCTGTTT-3′ | 33 |
| Target-R10 | 5′-GCATACTTTTTTTAATGGAA-3′ | 34 |
| Target-R11 | 5′-CTCAACACACAGAAACAAAT-3′ | 35 |
| Target-R12 | 5′-GAATAGATAATAAGGAAATA-3′ | 36 |
| Target-R13 | 5′-TTTCTTTTTTCTTCTACTAT-3′ | 37 |
| Target-R14 | 5′-CCTGTGTTTCAAGGGGATCT-3′ | 38 |
| Target-R15 | 5′-CCCTTGAAACACAGGAATTG-3′ | 39 |
| Target-R16 | 5′-TAAGCAATGGATTTCAGTGA-3′ | 40 |
| Target-R17 | 5′-CAATGGATTTCAGTGATAAA-3º | 41 |
| Target-R18 | 5′-TATTTTCCAGGTACTCCACT-3′ | 42 |
| Target-R19 | 5′-TACAAACAGGGTAATGAGAG-3′ | 43 |
| Target-R20 | 5 -TCCTGTGTATTCATAGTATA-3′ | 44 |
| Target-R21 | 5′-GATAATCCTATTGTTTGTTA-3º | 45 |
| Target-R22 | 5′-CACTCTCTCCCTAGTAACTC-3′ | 46 |
| Target-R23 | 5′-GAAACTTCACTGGTCTCCTT-3′ | 47 |
| Target-R24 | 5′-CTGGGGGATTACCTGAGATC-3′ | 48 |
| Target-R25 | 5′-CTATAATTATGTACTTTACT-3′ | 49 |
| Target-R26 | 5 -CATGTTGGCCGGGCTGGTCT-3′ | 50 |

### 2. Guide RNA

The CRISPR/Cas12f1 system for treatment of LCA10 disease comprises a guide RNA. The guide RNA comprises a trans-activating CRISPR RNA (tracrRNA) and a CRISPR RNA (crRNA). Here, the crRNA comprises a guide sequence capable of complementarily binding to the target sequence. The crRNA comprises a scaffold sequence that binds to and/or interacts with the tracrRNA and/or the Cas12f1 protein. Here, the tracrRNA has a scaffold sequence that binds to and/or interacts with the crRNA and/or the Cas12f1 protein. Thus, the guide RNA comprises a guide region and a scaffold region. Here, the guide region has a guide sequence, and the scaffold region comprises a scaffold sequence of the crRNA (hereinafter referred to as crRNA scaffold sequence) and a scaffold sequence of the tracrRNA (hereinafter referred to as tracrRNA scaffold sequence).

The guide RNA may optionally further comprise a U-rich tail sequence at the 3′ end. Here, the U-rich tail sequence is a sequence rich in uridine (U), and the U-rich tail sequence is known to play a role in increasing editing efficiency of a target gene (target nucleic acid) which is achieved with an engineered guide RNA (or engineered CRISPR/Cas12f1 (Cas14a1) system) (PCT/KR2020/014961).

Hereinafter, the respective components of the guide RNA are described in detail.

### 2-1) Guide Region

The guide RNA comprises a guide region. The guide region serves to allow the guide RNA to specifically bind to a target gene. Here, the guide region has a guide sequence. The guide sequence is an RNA sequence that recognizes, binds to, or targets a target gene. More specifically, the guide sequence is an RNA sequence that binds complementarily to a target sequence, an RNA sequence capable of forming a complementary bond with a target sequence, or an RNA sequence having complementarity to a target sequence. Alternatively, the guide sequence is an RNA sequence identical to, similar to or corresponding to a protospacer sequence. Here, the protospacer sequence has a systematic relationship with a target sequence, and the description therefor is as described in “Target sequence, target strand, non-target strand” in the definition of terms section. The guide sequence is a sequence that changes depending on a target sequence, and the guide sequence varies depending on the target sequence. In addition, the guide sequence is an RNA sequence, and comprises uridine (U) capable of forming a complementary bond with adenosine (A) present in a target sequence of a target gene. Alternatively, for thymidine (T) present in a protospacer sequence of a target gene, the guide sequence comprises uridine (U) instead of thymidine (T). In addition, the guide sequence is also referred to as a spacer sequence; hereinafter, the guide sequence and the spacer sequence are used interchangeably.

The guide sequence is located at the 3′ end of the crRNA. Alternatively, the guide sequence is located at the 3′ end of the crRNA scaffold sequence.

The guide sequence is covalently linked to the 3′ end of the crRNA scaffold sequence.

The guide sequence may be a sequence of 15 to 40 nucleotides.

In an embodiment, the guide sequence may be a sequence of 15 to 20, 15 to 25, 15 to 30, 15 to 35, or 15 to 40 nucleotides. Alternatively, the guide sequence may be a sequence of 20 to 25, 20 to 30, 20 to 35, or 20 to 40 nucleotides. Alternatively, the guide sequence may be a sequence of 25 to 30, 25 to 35, or 25 to 40 nucleotides. Alternatively, the guide sequence may be a sequence of 30 to 35 or 30 to 40 nucleotides. Alternatively, the guide sequence may be a sequence of 35 to 40 nucleotides.

In another embodiment, the guide sequence may be a sequence of 15, 16, 17, 18, 19, 20, 21, 22, 23, 24, 25, 26, 27, 28, 29, 30, 31, 32, 33, 34, 35, 36, 37, 38, 39, or 40 nucleotides.

The guide sequence may be a sequence that forms a complementary bond with a target sequence. Here, the complementary bond may optionally comprise at least one mismatched bond. For example, the guide sequence is a sequence that forms a complementary bond with a target sequence, wherein the complementary bond may comprise 0 to 5 mismatched bonds, or the complementary bond may comprise 0 to 5 mismatches.

The guide sequence may be a sequence complementary to a target sequence. Here, the complementary sequence may comprise a sequence of 0 to 5 mismatched nucleotides with respect to the target sequence. Alternatively, the guide sequence may be a nucleotide sequence that is at least 70% complementary to a target sequence. Here, in a case where the target sequence is DNA, for adenosine (A) present in the target sequence, the guide sequence may comprise uridine (U) capable of forming a complementary bond with the adenosine (A).

In an embodiment, the guide sequence may be a sequence that is at least 70% to 75%, at least 70% to 80%, at least 70% to 85%, at least 70% to 90%, at least 70% to 95%, at least 70% to 100%, at least 75% to 80%, at least 75% to 85%, at least 75% to 90%, at least 75% to 95%, or at least 75% to 100% to the target sequence. Alternatively, the guide sequence may be a sequence that is at least 80% to 85%, at least 80% to 90%, at least 80% to 95%, at least 80% to 100%, at least 85% to 90%, at least 85% to 95%, or at least 85% to 100% complementary to the target sequence. Alternatively, the guide sequence may be a sequence that is at least 90% to 95%, at least 90% to 100%, or at least 95% to 100% complementary to the target sequence. Alternatively, the guide sequence may be a sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% complementary to the target sequence.

The guide sequence may be a sequence that is identical or similar to a protospacer sequence. Alternatively, the guide sequence may be a sequence having sequence identity or similarity to the protospacer sequence. Here, the sequence identity or similarity may be at least 70%. Here, for thymidine (T) present in the protospacer sequence, the guide sequence may comprise uridine (U) instead of thymidine (T).

In an embodiment, the guide sequence may be a sequence that is at least 70% to 75%, at least 70% to 80%, at least 70% to 85%, at least 70% to 90%, at least 70% to 95%, at least 70% to 100%, at least 75% to 80%, at least 75% to 85%, at least 75% to 90%, at least 75% to 95%, or at least 75% to 100% identical or similar to the protospacer sequence. Alternatively, the guide sequence may be a sequence that is at least 80% to 85%, at least 80% to 90%, at least 80% to 95%, at least 80% to 100%, at least 85% to 90%, at least 85% to 95%, or at least 85% to 100% identical or similar to the protospacer sequence. Alternatively, the guide sequence may be a sequence that is at least 90% to 95%, at least 90% to 100%, or at least 95% to 100% identical or similar to the protospacer sequence. Alternatively, the guide sequence may be a sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical or similar to the protospacer sequence.

In another embodiment, the guide sequence may be a sequence having sequence identity or similarity of at least 70% to 75%, at least 70% to 80%, at least 70% to 85%, at least 70% to 90%, at least 70% to 95%, 70% to 100%, at least 75% to 80%, at least 75% to 85%, at least 75% to 90%, at least 75% to 95%, or at least 75% to 100% to the protospacer sequence. Alternatively, the guide sequence may be a sequence having sequence identity or similarity of at least 80% to 85%, at least 80% to 90%, at least 80% to 95%, at least 80% to 100%, at least 85% to 90%, at least 85% to 95%, or at least 85% to 100% to the protospacer sequence. Alternatively, the guide sequence may be a sequence having sequence identity or similarity of at least 90% to 95%, at least 90% to 100%, or at least 95% to 100% to the protospacer sequence. Alternatively, the guide sequence may be a sequence having sequence identity or similarity of at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% to the protospacer sequence.

In an embodiment, the guide sequence may be a sequence of 15 to 40 nucleotides that forms a complementary bond with a target sequence present in a partial region of the intron 26 region containing the IVS26 mutation (c.2991+1655A>G) of the CEP290 gene.

In an embodiment, the guide sequence may be a sequence of 15 to 40 nucleotides that forms a complementary bond with the target sequence present in an upstream region based on the IVS26 mutation (c.2991+1655A>G) in the intron 26 region of the CEP290 gene. Alternatively, the guide sequence may be a sequence of 15 to 40 nucleotides that forms a complementary bond with the target sequence present in a region between the 3′ end of exon 26 of the CEP290 gene and the IVS26 mutation (c.2991+1655A>G). In an embodiment, the guide sequence may be a sequence that forms a complementary bond with any one sequence selected from the target sequences listed in Table 1. Here, the guide sequences are summarized in Table 3. In Table 3, the guide sequences are summarized which bind complementarily to target sequences present in a forward region (F region) that is an upstream 1654 bp portion based on the c.2991+1655A>G gene mutation. In order to easily distinguish the respective sequences, the guide sequences targeting the F region were classified as F and numbered, and are summarized in such a manner in Table 3.

TABLE 3
Guide sequences

| | Guide sequence (20 nt) | |
|---|---|---|
| Name | (5′ to 3′) | SEQ ID NO |
| Guide-F01 | UAGAAAAUUAUGCCUAUUUA | 51 |
| Guide-F02 | GCAAAAGCAGCAGAAAGCAA | 52 |
| Guide-F03 | GUAGGAAUCCUGAAAGCUAC | 53 |
| Guide-F04 | CUGAGGACAGAACAAGCUCC | 54 |
| Guide-F05 | GGAAAAUGAUAACGUUAAAC | 55 |
| Guide-F06 | CUGAAUGUGUCUCUAUGAGC | 56 |
| Guide-F07 | UUCAUCUUCCUCAUCAGAAA | 57 |
| Guide-F08 | GAUUAUUCUUAUCUAAGAUC | 58 |
| Guide-F09 | UCAAUAUUAUUUAGAAGUAA | 59 |
| Guide-F10 | CAGAGUGCAUCCAUGGUCCA | 60 |
| Guide-F11 | UUCCUUGGAAUGGAUGUAGC | 61 |
| Guide-F12 | ACCAGACAUCUAAGAGAAAA | 62 |
| Guide-F13 | UUCUACAUUAAAAUUCUACA | 63 |
| Guide-F14 | UUCUGGGUACAGGGGUAAGA | 64 |
| Guide-F15 | AUUCAGCUAAAUCAUGCAAG | 65 |
| Guide-F16 | UAGUGGCUGAAUGACUUCUG | 66 |
| Guide-F17 | UUCCCACAUAACACACACAC | 67 |
| Guide-F18 | UAACUAAACUGAAUGAAUAA | 68 |
| Guide-F19 | AUUAAAAUCUGCCAAAUCCC | 69 |
| Guide-F20 | UGUUUGGGGGGAUUUGGCAG | 70 |
| Guide-F21 | UGUGAUUCUUGAACCUUGUG | 71 |
| Guide-F22 | UUCUACAAUAAAAAAUGAUG | 72 |
| Guide-F23 | CUGGCUCAUAGAGACACAUUC | 73 |
| Guide-F24 | AGCUAAUAGAAUUAGAUCUUA | 74 |

In another embodiment, the guide sequence may be a sequence of 15 to 40 nucleotides that forms a complementary bond with the target sequence present in a downstream region based on the IVS26 mutation (c.2991+1655A>G) in the intron 26 region of the CEP290 gene. Alternatively, the guide sequence may be a sequence of 15 to 40 nucleotides that forms a complementary bond with the target sequence present in a region between the IVS26 mutation (c.2991+1655A>G) and the 5′ end of exon 27 of the CEP290 gene. In an embodiment, the guide sequence may be a sequence that forms a complementary bond with any one sequence selected from the target sequences listed in Table 2. Here, the guide sequences are summarized in Table 4. In Table 4, the guide sequences are summarized which bind complementarily to target sequences present in a reverse region (R region) that is a downstream 2000 bp portion based on the c.2991+1655A>G gene mutation. In order to easily distinguish the respective sequences, the guide sequences targeting the R region were classified as R and numbered, and are summarized in such a manner in Table 4.

TABLE 4
Guide sequences

| | Guide sequence (20 nt) | |
|---|---|---|
| Name | (5′ to 3′) | SEQ ID NO |
| Guide-R01 | AGAGGUAAAGGUUCAUGAGA | 75 |
| Guide-R02 | GCACAGAGUUCAAGCUAAUA | 76 |
| Guide-R03 | CAUAUCUGUCUUCCUUAAUG | 77 |
| Guide-R04 | AUGGAACAAAGUCACUCCUG | 78 |
| Guide-R05 | CCCUUCAGGUAACCGGUAGC | 79 |
| Guide-R06 | GAAUGAUCAUUCUUGUGGCA | 80 |
| Guide-R07 | AGGCGGGGAGUCACAUGGGA | 81 |
| Guide-R08 | CCUCUCAAAAAGCAACAAUA | 82 |
| Guide-R09 | AAACAGGAAUAGAAAUUCAC | 83 |
| Guide-R10 | UUCCAUUAAAAAAAGUAUGC | 84 |
| Guide-R11 | AUUUGUUUCUGUGUGUUGAG | 85 |
| Guide-R12 | UAUUUCCUUAUUAUCUAUUC | 86 |
| Guide-R13 | AUAGUAGAAGAAAAAAGAAA | 87 |
| Guide-R14 | AGAUCCCCUUGAAACACAGG | 88 |
| Guide-R15 | CAAUUCCUGUGUUUCAAGGG | 89 |
| Guide-R16 | UCACUGAAAUCCAUUGCUUA | 90 |
| Guide-R17 | UUUAUCACUGAAAUCCAUUG | 91 |
| Guide-R18 | AGUGGAGUACCUGGAAAAUA | 92 |
| Guide-R19 | GUCUCAUUACCCUGUUUGUA | 93 |
| Guide-R20 | UAUACUAUGAAUACACAGGA | 94 |
| Guide-R21 | UAACAAACAAUAGGAUUAUC | 95 |
| Guide-R22 | GAGUUACUAGGGAGAGAGUG | 96 |
| Guide-R23 | AAGGAGACCAGUGAAGUUUC | 97 |
| Guide-R24 | GAUCUCAGGUAAUCCGCCAG | 98 |
| Guide-R25 | AGUAAAGUACAUAAUUAUAG | 99 |
| Guide-R26 | AGACCAGCCCGGCCAACAUG | 100 |

### 2-2) Scaffold Region

The guide RNA comprises a scaffold region. The scaffold region serves to interact with a Cas12f1 protein and form a CRISPR/Cas12f1 complex. Here, the scaffold region comprises a crRNA scaffold sequence and a tracrRNA scaffold sequence. The scaffold region is located at the 5′ end of the guide region.

The scaffold region may have a dual scaffold sequence or a single scaffold sequence. Here, the dual scaffold sequence is composed of two molecules and may comprise a crRNA scaffold sequence and a tracrRNA scaffold sequence as separate molecules. Here, the guide RNA is a dual guide RNA and may be composed of two molecules. That is, in the dual guide RNA, crRNA and tracrRNA may each independently exist. Here, the single scaffold sequence is composed of one molecule and may comprise a crRNA scaffold sequence, a linker, and a tracrRNA scaffold sequence. The single scaffold sequence may be a single RNA molecule in which a crRNA scaffold sequence, a linker, and a tracrRNA scaffold sequence are linked to each other, and may be a sequence of 5′-[tracrRNA scaffold sequence]-[linker]-[crRNA scaffold sequence]-3′. Here, the guide RNA is a single guide RNA, and may be composed of one RNA molecule and have a sequence of 5′-[tracrRNA]-[linker]-[crRNA]-3′ in which the tracrRNA, the linker, and the crRNA are linked to each other.

Hereinafter, the crRNA scaffold sequence, the tracrRNA scaffold sequence, and the linker are described in detail.

i) crRNA Scaffold Sequence

The scaffold region comprises a crRNA scaffold sequence. The crRNA scaffold sequence is a partial sequence present in the crRNA that binds to and/or interacts with the tracrRNA and/or the Cas12f1 protein.

The crRNA scaffold sequence may be a wild-type crRNA scaffold sequence or an engineered crRNA scaffold sequence. Here, the engineered crRNA scaffold sequence may be an engineered form of the wild-type crRNA scaffold sequence in which artificial modification (substitution, deletion, or insertion) is made at a partial nucleotide sequence in the wild-type crRNA scaffold sequence, or in which modification is made to have a shorter length than the wild-type crRNA scaffold sequence.

The crRNA scaffold sequence may be located at the 5′ end of the guide sequence. Alternatively, the crRNA scaffold sequence may be located at the 5′ end of the crRNA.

The crRNA scaffold sequence may covalently bind to the 5′ end of the guide sequence.

In an embodiment, the crRNA scaffold sequence may be a wild-type crRNA scaffold sequence. Here, the wild-type crRNA scaffold sequence may be 5′-GUUGCAGAACCCGAAUAGACGAAUGAAGGAAUGCAAC-3′ (SEQ ID NO: 101).

In another embodiment, the crRNA scaffold sequence may be an engineered crRNA scaffold sequence. Here, the engineered crRNA scaffold sequence may be an engineered form of SEQ ID NO: 101 in which a partial nucleotide sequence in SEQ ID NO: 101 is subjected to artificial substitution, deletion, or insertion. Alternatively, the engineered crRNA scaffold sequence may be an engineered form of SEQ ID NO: 101 in which modification is made to have a shorter length than SEQ ID NO: 101.

In an embodiment, the engineered crRNA scaffold sequence is a partial sequence of SEQ ID NO: 101, and this sequence may be a sequence of SEQ ID NO: 101 that does not contain a partial sequence of the 5′ end or 3′ end of SEQ ID NO: 101. For example, the engineered crRNA scaffold sequence may be 5′-UGCAGAACCCGAAUAGACGAAUGAAGGAAUGCAAC-3′ (SEQ ID NO: 102), 5′-CAGAACCCGAAUAGACGAAUGAAGGAAUGCAAC-3′ (SEQ ID NO: 103), 5′-GAAUAGACGAAUGAAGGAAUGCAAC-3′ (SEQ ID NO: 104), 5′-ACGAAUGAAGGAAUGCAAC-3′ (SEQ ID NO: 105), or 5′-GGAAUGCAAC-3′ (SEQ ID NO: 106). These examples are provided for illustrative purposes only and are not intended to limit the disclosure.

In another embodiment, the engineered crRNA scaffold sequence is an engineered form of SEQ ID NO: 101 in which an artificial modification is made at a partial nucleotide sequence in SEQ ID NO: 101, and this sequence may be an engineered form of SEQ ID NO: 101 in which substitution, deletion, and/or addition are made at one or more nucleotides in SEQ ID NO: 101. For example, the engineered crRNA scaffold sequence may be 5′-GUUGCAGAACCCGAAUAGAGCAAUGAAGGAAUGCAAC-3′ (SEQ ID NO: 107), 5′-GAAUAGAGCAAUGAAGGAAUGCAAC-3′ (SEQ ID NO: 108), or 5′-AGCAAUGAAGGAAUGCAAC-3′ (SEQ ID NO: 109). These examples are provided for illustrative purposes only and are not intended to limit the disclosure.

ii) tracrRNA Scaffold Sequence

The scaffold region comprises a tracrRNA scaffold sequence. The tracrRNA scaffold sequence is all or part of the tracrRNA that binds to and/or interacts with the crRNA and/or the Cas12f1 protein.

The tracrRNA scaffold sequence may be a wild-type tracrRNA scaffold sequence or an engineered tracrRNA scaffold sequence. Here, the engineered tracrRNA scaffold sequence may be an engineered form of the wild-type tracrRNA scaffold sequence in which artificial modification (substitution, deletion, or insertion) is made at a partial nucleotide sequence in the wild-type tracrRNA scaffold sequence, or in which modification is made to have a shorter length than the wild-type tracrRNA scaffold sequence.

In an embodiment, the tracrRNA scaffold sequence may be a wild-type tracrRNA scaffold sequence. Here, the wild-type tracrRNA scaffold sequence may be 5′-CUUCACUGAUAAAGUGGAGAACCGCUUCACCAAAAGCUGUCCCUUAG GGGAUUAGAACUUGAGUGAAGGUGGGCUGCUUGCAUCAGCCUAAUGUC GAGAAGUGCUUUCUUCGGAAAGUAACCCUCGAAACAAAUUCAUUUUUC CUCUCCAAUUCUGCACAA-3′ (SEQ ID NO: 110).

In another embodiment, the tracrRNA scaffold sequence may be an engineered tracrRNA scaffold sequence. Here, the engineered tracrRNA scaffold sequence may be an engineered form of SEQ ID NO: 110 in which artificial modification (substitution, deletion, or insertion) is made at a partial nucleotide sequence in SEQ ID NO: 110. Alternatively, the engineered tracrRNA scaffold sequence may be an engineered form of SEQ ID NO: 110 in which modification is made to have a shorter length than SEQ ID NO: 110.

In an embodiment, the engineered tracrRNA scaffold sequence is a partial sequence of SEQ ID NO: 110, and this sequence may be a sequence of SEQ ID NO: 110 that does not contain a partial sequence of the 5′ end or 3′ end of SEQ ID NO: 110. For example, the engineered tracrRNA scaffold sequence may be 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUUCGGAA AGUAACCCUCGAAACAAAUUCAUUUUUCCUCUCCAAUUCUGCACAA-3′ (SEQ ID NO: 111), 5′-CUUCACUGAUAAAGUGGAGAACCGCUUCACCAAAAGCUGUCCCUUAG GGGAUUAGAACUUGAGUGAAGGUGGGCUGCUUGCAUCAGCCUAAUGUC GAGAAGUGCUUUCUUCGGAAAGUAACCCUCGAAACAAAUUCAUUU-3′ (SEQ ID NO: 112), 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUUCGGAA AGUAACCCUCGAAACAAAUUCAUUU-3′ (SEQ ID NO: 113), 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUUCGGAA AGUAACCCUCGAAACAAA-3′ (SEQ ID NO: 114), 5′-ACCGCUUCACCUUAGGAGUGAAGGUGGGCUGCUUGCAUCAGCCUAAU GUCGAGAAGUGCUUUCUUCGGAAAGUAACCCUCGAAACAAAUUCAUUU-3′ (SEQ ID NO: 115), or 5′-ACCGCUUCACUUAGAGUGAAGGUGGGCUGCUUGCAUCAGCCUAAUGU CGAGAAGUGCUUUCUUCGGAAAGUAACCCUCGAAACAAA-3′ (SEQ ID NO: 116). These examples are provided for illustrative purposes only and are not intended to limit the disclosure.

In another embodiment, the engineered tracrRNA scaffold sequence is an engineered form of SEQ ID NO: 110 in which an artificial modification is made at a partial nucleotide sequence in SEQ ID NO: 110, and this sequence may be an engineered form of SEQ ID NO: 110 in which substitution, deletion, and/or addition are made at one or more nucleotides in SEQ ID NO: 110. For example, the engineered tracrRNA scaffold sequence may be 5′-CUUCACUGAUAAAGUGGAGAACCGCUUCACCAAAAGCUGUCCCUUAG GGGAUUAGAACUUGAGUGAAGGUGGGCUGCUUGCAUCAGCCUAAUGUC GAGAAGUGCUUUCUUCGGAAAGUAACCCUCGAAACAAAUUCAGUGCUC CUCUCCAAUUCUGCACAA-3′ (SEQ ID NO: 117), 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUUCGGAA AGUAACCCUCGAAACAAAUUCAGUGCUCCUCUCCAAUUCUGCACAA-3′ (SEQ ID NO: 118), 5′-ACCGCUUCACCAAUUAGUUGAGUGAAGGUGGGCUGCUUGCAUCAGCC UAAUGUCGAGAAGUGCUUUCUUCGGAAAGUAACCCUCGAAACAAAUUC AGUGCUCCUCUCCAAUUCUGCACAA-3′ (SEQ ID NO: 119), 5′-ACCGCUUCACCAAUUAGUUGAGUGAAGGUGGGCUGCUUGCAUCAGCC UAAUGUCGAGAAGUGCUUUCUUCGGAAAGUAACCCUCGAAACAAAUUC AGUGCUCCUCUC-3′ (SEQ ID NO: 120), or 5′-ACCGCUUCACCAAUUAGUUGAGUGAAGGUGGGCUGCUUGCAUCAGCC UAAUGUCGAGAAGUGCUUUCUUCGGAAAGUAACCCUCGAAACAAAUUC AGUGCU-3′ (SEQ ID NO: 121). These examples are provided for illustrative purposes only and are not intended to limit the disclosure.

iii) Linker

In a case where the scaffold region is a single scaffold sequence, the scaffold region further comprises a linker. The linker is a sequence that serves to link the tracrRNA scaffold sequence to the crRNA scaffold sequence. That is, the scaffold region is a single RNA molecule in which tracrRNA scaffold sequence and the crRNA scaffold sequence are connected to each other via a linker.

The linker may be a sequence that does not affect functions of the tracrRNA scaffold sequence and/or the crRNA scaffold sequence. Alternatively, the linker may be a sequence that does not form an RNA duplex with the tracrRNA scaffold sequence and/or the crRNA scaffold sequence.

The linker may be a sequence of 1 to 30 nucleotides.

In an embodiment, the linker may be a sequence of 1 to 5, 5 to 10, 10 to 15, 15 to 20, 20 to 25, or 25 to 30 nucleotides.

In another embodiment, the linker may be a sequence of 1 to 30, 5 to 30, 10 to 30, 15 to 30, 20 to 30, or 25 to 30 nucleotides.

In yet another embodiment, the linker may be a sequence of 1, 2, 3, 4, 5, 6, 7, 8, 9, 10, 11, 12, 13, 14, 15, 16 17, 18, 19, 20, 21, 22, 23, 24, 25, 26, 27, 28, 29, or 30 nucleotides. For example, the linker may be a sequence of 5′-GAAA-3′, but is not limited thereto.

### iv) Examples of Scaffold Region

Based on the previous description, examples of the scaffold region will be described. Specific descriptions of the components included in the examples below are the same as those described above for the corresponding components. The examples are for illustrative purposes only, and the scope of the present disclosure is not limited thereto.

In an embodiment, the scaffold region may comprise a wild-type crRNA scaffold sequence and a wild-type tracrRNA scaffold sequence. The scaffold region may have a dual scaffold sequence. Here, the dual scaffold sequence may comprise the wild-type crRNA scaffold sequence and the wild-type tracrRNA scaffold sequence as separate RNA molecules. Alternatively, the scaffold region may have a single scaffold sequence. Here, the scaffold region may further comprise a linker, and the wild-type crRNA scaffold sequence and the wild-type tracrRNA scaffold sequence may be connected to each other via the linker.

In an embodiment, the scaffold region may comprise SEQ ID NOS: 101 and 110. Here, the scaffold region may optionally further comprise a linker. As an example, the scaffold region may comprise SEQ ID NO: 110, a linker, and SEQ ID NO: 101 sequentially in a 5′ to 3′ direction. For example, the scaffold region may have

(SEQ ID NO: 122)
5′-CUUCACUGAUAAAGUGGAGAACCGCUUCACCAAAAGCUGUCCCUUAG
GGGAUUAGAACUUGAGUGAAGGUGGGCUGCUUGCAUCAGCCUAAUGUC
GAGAAGUGCUUUCUUCGGAAAGUAACCCUCGAAACAAAUUCAUUUUUC
CUCUCCAAUUCUGCACAAGAAAGUUGCAGAACCCGAAUAGACGAAUGA
AGGAAUGCAAC-3′.

In another embodiment, the scaffold region may comprise a wild-type crRNA scaffold sequence and an engineered tracrRNA scaffold sequence. The scaffold region may have a dual scaffold sequence. Here, the dual scaffold sequence may comprise the wild-type crRNA scaffold sequence and the engineered tracrRNA scaffold sequence as separate RNA molecules. Alternatively, the scaffold region may have a single scaffold sequence. Here, the scaffold region may further comprise a linker, and the wild-type crRNA scaffold sequence and the engineered tracrRNA scaffold sequence may be connected to each other via the linker.

In an embodiment, the scaffold region may comprise i) a sequence of SEQ ID NO: 101, and ii) a sequence of SEQ ID NO: 110, in each of which substitution and/or deletion of one or more nucleotides are made, and/or insertion of one or more other nucleotides is made. Here, the scaffold region may optionally further comprise a linker. For example, the scaffold region may have SEQ ID NO: 101 and SEQ ID NO: 117. Alternatively, the scaffold region may comprise SEQ ID NO: 117, a linker, and SEQ ID NO: 101 sequentially in a 5′ to 3′ direction. For example, the scaffold region may have

(SEQ ID NO: 123)
5′-CUUCACUGAUAAAGUGGAGAACCGCUUCACCAAAAGCUGUCCCUUAG
GGGAUUAGAACUUGAGUGAAGGUGGGCUGCUUGCAUCAGCCUAAUGUC
GAGAAGUGCUUUCUUCGGAAAGUAACCCUCGAAACAAAUUCAGUGCUC
CUCUCCAAUUCUGCACAAGAAAGUUGCAGAACCCGAAUGAAGGAAUGC
AAC-3′.

In another embodiment, the scaffold region may comprise an engineered crRNA scaffold sequence and an engineered tracrRNA scaffold sequence. The scaffold region may have a dual scaffold sequence. Here, the dual scaffold sequence may comprise the engineered crRNA scaffold sequence and the engineered tracrRNA scaffold sequence as separate RNA molecules. Alternatively, the scaffold region may have a single scaffold sequence. Here, the scaffold region may further comprise a linker, and the engineered crRNA scaffold sequence and the engineered tracrRNA scaffold sequence may be connected to each other via the linker.

In an embodiment, the scaffold region may comprise i) a sequence of SEQ ID NO: 101 in which substitution and/or deletion of one or more nucleotides are made, and/or insertion of one or more other nucleotides is made, and ii) a sequence of SEQ ID NO: 110 in which substitution and/or deletion of one or more nucleotides are made, and/or insertion of one or more other nucleotides is made. Here, the scaffold region may optionally further comprise a linker. For example, the scaffold region may have SEQ ID NO: 106 and SEQ ID NO: 116. Alternatively, the scaffold region may comprise SEQ ID NO: 116, a linker, and SEQ ID NO: 106 sequentially in a 5′ to 3′ direction. For example, the scaffold region may have 5′-ACCGCUUCACUUAGAGUGAAGGUGGGCUGCUUGCAUCAGCCUAAUGU CGAGAAGUGCUUUCUUCGGAAAGUAACCCUCGAAACAAAGAAAGGAAU GCAAC-3′ (SEQ ID NO: 124). As another example, the scaffold region may have SEQ ID NO: 106 and SEQ ID NO: 114. Alternatively, the scaffold region may comprise SEQ ID NO: 114, a linker, and SEQ ID NO: 106 sequentially in a 5′ to 3′ direction. For example, the scaffold region may have 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUUCGGAA AGUAACCCUCGAAACAAAGAAAGGAAUGCAAC-3′ (SEQ ID NO: 125). As yet another example, the scaffold region may have SEQ ID NO: 107 and SEQ ID NO: 118. Alternatively, the scaffold region may comprise SEQ ID NO: 118, a linker, and SEQ ID NO: 107 sequentially in a 5′ to 3′ direction. For example, the scaffold region may have

(SEQ ID NO: 126)
5′-ACCGCUUCACCAAAAGCUGUCCCUUAGGGGAUUAGAACUUGAGUGAA
GGUGGGCUGCUUGCAUCAGCCUAAUGUCGAGAAGUGCUUUCUUCGGAA
AGUAACCCUCGAAACAAAUUCAGUGCUCCUCUCCAAUUCUGCACAAGAA
AGUUGCAGAACCCGAAUAGAGCAAUGAAGGAAUGCAAC-3′.

### 2-3) U-Rich Tail Sequence

The guide RNA may optionally further comprise a U-rich tail sequence. The U-rich tail sequence is a uridine (U)-rich sequence added to the 3′ end of the guide RNA.

The U-rich tail sequence may be 5′-(UₐN)_{d}Uₑ-3′, 5′-UₐVUₐVUₑ-3′, or 5′-UₐVUₐVUₐVUₑ-3′. Here, N may be A, C, G or U, and each V may be independently A, C or G. Here, a may be an integer of 0 to 4, d may be an integer of 0 to 3, and e may be an integer of 1 to 10.

In an embodiment, the U-rich tail sequence may be 5′-(UₐU)_{d}Uₑ-3′, that is, Uₓ. Here, x may be an integer of 0 to 22. For example, the U-rich tail sequence may be U, UU, UUU, UUUU, UUUUU, UUUUUU, UUUUUUU, UUUUUUUU or UUUUUUUUU.

In another embodiment, the U-rich tail sequence may be 5′-(UₐA)_{d}Uₑ-3′, 5′-(UₐG)_{d}Uₑ-3′, or 5′-(UₐC)_{d}Uₑ-3′. For example, the U-rich tail sequence may be UUUUAUUUU, UUUAUUUUU, UUUUGUUUU, UGUGUGUU, UUUUCUUUU, or UUCUUCUUU.

In yet another embodiment, the U-rich tail sequence may be 5′-UₐAUₐAUₑ-3′, 5′-UₐAUₐCUₑ-3′, 5′-UₐAUₐGUₑ-3′, 5′-UₐCUₐAUₑ-3′, 5′-UₐCUₐCUₑ-3′, 5′-UₐCUₐGUₑ-3′, 5′-UₐGUₐAUₑ-3′, 5′-UₐGUₐCUₑ-3′, or 5′-UₐGUₐGUₑ-3′. For example, the U-rich tail sequence may be UUAUUUAUU, UUUCUAUUUU (SEQ ID NO: 127), UGUCU, UUAUGUUUUU (SEQ ID NO: 128), or UCUUUUGUU.

In still yet another embodiment, the U-rich tail sequence may be 5′-UₐAUₐAUₐAUₑ-3′, 5′-UₐAUₐAUₐCUₑ-3′, 5′-UₐAUₐAUₐGUₑ-3′, 5′-UₐAUₐCUₐAUₑ- 3′, 5′-UₐAUₐCUₐCUₑ-3′, 5′-UₐAUₐCUₐGUₑ-3′, 5′-UₐAUₐGUₐAUₑ-3′, 5′-UₐAUₐGUₐCUₑ-3′, 5′-UₐAUₐGUₐGUₑ-3′, 5′-UₐCUₐAUₐAUₑ-3′, 5′-UₐCUₐAUₐCUₑ-3′, 5′-UₐCUₐAUₐGUₑ-3′, 5′-UₐCUₐCUₐAUₑ-3′, 5′-UₐCUₐCUₐCUₑ-3′, 5′-UₐCUₐCUₐGUₑ-3′, 5′-UₐCUₐGUₐAUₑ-3′, 5′-UₐCUₐGUₐCUₑ-3′, 5′-UₐCUₐGUₐGUₑ-3′, 5′-UₐGUₐAUₐAUₑ-3′, 5′-UₐGUₐAUₐCUₑ3′, 5′-UₐGUₐAUₐGUₑ-3′, 5′-UₐGUₐCUₐAUₑ-3′, 5′-UₐGUₐCUₐCUₑ-3′, 5′-UₐGUₐCUₐGUₑ-3′, 5′-UₐGUₐGUₐAUₑ-3′, 5′-UₐGUₐGUₐCUₑ-3′, or 5′-UₐGUₐGUₐGUₑ-3′. For example, the U-rich tail sequence may be UUAUUCUG, UUCUCUUCU, or UGUUAUAUU.

### 2-4) Examples of Guide RNA

Based on the previous description, examples of the guide RNA will be described. Specific descriptions of the components included in the examples below are the same as those described above for the corresponding components. The examples are for illustrative purposes only, and the scope of the present disclosure is not limited thereto.

In an embodiment, the guide RNA may comprise a guide sequence, a wild-type crRNA scaffold sequence, and a wild-type tracrRNA scaffold sequence. Alternatively, the guide RNA may comprise a guide sequence, a wild-type crRNA scaffold sequence, a linker, and a wild-type tracrRNA scaffold sequence. The guide RNA may optionally further comprise a U-rich tail sequence.

In an embodiment, the guide RNA may comprise a wild-type tracrRNA scaffold sequence, a wild-type crRNA scaffold sequence, and a guide sequence. Here, the guide sequence may be covalently linked to the 3′ end of the wild-type crRNA scaffold sequence. Here, the wild-type tracrRNA scaffold sequence and the wild-type crRNA scaffold sequence may be each independently an RNA molecule. Here, the guide RNA may be a dual guide RNA. The guide RNA may optionally further comprise a U-rich tail sequence. Here, the U-rich tail sequence may be covalently linked to the 3′ end of the guide sequence. As an example, the guide RNA may comprise SEQ ID NO: 110 and 5′-[SEQ ID NO: 101]-[guide sequence]-3′. Here, the guide sequence may be any one sequence selected from the sequences described in Table 3 or Table 4. As another example, the guide RNA may comprise SEQ ID NO: 110 and 5′-[SEQ ID NO: 101]-[guide sequence]-[U-rich-tail sequence]-3′. Here, the guide sequence may be any one sequence selected from the sequences described in Table 3 or Table 4.

In another embodiment, the guide RNA may comprise a wild-type tracrRNA scaffold sequence, a linker, a wild-type crRNA scaffold sequence, and a guide sequence. Here, the wild-type tracrRNA scaffold sequence, the linker, the wild-type crRNA scaffold sequence, and the guide sequence may be located sequentially in a 5′ to 3′ direction (5′-[wild-type tracrRNA scaffold sequence]-[linker]-[wild-type crRNA scaffold sequence]-[guide sequence]-3′). The guide RNA may be a single guide RNA. The guide RNA may optionally further comprise a U-rich tail sequence. Here, the U-rich tail sequence may be covalently linked to the 3′ end of the guide sequence. As an example, the guide RNA may comprise 5′-[SEQ ID NO: 110]-[linker]-[SEQ ID NO: 101]-[guide sequence]-3′. Here, the guide sequence may be any one sequence selected from the sequences described in Table 3 or Table 4. In another example, the guide RNA may comprise 5′-[SEQ ID NO: 110]-[linker]-[SEQ ID NO: 101]-[guide sequence]-[U-rich tail sequence]-3′. Here, the guide sequence may be any one sequence selected from the sequences described in Table 3 or Table 4.

In another embodiment, the guide RNA may comprise a guide sequence, an engineered crRNA scaffold sequence, and an engineered tracrRNA scaffold sequence. Alternatively, the guide RNA may comprise a guide sequence, an engineered crRNA scaffold sequence, a linker, and an engineered tracrRNA scaffold sequence. The guide RNA may optionally further comprise a U-rich tail sequence.

In an embodiment, the guide RNA may comprise an engineered tracrRNA scaffold sequence, an engineered crRNA scaffold sequence, and a guide sequence. Here, the guide sequence may be covalently linked to the 3′ end of the engineered crRNA scaffold sequence. Here, the engineered tracrRNA scaffold sequence and the engineered crRNA scaffold sequence may be each independently an RNA molecule. Here, the guide RNA may be a dual guide RNA. The guide RNA may optionally further comprise a U-rich tail sequence. Here, the U-rich tail sequence may be covalently linked to the 3′ end of the guide sequence. As an example, the guide RNA may comprise SEQ ID NO: 116 and 5′-[SEQ ID NO: 106]-[guide sequence]-3′. Alternatively, the guide RNA may comprise SEQ ID NO: 116 and 5′-[SEQ ID NO: 106]-[guide sequence]-[U-rich tail sequence]-3′. Here, the guide sequence may be any one sequence selected from the sequences described in Table 3 or Table 4. As another example, the guide RNA may comprise SEQ ID NO: 114 and 5′-[SEQ ID NO: 106]-[guide sequence]-3′. Alternatively, the guide RNA may comprise SEQ ID NO: 114 and 5′-[SEQ ID NO: 106]-[guide sequence]-[U-rich tail sequence]-3′. Here, the guide sequence may be any one sequence selected from the sequences described in Table 3 or Table 4. As yet another example, the guide RNA may comprise SEQ ID NO: 118 and 5′-[SEQ ID NO: 107]-[guide sequence]-3′. Alternatively, the guide RNA may comprise SEQ ID NO: 118 and 5′-[SEQ ID NO: 107]-[guide sequence]-[U-rich tail sequence]-3′. Here, the guide sequence may be any one sequence selected from the sequences described in Table 3 or Table 4.

In another embodiment, the guide RNA may comprise an engineered tracrRNA scaffold sequence, a linker, an engineered crRNA scaffold sequence, and a guide sequence. Here, the engineered tracrRNA scaffold sequence, the linker, the engineered crRNA scaffold sequence, and the guide sequence may be located sequentially in a 5′ to 3′ direction (5′-[engineered tracrRNA scaffold sequence]-[linker]-[engineered crRNA scaffold sequence]-[guide sequence]-3′). The guide RNA may be a single guide RNA. The guide RNA may optionally further comprise a U-rich tail sequence. Here, the U-rich tail sequence may be covalently linked to the 3′ end of the guide sequence. As an example, the guide RNA may comprise 5′-[SEQ ID NO: 116]-[linker]-[SEQ ID NO: 106]-[guide sequence]-3′. Alternatively, the guide RNA may comprise 5′-[SEQ ID NO: 116]-[linker]-[SEQ ID NO: 106]-[guide sequence]-[U-rich tail sequence]-3′. Here, the guide sequence may be any one sequence selected from the sequences described in Table 3 or Table 4. As another example, the guide RNA may comprise 5′-[SEQ ID NO: 114]-[linker]-[SEQ ID NO: 106]-[guide sequence]-3′. Alternatively, the guide RNA may comprise 5′-[SEQ ID NO: 114]-[linker]-[SEQ ID NO: 106]-[guide sequence]-[U-rich tail sequence]-3′. Here, the guide sequence may be any one sequence selected from the sequences described in Table 3 or Table 4. As yet another example, the guide RNA may comprise 5′-[SEQ ID NO: 118]-[linker]-[SEQ ID NO: 107]-[guide sequence]-3′. Alternatively, the guide RNA may comprise 5′-[SEQ ID NO: 118]-[linker]-[SEQ ID NO: 107]-[guide sequence]-[U-rich tail sequence]-3′. Here, the guide sequence may be any one sequence selected from the sequences described in Table 3 or Table 4.

### 3. Cas12f1 (Cas14a1) Protein

The CRISPR/Cas12f1 system for treatment of LCA10 disease comprises a Cas12f1 (Cas14a1) protein. The Cas12f1 protein, which is a major protein component of the CRISPR/Cas12f1 (Cas14a1) system, is one of the effector proteins named Cas14 in a previous study (Harrington et al., Science, 362, 839-842 (2018)) and is also called a Cas14a1 protein. In the present specification, the Cas12f1 protein is used interchangeably with the Cas14a1 protein or Cas12f1 (Cas14a1) protein. The Cas12f1 protein disclosed herein may be a wildtype Cas12f1 protein (wildtype Cas14a1 protein) existing in nature. Alternatively, the Cas12f1 protein may be a variant of the wildtype Cas12f1 protein, wherein the variant is referred to as “Cas12f1 variant” or “Cas14a1 variant.” The Cas12f1 variant may be a variant having the same function as the wildtype Cas12f1 protein, a variant of which some or all functions are modified as compared with the wildtype Cas12f1 protein, and/or a variant to which an additional function is added as compared with the wildtype Cas12f1 protein. The meaning of the Cas12f1 protein may be appropriately interpreted according to the context, and is interpreted in the broadest sense except for a particular case. Hereinafter, the Cas12f1 protein will be described in detail.

### 3-1) Wildtype Cas12f1 (Cas14a1) Protein

The Cas12f1 protein may be a wild-type Cas12f1 protein. Here, the Cas12f1 protein is capable of cleaving a double-strand or single-strand of a target nucleic acid or target gene. The Cas12f1 protein is capable of recognizing a protospacer adjacent motif (PAM) sequence present in a target nucleic acid or target gene. Here, the PAM sequence is a unique sequence determined depending on the Cas14a1 protein. The PAM sequence for the Cas12f1 protein may be a T-rich sequence. The PAM sequence for the Cas12f1 protein may be 5′-TTTR-3′. Here, R may be A or G. For example, the PAM sequence may be 5′-TTTA-3′ or 5′-TTTG-3′.

In an embodiment, the Cas12f1 protein may be derived from the Cas14 family (Harrington et al., Science 362, 839-842 (2018); US 2020/0172886 A1).

In another embodiment, the Cas12f1 protein may be a Cas14a1 protein derived from an uncultured archaeon (Harrington et al., Science 362, 839-842 (2018); US 2020/0172886 A1). For example, the Cas14a1 protein may be the amino acid sequence of SEQ ID NO: 129.

TABLE 5
Amino acid sequence of Cas14a1 protein

| Name | Amino acid sequence |
|---|---|
| Cas12f1 | MAKNTITKTLKLRIVRPYNSAEVEKIVADEKNNREKI |
| protein | ALEKNKDKVKEACSKHLKVAAYCTTQVERNACLFC |
| | KARKLDDKFYQKLRGQFPDAVFWQEISEIFRQLQK |
| | QAAEIYNQSLIELYYEIFIKGKGIANASSVEHYLSDVC |
| | YTRAAELFKNAAIASGLRSKIKSNFRLKELKNMKSGL |
| | PTTKSDNFPIPLVKQKGGQYTGFEISNHNSDFIIKIPF |
| | GRWQVKKEIDKYRPWEKFDFEQVQKSPKPISLLLST |
| | QRRKRNKGWSKDEGTEAEIKKVMNGDYQTSYIEVK |
| | RGSKIGEKSAWMLNLSIDVPKIDKGVDPSIIGGIDVG |
| | VKSPLVCAINNAFSRYSISDNDLFHFNKKMFARRRIL |
| | LKKNRHKRAGHGAKNKLKPITILTEKSERFRKKLIER |
| | WACEIADFFIKNKVGTVQMENLESMKRKEDSYFNIR |
| | LRGFWPYAEMQNKIEFKLKQYGIEIRKVAPNNTSKT |
| | CSKCGHLNNYFNFEYRKKNKFPHFKCEKCNFKENA |
| | DYNAALNISNPKLKSTKEEP (SEQ ID NO: 129) |

### 3-2) Cas12f1 (Cas14a1) Variant—Cas12f1 (Cas14a1) Mutant

The Cas12f1 protein may be a Cas12f1 variant. The Cas12f1 variant may be a variant of a wildtype Cas12f1 protein in which at least one amino acid in the amino acid sequence of the wildtype Cas12f1 protein is modified. Here, the modification may be deletion and/or substitution. Alternatively, the Cas12f1 variant may be a variant of a wildtype Cas12f1 protein in which at least one amino acid sequence is added to both ends of the amino acid sequence of the wild-type Cas12f1 protein and/or within the amino acid sequence thereof. Here, the modification may be insertion. Here, the Cas12f1 variant is referred to as “Cas12f1 mutant” or “Cas14a1 mutant.”

In an embodiment, the Cas12f1 mutant may be obtained by deleting at least one amino acid in an amino acid sequence of a wildtype Cas12f1 protein. For example, the Cas12f1 mutant may be obtained by deleting at least one amino acid in a RuvC domain included in a wildtype Cas12f1 protein. Alternatively, the Cas12f1 mutant may be obtained by deleting at least one amino acid in a domain that recognizes a PAM included in a wildtype Cas12f1 protein. Alternatively, the Cas12f1 mutant may be obtained by deleting at least one amino acid in the amino acid sequence of SEQ ID NO: 129.

In another embodiment, the Cas12f1 mutant may be obtained by substituting at least one amino acid in an amino acid sequence of a wildtype Cas12f1 protein with other amino acid(s). Here, the substitution may be such that one amino acid is substituted with one other amino acid. Alternatively, the substitution may be such that one amino acid is substituted with a plurality of other amino acids. Alternatively, the substitution may be such that a plurality of amino acids are substituted with one other amino acid. Alternatively, the substitution may be such that a plurality of amino acids are substituted with a plurality of other amino acids wherein the number of amino acids to be substituted and the number of substituting amino acids may be the same or different from each other. For example, the Cas12f1 mutant may be obtained by substituting at least one amino acid in a RuvC domain included in a wildtype Cas12f1 protein with other amino acid(s). Alternatively, the Cas12f1 mutant may be obtained by substituting at least one amino acid in a domain, which recognizes a PAM included in a wildtype Cas12f1 protein, with other amino acid(s). Alternatively, the Cas12f1 mutant may be obtained by substituting at least one amino acid in the amino acid sequence of SEQ ID NO: 129 with other amino acid(s).

The Cas12f1 mutant may be a variant having the same function as a wildtype Cas12f1 protein or a variant of which some or all functions are modified as compared with a wildtype Cas12f1 protein. For example, the Cas12f1 mutant may be a variant in which modification is made to cleave only one strand in a double-strand of a target nucleic acid. Alternatively, the Cas12f1 mutant may be a variant in which modification is made to recognize a PAM sequence other than 5′-TTTA-3′ or 5′-TTTG-3′.

The inventors of the present application made research and have found that among the mutants in which amino acids are added to the N-terminus and/or C-terminus of the amino acid sequence of the wild-type Cas12f1 protein, there is a Cas12f1 mutant having the same function as the Cas12f1 protein. Furthermore, a Cas12f1 mutant having the same function as the wild-type Cas12f1 protein was invented by adding an amino acid sequence of a certain length to the N-terminus of the wild-type Cas12f1 protein. The Cas12f1 variant is disclosed in detail in Korean Patent Application No. 10-2021-0181875, and the contents thereof regarding the Cas12f1 variant or functional analog are also incorporated herein by reference.

In an embodiment, the Cas12f1 variant may be a variant of a wild-type Cas12f1 protein in which 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids are added to the N-terminus and/or C-terminus of the wild-type Cas12f1 protein.

In an embodiment, the Cas12f1 variant may be a variant of a wild-type Cas12f1 protein in which the number of amino acids within the above-mentioned two numerical ranges is added to the N-terminus and/or C-terminus of the wild-type Cas12f1 protein. For example, the Cas12f1 variant may be a variant of a wild-type Cas12f1 protein in which 26 to 28 amino acids are added to the N-terminus of the wild-type Cas12f1 protein.

In an embodiment, the Cas12f1 variant may be a protein represented by an amino acid sequence selected from SEQ ID NOS: 288 to 291. Among them, the protein represented by SEQ ID NO: 288 is also known as transposon-associated transposase B (TnpB) derived from Candidatus Woesearchaeota archaeon, and the terms such as Cas12f1 protein, Cas12f1 variant, Cas12f1 functional analog, TnpB, and TnpB functional analog may be used interchangeably in the present specification.

### 3-3) Cas12f1 (Cas14a1) Variant—Cas12f1 (Cas14a1) Fusion Protein

The Cas12f1 protein may be a Cas12f1 variant. The Cas12f1 variant may be a variant obtained by adding a domain, peptide or protein having an additional function to a wildtype Cas12f1 protein or a Cas12f1 mutant. Here, the variant, to which the domain, peptide or protein having an additional function is added, is referred to as “Cas12f1 fusion protein” or “Cas14a1 fusion protein.” The domain, peptide or protein having an additional function may be added to the N-terminus and/or C-terminus of a wildtype Cas12f1 protein or a Cas12f1 mutant, and/or within the amino acid sequence thereof. The domain, peptide or protein having an additional function may be a domain, peptide or protein having the same or different function as a wildtype Cas12f1 protein. For example, the domain, peptide or protein having an additional function may be a domain, peptide or protein having methylase activity, demethylase activity, transcription activation activity, transcription repression activity, transcription release factor activity, histone modification activity, RNA cleavage activity or nucleic acid binding activity, or may be a tag or reporter protein used for isolation and purification of a protein (including a peptide), but is not limited thereto. Alternatively, the domain, peptide or protein having an additional function may be reverse transcriptase or deaminase.

### 3-4) Cas12f1 (Cas14a1) Variant—Others

The Cas12f1 protein may be a Cas12f1 variant. The Cas12f1 variant may be a variant of a wildtype Cas12f1 protein, a Cas12f1 mutant, or a Cas12f1 fusion protein, in which a nuclear localization sequence (NLS) or a nuclear export sequence (NES) is optionally further included. The NLS and NES are signal peptides for positioning the Cas12f1 protein in or outside a cell. These terms include all meanings recognized by those skilled in the art, and may be appropriately interpreted according to the context. For example, the NLS may be, but is not limited to, an NLS sequence derived from the NLS of an SV40 virus large T-antigen, having the amino acid sequence PKKKRKV (SEQ ID NO: 130); the NLS from a nucleoplasmin (for example, the nucleoplasmin bipartite NLS having the sequence KRPAATKKAGQAKKKK (SEQ ID NO: 131)); the c-myc NLS having the amino acid sequence PAAKRVKLD (SEQ ID NO: 132) or RQRRNELKRSP (SEQ ID NO: 133); the hRNPA1 M9 NLS having the sequence NQSSNFGPMKGGNFGGRSSGPYGGGGQYFAKPRNQGGY (SEQ ID NO: 134); the sequence RMRIZFKNKGKDTAELRRRRVEVSVELRKAKKDEQILKRRNV (SEQ ID NO: 135) of an IBB domain from importin alpha; the sequences VSRKRPRP (SEQ ID NO: 136) and PPKKARED (SEQ ID NO: 137) of myoma T protein; the sequence PQPKKKPL (SEQ ID NO: 138) of human p53; the sequence SALIKKKKKMAP (SEQ ID NO: 139) of mouse c-abl IV; the sequences DRLRR (SEQ ID NO: 140) and PKQKKRK (SEQ ID NO: 141) of influenza virus NS1; the sequence RKLKKKIKKL (SEQ ID NO: 142) of hepatitis virus delta antigen; the sequence REKKKFLKRR (SEQ ID NO: 143) of mouse Mxl protein; the sequence KRKGDEVDGVDEVAKKKSKK (SEQ ID NO: 144) of human poly (ADP-ribose) polymerase; or the sequence RKCLQAGMNLEARKTKK (SEQ ID NO: 145) of steroid hormone receptor (human) glucocorticoid.

In addition, the Cas12f1 variant may be a variant of a wildtype Cas12f1 protein, a Cas12f1 mutant, or a Cas12f1 fusion protein, in which a tag is optionally further included. The tag is a functional domain, peptide or protein for isolation and purification and/or tracking of the Cas12f1 protein, and the tag includes, but is not limited to, a tag protein such as a histidine (His) tag, a V5 tag, a FLAG tag, an influenza hemagglutinin (HA) tag, a Myc tag, a VSV-G tag, and a thioredoxin (Trx) tag; a fluorescent protein such as green fluorescent protein (GFP), yellow fluorescent protein (YFP), cyan fluorescent protein (CFP), blue fluorescent protein (BFP), HcRED, and DsRed; and a reporter protein (enzyme) such as glutathione-S-transferase (GST), horseradish peroxidase (HRP), chloramphenicol acetyltransferase (CAT) beta-galactosidase, beta-glucuronidase, and luciferase.

### 4. Guide RNA-Cas12f1(Cas14a1) Protein(s) Complex (CRISPR/Cas12f1 Complex or CRISPR Complex)

The CRISPR/Cas12f1 system for treatment of LCA10 disease may operate in a form of a guide RNA-Cas12f1 (Cas14a1) protein(s) complex (CRISPR/Cas12f1 complex or CRISPR complex). The CRISPR complex comprises a guide RNA and a Cas12f1 protein. Here, the CRISPR complex comprises a guide RNA and two Cas12f1 proteins (Satoru N. Takeda et al., Molecular Cell, 81, 1-13, (2021)). The CRISPR complex may be a ribonucleoprotein (RNP) formed by interaction of a guide RNA with a Cas12f1 protein. Here, the CRISPR complex may be a ribonucleoprotein (RNP) formed by interaction of a guide RNA with one Cas12f1 protein or an RNP formed by interaction of a guide RNA with two Cas12f1 proteins (Satoru N. Takeda et al., Molecular Cell, 81, 1-13, (2021)). The CRISPR complex is referred to as “guide RNA-Cas12f1 (Cas14a1) protein complex”, “CRISPR/Cas12f1 complex,” or “CRISPR/Cas14a1 complex”, and the terms are used interchangeably herein. Specific descriptions of the components included in the examples below are the same as those described above for the corresponding components. The examples are for illustrative purposes only, and the scope of the present disclosure is not limited thereto.

In an embodiment, the CRISPR complex may be an RNP formed by combination of a wildtype Cas12f1 protein and a guide RNA. Here, the CRISPR complex may comprise two wildtype Cas12f1 proteins and a guide RNA. Here, the wild-type Cas12f1 protein may have the amino acid sequence of SEQ ID NO: 129. Here, the guide RNA may be one of the examples described in the section “2-4) Examples of guide RNA” described above.

In another embodiment, the CRISPR complex may be an RNP formed by combination of a Cas12f1 variant and a guide RNA. Here, the CRISPR complex may comprise two Cas12f1 variants and a guide RNA. Here, the Cas12f1 variant may be a Cas12f1 mutant or a Cas12f1 fusion protein. Here, the guide RNA may be one of the examples described in the section “2-4) Examples of guide RNA” described above.

In yet another embodiment, the CRISPR complex may be an RNP formed by combination of a wild-type Cas12f1 protein, a Cas12f1 mutant, and a guide RNA. Here, the wild-type Cas12f1 protein may have the amino acid sequence of SEQ ID NO: 129. Here, the Cas12f1 variant may be a Cas12f1 mutant or a Cas12f1 fusion protein. Here, the guide RNA may be one of the examples described in the section “2-4) Examples of guide RNA” described above.

### <Composition for Treatment of LCA10 Disease>

In an aspect disclosed herein, there is provided a composition for treatment of LCA10 disease. The composition comprises a CRISPR/Cas12f1 system. The CRISPR/Cas12f1 system is as described in the section “<CRISPR/Cas12f1 system for treatment of LCA10 disease>” described above, and has the same characteristics and structure as each configuration described in the section. The CRISPR/Cas12f1 system is used to induce deletion or inversion of all or part of the 128 base pair cryptic exon. Therefore, the composition effectively deletes or inverts all or part of the cryptic exon that causes LCA10 disease, and thus may be used as an effective therapeutic agent for LCA10 disease.

More specifically, the composition may comprise:
at least one guide RNA targeting the CEP290 gene or a nucleic acid encoding the same; and
a Cas12f1 protein or a nucleic acid encoding the same.

Here, the at least one guide RNA targeting the CEP290 gene may complementarily bind to a target sequence present in the CEP290 gene.

Here, the nucleic acid encoding the guide RNA may be DNA encoding the guide RNA to transcribe the guide RNA.

Here, the nucleic acid encoding the Cas12f1 protein may be a nucleic acid encoding a wild-type Cas12f1 protein or a nucleic acid encoding a Cas12f1 variant. The nucleic acid encoding the Cas12f1 protein may be codon-optimized according to a target into which the Cas12f1 protein is to be introduced. “Codon optimization” refers to a process of modifying a native nucleic acid sequence for enhanced expression in a cell of interest by replacing at least one codon in the native sequence with a codon, which is used more frequently or most frequently in a gene of a target cell, while maintaining its native amino acid sequence. Different species have specific biases for specific codons of specific amino acids, and codon bias (differences in codon usage between organisms) is often correlated with translation efficiency of an mRNA, which is considered to be dependent on the nature of codons being translated and availability of specific tRNA molecules. Predominance of tRNA selected in a cell generally reflects the most frequently used codon in peptide synthesis. Thus, genes may be tailored for optimal gene expression in a given organism based on codon optimization. In an embodiment, the nucleic acid encoding the Cas12f1 protein may be a nucleic acid encoding a human codon-optimized Cas12f1 protein. For example, the nucleic acid encoding the human codon-optimized Cas12f1 protein may be SEQ ID NO: 146.

TABLE 6
Human codon-optimized nucleic acid encoding Cas12f1 protein

| Name | Sequence |
|---|---|
| Human | ATGGCCAAGAACACAATTACAAAGACACTGAAGCTGAGGATCGTGAGACCATACAACAGCGC |
| codon- | TGAGGTCGAGAAGATTGTGGCTGATGAAAAGAACAACAGGGAAAAGATCGCCCTCGAGAAGA |
| optimized | ACAAGGATAAGGTGAAGGAGGCCTGCTCTAAGCACCTGAAAGTGGCCGCCTACTGCACCACAC |
| Cas12f1 | AGGTGGAGAGGAACGCCTGTCTGTTTTGTAAAGCTCGGAAGCTGGATGATAAGTTTTACCAGA |
| encoding | AGCTGCGGGGCCAGTTCCCCGATGCCGTCTTTTGGCAGGAGATTAGCGAGATCTTCAGACAGC |
| DNA | TGCAGAAGCAGGCCGCCGAGATCTACAACCAGAGCCTGATCGAGCTCTACTACGAGATCTTCA |
| | TCAAGGGCAAGGGCATTGCCAACGCCTCCTCCGTGGAGCACTACCTGAGCGACGTGTGCTACA |
| | CAAGAGCCGCCGAGCTCTTTAAGAACGCCGCTATCGCTTCCGGGCTGAGGAGCAAGATTAAGA |
| | GTAACTTCCGGCTCAAGGAGCTGAAGAACATGAAGAGCGGCCTGCCCACTACAAAGAGCGACA |
| | ACTTCCCAATTCCACTGGTGAAGCAGAAGGGGGGCCAGTACACAGGGTTCGAGATTTCCAACC |
| | ACAACAGCGACTTTATTATTAAGATCCCCTTTGGCAGGTGGCAGGTCAAGAAGGAGATTGACA |
| | AGTACAGGCCCTGGGAGAAGTTTGATTTCGAGCAGGTGCAGAAGAGCCCCAAGCCTATTTCCC |
| | TGCTGCTGTCCACACAGCGGCGGAAGAGGAACAAGGGGTGGTCTAAGGATGAGGGGACCGAG |
| | GCCGAGATTAAGAAAGTGATGAACGGCGACTACCAGACAAGCTACATCGAGGTCAAGCGGGG |
| | CAGTAAGATTGGCGAGAAGAGCGCCTGGATGCTGAACCTGAGCATTGACGTGCCAAAGATTG |
| | ATAAGGGCGTGGATCCCAGCATCATCGGAGGGATCGATGTGGGGGTCAAGAGCCCCCTCGTGT |
| | GCGCCATCAACAACGCCTTCAGCAGGTACAGCATCTCCGATAACGACCTGTTCCACTTTAACAA |
| | GÅAGATGTTCGCCCGGCGGAGGATTTTGCTCAAGAAGAACCGGCACAAGCGGGCCGGACACG |
| | GGGCCAAGAACAAGCTCAAGCCCATCACTATCCTGACCGAGAAGAGCGAGAGGTTCAGGAAG |
| | AAGCTCATCGAGAGATGGGCCTGCGAGATCGCCGATTTCTTTATTAAGAACAAGGTCGGAACA |
| | GTGCAGATGGAGAACCTCGAGAGCATGAAGAGGAAGGAGGATTCCTACTTCAACATTCGGCT |
| | GAGGGGGTTCTGGCCCTACGCTGAGATGCAGAACAAGATTGAGTTTAAGCTGAAGCAGTACG |
| | GGATTGAGATCCGGAAGGTGGCCCCCAACAACACCAGCAAGACCTGCAGCAAGTGCGGGCACC |
| | TCAACAACTACTTCAACTTCGAGTACCGGAAGAAGAACAAGTTCCCACACTTCAAGTGCGAGA |
| | AGTGCAACTTTAAGGAGAACGCCGATTACAACGCCGCCCTGAACATCAGCAACCCTAAGCTGA |
| | AGAGCACTAAGGAGGAGCCC (SEQ ID NO: 146) |

Here, the composition may optionally further comprise at least one guide RNA or a nucleic acid encoding the same. Here, the composition may comprise a plurality of guide RNAs, and each of the plurality of guide RNAs may recognize a different target sequence. Here, the composition optionally further comprises at least one Cas12f1 protein or a nucleic acid encoding the same. Here, the composition may comprise two or more Cas12f1 proteins having different amino acid sequences. Here, the two or more Cas12f1 proteins having different amino acid sequences may be a wild-type Cas12f1 protein and a Cas14a1 variant.

The guide RNA is as described in the section “2. Guide RNA” described above, and has the same characteristics and structure as each configuration described in the section. In addition, the Cas12f1 protein is as described in the section “3. Cas12f1 (Cas14a1) protein” described above, and has the same characteristics and structure as each configuration described in the section.

The composition may be in a form of a nucleic acid. Alternatively, the composition may be in a form of a mixed form of a nucleic acid and a protein. Hereinafter, the form of the composition will be described in detail.

### 1. Form of Nucleic Acid

The composition may be in a form of a nucleic acid. The nucleic acid may be in a form of DNA or RNA, or in a mixed form thereof. The nucleic acid may be in a form of a vector. Here, the vector may be a viral vector or a non-viral vector.

In addition, a shape of the nucleic acid may be circular or linear.

In addition, a shape of the nucleic acid may be double-stranded or single-stranded.

### 1-1) Viral Vector

The composition may be in a form of a viral vector. For example, the viral vector may be one or more viral vectors selected from the group consisting of a retroviral (retrovirus) vector, a lentiviral (lentivirus) vector, an adenoviral (adenovirus) vector, an adeno-associated viral (adeno-associated virus; AAV) vector, a vaccinia viral (vaccinia virus) vector, a poxviral (poxvirus) vector, and a herpes simplex viral (herpes simplex virus) vector.

In an embodiment, the composition may be in a form of a viral vector. That is, the composition may comprise a nucleic acid encoding a guide RNA and a nucleic acid encoding a Cas12f1 protein in a form of a viral vector. Here, the composition may comprise a first viral vector comprising a nucleic acid encoding a guide RNA and a second viral vector comprising a nucleic acid encoding a Cas12f1 protein. Alternatively, the composition may comprise a viral vector comprising both a nucleic acid encoding a guide RNA and a nucleic acid encoding a Cas12f1 protein. In an embodiment, the composition may comprise an adeno-associated viral vector comprising both a nucleic acid encoding a guide RNA and a nucleic acid encoding a Cas12f1 protein.

In another embodiment, in a case where the composition comprises nucleic acids encoding a plurality of guide RNAs and a nucleic acid encoding a Cas12f1 protein, the composition may comprise a first viral vector comprising all of the nucleic acids encoding the plurality of guide RNAs and a second viral vector comprising the nucleic acid encoding the Cas12f1 protein. Alternatively, the composition may be in a form of a single viral vector comprising all of nucleic acids encoding a plurality of guide RNAs and a nucleic acid encoding a Cas12f1 protein. Alternatively, the composition may comprise a plurality of (as much as the number of guide RNAs) viral vectors, each of which comprises a nucleic acid encoding one guide RNA and a nucleic acid encoding a Cas12f1 protein.

In another embodiment, in a case where the composition comprises a nucleic acid encoding a guide RNA and nucleic acids encoding two or more Cas12f1 proteins (for example, a first Cas12f1 protein and a second Cas12f1 protein), the composition may comprise a first viral vector comprising a nucleic acid encoding a guide RNA, a second viral vector comprising a nucleic acid encoding a first Cas12f1 protein, and a third viral vector comprising a nucleic acid encoding a second Cas12f1 protein. Alternatively, the composition may be in a form of a single viral vector comprising both a nucleic acid encoding a guide RNA and nucleic acids encoding two or more Cas12f1 proteins (for example, a first Cas12f1 protein and a second Cas12f1 protein). Alternatively, the composition may comprise a first viral vector comprising a nucleic acid encoding a guide RNA and a nucleic acid encoding a first Cas12f1 protein, and a second viral vector comprising a nucleic acid encoding a second Cas12f1 protein.

### 1-2) Non-Viral Vector

The composition may be in a form of a non-viral vector. Here, the guide RNA in the composition may be in a form of RNA or a nucleic acid encoding the same.

The non-viral vector may be a plasmid, a phage, a naked DNA, a DNA complex, an mRNA (transcript) or a PCR amplicon, but is not limited thereto. For example, the plasmid may be selected from the group consisting of: pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19. For example, the phage may be selected from the group consisting of λgt4λB, λ-Charon, λΔz1, and M13.

In an embodiment, the composition may be in a form of a non-viral vector. That is, the composition may comprise a nucleic acid encoding a guide RNA and a nucleic acid encoding a Cas12f1 protein in a form of a non-viral vector. Here, the composition may comprise a first non-viral vector comprising a nucleic acid encoding a guide RNA and a second non-viral vector comprising a nucleic acid encoding a Cas12f1 protein. Alternatively, the composition may comprise a non-viral vector comprising both a nucleic acid encoding a guide RNA and a nucleic acid encoding a Cas12f1 protein. In an embodiment, the composition may comprise a plasmid comprising both a nucleic acid encoding a guide RNA and a nucleic acid encoding a Cas12f1 protein. In another embodiment, the composition may comprise a PCR amplicon comprising a nucleic acid encoding a guide RNA and an mRNA encoding a Cas12f1 protein. In another embodiment, the composition may comprise a PCR amplicon comprising a nucleic acid encoding a guide RNA and a naked DNA comprising a nucleic acid encoding a Cas12f1 protein.

In another embodiment, the composition may be in a form of a non-viral vector. That is, the composition may comprise nucleic acids encoding a guide RNA and a Cas12f1 protein in a form of a non-viral vector. In an embodiment, the composition may comprise a guide RNA, and an mRNA encoding a Cas12f1 protein. In another embodiment, the composition may comprise a guide RNA, and a plasmid comprising a nucleic acid encoding a Cas12f1 protein.

### 1-3) Mixing of Viral and Non-Viral Vectors

The composition may be in a mixed form of a viral vector and a non-viral vector. The description of the viral vector and the non-viral vector is as described above. Here, the guide RNA in the composition may be in a form of RNA or a nucleic acid encoding the same.

In an embodiment, the composition may comprise a viral vector comprising a nucleic acid encoding a guide RNA and a non-viral vector comprising a nucleic acid encoding a Cas12f1 protein. In an embodiment, the composition may comprise an adeno-associated viral vector comprising a nucleic acid encoding a guide RNA and an mRNA encoding a Cas12f1 protein. In another embodiment, the composition may comprise a lentiviral vector comprising a nucleic acid encoding a guide RNA and a plasmid comprising a nucleic acid encoding a Cas12f1 protein.

In another embodiment, the composition may comprise a non-viral vector comprising a nucleic acid encoding a guide RNA and a viral vector comprising a nucleic acid encoding a Cas12f1 protein. In an embodiment, the composition may comprise a PCR amplicon comprising a nucleic acid encoding a guide RNA and an adeno-associated viral vector comprising a nucleic acid encoding a Cas12f1 protein. In another embodiment, the composition may comprise a plasmid comprising a nucleic acid encoding a guide RNA and a lentiviral vector comprising a nucleic acid encoding a Cas12f1 protein.

In yet another embodiment, the composition may comprise a guide RNA, and a viral vector comprising a nucleic acid encoding a Cas12f1 protein. In an embodiment, the composition may comprise a guide RNA, and an adeno-associated viral vector comprising a nucleic acid encoding a Cas12f1 protein.

In another embodiment, the composition may comprise a guide RNA, and a non-viral vector comprising a nucleic acid encoding a Cas12f1 protein. In an embodiment, the composition may comprise a guide RNA, and an mRNA encoding a Cas12f1 protein. In another embodiment, the composition may comprise a guide RNA, and a plasmid comprising a nucleic acid encoding a Cas12f1 protein.

### 1-4) Others—Additional Component of Vector

The vectors described above may optionally further comprise a regulatory/control element, a promoter and/or an additionally expressed element.

### Regulating/Control Element

The vector may optionally comprise a regulatory/control element. Here, the regulatory/control element may be operably linked to a sequence encoding each component included in the vector (that is, a nucleic acid encoding a guide RNA and/or a nucleic acid encoding a Cas12f1 protein). The regulatory/control element may be, but is not limited to, an enhancer, an intron, a termination signal, a polyadenylation signal, a Kozak consensus sequence, an internal ribosome entry site (IRES), a splice acceptor, a 2A sequence and/or an origin of replication. Here, the origin of replication may be, but is not limited to, an f1 origin of replication, an SV40 origin of replication, a pMB1 origin of replication, an adeno origin of replication, an AAV origin of replication, and/or a BBV origin of replication.

### Promoter

The vector may optionally comprise a promoter. Here, the promoter may be operably linked to a sequence encoding each component included in the vector (that is, a nucleic acid encoding a guide RNA and/or a nucleic acid encoding a Cas12f1 protein). The promoter is not limited as long as it is capable of properly expressing a sequence encoding each component included in the vector (that is, a nucleic acid encoding a guide RNA and/or a nucleic acid encoding a Cas12f1 protein). In an embodiment, the promoter sequence may be a promoter that promotes transcription of an RNA polymerase (for example, Pol I, Pol II, or Pol III). For example, the promoter may be, but is not limited to, any one selected from: an SV40 early promoter, a mouse mammary tumor virus long terminal repeat (LTR) promoter, an adenovirus major late promoter (Ad MLP), a herpes simplex virus (HSV) promoter, a cytomegalovirus (CMV) promoter such as CMV immediate early promoter region (CMVIE), a chicken b-actin (CBA) promoter, a rous sarcoma virus (RSV) promoter, a human U6 small nuclear promoter (U6) (Miyagishi et al., Nature Biotechnology 20, 497-500 (2002)), an enhanced U6 promoter (for example, Xia et al., Nucleic Acids Res. 2003 September 1:31(17)), a human H1 promoter (H1), and a 7SK promoter.

### Additionally Expressed Element

The vector may optionally comprise an additionally expressed element. The vector may comprise a nucleic acid sequence encoding an additionally expressed element to be expressed as necessary by those skilled in the art in addition to a nucleic acid encoding a guide RNA and/or a nucleic acid encoding a Cas12f1 protein. For example, the additionally expressed element may be one of the tags described in the section “Tag” in definition of terms as described above, but is not limited thereto. For example, the additionally expressed element may be a herbicide resistance gene such as glyphosate, glufosinate ammonium, or phosphinothricin; or an antibiotic resistance gene such as ampicillin, kanamycin, G418, bleomycin, hygromycin, or chloramphenicol, but is not limited thereto.

### 2. Mixed Form of Nucleic Acid and Protein

The composition may be in a mixed form of a nucleic acid and a protein. Here, the nucleic acid is as described above in the section “1) Form of nucleic acid.” Here, the composition comprises a Cas12f1 protein. Here, the composition may comprise two Cas12f1 proteins, and the two Cas12f1 proteins may be Cas12f1 proteins having the same amino acid sequence or Cas12f1 proteins having different amino acid sequences.

In an embodiment, the composition may comprise a viral vector comprising a nucleic acid encoding a guide RNA and a Cas12f1 protein. In an embodiment, the composition may comprise an adeno-associated viral vector comprising a nucleic acid encoding a guide RNA and a Cas12f1 protein. In another embodiment, the composition may comprise an adeno-associated viral vector comprising nucleic acids encoding a plurality of guide RNAs and a Cas12f1 protein.

In another embodiment, the composition may comprise a non-viral vector comprising a nucleic acid encoding a guide RNA and a Cas12f1 protein. In an embodiment, the composition may comprise a plasmid comprising a nucleic acid encoding a guide RNA and a Cas12f1 protein. In another embodiment, the composition may comprise a PCR amplicon comprising a nucleic acid encoding a guide RNA and a Cas12f1 protein. In yet another embodiment, the composition may comprise a PCR amplicon comprising nucleic acids encoding a plurality of guide RNAs and a Cas12f1 protein.

In yet another embodiment, the composition may comprise a guide RNA and a Cas12f1 protein. Here, the composition may be in a form of an RNP which is in a form of a CRISPR/Cas12f1 complex. In a case where a composition comprises a plurality of guide RNAs and Cas12f1 proteins, the composition may comprise a plurality of CRISPR/Cas12f1 complexes, and the plurality of CRISPR/Cas12f1 complexes each may recognize a different target sequence.

### 3. Examples (1) of Composition

In an embodiment, the composition may comprise:
two or more guide RNAs targeting the CEP290 gene or nucleic acids encoding the same; and
a Cas12f1 protein or a nucleic acid encoding the same.

Here, the guide RNA may complementarily bind to a target sequence present in the CEP290 gene.

The two or more guide RNAs may comprise a first guide RNA and a second guide RNA. Here, the nucleic acids encoding the two or more guide RNAs may comprise a nucleic acid encoding the first guide RNA and a nucleic acid encoding the second guide RNA.

Here, the first guide RNA and the second guide RNA may target different target sequences present in the CEP gene.

Here, the first guide RNA may target an upstream region based on the IVS26 mutation (c.2991+1655A>G) in the intron 26 region of the CEP gene. Alternatively, the first guide RNA may target a region between the 3′ end of exon 26 of the CEP290 gene and the IVS26 mutation (c.2991+1655A>G).

Here, the second guide RNA may target a downstream region based on the IVS26 mutation (c.2991+1655A>G) in the intron 26 region of the CEP gene. Alternatively, the second guide RNA may target a region between the IVS26 mutation (c.2991+1655A>G) and the 5′ end of exon 27 of the CEP290 gene.

Here, the first guide RNA may comprise a first guide region and a first scaffold region. Here, the first guide region may have a first guide sequence. Here, the first guide sequence may be a sequence of 15 to 40 nucleotides capable of forming a complementary bond with a target sequence present in an upstream region based on the IVS26 mutation (c.2991+1655A>G) in the intron 26 region of the CEP290 gene. Alternatively, the first guide sequence may be a sequence of 15 to 40 nucleotides capable of forming a complementary bond with a target sequence present in a region between the 3′ end of exon 26 of the CEP290 gene and the IVS26 mutation (c.2991+1655A>G). Here, the complementary bond may optionally contain 0 to 5 mismatched bonds. As an example, the first guide sequence may be any one sequence selected from the guide sequences listed in Table 3. Here, the first scaffold region may comprise a crRNA scaffold sequence and a tracrRNA scaffold sequence. The first scaffold region may optionally further comprise a linker, and the linker may be located between the tracrRNA scaffold sequence and the crRNA scaffold sequence. Here, the crRNA scaffold sequence may be a wild-type crRNA scaffold sequence or an engineered crRNA scaffold sequence. As an example, the wild-type crRNA scaffold sequence may be SEQ ID NO: 101. As another example, the engineered crRNA scaffold sequence is an artificially modified sequence of SEQ ID NO: 101, and may be any one selected from the examples described in the section “i) crRNA scaffold sequence” described above. Here, the tracrRNA scaffold sequence may be a wild-type tracrRNA scaffold sequence or an engineered tracrRNA scaffold sequence. As an example, the wild-type tracrRNA scaffold sequence may be SEQ ID NO: 110. As another example, the engineered tracrRNA scaffold sequence is an artificially modified sequence of SEQ ID NO: 110, and may be any one selected from the examples described in the section “ii) tracrRNA scaffold sequence” described above. As an example, the first scaffold region may be any one selected from the examples described in the section “iv) Examples of scaffold region” described above.

Here, the first guide RNA may optionally further comprise a U-rich tail sequence. The U-rich tail sequence may be located at the 3′ end of the first guide region. The U-rich tail sequence may be 5′-(UₐN)_{d}Uₑ-3′, 5′-UₐVUₐVUₑ-3′, or 5′-UₐVUₐVUₐVUₑ-3′. Here, N may be A, C, G or U, and each V may be independently A, C or G. Here, a may be an integer of 0 to 4, d may be an integer of 0 to 3, and e may be an integer of 1 to 10. As an example, the U-rich tail sequence may be any one selected from the examples described in the section “2-3) U-rich tail sequence” described above.

Here, the second guide RNA may comprise a second guide region and a second scaffold region. Here, the second guide region may have a second guide sequence. Here, the second guide sequence may be a sequence of 15 to 40 nucleotides capable of forming a complementary bond with a target sequence present in a downstream region based on the IVS26 mutation (c.2991+1655A>G) in the intron 26 region of the CEP290 gene. Alternatively, the second guide sequence may be a sequence of 15 to 40 nucleotides capable of forming a complementary bond with a target sequence present in a region between the IVS26 mutation (c.2991+1655A>G) and the 5′ end of exon 27 of the CEP290 gene. Here, the complementary bond may optionally contain 0 to 5 mismatched bonds. As an example, the second guide sequence may be any one sequence selected from the guide sequences listed in Table 4. Here, the second scaffold region may comprise a crRNA scaffold sequence and a tracrRNA scaffold sequence. The second scaffold region may optionally further comprise a linker, and the linker may be located between the tracrRNA scaffold sequence and the crRNA scaffold sequence. Here, the crRNA scaffold sequence may be a wild-type crRNA scaffold sequence or an engineered crRNA scaffold sequence. As an example, the wild-type crRNA scaffold sequence may be SEQ ID NO: 101. As another example, the engineered crRNA scaffold sequence is an artificially modified sequence of SEQ ID NO: 101, and may be any one selected from the examples described in the section “i) crRNA scaffold sequence” described above. Here, the tracrRNA scaffold sequence may be a wild-type tracrRNA scaffold sequence or an engineered tracrRNA scaffold sequence. As an example, the wild-type tracrRNA scaffold sequence may be SEQ ID NO: 110. As another example, the engineered tracrRNA scaffold sequence is an artificially modified sequence of SEQ ID NO: 110, and may be any one selected from the examples described in the section “ii) tracrRNA scaffold sequence” described above. As an example, the second scaffold region may be any one selected from the examples described in the section “iv) Examples of scaffold region” described above.

Here, the second guide RNA may optionally further comprise a U-rich tail sequence. The U-rich tail sequence may be located at the 3′ end of the second guide region. The U-rich tail sequence may be 5′-(UₐN)_{d}Uₑ-3′, 5′-UₐVUₐVUₑ-3′, or 5′-UₐVUₐVUₐVUₑ-3′. Here, N may be A, C, G or U, and each V may be independently A, C or G. Here, a may be an integer of 0 to 4, d may be an integer of 0 to 3, and e may be an integer of 1 to 10. As an example, the U-rich tail sequence may be any one selected from the examples described in the section “2-3) U-rich tail sequence” described above.

The Cas12f1 protein may be a wild-type Cas12f1 protein or a Cas12f1 variant. Here, the Cas12f1 protein is as described in the section “3. Cas12f1 (Cas14a1) protein” described above, and has the same characteristics and structure as each configuration described in the section.

The composition may optionally further comprise at least one guide RNA or a nucleic acid encoding the same. Here, the further included guide RNA may recognize a target sequence different from those recognized by the first guide RNA and the second guide RNA.

The composition may optionally further comprise at least one Cas12f1 (Cas14a1) protein or a nucleic acid encoding the same. Here, the composition may comprise two or more Cas12f1 (Cas14a1) proteins having different amino acid sequences. Here, the two or more Cas12f1 (Cas14a1) proteins having different amino acid sequences may be a wild-type Cas12f1 (Cas14a1) protein and a Cas12f1 variant.

The composition may be in a form of a nucleic acid. Here, the composition may comprise nucleic acids encoding two or more guide RNAs targeting the CEP290 gene and a nucleic acid encoding a Cas12f1 protein in one vector. Alternatively, the composition may comprise nucleic acids encoding two or more guide RNAs targeting the CEP290 gene and a nucleic acid encoding a Cas12f1 protein in separate vectors. Here, the vector may be a plasmid, an mRNA (transcript), a PCR amplicon, or a viral vector. Here, the viral vector may be at least one viral vector selected from the group consisting of a retroviral (retrovirus) vector, a lentiviral (lentivirus) vector, an adenoviral (adenovirus vector), an adeno-associated viral vector (adeno-associated virus (AAV) vector), a vaccinia viral (vaccinia virus) vector, a poxviral (poxvirus) vector, and a herpes simplex viral (herpes simplex virus) vector. Here, the vector may further comprise a promoter for a nucleic acid encoding the guide RNA and a promoter for a nucleic acid encoding the Cas12f1 protein. Here, the promoter may be a U6 promoter, an H1 promoter, a 7SK promoter, a CMV promoter, a CBA promoter, an LTR promoter, an Ad MLP promoter, an HSV promoter, an SV40 promoter, or an RSV promoter. As an example, the composition may comprise a first vector comprising a nucleic acid encoding the first guide RNA and a nucleic acid encoding the second guide RNA, and a second vector comprising a nucleic acid encoding the Cas12f1 protein. Here, each of the first vector and the second vector may be a viral vector. Here, the viral vector may be an adeno-associated virus (AAV) vector. Here, the first vector may comprise a promoter for each of the nucleic acid encoding the first guide RNA and the nucleic acid encoding the second guide RNA, and each promoter may be a U6 promoter, an H1 promoter, or a 7SK promoter. Here, the second vector may comprise a promoter for the nucleic acid encoding the Cas12f1 protein, and the promoter may be a CMV promoter, a CBA promoter, an LTR promoter, an Ad MLP promoter, an HSV promoter, an SV40 promoter, or an RSV promoter. As another example, the composition may comprise a vector that contains a nucleic acid encoding the first guide RNA, a nucleic acid encoding the second guide RNA, and a nucleic acid encoding the Cas12f1 protein. Here, the vector may be a viral vector. Here, the viral vector may be an adeno-associated virus (AAV) vector. Here, the vector may comprise a promoter for each of the nucleic acid encoding the first guide RNA, the nucleic acid encoding the second guide RNA, and the nucleic acid encoding the Cas12f1 protein. Here, each promoter may be a U6 promoter, an H1 promoter, a 7SK promoter, a CMV promoter, a CBA promoter, an LTR promoter, an Ad MLP promoter, an HSV promoter, an SV40 promoter, or an RSV promoter.

The composition may be in a mixed form of a nucleic acid and a protein. Here, the composition may comprise a CRISPR/Cas12f1 complex in which the guide RNA and the Cas12f1 protein are bound to each other. As an example, the composition may comprise a first CRISPR/Cas12f1 complex in which the first guide RNA and the Cas12f1 protein are bound to each other, and a second CRISPR/Cas12f1 complex in which the second guide RNA and the Cas12f1 protein are bound to each other.

### 4. Examples (2) of Composition

In an embodiment, the composition may comprise:
at least one guide RNA targeting the CEP290 gene or a nucleic acid encoding the same; and
a Cas12f1 protein or a nucleic acid encoding the same.

Here, the guide RNA may complementarily bind to a target sequence present in the CEP290 gene.

Here, the guide RNA may target a partial region of the intron 26 region containing the IVS26 mutation (c.2991+1655A>G) of the CEP gene.

Here, the guide RNA may comprise a guide region and a scaffold region. Here, the guide region may have a guide sequence. Here, the guide sequence may be a sequence of 15 to 40 nucleotides capable of forming a complementary bond with a target sequence present in a partial region of the intron 26 region containing the IVS26 mutation (c.2991+1655A>G) of the CEP gene. Here, the complementary bond may optionally contain 0 to 5 mismatched bonds. As an example, the guide sequence may be any one sequence selected from the guide sequences described in Tables 3 and 4. Here, the scaffold region may comprise a crRNA scaffold sequence and a tracrRNA scaffold sequence. The scaffold region may optionally further comprise a linker, and the linker may be located between the tracrRNA scaffold sequence and the crRNA scaffold sequence. Here, the crRNA scaffold sequence may be a wild-type crRNA scaffold sequence or an engineered crRNA scaffold sequence. As an example, the wild-type crRNA scaffold sequence may be SEQ ID NO: 101. As another example, the engineered crRNA scaffold sequence is an artificially modified sequence of SEQ ID NO: 101, and may be any one selected from the examples described in the section “i) crRNA scaffold sequence” described above. Here, the tracrRNA scaffold sequence may be a wild-type tracrRNA scaffold sequence or an engineered tracrRNA scaffold sequence. As an example, the wild-type tracrRNA scaffold sequence may be SEQ ID NO: 110. As another example, the engineered tracrRNA scaffold sequence is an artificially modified sequence of SEQ ID NO: 110, and may be any one selected from the examples described in the section “ii) tracrRNA scaffold sequence” described above. As an example, the first scaffold region may be any one selected from the examples described in the section “iv) Examples of scaffold region” described above.

Here, the guide RNA may optionally further comprise a U-rich tail sequence. The U-rich tail sequence may be located at the 3′ end of the guide region. The U-rich tail sequence may be 5′-(UₐN)_{d}Uₑ-3′, 5′-UₐVUₐVUₑ-3′, or 5′-UₐVUₐVUₐVUₑ-3′. Here, N may be A, C, G or U, and each V may be independently A, C or G. Here, a may be an integer of 0 to 4, d may be an integer of 0 to 3, and e may be an integer of 1 to 10. As an example, the U-rich tail sequence may be any one selected from the examples described in the section “2-3) U-rich tail sequence” described above.

The Cas12f1 protein may be a wild-type Cas12f1 protein or a Cas12f1 variant. Here, the Cas12f1 protein is as described in the section “3. Cas12f1 (Cas14a1) protein” described above, and has the same characteristics and structure as each configuration described in the section.

The composition may optionally further comprise at least one guide RNA or a nucleic acid encoding the same. Here, the further included guide RNA may recognize a target sequence different from that recognized by the guide RNA.

The composition may optionally further comprise at least one Cas12f1 (Cas14a1) protein or a nucleic acid encoding the same. Here, the composition may comprise two or more Cas12f1 (Cas14a1) proteins having different amino acid sequences. Here, the two or more Cas12f1 (Cas14a1) proteins having different amino acid sequences may be a wild-type Cas12f1 (Cas14a1) protein and a Cas12f1 variant.

The composition may be in a form of a nucleic acid. Here, the composition may comprise a nucleic acid encoding at least one guide RNA targeting the CEP290 gene and a nucleic acid encoding a Cas12f1 protein in one vector. Alternatively, the composition may comprise a nucleic acid encoding at least one guide RNA targeting the CEP290 gene and a nucleic acid encoding a Cas12f1 protein in separate vectors. Here, the vector may be a plasmid, an mRNA (transcript), a PCR amplicon, or a viral vector. Here, the viral vector may be at least one viral vector selected from the group consisting of a retroviral (retrovirus) vector, a lentiviral (lentivirus) vector, an adenoviral (adenovirus vector), an adeno-associated viral vector (adeno-associated virus (AAV) vector), a vaccinia viral (vaccinia virus) vector, a poxviral (poxvirus) vector, and a herpes simplex viral (herpes simplex virus) vector. Here, the vector may further comprise a promoter for a nucleic acid encoding the guide RNA and a promoter for a nucleic acid encoding the Cas12f1 protein. Here, the promoter may be a U6 promoter, an H1 promoter, a 7SK promoter, a CMV promoter, a CBA promoter, an LTR promoter, an Ad MLP promoter, an HSV promoter, an SV40 promoter, or an RSV promoter.

The composition may be in a mixed form of a nucleic acid and a protein. Here, the composition may comprise a CRISPR/Cas12f1 complex in which the guide RNA and the Cas12f1 protein are bound to each other.

### <Method for Treating LCA10 Disease>

In an aspect disclosed by the present specification, there is provided a method for treating LCA10 disease. The method for treating LCA10 disease uses a CRISPR/Cas12f1 system. More specifically, the method relates to a method of introducing (or delivering) a composition comprising the CRISPR/Cas12f1 (Cas14a1) system to a subject and/or a target cell, thereby modifying the CEP290 gene that is a target gene present in the subject and/or target cell. Here, the subject is an animal or some tissue thereof, and the animal may be a human or a non-human animal. Here, the target cells may be non-human animal cells or human cells. The method may be performed in vitro, ex vivo or in vivo. Here, the in vivo may be in a human body or in a non-human animal body.

The method may be a method that comprises introducing (or delivering, applying, injecting, or administering) the CRISPR/Cas12f1 (Cas14a1) system into a subject and/or a target cell. The CRISPR/Cas12f1 system is as described in the section “<CRISPR/Cas12f1 system for treatment of LCA10 disease>” described above, and has the same characteristics and structure as each configuration described in the section.

The method induces deletion or inversion of a partial nucleic acid sequence in the intron 26 region of the CEP290 gene using the CRISPR/Cas12f1 system. Here, the partial nucleic acid sequence contains the IVS26 mutation (c.2991+1655A>G). This allows removal of all or part of the 128 base pair cryptic exon that is transcribed by the CEP290 gene. Therefore, the method effectively removes all or part of the cryptic exon that causes LCA10 disease, and thus may be used as an effective treatment method for LCA10 disease.

Hereinafter, embodiments of the method will be described in detail.

### 1. Examples of Method

### 1-1) Example (1) of Method

In an embodiment, the method may be a method of artificially modifying the CEP290 gene in a cell. Here, the method may comprise treating a cell with a composition or introducing a composition into a cell.

Here, the composition may comprise:
at least one guide RNA targeting the CEP290 gene or a nucleic acid encoding the same; and
a Cas12f1 protein or a nucleic acid encoding the same.

Alternatively, the composition may comprise:
two or more guide RNAs targeting the CEP290 gene or nucleic acids encoding the same; and
a Cas12f1 protein or a nucleic acid encoding the same. Here, each of the two or more guide RNAs may target a different target sequence present in the CEP290 gene.

The composition is as described in the section “<Composition for treatment of LCA10 disease>” described above, and has the same characteristics and structure as each configuration described in the section. In addition, the guide RNA is as described in the section “2. Guide RNA” of <CRISPR/Cas12f1 system for treatment of LCA10 disease> described above, and has the same characteristics and structure as each configuration described in the section. In addition, the Cas12f1 protein is as described in the section “3. Cas12f1 (Cas14a1) protein” in <CRISPR/Cas12f1 system for treatment of LCA10 disease>, and has the same characteristics and structure as each configuration described in the section.

The composition may be a viral vector or a non-viral vector, or in a mixed form thereof. Alternatively, the composition may be in a mixed form of a nucleic acid and a protein. The viral vector, the non-viral vector, or the mixed form thereof is as described in the section “1. Form of nucleic acid” in <Composition for treatment of LCA10 disease> described above, and has the same characteristics and structure as each configuration described in the section. In addition, the mixed form of a nucleic acid and a protein is as described in the section “2. Mixed form of nucleic acid and protein” in <Composition for treatment of LCA10 disease> described above, and has the same characteristics and structure as each configuration described in the section.

In an embodiment, the composition may be in a form of a viral vector comprising nucleic acids encoding two or more guide RNAs and a nucleic acid encoding a Cas12f1 protein. Here, the viral vector may be one or more selected from the group consisting of a retroviral vector, a lentiviral vector, an adenoviral vector, an adeno-associated viral vector, a vaccinia virus vector, a poxvirus vector, and a herpes simplex virus vector. Here, the composition may comprise one viral vector comprising both the nucleic acids encoding two or more guide RNAs and the nucleic acid encoding the Cas12f1 protein. Alternatively, the composition may comprise two viral vectors which respectively comprise the nucleic acids encoding two or more guide RNAs and the nucleic acid encoding the Cas12f1 protein.

In another embodiment, the composition may be in a form of a non-viral vector comprising a nucleic acid encoding a guide RNA and a nucleic acid encoding a Cas12f1 protein. Here, the non-viral vector may be a plasmid. Here, the composition may comprise one plasmid comprising both the nucleic acid encoding the guide RNA and the nucleic acid encoding the Cas12f1 protein. Alternatively, the composition may comprise two plasmids which respectively comprise the nucleic acid encoding the guide RNA and the nucleic acid encoding the Cas12f1 protein.

In yet another embodiment, the composition may comprise a PCR amplicon comprising the nucleic acid encoding the guide RNA and an mRNA encoding the Cas12f1 protein.

In still yet another embodiment, the composition may comprise a PCR amplicon comprising the nucleic acid encoding the guide RNA and the Cas12f1 protein. Alternatively, the composition may comprise a CRISPR/Cas12f1 (Cas14a1) complex in which the guide RNA and the Cas12f1 protein are bound to each other.

The cell may be a cell containing the CEP290 gene. The cell may be a non-human animal cell or human cell.

The cells may be cells obtained from a subject with LCA10 disease. Here, the subject may be a human or a non-human animal. Here, the non-human animal may be a non-human animal susceptible to LCA10 disease.

Treating the cell with the composition or introducing the composition into the cell may be performed to introduce the CRISPR/Cas12f1 (Cas14a1) system into the cell. Alternatively, treating the cell with the composition or introducing the composition into the cell may be performed to bring the CRISPR/Cas12f1 (Cas14a1) system in contact with the CEP290 gene present in the cell. Treating the cell with the composition may be performed using electroporation, gene gun, sonoporation, magnetofection, nanoparticles, and/or transient cell compression or squeezing. Alternatively, treating the eukaryotic cell with the composition may be performed using cationic liposome, lithium acetate-DMSO, lipid-mediated transfection, calcium phosphate precipitation, lipofection, polyethyleneimine (PEI)-mediated transfection, DEAE-dextran-mediated transfection, and/or nanoparticle-mediated nucleic acid delivery (see Panyam et al., Adv Drug Deliv Rev. 2012 Sep. 13. pii: S0169-409X(12)00283-9. doi: 10.1016/j.addr.2012.09.023), but the treating method is not limited thereto.

As a result of treatment of the cell with the composition or introduction of the composition into the cell, deletion or inversion of a partial nucleic acid sequence in the intron 26 region of the CEP290 gene may be induced. Here, the partial nucleic acid sequence may contain the IVS26 mutation (c.2991+1655A>G). This allows removal or inversion of all or part of the 128 base pair cryptic exon that is transcribed by the CEP290 gene. Alternatively, as a result of treatment of the cell with the composition or introduction of the composition into the cell, modification caused by non-homologous end joining (NHEJ) may occur in a partial nucleic acid sequence in the intron 26 region of the CEP290 gene. Here, the partial nucleic acid sequence may contain the IVS26 mutation (c.2991+1655A>G). Here, the modification caused by NHEJ may be deletion, insertion, or indel of one or more nucleotides.

### 2-2) Example (2) of Method

In another embodiment, the method is a method for treating LCA10 disease, and may comprise introducing or administering a composition to a subject.

Here, the composition may comprise:
at least one guide RNA targeting the CEP290 gene or a nucleic acid encoding the same; and
a Cas12f1 protein or a nucleic acid encoding the same.

Alternatively, the composition may comprise:
two or more guide RNAs targeting the CEP290 gene or nucleic acids encoding the same; and
a Cas12f1 protein or a nucleic acid encoding the same. Here, each of the two or more guide RNAs may target a different target sequence present in the CEP gene.

The composition is as described in the section “<Composition for treatment of LCA10 disease>” described above, and has the same characteristics and structure as each configuration described in the section. In addition, the guide RNA is as described in the section “2. Guide RNA” of <CRISPR/Cas12f1 system for treatment of LCA10 disease> described above, and has the same characteristics and structure as each configuration described in the section. In addition, the Cas12f1 protein is as described in the section “3. Cas12f1 (Cas14a1) protein” in <CRISPR/Cas12f1 system for treatment of LCA10 disease>, and has the same characteristics and structure as each configuration described in the section.

The composition may be a viral vector or a non-viral vector, or in a mixed form thereof. Alternatively, the composition may be in a mixed form of a nucleic acid and a protein. The viral vector, the non-viral vector, or the mixed form thereof is as described in the section “1. Form of nucleic acid” in <Composition for treatment of LCA10 disease> described above, and has the same characteristics and structure as each configuration described in the section. In addition, the mixed form of a nucleic acid and a protein is as described in the section “2. Mixed form of nucleic acid and protein” in <Composition for treatment of LCA10 disease> described above, and has the same characteristics and structure as each configuration described in the section.

In an embodiment, the composition may be in a form of a viral vector comprising nucleic acids encoding two or more guide RNAs and a nucleic acid encoding a Cas12f1 protein. Here, the viral vector may be one or more selected from the group consisting of a retroviral vector, a lentiviral vector, an adenoviral vector, an adeno-associated viral vector, a vaccinia virus vector, a poxvirus vector, and a herpes simplex virus vector. Here, the composition may comprise one viral vector comprising both the nucleic acids encoding two or more guide RNAs and the nucleic acid encoding the Cas12f1 protein. Alternatively, the composition may comprise two viral vectors which respectively comprise the nucleic acids encoding two or more guide RNAs and the nucleic acid encoding the Cas12f1 protein.

In another embodiment, the composition may be in a form of a non-viral vector comprising a nucleic acid encoding a guide RNA and a nucleic acid encoding a Cas12f1 protein. Here, the non-viral vector may be a plasmid. Here, the composition may comprise one plasmid comprising both the nucleic acid encoding the guide RNA and the nucleic acid encoding the Cas12f1 protein. Alternatively, the composition may comprise two plasmids which respectively comprise the nucleic acid encoding the guide RNA and the nucleic acid encoding the Cas12f1 protein.

In yet another embodiment, the composition may comprise a PCR amplicon comprising a nucleic acid encoding a guide RNA and an mRNA encoding a Cas12f1 protein.

In still yet another embodiment, the composition may comprise a PCR amplicon comprising a nucleic acid encoding a guide RNA and a Cas12f1 protein. Alternatively, the composition may comprise a CRISPR/Cas12f1 (Cas14a1) complex in which the guide RNA and the Cas12f1 protein are bound to each other.

The subject may be a human or non-human animal with LCA10 disease. Alternatively, the subject may be a human or non-human animal having the CEP290 gene containing a cryptic exon.

Introducing or administering the composition to the subject may be performed to introduce the CRISPR/Cas12f1 (Cas14a1) system into one or more cells present in the subject. Alternatively, introducing or administering the composition to the subject may be performed to bring the CRISPR/Cas12f1 (Cas14a1) system in contact with the CEP290 gene present in one or more cells present in the subject. Introducing or administering the composition to the subject may be performed using microinjection, cationic liposome, lithium acetate-DMSO, lipid-mediated transfection, calcium phosphate precipitation, lipofection, polyethyleneimine (PEI)-mediated transfection, DEAE-dextran-mediated transfection, and/or nanoparticle-mediated nucleic acid delivery (see Panyam et al., Adv Drug Deliv Rev. 2012 Sep. 13. pii: S0169-409X(12)00283-9. doi: 10.1016/j.addr.2012.09.023), but the treating method is not limited thereto. Alternatively, administering the composition to the subject may be performed by injection, transfusion, implantation, or transplantation. Here, administering the composition to the subject may be performed by a route of administration selected from subretinal, subcutaneous, intradermal, intraocular, intravitreal, intratumoral, intranodal, intramedullary, intramuscular, intravenous, intralymphatic, or intraperitoneal.

A single dose of the composition (a pharmaceutically effective amount to obtain a predetermined desired effect) may be appropriately prescribed by taking into account the age, health and body weight of the subject to be administered, the type of concurrent treatment, if any, the treatment frequency, characteristics of a desired effect, and the like.

As a result of introducing or administering the composition to the subject, removal or inversion of a partial nucleic acid sequence in the intron 26 region of the CEP290 gene may be induced. Here, the partial nucleic acid sequence may contain the IVS26 mutation (c.2991+1655A>G). This allows removal or inversion of all or part of the 128 base pair cryptic exon that is transcribed by the CEP290 gene.

Alternatively, as a result of introducing or administering the composition to the subject, modification caused by non-homologous end joining (NHEJ) may occur in a partial nucleic acid sequence in the intron 26 region of the CEP290 gene in one or more cells present in the subject. Here, the partial nucleic acid sequence may contain the IVS26 mutation (c.2991+1655A>G). Here, the modification caused by NHEJ may be deletion, insertion, or indel of one or more nucleotides.

Therefore, the method effectively deletes or inverts all or part of the cryptic exon that causes LCA10 disease, and thus may be used as an effective treatment method for LCA10 disease.

### 2. Therapeutic Effect—Modification of Target Gene

The composition and the method disclosed herein allow the CEP290 gene to be modified. More specifically, the CEP290 gene may be modified by the CRISPR/Cas12f1 system. Here, the modification may comprise both i) cleavage or damage of the CEP290 gene caused by the CRISPR/Cas12f1 (Cas14a1) system and ii) repair or recovery of the cleaved (or damaged) CEP290 gene. Here, the modification may be inversion of a partial nucleic acid sequence in intron 26 present in the CEP290 gene, which may result in inversion of all or part of the cryptic exon that is transcribed and generated by the CEP290 gene. Alternatively, the modification may be deletion of a partial nucleic acid sequence in intron 26 present in the CEP290 gene, which may result in deletion of a partial nucleic acid sequence in the cryptic exon that is transcribed and generated by the CEP290 gene. Alternatively, the modification may be insertion of an additional nucleic acid sequence into the intron 26 present in the CEP290 gene, which may result in insertion of an additional nucleic acid sequence into the cryptic exon that is transcribed and generated by the CEP290 gene. Alternatively, the modification may be insertion and deletion (indel) in which a partial nucleic acid sequence in intron 26 present in the CEP290 gene is deleted and an additional sequence is inserted thereinto, which may result in deletion of a partial sequence in the cryptic exon and insertion of an additional sequence thereinto, the cryptic exon being transcribed and generated by the CEP290 gene. Alternatively, the modification may be substitution of at least one nucleic acid sequence present in intron 26 of the CEP290 gene, which may result in modification of a sequence of mRNA transcribed by the CEP290 gene. Here, the partial nucleic acid sequence in intron 26 may contain the IVS26 mutation (c.2991+1655A>G). Here, at least one nucleic acid sequence present in intron 26 may contain the IVS26 mutation (c.2991+1655A>G) sequence. Hereinafter, the modification will be described in detail.

### 2-1) Deletion

As a result of performing the method provided herein, the modification may be removal (or deletion) of all or part of the nucleic acid sequence of the intron 26 present in the CEP290 gene. The modification may mean deleting a partial nucleotide sequence in intron 26 present in the CEP290 gene. In an embodiment, the method comprises introducing, into a cell containing the CEP290 gene or a subject having the cell, a Cas12f1 protein or a nucleic acid encoding the same, a first engineered guide RNA or a nucleic acid encoding the same, and a second engineered guide RNA or a nucleic acid encoding the same. This results in removal of a specific sequence in intron 26 present in the CEP290 gene. Here, the specific sequence may be a nucleic acid sequence containing the IVS26 mutation (c.2991+1655A>G).

### 2-2) Inversion

As a result of performing the method provided herein, the modification may be inversion of all or part of intron 26 present in the CEP290 gene. The inversion may be a change in the direction of transcription of all or a partial region of intron 26 present in the CEP290 gene. That is, the inversion may mean that all or a partial region of intron 26 in the CEP290 gene is recombined and reversed, indicating that all or a partial region of intron 26 is reversed from one end to the other. In an embodiment, the method comprises introducing, into a cell containing the CEP290 gene or a subject having the cell, a Cas12f1 protein or a nucleic acid encoding the same, a first engineered guide RNA or a nucleic acid encoding the same, and a second engineered guide RNA or a nucleic acid encoding the same. This results in inversion of all or a partial region of intron 26 present in the CEP290 gene. Here, the partial region may be a region containing the IVS26 mutation (c.2991+1655A>G).

### 2-3) Insertion and Deletion (or Indel)

As a result of performing the method provided herein, deletion and insertion (indel) may occur in intron 26 of the CEP290 gene. The indel may be caused by non-homologous end joining (NHEJ). In general, NHEJ is a method of repairing or recovering a break (for example, cleavage) in a double-strand in DNA in which two compatible ends formed by the break repeatedly contact each other. Repair of a damaged gene or nucleic acid using NHEJ results in partial insertion and/or deletion (indel) of a nucleic acid sequence at the NHEJ repair site. This insertion and/or deletion alters its reading frame and produces a frameshifted transcript mRNA, which, in turn, undergoes nonsense-mediated decay or fails to synthesize a normal protein, thereby losing its original function. Therefore, when indel occurs in intron 26 of the CEP290 gene, the cryptic exon, which is transcribed and generated by the CEP290 gene, may be inactivated. In an embodiment, as a result of performing the above method, deletion and/or addition of one or more bases may occur in intron 26 of the CEP290 gene.

### 2-4) Substitution or Base Editing

As a result of performing the method provided herein, base editing may occur in the intron 26 region containing the point mutation (c.2991+1655A>G) of the CEP290 gene. Unlike indel in which deletion or addition of any nucleotide occurs in a target gene or target nucleic acid, the base editing means altering one or more specific bases in a nucleic acid as intended. In other words, the base editing causes a pre-intended point mutation at a specific position in a target gene or target nucleic acid. In an embodiment, as a result of performing the method, the point mutation (c.2991+1655A>G) in the CEP290 gene may undergo substitution with A again.

### 2-5) Insertion

As a result of performing the method provided herein, knockin may occur in the CEP290 gene. The knockin means insertion of an additional nucleic acid sequence into the CEP290 gene. To cause the knockin, a donor comprising the additional nucleic acid sequence is further required in addition to a CRISPR/Cas12f1 (Cas14a1) system. When the CRISPR/Cas12f1 (Cas14a1) system cleaves the CEP290 gene in a cell, repair of the cleaved CEP290 gene occurs. Here, the repair may be performed using homology directed repairing (HDR), wherein the donor participates in the repair process so that the additional nucleic acid sequence is inserted into the CEP290 gene. In an embodiment, the method may further comprise delivering a donor into a cell. Here, the donor comprises an additional nucleic acid of interest, and induces insertion of the additional nucleic acid into the CEP290 gene. Here, when the donor is delivered into the cell, one of the above-described treating methods for the CRISPR/Cas12f1 (Cas14a1) system may be used.

### 2-6) Phenotype by Modification of Target Gene

Modification of the CEP290 gene may result in effects of knockout, knockdown, or knockin. The “knockout” means inactivation of the CEP290 gene, and the “inactivation of the CEP290 gene” means a state in which transcription and/or translation of the CEP290 gene does not occur. The knockout may suppress transcription and translation of a gene, which causes a disease or has an abnormal function, thereby preventing expression of a protein. The “knockdown” means decreasing transcription and/or translation of the CEP290 gene, or decreasing expression of the CEP290 protein. The knockdown may regulate expression of a gene or protein to be overexpressed, thereby preventing occurrence of or treating a disease. The “knockin” means inserting a specific nucleic acid or gene into the CEP290 gene. Here, the “specific nucleic acid or gene” means a gene or nucleic acid to be inserted or expressed. The knockin may be used to treat a disease by correcting a mutant gene, which causes a disease, or inserting a normal gene so that expression of the normal gene is induced.

In an embodiment, when the CEP290 gene is modified by the method, the cell may have suppressed or decreased transcription and/or translation of the CEP290 gene containing the IVS26 mutation (c.2991+1655A>G). Alternatively, the cell may have increased transcription and/or translation of the normal CEP290 gene.

In another embodiment, when the CEP290 gene is modified by the method, the subject may have suppressed or decreased transcription and/or translation of the CEP290 gene containing the IVS26 mutation (c.2991+1655A>G). Alternatively, the subject may have increased transcription and/or translation of the normal CEP290 gene.

### <Factor for Decreasing Non-Homologous End Joining DNA Repair Pathway (NHEJ) Activity>

The composition for treatment of LCA10 disease provided herein may further comprise a factor capable of decreasing non-homologous end joining DNA repair pathway (NHEJ) activity. In addition, the method for treating LCA10 disease provided herein may be performed using the CRISPR/Cas12f1 system together with the factor for decreasing NHEJ activity.

The factor for decreasing NHEJ activity acts in a cell and serves to decrease non-homologous end-joining activity for cleavage of double-stranded DNA.

The non-homologous end-joining activity may be, for example, canonical NHEJ pathway (Kasparek & Humphrey (2011) Seminars in Cell & Dev. Biol. 22:886-897), or alternative NHEJ pathway. Specifically, the NHEJ activity may be canonical NHEJ pathway caused by expression or activity of MRE11, RAD50, NBS1, DNA-PK, CtIP, Ku70, Ku80, Artemis (DCLRE1C), ligase IV (Lig4), PNKP, XRCC4, XLF (XRCC4-like factor), ATM (ATM serine/threonine kinase), CHK1/CHK2, CLF (CURLY LEAF), and/or Pol Mu (POLM), or alternative NHEJ pathway caused by expression or activity of XRCC1, PARP (for example, PARP1), LigI, and/or Lig3.

The factor for decreasing NHEJ activity may be, for example, a small molecule or inhibitory nucleic acid such as a short interfering nucleic acid (for example, short interfering RNA (siRNA), double-stranded RNA (dsRNA), micro-RNA (miRNA), and short hairpin RNA (shRNA) specific for gene transcript), or an antisense oligonucleotide. The factor for decreasing NHEJ activity functions to decrease or inhibit expression and/or activity of factors involved in the aforementioned canonical NHEJ pathway and/or alternative NHEJ pathway.

In a case where this factor for decreasing NHEJ activity is used together with the composition for treatment of LCA10 disease, intracellular non-homologous end joining activity may be decreased to increase efficiency of the composition for treatment of LCA10 disease for modifying the CEP290 gene.

### Possible Embodiments (Aspects) of Disclosure

### Cas12f1 Protein

### Example 1, Cas12f1 Protein

A Cas12f1 protein that belongs to the Cas14 family.

### Example 2, Cas14a1 Protein

The Cas12f1 protein of Example 1, wherein the Cas12f1 protein is a Cas14a1 protein.

### Example 3, Wild-Type Protein

The Cas12f1 protein of any one of Examples 1 to 2, wherein the Cas12f1 protein is a wild-type protein existing in nature.

### Example 4, Wild-Type Cas14 Family Protein

The Cas12f1 protein of Example 3, wherein the Cas12f1 protein is derived from the Cas14 family (Harrington et al., Science 5, 839-842 (2018); US 2020/0172886 A1).

### Example 5, Wild-Type Cas14 Family Protein, Whose Sequence Defined

The Cas12f1 protein of Example 3, wherein the Cas12f1 protein is represented by the following sequence:

(SEQ ID NO: 129)
mAKNTITKTLKLRIVRPYNSAEVEKIVADEKNNREKIALEKNKDKVKEAC
SKHLKVAAYCTTQVERNACLFCKARKLDDKFYQKLRGQFPDAVFWQEISE
IFRQLQKQAAEIYNQSLIELYYEIFIKGKGIANASSVEHYLSDVCYTRAA
ELFKNAAIASGLRSKIKSNFRLKELKNMKSGLPTTKSDNFPIPLVKQKGG
QYTGFEISNHNSDFIIKIPFGRWQVKKEIDKYRPWEKFDFEQVQKSPKPI
SLLLSTQRRKRNKGWSKDEGTEAEIKKVMNGDYQTSYIEVKRGSKIGEKS
AWMLNLSIDVPKIDKGVDPSIIGGIDVGVKSPLVCAINNAFSRYSISDND
LFHFNKKMFARRRILLKKNRHKRAGHGAKNKLKPITILTEKSERFRKKLI
ERWACEIADFFIKNKVGTVQMENLESMKRKEDSYFNIRLRGFWPYAEMQN
KIEFKLKQYGIEIRKVAPNNTSKTCSKCGHLNNYFNFEYRKKNKFPHFKC
EKCNFKENADYNAALNISNPKLKSTKEEP.

### Example 6, Cas12f1 Protein Variant

The Cas12f1 protein of any one of Examples 1 to 2, wherein the Cas12f1 protein is a variant of a wild-type Cas12f1 protein.

### Example 7, Cas12f1 Protein Variant, in Terms of Function

The Cas12f1 protein of Example 6, wherein the Cas12f1 variant is a variant having the same function as the wild-type Cas12f1 protein, a variant in which a part of the function is modified as compared with the wild-type Cas12f1 protein, and/or a variant to which an additional function is added as compared with the wild-type Cas12f1 protein.

### Example 8, Addition of Polypeptide to N-Terminus/C-Terminus

The Cas12f1 protein of Example 7, wherein the Cas12f1 protein variant has the same function as the wild-type Cas12f1 protein, and is such that a polypeptide having a sequence of 50 or fewer amino acids is linked to the N-terminus and/or C-terminus of the wild-type Cas12f1 protein.

### Example 9, Example of Cas12f1 Protein Variant

The Cas12f1 protein of Example 8, wherein the Cas12f1 protein variant is represented by an amino acid sequence selected from:

(SEQ ID NO: 288)
MGEKSSRRRRNGKSGAWTAAITSCVGGKMAKNTITKTLKLRIVRPYNSAEVE
KIVADEKNNREKIALEKNKDKVKEACSKHLKVAAYCTTQVERNACLFCKARK
LDDKFYQKLRGQFPDAVFWQEISEIFRQLQKQAAEIYNQSLIELYYEIFIKGKGI
ANASSVEHYLSDVCYTRAAELFKNAAIASGLRSKIKSNFRLKELKNMKSGLPT
TKSDNFPIPLVKQKGGQYTGFEISNHNSDFIIKIPFGRWQVKKEIDKYRPWEKF
DFEQVQKSPKPISLLLSTQRRKRNKGWSKDEGTEAEIKKVMNGDYQTSYIEV
KRGSKIGEKSAWMLNLSIDVPKIDKGVDPSIIGGIDVGVKSPLVCAINNAFSRYS
ISDNDLFHFNKKMFARRRILLKKNRHKRAGHGAKNKLKPITILTEKSERFRKK
LIERWACEIADFFIKNKVGTVQMENLESMKRKEDSYFNIRLRGFWPYAEMQN
KIEFKLKQYGIEIRKVAPNNTSKTCSKCGHLNNYFNFEYRKKNKFPHFKCEKC
NFKENADYNAALNISNPKLKSTKEEP;
(SEQ ID NO: 289)
MEKRINKIRKKLSADNATKPVSRSGPMAKNTITKTLKLRIVRPYNSAEVEKIVA
DEKNNREKIALEKNKDKVKEACSKHLKVAAYCTTQVERNACLFCKARKLDD
KFYQKLRGQFPDAVFWQEISEIFRQLQKQAAEIYNQSLIELYYEIFIKGKGIANA
SSVEHYLSDVCYTRAAELFKNAAIASGLRSKIKSNFRLKELKNMKSGLPTTKS
DNFPIPLVKQKGGQYTGFEISNHNSDFIIKIPFGRWQVKKEIDKYRPWEKFDFE
QVQKSPKPISLLLSTQRRKRNKGWSKDEGTEAEIKKVMNGDYQTSYIEVKRG
SKIGEKSAWMLNLSIDVPKIDKGVDPSIIGGIDVGVKSPLVCAINNAFSRYSISD
NDLFHFNKKMFARRRILLKKNRHKRAGHGAKNKLKPITILTEKSERFRKKLIE
RWACEIADFFIKNKVGTVQMENLESMKRKEDSYFNIRLRGFWPYAEMQNKIE
FKLKQYGIEIRKVAPNNTSKTCSKCGHLNNYFNFEYRKKNKFPHFKCEKCNFK
ENADYNAALNISNPKLKSTKEEP;
(SEQ ID NO: 290)
MAGGPGAGSAAPVSSTSSLPLAALNMRVMAKNTITKTLKLRIVRPYNSAEVE
KIVADEKNNREKIALEKNKDKVKEACSKHLKVAAYCTTQVERNACLFCKARK
LDDKFYQKLRGQFPDAVFWQEISEIFRQLQKQAAEIYNQSLIELYYEIFIKGKGI
ANASSVEHYLSDVCYTRAAELFKNAAIASGLRSKIKSNFRLKELKNMKSGLPT
TKSDNFPIPLVKQKGGQYTGFEISNHNSDFIIKIPFGRWQVKKEIDKYRPWEKF
DFEQVQKSPKPISLLLSTQRRKRNKGWSKDEGTEAEIKKVMNGDYQTSYIEV
KRGSKIGEKSAWMLNLSIDVPKIDKGVDPSIIGGIDVGVKSPLVCAINNAFSRYS
ISDNDLFHFNKKMFARRRILLKKNRHKRAGHGAKNKLKPITILTEKSERFRKK
LIERWACEIADFFIKNKVGTVQMENLESMKRKEDSYFNIRLRGFWPYAEMQN
KIEFKLKQYGIEIRKVAPNNTSKTCSKCGHLNNYFNFEYRKKNKFPHFKCEKC
NFKENADYNAALNISNPKLKSTKEEP;
and
(SEQ ID NO: 291)
MAGGPGAGSAAPVSSTSSLPLAALNMMAKNTITKTLKLRIVRPYNSAEVEKIV
ADEKNNREKIALEKNKDKVKEACSKHLKVAAYCTTQVERNACLFCKARKLD
DKFYQKLRGQFPDAVFWQEISEIFRQLQKQAAEIYNQSLIELYYEIFIKGKGIAN
ASSVEHYLSDVCYTRAAELFKNAAIASGLRSKIKSNFRLKELKNMKSGLPTTK
SDNFPIPLVKQKGGQYTGFEISNHNSDFIIKIPFGRWQVKKEIDKYRPWEKFDF
EQVQKSPKPISLLLSTQRRKRNKGWSKDEGTEAEIKKVMNGDYQTSYIEVKR
GSKIGEKSAWMLNLSIDVPKIDKGVDPSIIGGIDVGVKSPLVCAINNAFSRYSIS
DNDLFHFNKKMFARRRILLKKNRHKRAGHGAKNKLKPITILTEKSERFRKKLI
ERWACEIADFFIKNKVGTVQMENLESMKRKEDSYFNIRLRGFWPYAEMQNKI
EFKLKQYGIEIRKVAPNNTSKTCSKCGHLNNYFNFEYRKKNKFPHFKCEKCNF
KENADYNAALNISNPKLKSTKEEP.

### Example 10, Inclusion of Additional Active Domain

The Cas12f1 protein of Example 7, wherein the Cas12f1 variant is a variant in which an additional function is added as compared with the wild-type Cas12f1 protein, and comprises a domain, peptide, or protein having at least one activity selected from:
methylase activity; demethylase activity; transcription activation activity; transcription repression activity; transcription release factor activity; histone modification activity; RNA cleavage activity; and nucleic acid binding activity.

### Example 11, Inclusion of Domain Having Additional Function

The Cas12f1 protein of Example 7, wherein the Cas12f1 variant further comprises reverse transcriptase and/or deaminase.

### Example 12, Inclusion of NLS/NES

The Cas12f1 protein of any one of Examples 1 to 11, wherein the Cas12f1 protein further comprises at least one peptide selected from:
NLS sequences derived from NLS of an SV40 virus large T-antigen, having the amino acid sequence PKKKRKV (SEQ ID NO: 130); NLS (for example, the nucleoplasmin bipartite NLS having the sequence KRPAATKKAGQAKKKK (SEQ ID NO: 131)); c-myc NLS having the amino acid sequence PAAKRVKLD (SEQ ID NO: 132) or RQRRNELKRSP (SEQ ID NO: 133); hRNPA1 M9 NLS having the sequence NQSSNFGPMKGGNFGGRSSGPYGGGGQYFAKPRNQGGY (SEQ ID NO: 134); the sequence RMRIZFKNKGKDTAELRRRRVEVSVELRKAKKDEQILKRRNV (SEQ ID NO: 135) of an IBB domain from importin alpha; the sequences VSRKRPRP (SEQ ID NO: 136) and PPKKARED (SEQ ID NO: 137) of myoma T protein; the sequence PQPKKKPL (SEQ ID NO: 138) of human p53; the sequence SALIKKKKKMAP (SEQ ID NO: 139) of mouse c-abl IV; the sequences DRLRR (SEQ ID NO: 140) and PKQKKRK (SEQ ID NO: 141) of influenza virus NS1; the sequence RKLKKKIKKL (SEQ ID NO: 142) of hepatitis virus delta antigen; the sequence REKKKFLKRR (SEQ ID NO: 143) of mouse Mxl protein; the sequence KRKGDEVDGVDEVAKKKSKK (SEQ ID NO: 144) of human poly (ADP-ribose) polymerase; and the sequence RKCLQAGMNLEARKTKK (SEQ ID NO: 145) of steroid hormone receptor (human) glucocorticoid.

### Example 13, Inclusion of Tag

The Cas12f1 protein of any one of Examples 1 to 12, wherein the Cas12f1 protein further comprises at least one functional domain, peptide, or protein selected from:
a tag protein such as a histidine (His) tag, a V5 tag, a FLAG tag, an influenza hemagglutinin (HA) tag, a Myc tag, a VSV-G tag, and a thioredoxin (Trx) tag; a fluorescent protein such as green fluorescent protein (GFP), yellow fluorescent protein (YFP), cyan fluorescent protein (CFP), blue fluorescent protein (BFP), HcRED, and DsRed; and a reporter protein (enzyme) such as glutathione-S-transferase (GST), horseradish peroxidase (HRP), chloramphenicol acetyltransferase (CAT) beta-galactosidase, beta-glucuronidase, and luciferase.

### Example 14, PAM Sequence

The Cas12f1 protein of any one of Examples 1 to 13, wherein a protospacer adjacent motif (PAM) sequence that can be recognized by the Cas12f1 protein is 5′-TTTR-3′ where R is A or G.

### Example 15, Inclusion of Similar Sequences

A Cas12f1 protein, represented by an amino acid sequence having an identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with the amino acid sequence of the Cas12f1 protein of any one of Examples 1 to 14.

tracrRNA Scaffold

### Example 16, Wild-Type tracrRNA Scaffold

A wild-type tracrRNA scaffold, represented by a sequence selected from:

(SEQ ID NO: 110)
5′-CUUCACUGAUAAAGUGGAGAACCGCUUCACCAAAAGCUGUCCCUUA
GGGGAUUAGAACUUGAGUGAAGGUGGGCUGCUUGCAUCAGCCUAAUGUC
GAGAAGUGCUUUCUUCGGAAAGUAACCCUCGAAACAAAUUCAUUUUUC
CUCUCCAAUUCUGCACAA-3′;
and
(SEQ ID NO: 292)
5′-CUUCACUGAUAAAGUGGAGAACCGCUUCACCAAAAGCUGUCCCUUA
GGGGAUUAGAACUUGAGUGAAGGUGGGCUGCUUGCAUCAGCCUAAUGUC
GAGAAGUGCUUUCUUCGGAAAGUAACCCUCGAAACAAAUUCAUUU-3′

### Example 17, Engineered tracrRNA Scaffold, with Respect to WT

An engineered tracrRNA scaffold, represented by the following sequence:

5′-[first sequence]-[second sequence]-[third
sequence]-[fourth sequence]-[fifthsequence]-3′,

wherein the first sequence is selected 5′-CAAAUUCANNNCNCCUCUCCAAUUCUGCACAA-3′ (SEQ ID NO: 293), 5′-CAAAUUCANNNCNCCUCUCCAAUUCUGCACA-3′ (SEQ ID NO: 294), 5′-CAAAUUCANNNCNCCUCUCCAAUUCUGCAC-3′ (SEQ ID NO: 295), 5′-CAAAUUCANNNCNCCUCUCCAAUUCUGCA-3′ (SEQ ID NO: 296), 5′-CAAAUUCANNNCNCCUCUCCAAUUCUGC-3′ (SEQ ID NO: 297), 5′-CAAAUUCANNNCNCCUCUCCAAUUCUG-3′ (SEQ ID NO: 298), 5′-CAAAUUCANNNCNCCUCUCCAAUUCU-3′ (SEQ ID NO: 299), 5′-CAAAUUCANNNCNCCUCUCCAAUUC-3′ (SEQ ID NO: 300), 5′-CAAAUUCANNNCNCCUCUCCAAUU-3′ (SEQ ID NO: 4), 5′-CAAAUUCANNNCNCCUCUCCAAU-3′ (SEQ ID NO: 5), 5′-CAAAUUCANNNCNCCUCUCCAA-3′ (SEQ ID NO: 303), 5′-CAAAUUCANNNCNCCUCUCCA-3′ (SEQ ID NO: 304), 5′-CAAAUUCANNNCNCCUCUCC-3′ (SEQ ID NO: 305), 5′-CAAAUUCANNNCNCCUCUC-3′ (SEQ ID NO: 306), 5′-CAAAUUCANNNCNCCUCU-3′ (SEQ ID NO: 307), 5′-CAAAUUCANNNCNCCUC-3′ (SEQ ID NO: 308), 5′-CAAAUUCANNNCNCCU-3′ (SEQ ID NO: 309), 5′-CAAAUUCANNNCNCC-3′ (SEQ ID NO: 310), 5′-CAAAUUCANNNCNC-3′ (SEQ ID NO: 4), and 5′-CAAAUUCANNNCN-3′ (SEQ ID NO: 5),
the second sequence is 5′-AUGUCGAGAAGUGCUUUCUUCGGAAAGUAACCCUCGAAA-3′ (SEQ ID NO: 313),
the third sequence is 5′-GGCUGCUUGCAUCAGCCUA-3′ (SEQ ID NO: 314),
the fourth sequence is a sequence selected from 5′-CCGCUUCACCAAAAGCUGUCCCUUAGGGGAUUAGAACUUGAGUGAAG GUG-3′ (SEQ ID NO: 315), 5′-CCGCUUCACCAAAAGCUGUCCUUAGGGAUUAGAACUUGAGUGAAGGU G-3′ (SEQ ID NO: 316), 5′-CCGCUUCACCAAAAGCUGUCUUAGGAUUAGAACUUGAGUGAAGGUG-3 (SEQ ID NO: 317), 5′-CCGCUUCACCAAAAGCUGUUUAGAUUAGAACUUGAGUGAAGGUG-3′ (SEQ ID NO: 318), 5′-CCGCUUCACCAAAAGCUGUUAGUUAGAACUUGAGUGAAGGUG-3′ (SEQ ID NO: 319), 5′-CCGCUUCACCAAAAGCUGUUAGUAGAACUUGAGUGAAGGUG-3′ (SEQ ID NO: 320), 5′-CCGCUUCACCAAAAGCUUUAGAGAACUUGAGUGAAGGUG-3′ (SEQ ID NO: 4), 5′-CCGCUUCACCAAAAGCUUAGGAACUUGAGUGAAGGUG-3′ (SEQ ID NO: 5), 5′-CCGCUUCACCAAAAGUUAGAACUUGAGUGAAGGUG-3′ (SEQ ID NO: 323), 5′-CCGCUUCACCAAAAUUAGACUUGAGUGAAGGUG-3′ (SEQ ID NO: 324), 5′-CCGCUUCACCAAAUUAGCUUGAGUGAAGGUG-3′ (SEQ ID NO: 325), 5′-CCGCUUCACCAAUUAGUUGAGUGAAGGUG-3′ (SEQ ID NO: 326), 5′-CCGCUUCACCAUUAGUGAGUGAAGGUG-3′ (SEQ ID NO: 327), 5′-CCGCUUCACCUUAGGAGUGAAGGUG-3′ (SEQ ID NO: 328), 5′-CCGCUUCACUUAGAGUGAAGGUG-3′ (SEQ ID NO: 329), 5′-CCGCUUCACUUAGGUGAAGGUG-3′ (SEQ ID NO: 330), 5′-CCGCUUCAUUAGUGAAGGUG-3′ (SEQ ID NO: 4), 5′-CCGCUUCUUAGGAAGGUG-3′ (SEQ ID NO: 5), 5′-CCGCUUUUAGAAGGUG-3′ (SEQ ID NO: 333), 5′-CCGCUUUAGAGGUG-3′ (SEQ ID NO: 334), 5′-CCGCUUAGGGUG-3′ (SEQ ID NO: 335), 5′-CCGUUAGGUG-3′ (SEQ ID NO: 336), 5′-CCUUAGGUG-3′, 5′-CUUAGUG-3′, 5′-CUUAGG-3′, and 5′-UUAG-3′, and
the fifth sequence is a sequence selected from 5′-CUUCACUGAUAAAGUGGAGAA-3′ (SEQ ID NO: 337), 5′-A-3′, 5′-AA-3′, 5′-GAA-3′, 5′-AGAA-3′, 5′-GAGAA-3′, 5′-GGAGAA-3′, 5′-UGGAGAA-3′, 5′-GUGGAGAA-3′, 5′-AGUGGAGAA-3′, 5′-AAGUGGAGAA-3′ (SEQ ID NO: 338), 5′-AAAGUGGAGAA-3′ (SEQ ID NO: 339), 5′-UAAAGUGGAGAA-3′ (SEQ ID NO: 340), 5′-AUAAAGUGGAGAA-3′ (SEQ ID NO: 4), 5′-GAUAAAGUGGAGAA-3′ (SEQ ID NO: 5), 5′-UGAUAAAGUGGAGAA-3′ (SEQ ID NO: 343), 5′-CUGAUAAAGUGGAGAA-3′ (SEQ ID NO: 344), 5′-ACUGAUAAAGUGGAGAA-3′ (SEQ ID NO: 345), 5′-CACUGAUAAAGUGGAGAA-3′ (SEQ ID NO: 346), 5′-UCACUGAUAAAGUGGAGAA-3′ (SEQ ID NO: 347), and 5′-UUCACUGAUAAAGUGGAGAA-3′ (SEQ ID NO: 348),
wherein each N is independently A, C, G or U, and
the sequence is characterized by being different from 5′-CUUCACUGAUAAAGUGGAGAACCGCUUCACCAAAAGCUGUCCCUUAG GGGAUUAGAACUUGAGUGAAGGUGGGCUGCUUGCAUCAGCCUAAUGUC GAGAAGUGCUUUCUUCGGAAAGUAACCCUCGAAACAAAUUCAUUUUUC CUCUCCAAUUCUGCACAA-3′ (SEQ ID NO: 110) and 5′-CUUCACUGAUAAAGUGGAGAACCGCUUCACCAAAAGCUGUCCCUUAG GGGAUUAGAACUUGAGUGAAGGUGGGCUGCUUGCAUCAGCCUAAUGUC GAGAAGUGCUUUCUUCGGAAAGUAACCCUCGAAACAAAUUCAUUUUUC CUCUCCAAUUCUGCACAA-3′ (SEQ ID NO: 110).

### Example 18, Substitution of Linker Sequence

The engineered tracrRNA scaffold of Example 17, wherein the sequence 5′-UUAG-3′ included in the fourth sequence is substituted with the sequence 5′-GAAA-3′.

### Example 19, Engineered tracrRNA Scaffold, with Respect to MF

An engineered tracrRNA scaffold, represented by the following sequence:

5′-[first region]-[second region]-[third region]-
[fourth region]-3′,

wherein the first region is absent or selected from the group consisting of 5′-A-3′, 5′-AA-3′, 5′-GAA-3′, 5′-AGAA-3′, 5′-GAGAA-3′, 5′-GGAGAA-3′, 5′-UGGAGAA-3′, 5′-GUGGAGAA-3′, 5′-AGUGGAGAA-3′, 5′-AAGUGGAGAA-3′ (SEQ ID NO: 349), 5′-AAAGUGGAGAA-3′ (SEQ ID NO: 350), 5′-UAAAGUGGAGAA-3′ (SEQ ID NO: 4), 5′-AUAAAGUGGAGAA-3′ (SEQ ID NO: 5), 5′-GAUAAAGUGGAGAA-3′ (SEQ ID NO: 353), 5′-UGAUAAAGUGGAGAA-3′ (SEQ ID NO: 354), 5′-CUGAUAAAGUGGAGAA-3′ (SEQ ID NO: 355), 5′-ACUGAUAAAGUGGAGAA-3′ (SEQ ID NO: 356), 5′-CACUGAUAAAGUGGAGAA-3′ (SEQ ID NO: 357), 5′-UCACUGAUAAAGUGGAGAA-3′ (SEQ ID NO: 358), 5′-UUCACUGAUAAAGUGGAGAA-3′ (SEQ ID NO: 359), and 5′-CUUCACUGAUAAAGUGGAGAA-3′ (SEQ ID NO: 360),
the second region is absent or selected from the group consisting of 5′-G-3′, 5′-UUAGG-3′, 5′-CUUAGGG-3′, 5′-CUUAGUGG-3′, 5′-CCUUAGGUGG-3′ (SEQ ID NO: 4), 5′-CCGUUAGGUGG-3′ (SEQ ID NO: 5), 5′-CCGCUUAGGGUGG-3′ (SEQ ID NO: 363), 5′-CCGCUUUAGAGGUGG-3′ (SEQ ID NO: 364), 5′-CCGCUUUUAGAAGGUGG-3′ (SEQ ID NO: 365), 5′-CCGCUUCUUAGGAAGGUGG-3′ (SEQ ID NO: 366), 5′-CCGCUUCAUUAGUGAAGGUGG-3′ (SEQ ID NO: 367), 5′-CCGCUUCACUUAGGUGAAGGUGG-3′ (SEQ ID NO: 368), 5′-CCGCUUCACUUAGAGUGAAGGUGG-3′ (SEQ ID NO: 369), 5′-CCGCUUCACCUUAGGAGUGAAGGUGG-3′ (SEQ ID NO: 370), 5′-CCGCUUCACCAUUAGUGAGUGAAGGUGG-3′ (SEQ ID NO: 4), 5′-CCGCUUCACCAAUUAGUUGAGUGAAGGUGG-3′ (SEQ ID NO: 5), 5′-CCGCUUCACCAAAUUAGCUUGAGUGAAGGUGG-3′ (SEQ ID NO: 373), 5′-CCGCUUCACCAAAAUUAGACUUGAGUGAAGGUGG-3′ (SEQ ID NO: 374), 5′-CCGCUUCACCAAAAGUUAGAACUUGAGUGAAGGUGG-3′ (SEQ ID NO: 375), 5′-CCGCUUCACCAAAAGCUUAGGAACUUGAGUGAAGGUGG-3′ (SEQ ID NO: 376), 5′-CCGCUUCACCAAAAGCUUUAGAGAACUUGAGUGAAGGUGG-3′ (SEQ ID NO: 377), 5′-CCGCUUCACCAAAAGCUGUUAGUAGAACUUGAGUGAAGGUGG-3′ (SEQ ID NO: 378), 5′-CCGCUUCACCAAAAGCUGUUAGUUAGAACUUGAGUGAAGGUGG-3′ (SEQ ID NO: 379), 5′-CCGCUUCACCAAAAGCUGUUUAGAUUAGAACUUGAGUGAAGGUGG-3′ (SEQ ID NO: 380), 5′-CCGCUUCACCAAAAGCUGUCUUAGGAUUAGAACUUGAGUGAAGGUGG-3′ (SEQ ID NO: 4), and 5′-CCGCUUCACCAAAAGCUGUCCCUUAGGGGAUUAGAACUUGAGUGAAG GUGG-3′ (SEQ ID NO: 5),
the third region is selected from the group consisting of 5′-GCUGCUUGCAUCAGCCUAAUGUCGAGAAGUGCUUUCUUCGGAAAGUA ACCCUCGA-3′ (SEQ ID NO: 383), 5′-GCUGCUUGCAUCAGCCUAAUGUCGAGAAGUGCUUUUUCGAAAGUAAC CCUCGA-3′ (SEQ ID NO: 384), 5′-GCUGCUUGCAUCAGCCUAAUGUCGAGAAGUGCUUUUCGAAGUAACCC UCGA-3′ (SEQ ID NO: 385), 5′-GCUGCUUGCAUCAGCCUAAUGUCGAGAAGUGCUCUUCGGAGUAACCC UCGA-3′ (SEQ ID NO: 386), 5′-GCUGCUUGCAUCAGCCUAAUGUCGAGAAGUGCUUUCGAGUAACCCUC GA-3′ (SEQ ID NO: 387), 5′-GCUGCUUGCAUCAGCCUAAUGUCGAGAAGUGCUUCGGUAACCCUCGA-3′ (SEQ ID NO: 388), 5′-GCUGCUUGCAUCAGCCUAAUGUCGAGAAGUGUUCGUAACCCUCGA-3′ (SEQ ID NO: 389), 5′-GCUGCUUGCAUCAGCCUAAUGUCGAGAAGUUUCGAACCCUCGA-3′ (SEQ ID NO: 390), 5′-GCUGCUUGCAUCAGCCUAAUGUCGAGAAGUUCGACCCUCGA-3′ (SEQ ID NO: 4), 5′-GCUGCUUGCAUCAGCCUAAUGUCGAGAAGUUCGCCCUCGA-3′ (SEQ ID NO: 5), 5′-GCUGCUUGCAUCAGCCUAAUGUCGAGAAUUCGCCCUCGA-3′ (SEQ ID NO: 393), 5′-GCUGCUUGCAUCAGCCUAAUGUCGAGAAUUCGCCUCGA-3′ (SEQ ID NO: 394), 5′-GCUGCUUGCAUCAGCCUAAUGUCGAGAUUCGCCUCGA-3′ (SEQ ID NO: 395), 5′-GCUGCUUGCAUCAGCCUAAUGUCGAGUUCGCUCGA-3′ (SEQ ID NO: 396), and 5′-GCUGCUUGCAUCAGCCUAAUGUCGAGAAGUGCUUUCUUCGGAAAGUA ACCCUCGA-3′ (SEQ ID NO: 397), and
the fourth region is a sequence selected from 5′-AACAAA-3′ (SEQ ID NO: 398), 5′-AACAAAU-3′ (SEQ ID NO: 399), 5′-AACAAAUU-3′ (SEQ ID NO: 400), 5′-AACAAAUUC-3′ (SEQ ID NO: 401), 5′-AACAAAUUCA-3′ (SEQ ID NO: 402), 5′-AACAAAUUCAU-3′ (SEQ ID NO: 403), 5′-AACAAAUUCAUU-3′ (SEQ ID NO: 404), and 5′-AACAAAUUCAUUU-3′ (SEQ ID NO: 405),
wherein the sequence is charactered by being different from 5′-CUUCACUGAUAAAGUGGAGAACCGCUUCACCAAAAGCUGUCCCUUAG GGGAUUAGAACUUGAGUGAAGGUGGGCUGCUUGCAUCAGCCUAAUGUC GAGAAGUGCUUUCUUCGGAAAGUAACCCUCGAAACAAAUUCAUUU-3′ (SEQ ID NO: 292).

### Example 20, Inclusion of Similar Sequences

A tracrRNA scaffold, represented by a sequence having an identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with the sequence of the tracrRNA scaffold of any one of Examples 16 to 19.

crRNA scaffold

### Example 21, Wild-Type crRNA Scaffold

A crRNA scaffold, represented by a sequence selected from:

(SEQ ID NO: 101)
5′-GUUGCAGAACCCGAAUAGACGAAUGAAGGAAUGCAAC-3′;
and
(SEQ ID NO: 406)
5′-GAAUGAAGGAAUGCAAC-3′.

### Example 22, Engineered crRNA Scaffold, with Respect to WT

An engineered crRNA scaffold, represented by the following sequence:

5′-[sixth sequence]-[seventh sequence]-3′,

wherein the sixth sequence is a sequence selected from 5′-GUUGCAGAACCCGAAUAGNBNNNUGAAGGA-3′ (SEQ ID NO: 407), 5′-GUUGCAGAACCCGAAUAGNGNNNUGAAGGA-3′ (SEQ ID NO: 408), 5′-UUGCAGAACCCGAAUAGNGNNNUGAAGGA-3′ (SEQ ID NO: 409), 5′-UGCAGAACCCGAAUAGNGNNNUGAAGGA-3′ (SEQ ID NO: 410), 5′-GCAGAACCCGAAUAGNGNNNUGAAGGA-3′ (SEQ ID NO: 411), 5′-CAGAACCCGAAUAGNGNNNUGAAGGA-3′ (SEQ ID NO: 412), 5′-AGAACCCGAAUAGNGNNNUGAAGGA-3′ (SEQ ID NO: 413), 5′-GAACCCGAAUAGNGNNNUGAAGGA-3′ (SEQ ID NO: 414), 5′-AACCCGAAUAGNGNNNUGAAGGA-3′ (SEQ ID NO: 415), 5′-ACCCGAAUAGNGNNNUGAAGGA-3′ (SEQ ID NO: 416), 5′-CCCGAAUAGNGNNNUGAAGGA-3′ (SEQ ID NO: 417), 5′-CCGAAUAGNGNNNUGAAGGA-3′ (SEQ ID NO: 418), 5′-CGAAUAGNGNNNUGAAGGA-3′ (SEQ ID NO: 419), 5′-GAAUAGNGNNNUGAAGGA-3′ (SEQ ID NO: 420), 5′-AAUAGNGNNNUGAAGGA-3′ (SEQ ID NO: 421), 5′-AUAGNGNNNUGAAGGA-3′ (SEQ ID NO: 422), 5′-UAGNGNNNUGAAGGA-3′ (SEQ ID NO: 423), 5′-AGNGNNNUGAAGGA-3′ (SEQ ID NO: 424), and 5′-NGNNNUGAAGGA-3′ (SEQ ID NO: 425), and
the seventh sequence is 5′-AUGCAAC-3′,
wherein each N is independently A, C, G or U.

### Example 23, Engineered crRNA Scaffold, with Respect to MF

An engineered crRNA scaffold, represented by the following sequence:

5′-[fifth region]-[sixth region]-3′

wherein the fifth region is selected from the group consisting 5′-GGA-3′, 5′-AGGA-3′, 5′-AAGGA-3′, 5′-GAAGGA-3′, 5′-UGAAGGA-3′, 5′-AUGAAGGA-3′, 5′-AAUGAAGGA-3′, 5′-GAAUGAAGGA-3′ (SEQ ID NO: 426), 5′-CGAAUGAAGGA-3′ (SEQ ID NO: 427), 5′-ACGAAUGAAGGA-3′ (SEQ ID NO: 428), 5′-GACGAAUGAAGGA-3′ (SEQ ID NO: 429), 5′-AGACGAAUGAAGGA-3′ (SEQ ID NO: 430), 5′-UAGACGAAUGAAGGA-3′ (SEQ ID NO: 431), 5′-AUAGACGAAUGAAGGA-3′ (SEQ ID NO: 432), 5′-AAUAGACGAAUGAAGGA-3′ (SEQ ID NO: 433), 5′-GAAUAGACGAAUGAAGGA-3′ (SEQ ID NO: 434), 5′-CGAAUAGACGAAUGAAGGA-3′ (SEQ ID NO: 435), 5′-CCGAAUAGACGAAUGAAGGA-3′ (SEQ ID NO: 436), 5′-CCCGAAUAGACGAAUGAAGGA-3′ (SEQ ID NO: 437), 5′-ACCCGAAUAGACGAAUGAAGGA-3′ (SEQ ID NO: 438), 5′-AACCCGAAUAGACGAAUGAAGGA-3′ (SEQ ID NO: 439), 5′-GAACCCGAAUAGACGAAUGAAGGA-3′ (SEQ ID NO: 440), 5′-AGAACCCGAAUAGACGAAUGAAGGA-3′ (SEQ ID NO: 441), 5′-CAGAACCCGAAUAGACGAAUGAAGGA-3′ (SEQ ID NO: 442), 5′-GCAGAACCCGAAUAGACGAAUGAAGGA-3′ (SEQ ID NO: 443), 5′-UGCAGAACCCGAAUAGACGAAUGAAGGA-3′ (SEQ ID NO: 444), and 5′-UUGCAGAACCCGAAUAGACGAAUGAAGGA-3′ (SEQ ID NO: 445), and
the sixth region is 5′-AUGCAAC-3′,
wherein the sequence is characterized by being different from 5′-GUUGCAGAACCCGAAUAGACGAAUGAAGGAAUGCAAC-3′ (SEQ ID NO: 101) and 5′-GAAUGAAGGAAUGCAAC-3′ (SEQ ID NO: 406).

### Example 24, Inclusion of Similar Sequences

A crRNA scaffold, represented by a sequence having an identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with the sequence of the crRNA scaffold of any one of Examples 21 to 23.

### Guide Region

### Example 25, Guide Region (Spacer Region)

A guide region (spacer region), capable of recognizing, binding to, or targeting the CEP290 gene.

### Example 26, Intron 26 Region, Target Sequence

The guide region of Example 25, wherein the guide region has a guide sequence capable of complementarily binding to a target sequence present in a partial region of the intron 26 region containing the IVS26 mutation (c.2991+1655A>G) of the CEP290 gene.

### Example 27, Intron 26 Region, Protospacer Sequence

The guide region of Example 25, wherein the guide region has a guide sequence having a nucleotide sequence that is identical or equivalent to a protospacer sequence present in a partial region of the intron 26 region containing the IVS26 mutation (c.2991+1655A>G) of the CEP290 gene, in which the guide sequence is a sequence in which T is replaced with U in the nucleotide sequence of the protospacer sequence.

### Example 28, PAM Sequence, Adjacent

The guide region of Example 27, wherein the protospacer sequence is located at the 5′ end and/or the 3′ end of a PAM sequence recognized by the Cas12f1 protein of any one of Examples 1 to 15.

### Example 29, PAM Sequence, Defined

The guide region of Example 28, wherein the PAM sequence is 5′-TTTR-3′ where R is A or G.

### Example 30, IVS26 Upstream

The guide region of Example 26, wherein the partial region of the intron 26 region is a region between the 3′ end of exon 26 of the CEP290 gene and the IVS26 mutation (c.2991+1655A>G) of the CEP290 gene.

### Example 31, Upstream 1654 bp

The guide region of Example 30, wherein based on the IVS26 mutation (c.2991+1655A>G), the partial region of the intron 26 region is a region (upstream 1654 bp region) comprising nucleotides within a distance range of 1 bp to 1654 bp in a direction from the IVS26 mutation to the 5′-end.

### Example 32, IVS26 Downstream

The guide region of Example 26, wherein the partial region of the intron 26 region is a region between the IVS26 mutation (c.2991+1655A>G) of the CEP290 gene and the 5′ end of exon 27 of the CEP290 gene.

### Example 33, Downstream 2000 bp

The guide region of Example 32, wherein based on the IVS26 mutation (c.2991+1655A>G), the partial region of the intron 26 region is a region (downstream 2000 bp region) comprising nucleotides within a distance range of 1 bp to 2000 bp in a direction from the IVS26 mutation to the 3′-end.

### Example 34, Target Sequence, Defined

The guide region of Example 26, wherein the target sequence is a nucleotide sequence selected from:

(SEQ ID NO: 1)
5′-TAAATAGGCATAATTTTCTA-3′;
(SEQ ID NO: 2)
5′-TTGCTTTCTGCTGCTTTTGC-3′;
(SEQ ID NO: 3)
5′-GTAGCTTTCAGGATTCCTAC-3′;
(SEQ ID NO: 4)
5′-GGAGCTTGTTCTGTCCTCAG-3′;
(SEQ ID NO: 5)
5′-GTTTAACGTTATCATTTTCC-3′;
(SEQ ID NO: 6)
5′-GCTCATAGAGACACATTCAG-3′;
(SEQ ID NO: 7)
5′-TTTCTGATGAGGAAGATGAA-3′;
(SEQ ID NO: 8)
5′-GATCTTAGATAAGAATAATC-3′;
(SEQ ID NO: 9)
5′-TTACTTCTAAATAATATTGA-3′;
(SEQ ID NO: 10)
5′-TGGACCATGGATGCACTCTG-3′;
(SEQ ID NO: 11)
5′-GCTACATCCATTCCAAGGAA-3′;
(SEQ ID NO: 12)
5′-TTTTCTCTTAGATGTCTGGT-3′;
(SEQ ID NO: 13)
5′-TGTAGAATTTTAATGTAGAA-3′;
(SEQ ID NO: 14)
5′-TCTTACCCCTGTACCCAGAA-3′;
(SEQ ID NO: 15)
5′-CTTGCATGATTTAGCTGAAT-3′;
(SEQ ID NO: 16)
5′-CAGAAGTCATTCAGCCACTA-3′;
(SEQ ID NO: 17)
5′-GTGTGTGTGTTATGTGGGAA-3′;
(SEQ ID NO: 18)
5′-TTATTCATTCAGTTTAGTTA-3′;
(SEQ ID NO: 19)
5′-GGGATTTGGCAGATTTTAAT-3′;
(SEQ ID NO: 20)
5′-CTGCCAAATCCCCCCAAACA-3′;
(SEQ ID NO: 21)
5′-CACAAGGTTCAAGAATCACA-3′;
(SEQ ID NO: 22)
5′-CATCATTTTTTATTGTAGAA-3′;
(SEQ ID NO: 23)
5′-GAATGTGTCTCTATGAGCCAG-3′;
(SEQ ID NO: 24)
5′-TAAGATCTAATTCTATTAGCT-3′;
(SEQ ID NO: 25)
5′-TCTCATGAACCTTTACCTCT-3′;
(SEQ ID NO: 26)
5′-TATTAGCTTGAACTCTGTGC-3′;
(SEQ ID NO: 27)
5′-CATTAAGGAAGACAGATATG-3′;
(SEQ ID NO: 28)
5′-CAGGAGTGACTTTGTTCCAT-3′;
(SEQ ID NO: 29)
5′-GCTACCGGTTACCTGAAGGG-3′;
(SEQ ID NO: 30)
5′-TGCCACAAGAATGATCATTC-3′;
(SEQ ID NO: 31)
5′-TCCCATGTGACTCCCCGCCT-3′;
(SEQ ID NO: 32)
5′-TATTGTTGCTTTTTGAGAGG-3′;
(SEQ ID NO: 33)
5′-GTGAATTTCTATTCCTGTTT-3′;
(SEQ ID NO: 34)
5′-GCATACTTTTTTTAATGGAA-3′;
(SEQ ID NO: 35)
5′-CTCAACACACAGAAACAAAT-3′;
(SEQ ID NO: 36)
5′-GAATAGATAATAAGGAAATA-3′;
(SEQ ID NO: 37)
5′-TTTCTTTTTTCTTCTACTAT-3′;
(SEQ ID NO: 38)
5′-CCTGTGTTTCAAGGGGATCT-3′;
(SEQ ID NO: 39)
5′-CCCTTGAAACACAGGAATTG-3′;
(SEQ ID NO: 40)
5′-TAAGCAATGGATTTCAGTGA-3′;
(SEQ ID NO: 41)
5′-CAATGGATTTCAGTGATAAA-3′;
(SEQ ID NO: 42)
5′-TATTTTCCAGGTACTCCACT-3′;
(SEQ ID NO: 43)
5′-TACAAACAGGGTAATGAGAC-3′;
(SEQ ID NO: 44)
5′-TCCTGTGTATTCATAGTATA-3′;
(SEQ ID NO: 45)
5′-GATAATCCTATTGTTTGTTA-3′;
(SEQ ID NO: 46)
5′-CACTCTCTCCCTAGTAACTC-3′;
(SEQ ID NO: 47)
5′-GAAACTTCACTGGTCTCCTT-3′;
(SEQ ID NO: 48)
5′-CTGGCGGATTACCTGAGATC-3′;
(SEQ ID NO: 49)
5′-CTATAATTATGTACTTTACT-3′;
and
(SEQ ID NO: 50)
5′-CATGTTGGCCGGGCTGGTCT-3′.

### Example 35, Guide Sequence, Length

The guide region of any one of Examples 26 to 34, wherein the guide sequence has a length of 15 nts, 16 nts, 17 nts, 18 nts, 19 nts, 20 nts, 21 nts, 22 nts, 23 nts, 24 nts, 25 nts, 26 nts, 27 nts, 28 nts, 29 nts, 30 nts., 31 nts, 32 nts, 33 nts, 34 nts, 35 nts, 36 nts, 37 nts, 38 nts, 39 nts, or 40 nts, or a length falling within a range of the aforementioned two numbers.

### Example 36, Target Sequence, Mismatched

The guide region of any one of Examples 26 to 34, wherein the guide sequence comprises 0, 1, 2, 3, 4, or 5 mismatched nucleotide sequences, or mismatched nucleotide sequences whose number falls within a range of the aforementioned two numbers.

### Example 37, Example of Guide Sequence

The guide region of Example 26, wherein the guide sequence is a nucleotide sequence selected from:

(SEQ ID NO: 51)
5′-UAGAAAAUUAUGCCUAUUUA-3′;
(SEQ ID NO: 52)
5′-GCAAAAGCAGCAGAAAGCAA-3′;
(SEQ ID NO: 53)
5′-GUAGGAAUCCUGAAAGCUAC-3′;
(SEQ ID NO: 54)
5′-CUGAGGACAGAACAAGCUCC-3′;
(SEQ ID NO: 55)
5′-GGAAAAUGAUAACGUUAAAC-3′;
(SEQ ID NO: 56)
5′-CUGAAUGUGUCUCUAUGAGC-3′;
(SEQ ID NO: 57)
5′-UUCAUCUUCCUCAUCAGAAA-3′;
(SEQ ID NO: 58)
5′-GAUUAUUCUUAUCUAAGAUC-3′;
(SEQ ID NO: 59)
5′-UCAAUAUUAUUUAGAAGUAA-3′;
(SEQ ID NO: 60)
5′-CAGAGUGCAUCCAUGGUCCA-3′;
(SEQ ID NO: 61)
5′-UUCCUUGGAAUGGAUGUAGC-3′;
(SEQ ID NO: 62)
5′-ACCAGACAUCUAAGAGAAAA-3′;
(SEQ ID NO: 63)
5′-UUCUACAUUAAAAUUCUACA-3′;
(SEQ ID NO: 64)
5′-UUCUGGGUACAGGGGUAAGA-3′;
(SEQ ID NO: 65)
5′-AUUCAGCUAAAUCAUGCAAG-3′;
(SEQ ID NO: 66)
5′-UAGUGGCUGAAUGACUUCUG-3′;
(SEQ ID NO: 67)
5′-UUCCCACAUAACACACACAC-3′;
(SEQ ID NO: 68)
5′-UAACUAAACUGAAUGAAUAA-3′;
(SEQ ID NO: 69)
5′-AUUAAAAUCUGCCAAAUCCC-3′;
(SEQ ID NO: 70)
5′-UGUUUGGGGGGAUUUGGCAG-3′;
(SEQ ID NO: 71)
5′-UGUGAUUCUUGAACCUUGUG-3′;
(SEQ ID NO: 72)
5′-UUCUACAAUAAAAAAUGAUG-3′;
(SEQ ID NO: 73)
5′-CUGGCUCAUAGAGACACAUUC-3′;
(SEQ ID NO: 74)
5′-AGCUAAUAGAAUUAGAUCUUA-3′;
(SEQ ID NO: 75)
5′-AGAGGUAAAGGUUCAUGAGA-3′;
(SEQ ID NO: 76)
5′-GCACAGAGUUCAAGCUAAUA-3′;
(SEQ ID NO: 77)
5′-CAUAUCUGUCUUCCUUAAUG-3′;
(SEQ ID NO: 79)
5′-AUGGAACAAAGUCACUCCUG-3′;
(SEQ ID NO: 79)
5′-CCCUUCAGGUAACCGGUAGC-3′;
(SEQ ID NO: 80)
5′-GAAUGAUCAUUCUUGUGGCA-3′;
(SEQ ID NO: 81)
5′-AGGCGGGGAGUCACAUGGGA-3′;
(SEQ ID NO: 82)
5′-CCUCUCAAAAAGCAACAAUA-3′;
(SEQ ID NO: 83)
5′-AAACAGGAAUAGAAAUUCAC-3′;
(SEQ ID NO: 84)
5′-UUCCAUUAAAAAAAGUAUGC-3′;
(SEQ ID NO: 85)
5′-AUUUGUUUCUGUGUGUUGAG-3′;
(SEQ ID NO: 86)
5′-UAUUUCCUUAUUAUCUAUUC-3′;
(SEQ ID NO: 87)
5′-AUAGUAGAAGAAAAAAGAAA-3′;
(SEQ ID NO: 88)
5′-AGAUCCCCUUGAAACACAGG-3′;
(SEQ ID NO: 89)
5′-CAAUUCCUGUGUUUCAAGGG-3′;
(SEQ ID NO: 90)
5′-UCACUGAAAUCCAUUGCUUA-3′;
(SEQ ID NO: 91)
5′-UUUAUCACUGAAAUCCAUUG-3′;
(SEQ ID NO: 92)
5′-AGUGGAGUACCUGGAAAAUA-3′;
(SEQ ID NO: 93)
5′-GUCUCAUUACCCUGUUUGUA-3′;
(SEQ ID NO: 94)
5′-UAUACUAUGAAUACACAGGA-3′;
(SEQ ID NO: 95)
5′-UAACAAACAAUAGGAUUAUC-3′;
(SEQ ID NO: 96)
5′-GAGUUACUAGGGAGAGAGUG-3′;
(SEQ ID NO: 97)
5′-AAGGAGACCAGUGAAGUUUC-3′;
(SEQ ID NO: 98)
5′-GAUCUCAGGUAAUCCGCCAG-3′;
(SEQ ID NO: 99)
5′-AGUAAAGUACAUAAUUAUAG-3′;
(SEQ ID NO: 101)
5′-AGACCAGCCCGGCCAACAUG-3′;
(SEQ ID NO: 245)
5′-CAGAGUGCAUCCAUGGUCC-3′;
(SEQ ID NO: 246)
5′-CAGAGUGCAUCCAUGGUCCAG-3′;
(SEQ ID NO: 247)
5′-CAGAGUGCAUCCAUGGUCCAGG-3′;
(SEQ ID NO: 248)
5′-CAGAGUGCAUCCAUGGUCCAGGA-3′;
(SEQ ID NO: 249)
5′-CAGAGUGCAUCCAUGGUCCAGGAA-3′;
(SEQ ID NO: 250)
5′-CAGAGUGCAUCCAUGGUCCAGGAAG-3′;
(SEQ ID NO: 251)
5′-UUCUGGGUACAGGGGUAAG-3′;
(SEQ ID NO: 252)
5′-UUCUGGGUACAGGGGUAAGAG-3′;
(SEQ ID NO: 253)
5′-UUCUGGGUACAGGGGUAAGAGA-3′;
(SEQ ID NO: 254)
5′-UUCUGGGUACAGGGGUAAGAGAA-3′;
(SEQ ID NO: 255)
5′-UUCUGGGUACAGGGGUAAGAGAAA-3′;
(SEQ ID NO: 256)
5′-UUCUGGGUACAGGGGUAAGAGAAAG-3′;
(SEQ ID NO: 257)
5′-CCCUUCAGGUAACCGGUAG-3′;
(SEQ ID NO: 258)
5′-CCCUUCAGGUAACCGGUAGCUUUUG-3′;
(SEQ ID NO: 259)
5′-AGGCGGGGAGUCACAUGGG-3′;
(SEQ ID NO: 260)
5′-AGGCGGGGAGUCACAUGGGAG-3′;
(SEQ ID NO: 261)
5′-AGGCGGGGAGUCACAUGGGAGUC-3′;
(SEQ ID NO: 262)
5′-AGGCGGGGAGUCACAUGGGAGUCA-3′;
(SEQ ID NO: 263)
5′-AGGCGGGGAGUCACAUGGGAGUCAC-3′;
(SEQ ID NO: 264)
5′-AGUGGAGUACCUGGAAAAUAA-3′;
(SEQ ID NO: 265)
5′-AGUGGAGUACCUGGAAAAUAAG-3′;
(SEQ ID NO: 266)
5′-AGUGGAGUACCUGGAAAAUAAGC-3′;
(SEQ ID NO: 267)
5′-AGUGGAGUACCUGGAAAAUAAGCC-3′;
(SEQ ID NO: 268)
5′-AGUGGAGUACCUGGAAAAUAAGCCA-3′;
(SEQ ID NO: 269)
5′-GAGUUACUAGGGAGAGAGUGA-3′;
(SEQ ID NO: 270)
5′-GAGUUACUAGGGAGAGAGUGAUG-3′;
(SEQ ID NO: 271)
5′-GAGUUACUAGGGAGAGAGUGAUGA-3′;
and
(SEQ ID NO: 272)
5′-GAGUUACUAGGGAGAGAGUGAUGAA-3′.

### Example 38, Inclusion of Similar Sequences

A guide region, comprising a guide sequence having a nucleotide sequence with an identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with the guide sequence of any one of Examples 25 to 37.

### U-Rich Tail

### Example 39, Example of U-Rich Tail Sequence

A U-rich tail, represented by a sequence selected from:

5′-(UaN)dUe-3′;
5′-UaVUaVUe-3′;
and
5′-UaVUaVUaVUe-3′,

wherein N is A, C, G or U, and each V is independently A, C or G, and
a is an integer of 0 to 4, d is an integer of 0 to 3, and e is an integer of 1 to 10.

### Guide RNA

### Example 40, Dual Guide RNA

A guide RNA, comprising:
a scaffold region comprising a crRNA scaffold and a tracrRNA scaffold;
a guide region; and
optionally, a U-rich tail,
wherein the scaffold region is a region capable of interacting with a Cas12f1 protein,
the scaffold region is located at the 5′ end of the guide region,
the guide region has a guide sequence that forms a complementary bond with a target sequence present in the CEP290 gene, and
in a case where the guide RNA comprises the U-rich tail, the U-rich tail is located at the 3′ end of the guide RNA.

### Example 41, Single Guide RNA

The guide RNA of Example 40, wherein the scaffold region further comprises a linker, and the tracrRNA scaffold, the linker, and the crRNA scaffold are sequentially linked to each other in a 5′ to 3′ direction of the scaffold region.

### Example 42, Example of crRNA Scaffold

The guide RNA of any one of Examples 40 to 41, wherein the crRNA scaffold is the crRNA scaffold of any one of Examples 21 to 24.

### Example 43, Example of tracrRNA Scaffold

The guide RNA of any one of Examples 40 to 42, wherein the tracrRNA scaffold is the tracrRNA scaffold of any one of Examples 16 to 20.

### Example 44, Example of Guide Region

The guide RNA of any one of Examples 40 to 43, wherein the guide region is the guide region of any one of Examples 25 to 38.

### Example 45, Example of U-Rich Tail

The guide RNA of any one of Examples 40 to 44, wherein the U-rich tail is the U-rich tail of Example 39.

### Example 46, Guide RNA, Structure of Guide RNA

A guide RNA, represented by the following sequence:

5′-[tracrRNA scaffold sequence]-[linker
sequence]-[crRNA scaffold sequence]-[guide
sequence]-[U-rich tail sequence]-3′,

wherein the tracrRNA scaffold sequence is the sequence of any one of the tracrRNA scaffold of any one of Examples 16 to 20,
the linker sequence may be absent,
the crRNA scaffold sequence is the sequence of the crRNA scaffold of any one of Examples 21 to 24,
the guide sequence is a guide sequence in the guide region of any one of Examples 26 to 38, and
the U-rich tail sequence is absent or the sequence of the U-rich tail of Example 39,
wherein in a case where the linker sequence is absent, the guide RNA is a dual guide RNA of two molecules, and
in a case where the linker sequence is present, the guide RNA is a single guide RNA of one molecule.

### Example 47, Linker, Defined

The guide RNA of Example 46, wherein the linker is 5′-GAAA-3′.

### Example 48, Inclusion of Similar Sequences

A guide RNA, having a nucleotide sequence with an identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with the guide RNA of any one of Examples 40 to 47.

### Example 49, DNA Encoding Guide RNA

A DNA, encoding the guide RNA selected from any one of Examples 40 to 48.

### Additional Components

### Example 50, Factor for Decreasing Non-Homologous End-Binding Activity

A factor for decreasing non-homologous end joining DNA repair pathway (NHEJ) activity.

### Example 51, Target for Decreased Activity

The factor of Example 50, wherein the factor for decreasing NHEJ activity is capable of decreasing or removing expression or activity of at lease one gene selected from MRE11, RAD50, NBS1, DNA-PK, CtIP, Ku70, Ku80, Artemis (DCLRE1C), ligase IV (Lig4), PNKP, XRCC4, XLF (XRCC4-like factor), ATM (ATM serine/threonine kinase), CHK1/CHK2, CLF (CURLY LEAF), Pol Mu (POLM), ATM1, XRCC4, XLF, XRCC6, LIG4, and DCLRE1C.

### Example 52, Interference Nucleic Acid

The factor of Example 51, wherein the factor for decreasing NHEJ activity is selected from a short interfering RNA (siRNA), a double-stranded RNA (dsRNA), a micro RNA (miRNA), a short hairpin RNA (shRNA) specific for gene transcript, and an antisense oligonucleotide.

### Example 53, Sequence of shRNA

The factor of any one of Examples 50 to 52, wherein the factor for decreasing NHEJ activity is a shRNA represented by a sequence selected from:

(SEQ ID NO: 446)
5′-GGAGCCAGAUAGUUUGUAUUUCAAGAGAAUACAAACUAUCUGGCUCC
-3′;
(SEQ ID NO: 447)
5′-GCAAGCAGCUGAAACAAAUUUCAAGAGAAUUUGUUUCAGCUGCUUGC
-3′;
(SEQ ID NO: 448)
5′-GGAGCUGAUUGUAGCAACAUUCAAGAGAUGUUGCUACAAUCAGCUCC
-3′;
(SEQ ID NO: 449)
5′-GCACAGAAGUGCCUCCAAUUUCAAGAGAAUUGGAGGCACUUCUGUGC-
3′;
(SEQ ID NO: 450)
5′-GGACAUAGUUUCUGGGAGAUUCAAGAGAUCUCCCAGAAACUAUGUCC
-3′;
(SEQ ID NO: 451)
5′-GGAUGACACUGGCACAUUAUUCAAGAGAUAAUGUGCCAGUGUCAUCC
-3′;
(SEQ ID NO: 452)
5′-GGAGAGUACUGAUGAGGAAUUCAAGAGAUUCCUCAUCAGUACUCUCC
-3′;
(SEQ ID NO: 453)
5′-GAAUCCACCUUGUUUCUGAUUCAAGAGAUCAGAAACAAGGUGGAUUC
-3′;
(SEQ ID NO: 454)
5′-GUACAAGUAUCUUGGGAGAUUCAAGAGAUCUCCCAAGAUACUUGUAC
-3′;
(SEQ ID NO: 455)
5′-GAAUGCAGCUCAAGAACGAUUCAAGAGAUCGUUCUUGAGCUGCAUUC
-3′;
(SEQ ID NO: 456)
5′-GCAUGAGUCUGGCAUUACAUUCAAGAGAUGUAAUGCCAGACUCAUGC
-3′;
(SEQ ID NO: 457)
5′-GAAAGCCCUUUGUCAUGAAUUCAAGAGAUUCAUGACAAAGGGCUUUC
-3′;
(SEQ ID NO: 458)
5′-GAACAGUGCUUCCCUGCAAUUCAAGAGAUUGCAGGGAAGCACUGUUC-
3′;
(SEQ ID NO: 459)
5′-GGAAAGACCUAGAGAUCCAUUCAAGAGAUGGAUCUCUAGGUCUUUCC
-3′;
(SEQ ID NO: 460)
5′-GUAUGGCAGUCACCACACAUUCAAGAGAUGUGUGGUGACUGCCAUAC
-3′;
(SEQ ID NO: 461)
5′-GCAGCAUUGUGCAGAUACAUUCAAGAGAUGUAUCUGCACAAUGCUGC
-3′;
(SEQ ID NO: 462)
5′-GCAGGAACAUCCCUCCUUAUUCAAGAGAUAAGGAGGGAUGUUCCUGC
-3′;
(SEQ ID NO: 463)
5′-GCAGUGCUCUGCUCAUCAAUUCAAGAGAUUGAUGAGCAGAGCACUGC
-3′;
(SEQ ID NO: 464)
5′-GGAUCAUGCUGUUCACCAAUUCAAGAGAUUGGUGAACAGCAUGAUCC
-3′;
(SEQ ID NO: 465)
5′-GGAUCUGACUACUCACUCAUUCAAGAGAUGAGUGAGUAGUCAGAUCC
-3′;
(SEQ ID NO: 466)
5′-GCACAAAGAUGGAGAUGUAUUCAAGAGAUACAUCUCCAUCUUUGUGC
-3′;
(SEQ ID NO: 467)
5′-GCAGACACGUACUGUGUAAUUCAAGAGAUUACACAGUACGUGUCUGC
-3′;
(SEQ ID NO: 468)
5′-GGAGCAGACUCCUGAAGAAUUCAAGAGAUUCUUCAGGAGUCUGCUCC
-3′;
(SEQ ID NO: 469)
5′-GGAGGAUUCUGAUCUGCAAUUCAAGAGAUUGCAGAUCAGAAUCCUCC
-3′;
(SEQ ID NO: 470)
5′-GCAUGAUCCUUCUGUAGGAUUCAAGAGAUCCUACAGAAGGAUCAUGC
-3′;
(SEQ ID NO: 471)
5′-GGAGACUCCUACCCAGAUAUUCAAGAGAUAUCUGGGUAGGAGUCUCC
-3′;
(SEQ ID NO: 472)
5′-GGACAAAGCUGACUACAGAUUCAAGAGAUCUGUAGUCAGCUUUGUCC
-3′;
(SEQ ID NO: 473)
5′-GCAGAGCUCUCGUUUCACAUUCAAGAGAUGUGAAACGAGAGCUCUGC
-3′;
(SEQ ID NO: 474)
5′-GGACUCUGAUGGAGAAUCAUUCAAGAGAUGAUUCUCCAUCAGAGUCC
-3′;
and
(SEQ ID NO: 475)
5′-GCAGAAUUCUUCCCAGUCAUUCAAGAGAUGACUGGGAAGAAUUCUGC
-3′.

### Guide RNA-Cas12f1 Protein Complex

### Example 54, Number, not Limited

A guide RNA-Cas12f1 protein complex, comprising:
the Cas12f1 protein of any one of Examples 1 to 15; and
the guide RNA of any one of Examples 40 to 48.

### Example 55, Dimer Form

A guide RNA-Cas12f1 protein complex, comprising:
a first Cas12f1 protein;
a second Cas12f1 protein; and
the guide RNA of any one of Examples 40 to 48,
wherein the first Cas12f1 protein and the second Cas12f1 protein are each independently any one selected from Examples 1 to 15, and
the first Cas12f1 protein and the second Cas12f1 protein have a feature of forming a dimer.

### Vector for CRISPR/Cas12f1 System

### Example 56, at Least One Guide RNA

A vector, comprising:
a nucleic acid encoding the Cas12f1 protein of any one selected from Examples 1 to 15; and
a nucleic acid encoding at least one guide RNA selected from Examples 40 to 48.

### Example 57, Two Guide RNAs

A vector, comprising:
a nucleic acid encoding the Cas12f1 protein of any one selected from Examples 1 to 15;
a nucleic acid encoding a first guide RNA; and
a nucleic acid encoding a second guide RNA,
wherein the first guide RNA and the second guide RNA are each independently
the guide RNA selected from any one of Examples 40 to 48.

### Example 58, Target Region of Each Guide RNA, Defined

The vector of Example 57, wherein a guide region of the first guide RNA is capable of recognizing, binding to, or targeting a region between the 3′ end of exon 26 of the CEP290 gene and the IVS26 mutation (c.2991+1655A>G) of the CEP290 gene, and
a guide region of the second guide RNA is capable of recognizing, binding, or targeting a region between the IVS26 mutation (c.2991+1655A>G) of the CEP290 gene and the 5′ end of exon 27 of the CEP290 gene,
which allows induction of deletion or inversion of a partial nucleic acid sequence containing the IVS26 mutation (c.2991+1655A>G) in the intron 26 region of the CEP290 gene.

### Example 59, Target Sequence of Each Guide RNA, Defined

The vector of Example 58, wherein the guide region of the first guide RNA is represented by a sequence selected from SEQ ID NOS: 51 to 74 and SEQ ID NOS: 245 to 256, and
the guide region of the second guide RNA is represented by a sequence selected from SEQ ID NOS: 75 to 101 and SEQ ID NOS: 257 to 272.

### Example 60, Inclusion of Additional Component

The vector of any one of Examples 56 to 59, wherein the vector further comprises a nucleic acid encoding at least one factor for decreasing NHEJ activity, and the at least one factor for decreasing NHEJ activity is each independently selected from any one of Examples 50 to 53.

### Example 61, Additional Component, Expression of Number Defined

The vector of any one of Examples 56 to 57, further comprising:
a nucleic acid encoding a first factor for decreasing NHEJ activity;
optionally, a nucleic acid encoding a second factor for decreasing NHEJ activity;
optionally, a nucleic acid encoding a third factor for decreasing NHEJ activity;
optionally, a nucleic acid encoding a fourth factor for decreasing NHEJ activity; and
optionally, a nucleic acid encoding a fifth factor for decreasing NHEJ activity,
wherein the first factor for decreasing NHEJ activity, the second factor for decreasing NHEJ activity, the third factor for decreasing NHEJ activity, the fourth factor for decreasing NHEJ activity, and the fifth factor for decreasing NHEJ activity are each independently selected from any one of Examples 50 to 53.

### Example 62, Inclusion of Promoter

The vector of any one of Examples 56 to 61, further comprising:
a promoter for the nucleic acid encoding the Cas12f1 protein;
a promoter for the nucleic acid encoding the guide RNA,
wherein in a case where the nucleic acid encoding the guide RNA is a plurality of nucleic acids, a promoter for each nucleic acid encoding the guide RNAs is included; and
in a case where the vector comprises a nucleic acid encoding at least one factor for decreasing NHEJ activity, a promoter for the nucleic acid encoding the factor for decreasing NHEJ activity,
wherein in a case where the nucleic acid encoding the factor for decreasing NHEJ activity is a plurality of nucleic acids, a promoter for each nucleic acid encoding the factor for decreasing NHEJ activity is included.

### Example 63, Example of Promoter

The vector of Example 62, wherein the promoters are each independently selected from a U6 promoter, an H1 promoter, a 7SK promoter, a CMV promoter, an LTR promoter, an Ad MLP promoter, an HSV promoter, an SV40 promoter, a CBA promoter, and an RSV promoter.

### Example 64, Type of Vector, Defined

The vector of any one of Examples 56 to 63, wherein the vector is selected from a plasmid, an mRNA (transcript), a PCR amplicon, and a viral vector.

### Example 65, Type of Virus Vector, Defined

The vector of Example 64, wherein the vector is a viral vector selected from a retroviral (retrovirus) vector, a lentiviral (lentivirus) vector, an adenoviral (adenovirus) vector, an adeno-associated viral (adeno-associated virus; AAV) vector, a vaccinia viral (vaccinia virus) vector, a poxviral (poxvirus) vector, and a herpes simplex viral (herpes simplex virus) vector.

### Example 66, Single Vector, Defined

The vector of any one of Examples 56 to 65, wherein the vector is a single vector.

### Example 67, Plurality of Vectors

The vector of any one of Examples 56 to 65, wherein the vector comprises two or more vectors.

### CRISPR/Cas12f1 Composition

### Example 68, CRISPR/Cas12f1 Composition, at Least One Guide RNA

A CRISPR/Cas12f1 composition, comprising:
any one Cas12f1 protein selected from Examples 1 to 15, or a nucleic acid encoding the Cas12f1 protein; and
at least one guide RNA selected from Examples 40 to 48, or a nucleic acid encoding the guide RNA,
wherein the guide RNA is capable of recognizing the CEP290 gene, binding to
the CEP290 gene, and/or targeting the CEP290 gene.

### Example 69, CRISPR/Cas12f1 Composition, Two Guide RNAs

A CRISPR/Cas12f1 composition, comprising:
any one Cas12f1 protein selected from Examples 1 to 15, or a nucleic acid encoding the Cas12f1 protein;
a first guide RNA or a nucleic acid encoding the first guide RNA; and
a second guide RNA or a nucleic acid encoding the second guide RNA,
wherein the first guide RNA and the second guide RNA are each independently any one guide RNA selected from Examples 40 to 48, and
the first guide RNA and the second guide RNA are each independently capable of recognizing the CEP290 gene, binding to the CEP290 gene, and/or targeting the CEP290 gene.

### Example 70, Target Region, Defined

The CRISPR/Cas12f1 composition of Example 69, wherein a guide region of the first guide RNA is capable of recognizing, binding to, or targeting a region between the 3′ end of exon 26 of the CEP290 gene and the IVS26 mutation (c.2991+1655A>G) of the CEP290 gene, and
a guide region of the second guide RNA is capable of recognizing, binding to, or targeting a region between the IVS26 mutation (c.2991+1655A>G) of the CEP290 gene and the 5′ end of exon 27 of the CEP290 gene,
which allows induction of deletion or inversion of a partial nucleic acid sequence containing the IVS26 mutation (c.2991+1655A>G) in the intron 26 region of the CEP290 gene.

### Example 71, Target Sequence, Defined

The CRISPR/Cas12f1 composition of Example 69, wherein a guide region of the first guide RNA is represented by a sequence selected from SEQ ID NOS: 51 to 74 and SEQ ID NOS: 245 to 256, and
a guide region of the second guide RNA is represented by a sequence selected from SEQ ID NOS: 75 to 101 and SEQ ID NOS: 257 to 272.

### Example 72, RNP, One Type of Guide RNA

The CRISPR/Cas12f1 composition of Example 68, wherein the CRISPR/Cas12f1 composition comprises the Cas12f1 protein and the guide RNA, in which the Cas12f1 protein and the guide RNA form a ribonucleoprotein (RNP).

### Example 73, Example of RNP, Defined

The CRISPR/Cas12f1 composition of Example 72, wherein the ribonucleoprotein is the guide RNA-Cas12f1 protein complex selected from any one of Examples 54 to 55.

### Example 74, Vector, One Type of Guide RNA

The CRISPR/Cas12f1 composition of Example 68, wherein the CRISPR/Cas12f1 composition comprises a nucleic acid encoding the Cas12f1 protein and a nucleic acid encoding the guide RNA.

### Example 75, Example of Vector, Defined

The CRISPR/Cas12f1 composition of Example 73, wherein the nucleic acid encoding the Cas12f1 protein and the nucleic acid encoding the guide RNA are included in a vector selected from any one of Examples 56 to 67.

### Example 76, RNP, Two Types

The CRISPR/Cas12f1 composition of any one of Examples 69 to 71, wherein CRISPR/Cas12f1 composition comprises a first ribonucleoprotein (RNP) in which the Cas12f1 protein and the first guide RNA are bound to each other, and a second ribonucleoprotein (RNP) in which the Cas12f1 protein and the second guide RNA are bound to each other.

### Example 77, Example of RNP, Defined

The CRISPR/Cas12f1 composition of Example 76, wherein the first ribonucleoprotein and the second ribonucleoprotein are each independently the guide RNA-Cas12f1 protein complex of any one of Examples 54 to 55.

### Example 78, Vector, Two Types of Guide RNA

The CRISPR/Cas12f1 composition of any one of Examples 69 to 71, wherein the CRISPR/Cas12f1 composition comprises a nucleic acid encoding the Cas12f1 protein, a nucleic acid encoding the first guide RNA, and a nucleic acid encoding the second guide RNA.

### Example 79, Example of Vector, Defined

The CRISPR/Cas12f1 composition of Example 68, wherein the CRISPR/Cas12f1 composition comprises the vector selected from any one of Examples 56 to 66.

### Example 80, Inclusion of Additional Component, at Least One

The CRISPR/Cas12f1 composition of any one of Examples 68 to 79, further comprising:
at least one factor for decreasing NHEJ activity, or a nucleic acid encoding the factor for decreasing NHEJ activity,
wherein the at least one factor for decreasing NHEJ activity is each independently selected from Examples 50 to 53.

### Example 81, Inclusion of Additional Component, Expression of Number Defined

The CRISPR/Cas12f1 composition of any one of Examples 68 to 79, further comprising:
a first factor for decreasing NHEJ activity or a nucleic acid encoding the first factor for decreasing NHEJ activity;
optionally, a second factor for decreasing NHEJ activity or a nucleic acid encoding the second factor for decreasing NHEJ activity;
optionally, a third factor for decreasing NHEJ activity or a nucleic acid encoding the second factor for decreasing NHEJ activity;
optionally, a fourth factor for decreasing NHEJ activity or a nucleic acid encoding the fourth factor for decreasing NHEJ activity; and
optionally, a fifth factor for decreasing NHEJ activity or a nucleic acid encoding the fifth factor for decreasing NHEJ activity,
wherein the first factor for decreasing NHEJ activity, the second factor for decreasing NHEJ activity, the third factor for decreasing NHEJ activity, the fourth factor for decreasing NHEJ activity, and the fifth factor for decreasing NHEJ activity are each independently selected from any one of Examples 50 to 53.

### Pharmaceutical Composition for Treatment of LCA10 Disease

### Example 82, Pharmaceutical Composition

A pharmaceutical composition for treatment of LCA10 disease, comprising: the CRISPR/Cas12f1 composition of any one of Examples 68 to 81; and optionally, a pharmaceutically acceptable carrier.

### Example 83, Example of Pharmaceutically Acceptable Carrier

The pharmaceutical composition of Example 82, wherein the pharmaceutically acceptable carrier is at least one selected from:
carriers such as water, saline, ethanol, glycerol, lactose, sucrose, calcium phosphate, gelatin, dextran, agar, pectin, peanut oil, or sesame oil; binders such as lactose, saccharose, sorbitol, mannitol, starch, amylopectin, cellulose, or gelatin; excipients such as dicalcium phosphate; disintegrants such as corn starch or sweet potato starch; lubricating oil such as magnesium stearate, calcium stearate, sodium stearyl fumarate, or polyethylene glycol wax; sweeteners; fragrances; syrup; liquid carriers such as fatty oil; sterile aqueous solutions; propylene glycol; polyethylene glycol; injectable ester such as ethyl oleate; suspending agents; emulsions; freeze-dried preparations; agents for external use; stabilizers; buffers; animal oil; vegetable oil; wax; paraffin; starch; tracanth; cellulose derivatives; polyethylene glycol; silicon; bentonite; silica; talc; and zinc oxide.

### Method for Treating LCA10 Disease

### Example 84, Method for Treating LCA10 Disease

A method for treating LCA10 disease, comprising:
administering the pharmaceutical composition of any one of Examples 82 to 83 to a subject,
wherein the CRISPR/Cas12f1 composition included in the pharmaceutical composition comprises a first guide RNA having a guide region capable of recognizing, binding to, or targeting a region between the 3′ end of exon 26 of the CEP290 gene and the IVS26 mutation (c.2991+1655A>G) of the CEP290 gene, and
a second guide RNA having a guide region capable of recognizing, binding to, or targeting a region between the IVS26 mutation (c.2991+1655A>G) of the CEP290 gene and the 5′ end of exon 27 of the CEP290 gene.

### Example 85, Target Sequence, Defined

The method of Example 84, wherein the guide region of the first guide RNA of the CRISPR/Cas12f1 composition is represented by a sequence selected from SEQ ID NOS: 51 to 74 and SEQ ID NOS: 245 to 256, and
the guide region of the second guide RNA of the CRISPR/Cas12f1 composition is represented by a sequence selected from SEQ ID NOS: 75 to 101 and SEQ ID NOS: 257 to 272.

### Example 86, Subject, Defined

The method of any one of Examples 84 to 85, wherein the subject is selected from:
a human; a non-human animals carrying the CEP290 gene; a human with LCA10 disease; a non-human animal with LCA10 disease; a human cell; a non-human animal cell carrying the CEP290 gene; a cell obtained from a human with LCA10 disease; and a cell obtained from a non-human animal with LCA10 disease.

### Example 87, Administration Method, Defined Type 1

The method of any one of Examples 84 to 86, wherein the administering is performed by at least one method selected from:
microinjection; cationic liposome; lithium acetate-DMSO; lipid-mediated transfection; calcium phosphate precipitation; lipofection; polyethyleneimine (PEI)-mediated transfection; DEAE-dextran-mediated transfection; and nanoparticle-mediated nucleic acid delivery (see Panyam et al., Adv Drug Deliv Rev. 2012 Sep. 13. pii: S0169-409X(12)00283-9. doi: 10.1016/j.addr.2012.09.023).

### Example 88, Administration Method, Defined Type 2

The method of any one of Examples 84 to 86, wherein the administering is performed by at least one method selected from:
injection; transfusion; implantation; and transplantation.

### Example 89, Administration Method, Defined Type 3

The method of any one of Examples 84 to 86, wherein the administering is performed by at least one route of administration selected from:
subretinal; subcutaneous; intradermal; intraocular; intravitreal; intratumoral; intranodal; intramedullary; intramuscular; intravenous; intralymphatic; and intraperitoneal.

### Method for Modifying CEP290 Gene

### Example 90, Method for Modifying CEP290 Gene Containing IVS26 Mutation

A method for artificially modifying the CEP290 gene containing the IVS26 mutation (c.2991+1655A>G), comprising:
introducing the CRISPR/Cas12f1 composition of any one of Examples 68 to 81 into a target cell,
wherein the CRISPR/Cas12f1 composition comprises a first guide RNA having a guide region capable of recognizing, binding to, or targeting a region between the 3′ end of exon 26 of the CEP290 gene and the IVS26 mutation (c.2991+1655A>G) of the CEP290 gene, and
a second guide RNA having a guide region capable of recognizing, binding to, or targeting a region between the IVS26 mutation (c.2991+1655A>G) of the CEP290 gene and the 5′ end of exon 27 of the CEP290 gene,
which induces deletion or inversion of a partial nucleic acid sequence containing the IVS26 mutation (c.2991+1655A>G) in the intron 26 region of the CEP290 gene, and
which removes all or part of the 128 base pair cryptic exon generated by the IVS26 mutation (c.2991+1655A>G) in the CEP290 gene,
thereby resulting in inhibited or decreased transcription and/or translation of the CEP290 gene containing the IVS26 mutation (c.2991+1655A>G) and/or increased transcription and/or translation of the normal CEP290 gene in the target cell.

### Example 91, Target Sequence, Defined

The method of Example 90, wherein the guide region of the first guide RNA is represented by a sequence selected from SEQ ID NOS: 51 to 74 and SEQ ID NOS: 245 to 256, and
the guide region of the second guide RNA is represented by a sequence selected from SEQ ID NOS: 75 to 101 and SEQ ID NOS: 257 to 272.

### Example 92, Target Cell, Defined Type 1

The method according to Example 90, wherein the target cell is a human cell or non-human animal cell containing the CEP290 gene.

### Example 93, Target Cell, Defined Type 2

The method of Example 90, wherein the target cell is a cell obtained from a human with LCA10 disease or a non-human animal with LCA10 disease.

### Example 94, Introduction Method, Defined

The method of any one of Examples 90 to 93, wherein the introducing is performed by at least one method selected from:
electroporation; gene gun; sonoporation; magnetofection; nanoparticles; transient cell compression or squeezing; cationic liposome method; lithium acetate-DMSO; lipid-mediated transfection; calcium phosphate precipitation; lipofection; Polyethyleneimine (PEI)-mediated transfection; DEAE-dextran mediated transfection; and nanoparticle-mediated nucleic acid delivery (see Panyam et al.; Adv Drug Deliv Rev. 2012 Sep. 13. pii: S0169-409X(12)00283-9. doi: 10.1016/j.addr.2012.09.023).

### Example 95, In Vivo Modification Method

A method of artificially modifying the CEP290 gene comprising an IVS26 mutation (c.2991+1655A>G) in a subject, comprising:
introducing the CRISPR/Cas12f1 composition of any one of Examples 68 to 81 into a subject,
wherein the CRISPR/Cas12f1 composition comprises a first guide RNA having a guide region capable of recognizing, binding to, or targeting a region between the 3′ end of exon 26 of the CEP290 gene and the IVS26 mutation (c.2991+1655A>G) of the CEP290 gene, and
a second guide RNA having a guide region capable of recognizing, binding to, or targeting a region between the IVS26 mutation (c.2991+1655A>G) of the CEP290 gene and the 5′ end of exon 27 of the CEP290 gene,
which induces deletion or inversion of a partial nucleic acid sequence containing the IVS26 mutation (c.2991+1655A>G) in the intron 26 region of the CEP290 gene, and
which removes all or part of the 128 base pair cryptic exon generated by the IVS26 mutation (c.2991+1655A>G) in the CEP290 gene,
thereby resulting in inhibited or decreased transcription and/or translation of the CEP290 gene containing the IVS26 mutation (c.2991+1655A>G) and/or increased transcription and/or translation of the normal CEP290 gene in the subject.

### Example 96, Subject, Defined

The method according to Example 95, wherein the subject is selected from:
a human; a non-human animal carrying the CEP290 gene; a human with LCA10 disease; and a non-human animal with LCA10 disease.

### Use of Pharmaceutical Composition for Treatment of LCA10 Disease

### Example 97, Use of Composition, Swiss-Type Claim

A use of the pharmaceutical composition selected from any one of Examples 82 to 83, for manufacture of a therapeutic agent for treating a subject with LCA10 disease.

### Example 98, Therapeutic Use of Composition

A use of the pharmaceutical composition selected from any one of Examples 82 to 83, for treating a subject with LCA10 disease.

### Example 99, Therapeutic Use, Specified

The use of Example 98, wherein the use is for use in the method for treating LCA10 disease selected from any one of Examples 84 to 89.

### Example 100, Use of Composition for Editing CEP290 Gene

A use of the pharmaceutical composition selected from any one of Examples 82 to 83, for editing the CEP290 gene in a subject.

### Example 101, Use of Composition for Editing CEP290 Gene, Specified

The use of Example 100, wherein the use is for use in the method for modifying the CEP290 gene selected from any one of Examples 90 to 96.

### Example 102, Vector Comprising Nucleic Acid Encoding Codon-Optimized Cas12f1 Protein

The vector of any one of Examples 56 to 67, wherein the nucleic acid encoding the Cas12f1 protein is represented by SEQ ID NO: 480 to SEQ ID NO: 482.

### Example 103, Composition Comprising Nucleic Acid Encoding Codon-Optimized Cas12f1 Protein

The CRISPR/Cas12f1 composition of any one of Examples 68 to 81, wherein the nucleic acid encoding the Cas12f1 protein is represented by SEQ ID NO: 480 to SEQ ID NO: 482.

The present disclosure will be described in detail by way of the following examples.

These examples are only for describing the present disclosure in more detail, and it will be apparent to those skilled in the art that the scope of the present disclosure is not limited by these examples.

### EXAMPLES

### Experiment Methods

1. Extraction of gDNA

gDNA Prep was performed using the Genomic DNA Prep Kit (GCBL200, Nanohelix). For cells transfected in a 24-well plate, removal of media was performed. The cells were removed from the bottom with 200 μl of trypsin and placed in a 1.5 ml tube. Centrifugation was performed at 300×g for 5 minutes and the supernatant was removed. To the resultant were added 300 μl of NGD1 buffer and 2 μl of RNase A (50 mg/ml), and vortexing was performed for 1 minute. 8 μl of Proteinase K (10 mg/ml) was added thereto, and then reaction was allowed to occur at 60° C. for 10 minutes. Cooling was performed on ice for 5 minutes. 300 μl of NPS buffer was added thereto and the resultant was mixed well. Then, reaction was allowed to occur on ice for 5 minutes and centrifugation was performed at 12,000 rpm for 5 minutes. Columns were prepared depending on the number of samples. 100 μl of MaxBinder Solution was added thereto, and centrifugation was performed at 12,000 rpm for 30 seconds. The entire supernatant was collected, placed in the column, and centrifuged at 12,000 rpm for 1 minute. Then, the filtered solution was discarded. 500 μl of 80% EtOH was added to the column, centrifugation was performed at 10,000 rpm for 30 seconds, and the filtered solution was discarded. After repeating the procedure twice, centrifugation was performed at 13,000 rpm for 3 minutes. The column was placed in a new 1.5 ml tube, and 30 μl of EB solution was dropped in the center. Then, reaction was allowed to occur for 1 minute, and then centrifugation was performed at 12,000 rpm for 2 minutes. The eluted gDNA was quantified and stored at 4° C.

### 2. PCR & Gel Purification

This experiment was conducted using the GEL & PCR Purification System (GP104-200, Biofact). To the resulting PCT product was added UB buffer equivalent to 3 times the PCR product's volume, and mixed well. To the mixture was added isopropanol equivalent to 2 times the PCR product's volume, and mixed well. For the gels, the gel of the corresponding band was cut and weighed. Then, UB buffer equivalent to three times the gel's weight was added thereto, and reaction was allowed to occur at 65° C. for 10 minutes so that the gel was dissolved. Then, isopropanol was added thereto in an amount equivalent to 1 time the gel's volume and mixed well. Columns were prepared and 200 μl of HelpB buffer was added to each column. Then, centrifugation was performed at 13,000 rpm for 30 seconds, and then the filtered solution was discarded. The reaction solution was placed in the column, centrifugation was performed at 7,000 rpm for 1 minute, and then the filtered solution was discarded. To the resultant was added 750 μl of 80% EtOH and centrifugation was performed at 13,000 rpm for 30 seconds. Then, the filtered solution was discarded. After repeating the procedure twice, centrifugation was performed at 13,000 rpm for 3 minutes. After the centrifugation was completed, the column was placed in a 1.5 ml tube, 30 μl of EB buffer was dropped in the center, and reaction was allowed to occur at room temperature for 1 minute. Centrifugation was performed at 13,000 rpm for 1 minute. The DNA collected in the 1.5 ml tube was quantified, and then stored at 4° C.

### 3. Collection of Plasmid Vector

Vectors transformed into DH5a were used for transfection or produced for Sanger sequencing. The Plasmid Mini prep kit (PM105-200, Biofact) was used and the procedure was performed according to the manufacturer's manual. The vector-transformed DH5a culture was placed in a 1.5 ml tube, and then centrifugation was performed at 13,000 rpm for 5 minutes. After the centrifugation, the supernatant was discarded, and the pellet was sufficiently loosened by vortexing. To the resultant was added 350 μl of B1 buffer, and then the tube was shaken to allow sufficient reaction. 350 μl of A1 buffer containing RNase A was added thereto and the tube was inverted until the blue color disappeared. Then, centrifugation was performed at 13,000 rpm for 5 minutes. Columns were prepared, and 200 μl of HelpB buffer was added to each column. Then, centrifugation was performed at 13,000 rpm for 30 seconds, and the filtered solution was discarded. 750 μl of the supernatant was added to the column, centrifugation was performed at 7,000 rpm for 1 minute, and the filtered solution was discarded. To the resultant was added 750 μl of 80% EtOH and centrifugation was performed at 13,000 rpm for 30 seconds. Then, the filtered solution was discarded. After repeating the procedure twice, centrifugation was performed at 13,000 rpm for 3 minutes. After the centrifugation was completed, the column was placed in a 1.5 ml tube, 30 μl of EB buffer was dropped in the center, and reaction was allowed to occur at room temperature for 1 minute. Centrifugation was performed at 13,000 rpm for 1 minute. The plasmid vector collected in the 1.5 ml tube was quantified, and then stored at −20° C.

### 4. Production of Cassette DNA

To identify indel efficiency of targets that are spacers of Cas14a1, a cassette containing a U6 promoter and a scaffold sequence, a guide sequence, and a U-rich tail sequence (T₄AT₆) was amplified by PCR and used. The procedure was performed in the following manner.
1) Selection of spacer and order of oligo

For spacers, 20-mer sequences located after TTTR, which is PAM, were selected, and spacers ending in T were excluded. To decrease off-target, the spacer was designed in CRISPR RGEN TOOL in such a way that 2 or fewer mismatches were sorted. Reverse complement sequences containing a direct repeat and a U-rich sequence were ordered for use as R primers.
2) PCR

PCR was performed with the following composition and conditions.

TABLE 7
PCR-related information

| Reagent | | PCR conditions | |
|---|---|---|---|
| 2X pfu PCR | 205 [symbol] | Pre- | 95° C., 5 min |
| Master mix | | denaturation | |
| hU6 F primer (10 P) | 2.05 [symbol] | Denaturation | 95° C., 30 sec |
| Target oligo (10 P) | 2.05 [symbol] | Annealing | 60° C., 30 sec |
| Template | 1 [symbol] (200 ng) | Extension | 72° C., 2 min |
| DW | 199.9 [symbol] | D-E cycle | 30 cycles |
| Total | 410 [symbol] | Final extension | 72° C., 3 min |

| | | |
|---|---|---|
| Divide by 50 μl and proceed | Storage | 4° C., ∞ |
| in 8 PCR tubes | | |

400 μl of the mixture was placed in PCR tubes at 50 μl each, and one sample was amplified using 8 tubes.
3) Gel analysis

Amplification sizes were checked by electrophoresis on 1% agarose gel.
4) PCR purification & quantification

### 5. Cell Culture

HEK-293T cells and ARPE-19/HPV-16 cells used in the experiment were cultured with the following composition and conditions: HEK-293T cell media—DMEM, 10% FBS, 1% penicillin-streptomycin; and AREP-19/HPV-16 cell media—DEME/F12, 10% FBS, 1% penicillin-streptomycin. Frozen cells were rapidly thawed at 37° C., and then placed and dissolved well in 5 ml of pre-warmed cell media. Then, centrifugation was performed at 1,500 rpm for 3 minutes. After the centrifugation, the supernatant with remaining freezing liquid was rapidly removed, and the pellet was loosened well with the cell media. Then, the resultant was aliquoted into two 90 mm dishes each containing 10 ml of media and culture was performed. The next day, the cell media was replaced with new media and culture was performed continuously. When the confluency reached 80%, subcultures were performed at respective ratios (performed at a ratio of 1/15 for HEK-293T, and at a ratio of 1/4 for AREP-19/HPV-16).

### 6. Transfection (HEK-293T, ARPE19-HPV)

One day before transfection, the cells (80% confluency) were removed from the bottom of 100 mm dishes by treatment with trypsin. The removed cells were put into 50 ml of pre-warmed media (HEK-293T-DMEM, 10% FBS, 1% p/s, ARPE19-HPV-DMEM/F12, 10% FBS, 1% p/s) and loosened slowly with a pipette. 24-well plates were prepared depending on the number of samples and repetitions, and 500 μl of media in which the cells were loosened once was added to each well (1/100 dilution, 0.5 ml/50 ml). Then, incubation was performed overnight in a CO₂ incubator at 37° C. until transfection. Transfection was performed when the confluency reached about 70 to 80%. Out of 500 μl of media per well, 200 μl of media was removed and placed in the incubator. 1.5 ml tubes were prepared depending on the number of samples, and 200 μl of Opti-MEM was added to each tube. A corresponding amount of DNA was added to the tube containing Opti-MEM and vortexing was performed for 5 seconds (DNA−Cas14a1 1.5 μg+gRNA 0.5 μg). Then, FuGENE HD was added at a ratio of DNA:Reagent=1:3 (6 μl of FuGENE HD was added when DNA was 2 μg). Reaction was allowed to occur at room temperature for 20 minutes. The 24-well plate was taken out of the incubator, and 200 μl of the reaction solution of No. 8 was allowed to flow through the wall of the well. The plate was sufficiently shaken in an S shape, and incubation was performed in a CO₂ incubator at 37° C. up to 72 hours. After 72 hours, the cells were harvested, and gDNA was extracted therefrom.

### 7. NGS Sample PCR

Preparation of NGS samples for identifying indel efficiency of targets was done through PCR product purification after conducting 1st to 3rd PCRs.
1) Composition and conditions for PCR (first PCR)

F: CEP290-F-F #10, LCA10-2nd-9-R (1323 bp), R: CEP290-R-F #1, CEP290-R-R #8 (1879 bp)

TABLE 8
PCR-related information

| Reagent | | PCR conditions | |
|---|---|---|---|
| KAPA HiFi 2X PCR mix | 5 [symbol] | Pre-denaturation | 95° C., 3 min |
| Forward primer (10 P) | 0.5 [symbol] | Denaturation | 98° C., 20 sec |
| Reverse primer (10 P) | 0.5 [symbol] | Annealing | 60° C., 15 sec |
| Template (gDNA) | 1 [symbol] | Extension | 72° C., 2 min |
| DW | 3 [symbol] | D-E cycle | 30 cycles |
| Total | 10 [symbol] | Final extension | 72° C., 3 min |
| | | Storage | 4° C. ∞ |

2) Composition and conditions for PCR (second PCR)

TABLE 9
PCR-related primer information

| Primer set | Forward primer | Reverse primer | Protospacer sequence | | | |
|---|---|---|---|---|---|---|
| S1 | LCA10-2nd-1-F | LCA10-2nd-1-R | F21 | | | |
| S2 | LCA10-2nd-2-F | LCA10-2nd-2-R | F17 | F18 | F19 | F20 |
| S3 | LCA10-2nd-3-F | CEP-F-R#11-miseq-R | F12 | F15 | F16 | |
| S4 | LCA10-2nd-4-F | LCA10-2nd-4-R | F11 | F14 | | |
| S5 | CEP-F-F#9-miseq-F | CEP-F-R#8-miseq-R | F10 | F13 | F22 | |
| S6 | LCA10-2nd-6-F | LCA10-2nd-6-R | F04 | F07 | F08 | |
| S7 | CEP-F-F#11-miseq-F | CEP-F-R#10-miseq-R | F03 | F05 | F23 | F24 |
| S8 | CEP-F-F#3-miseq-F | CEP-F-R#3-miseq-R | F01 | F02 | F09 | |
| S9 | LCA10-2nd-9-F | CEP-F-R#5-miseq-R | F05 | | | |
| S10 | LCA10-2nd-10-F | LCA10-2nd-10-R | R05 | R06 | R07 | |
| S11 | LCA10-2nd-11-F | LCA10-2nd-11-R | R04 | R09 | R10 | |
| S12 | CEP-R-F#5-miseq-F | CEP-R-R#4-miseq-R | R03 | R11 | | |
| S13 | LCA10-2nd-13-F | LCA10-2nd-13-R | R01 | R02 | R08 | |
| S14 | LCA10-2nd-14-F | LCA10-2nd-14-R | R12 | R13 | | |
| 515 | LCA10-2nd-15-F | LCA10-2nd-15-R | R24 | R26 | | |
| S16 | LCA10-2nd-16-F | LCA10-2nd-16-R | R22 | R23 | | |
| S17 | LCA10-2nd-17-F | LCA10-2nd-17-R | R21 | | | |
| S18 | LCA10-2nd-18-F | LCA10-2nd-18-R | R20 | R25 | | |
| 519 | LCA10-2nd-19-F | LCA10-2nd-19-R | R19 | | | |
| S20 | LCA10-2nd-20-F | LCA10-2nd-20-R | R16 | R17 | R18 | |
| S21 | LCA10-2nd-21-F | LCA10-2nd-21-R | R14 | R15 | | |

TABLE 10
PCR-related information

| Reagent | | PCR conditions | |
|---|---|---|---|
| KAPA HiFi 2X PCR mix | 5 [symbol] | Pre-denaturation | 95° C., 3 min |
| Forward primer (10 P) | 0.5 [symbol] | Denaturation | 98° C., 20 sec |
| Reverse primer (10 P) | 0.5 [symbol] | Annealing | 60° C., 15 sec |
| Template (PCR product) | 0.5 [symbol] | Extension | 72° C., 30 sec |
| DW | 3.5 [symbol] | D-E cycle | 30 cycles |
| Total | 10 [symbol] | Final extension | 72° C. 3 min |
| | | Storage | 4° C. ∞ |

The samples R01, R02, R08, and S13 NT were annealed at 57° C., and the samples R20, R25, S18 NT, R19, and S19 NT were annealed at 53° C.
3) Composition and conditions for PCR (third PCR)

TABLE 11
PCR-related information

| Reagent | | PCR conditions | |
|---|---|---|---|
| KAPA HiFi 2X PCR mix | 5 [symbol] | Pre-denaturation | 95° C., 3 min |
| Forward primer (10 P) | 0.5 [symbol] | Denaturation | 98° C., 20 sec |
| Reverse primer (10 P) | 0.5 [symbol] | Annealing | 60° C., 15 sec |
| Template (PCR product) | 0.5 [symbol] | Extension | 72° C., 30 sec |
| DW | 3.5 [symbol] | D-E cycle | 30 cycles |
| Total | 10 [symbol] | Final extension | 72° C., 3 min |
| | | | 4° C. ∞ |

The samples R01, R02, R08, S13 NT, R20, R25, S18 NT, R19, and S19 NT were annealed at 57° C., and the samples F10, F13, F22, S5 NT, R12, R13, S14 NT, R24, R26, S15 NT, R16, R17, R18, and S20NT were annealed at 63° C.
4) Summary of PCR primers

TABLE 12
PCR-related primer information

| | Target | | | Di- | | Length |
|---|---|---|---|---|---|---|
| Use | region | No. | Name | rection | Sequence | (nt) |
| For 1st | F region | 1 | CEP290- | F | TGAACTGACTGCTAAGTACAGGGACA | 26 |
| PCR | | | F-F#10 | | (SEQ ID NO: 147) | |
| | | 2 | LCA10- | R | AAGCGTTAAGCCGAGATCGT | 20 |
| | | | 2^{nd}-9-R | | (SEQ ID NO: 148) | |
| | R region | 3 | CEP290- | F | TGTGGCAGCCATTATTCGTGTGTCTA | 26 |
| | | | R-F#1 | | (SEQ ID NO. 149) | |
| | | 4 | CEP290- | R | TCTGCCTTGACTAGCTGGTTAGGTAA | 26 |
| | | | R-R#8 | | (SEQ ID NO: 350) | |

TABLE 13
PCR-related primer information

| | Target | | | | | Length |
|---|---|---|---|---|---|---|
| Use | region* | NO. | Name | Direction | Sequence | (nt) |
| for 2^{nd} | F region* | 1 | LCA10-2nd-1-F | F | CACTCTTTCCCTACACGACGCTCTTCCGATCTGAACTGACTGCTAAGTACAGGGA | 56 |
| PCR | | | | | C (SEQ ID NO: 151) | |
| | | 2 | LCA10-2nd-1-R | R | GTGACTGGAGTTCAGACGTGTGCTCTTCCGATCTGCAGGCAAACTTTAATTAAAA | 61 |
| | | | | | TCTGCC (SEQ ID NO: 152) | |
| | | 3 | LCA10-2nd-2-F | F | CACTCTTTCCCTACACGACGCTCTTCCGATCTGGAACACCTGGAGGTAAGTTTG | 54 |
| | | | | | (SEQ ID NO: 153) | |
| | | 4 | LCA10-2nd-2-R | R | GTGACTGGAGTTCAGACGTGTGCTCTTCCGATCTCATTCAGAAGTCATTCAGCCA | 57 |
| | | | | | CT (SEG ID NO: 154) | |
| | | 5 | LCA10-2nd-3-F | F | CACTOTTTCCCTACACGACGCTCTTCCGATCTTTGTTCCCACATAACACACACA | 54 |
| | | | | | (SEQ ID NO: 55) | |
| | | 6 | CEP-F-R#13- | R | GTGACTGGAGTTCAGACGTGTGCTCTTCCGATCTTTCTGGGTACAGGGGTAAGAG | 60 |
| | | | miseq-R | | AAAGG (SEQ ID NO: 156) | |
| | | 7 | LCA10-2nd-4-F | F | CACTCTTTCCCTACACGACGCTCTTCCGATCTTGTAATCAAAGGAGGGAAGCA | 53 |
| | | | | | (SEQ ID NO: 157) | |
| | | 8 | LCA10-2nd-4-R | R | GTGACTGGAGTTCAGACGTGTGCTCTTCCGATCTGATGAGAATTTACAGAGTGCA | 58 |
| | | | | | TCC (SEQ ID NO: 158) | |
| | | 9 | CEP-F-F#9- | F | CACTCTTTCCCTACACGACGCTCTTCCGATCTACCCAGAACAAACTATTCTOCCA | 58 |
| | | | miseq-F | | TGG (SEQ ID NO: 159) | |
| | | 10 | CEP-F-R#8- | R | GTGACTGGAGTTCAGACGTGTGCTCTTCCGATCTGGGTCTGGTCCATATTCACAG | 50 |
| | | | miseq-R | | ATTGT (SEQ ID NO. 160) | |
| | | 11 | LCA10-2nd-6-F | F | CACTCTTTCCCTACACGACGCTCTTCCGATCTTGGACCAGACCCTTTGTAGTT | 53 |
| | | | | | (SEQ ID NO: 163) | |
| | | 12 | LCAID-2nd-6-R | R | GTGACTGGAGTTCAGACGTGTGCTCTTCCGATCTTGTCTCTATGAGCCAGCAAAA | 56 |
| | | | | | G (SEQ ID NO: 162) | |
| | | 13 | CEP-F-F#13- | F | CACTCTTTCCCTACACGACGCTCTTCCGATCTTCATCTTCCTCATCAGAAATAGA | 60 |
| | | | miseq-F | | GGCTT (SEQ ID NO: 163) | |
| | | 14 | CER-F-R#10- | R | GTGACTGGAGTTCAGACGTGTGCTCTTCCGATCTGCAAAAGCAGCAGAAAGCAAA | 60 |
| | | | miseq- | | CTGAT (SEQ ID NO: 164) | |
| | | 15 | CEP-F-F#3- | F | CACTCTTTCCCTACACGACGCTCTTCCGATCTTGCTGGCTCATAGAGACACATTC | 58 |
| | | | miseq-F | | AGT (SEQ ID NO: 165) | |
| | | 16 | CEP-F-R#3- | R | GTGACTGGAGTTCAGACGTGTGCTCTTCCGATCTTGCTGGAGAGGATAGGACAGA | 58 |
| | | | miseq-R | | GGA (SEQ ID NO. 166) | |
| | | 17 | LCA10-2nd-9-F | F | CACTCTTTCCCTACACGACGCTCTTCCGATCTTTCCATTCTCCATGTCCTCTG | 53 |
| | | | | | (SEQ ID NO: 167) | |
| | | 18 | CEP-F-R#5- | R | GTGACTGGAGTTCAGACGTGTGCTCTTCCGATCTGGGTGACAGAATGAAACTCCA | 60 |
| | | | miseq-R | | TCTCA (SEQ ID NO: 168) | |

TABLE 14
PCR-related primer information

| | Target | | | | | Length |
|---|---|---|---|---|---|---|
| Use | region* | NO. | Name | Direction | Sequence | (nt) |
| for 2nd | R region* | 1 | LCA10-2nd-10-F | F | CACTCTTTCCCTACACGACGCTCTTCCGATCTGTGGCAGCCATTATTCCTGT (SEQ | 52 |
| PCR | | | | | ID NO: 169) | |
| | | 2 | LCA10-2nd-10-R | R | GTGACTGGAGTTCAGACGTGTGCTCTTCCGATCTGAACACTGTGGTCAGAAAACT | 57 |
| | | | | | CA (SEQ ID NO: 170) | |
| | | 3 | LCA10-2nd-11-F | F | CACTCTTTCCCTACACGACGCTCTTCCGATCTATGTATGTTCTTTTGTACGTGCTC | 58 |
| | | | | | TT (SEQ ID NO: 171) | |
| | | 4 | LCA10-2nd-11-R | R | GTGACTGGAGTTCAGACGTGTGCTCTTCCGATCTAAGACAGATATGTAAACACAT | 64 |
| | | | | | ATAATTTGA (SEG ID NO: 172) | |
| | | 5 | CER-R-F#S-miseq-F | F | CACTCTTTCCCTACACGACGCTCTTCCGATCTGTCACTCCTGTCTACACTAGTTC | 58 |
| | | | | | TGC (SEQ ID NO: 173) | |
| | | 6 | CEP-R-R#4-misea-R | R | GTGACTGGAGTTCAGACGTGTGCTCTTCCGATTACCATATTTGACACCACATGC | 60 |
| | | | | | ACTGT (SEQ ID NO: 174) | |
| | | 7 | LCA10-2nd-13-F | F | CACTCTTTCCCTACACGACGCTCTTCCGATGTTTCCTTAATGGAATATAAGTCTTT | 59 |
| | | | | | TGA (SEQ ID NO: 175) | |
| | | 8 | LCA 10-2nd-13-R | R | GTGACTGGAGTTCAGACGTGTGCTCTTCCGATCTCCTTATTATCTATTCCATTCTT | 62 |
| | | | | | CACACA (SEQ ID NO: 176) | |
| | | 9 | LCA 10-2nd-14-F | F | CACTCTTTCCCTACACGACGCTCTTCCGATCTGGTTCATGAGACTAGAGGTCACG | S5 |
| | | | | | (SEQ ID NO: 177) | |
| | | 10 | LCA10-Znd-14-R | R | GTGACTGGAGTTCAGACGTGTGCTCTTCCGATCTCTGGGCAACACAGTGAGACT | 54 |
| | | | | | (SEQ ID NO: 178) | |
| | | 11 | LCA10-2nd-15-F | F | CACTCTTTCCCTACACGACGCTCTTCCGATCTGGTTCAAGCGATTCTTCTGC (SEQ | 52 |
| | | | | | ID NO: 179) | |
| | | 12 | LCA10-2nd-15-R | R | GTGACTGGAGTTCAGACGTGTGCTCTTCCGATCTCCTGAAACTTCACTGGTCTCC | 55 |
| | | | | | (SEQ ID NO: 180) | |
| | | 13 | LCA10-2nd-36-F | F | CACTCTTTCCCTACACGACGCTCTTCCGATCTAGCCTCAACTTCCCAAAGTG | 52 |
| | | | | | (SEQ ID NO: 181) | |
| | | 14 | LCA 30-2nd-15-R | R | GTGACTGGAGTTCAGACGTGTGCTCTTCCGATCTTCTTTTTCTCCCACCTACCC | 54 |
| | | | | | (SEQ ID NO: 182) | |
| | | 15 | LCA10-2nd-17-F | F | CACTCTTTCCCTACACGACGCTCTTCCGATCTTTTAGAGTTACTAGGGAGAGAGT | 60 |
| | | | | | GATGA (SEQ ID NO: 183) | |
| | | 16 | LCA10-2nd-17-R | R | GTGACTGGAGTTCAGACGTGTGCTCTTCCGATCTACACAGGAGGGAAAACAGGA | 54 |
| | | | | | (SEQ ID NO: 184) | |
| | | 17 | LCA10-2nd-18-F | F | CACTCTTTCCCTACACGACGCTCTTCCGATCGTAAAAAGGATGAAAGG | 54 |
| | | | | | (SEQ ID NO: 185) | |
| | | 18 | LCAIG-2nd-18-R | R | GTGACTGGAGTTCAGACGTGTGCTCTTCCGATCTTGAAGAATAATACAAACAGEG | 61 |
| | | | | | TAATGA (SEQ ID NO: 186) | |
| | | 19 | LCA 30-2nd- 19-F | F | CACTCTTTCCCTACACGACGCTCTTCCGATCTCTCCTGTTTTCCCTCCTGTG (SEQ | 52 |
| | | | | | ID NO: 187) | |
| | | 20 | LCA10-2nd-39-R | R | GTGACTGGAGTTCAGACGTGTGCTCTTCCGATCTTTTGAGTGGAGTACCTGGAAA | 55 |
| | | | | | (SEQ ID NO: 188) | |
| | | 21 | LCA30-2nd-20-F | F | CACTCTTTCCCTACACGACGCTCTTCCGATCTTGTCCTCTTTTTAGTCTCATTACC | 57 |
| | | | | | C (SEQ ID NO: 189) | |
| | | 22 | LCA10-2nd-20-R | R | GTGACTGGAGTTCAGACGTGTGCTCTTCCGATCTGGAAAAAGGCTGGCGATATAA | 55 |
| | | | | | (SEO ID NO: 190) | |
| | | 23 | LCA10-2nd-21-F | F | CACTCTTTCCCTACACGACGCTCTTCCGATCTATATCGCCAGCCTTTTTCCT (SEQ | S2 |
| | | | | | ID NO: 191) | |
| | | 24 | LCA10-2nd-21-R | R | GTGACTGGAGTTCAGACGTGTGCTCTTCCGATCTGCCTTGACTAGCTGGTTAGGT | 56 |
| | | | | | A (SEQ ID NO: 192) | |

*The target region is divided into a forward region (F region) and a reverse region (R region), in which the F region and the R region correspond to the upstream 1654 bp portion and the downstream 2000 bp portion, respectively, based on the c.2991 + 1655A > G gene mutation.

### 8. T-Blunt End Cloning

The target cassette or PCR product was cloned into a T-vector to identify vectorization of the cassette or the sequence of the PCR product. The All in One™ PCR Cloning Kit (VT202-020, Biofact) was used. Cloning was performed using the product or cassette DNA which was designed so that the length of DNA does not exceed 2 kb. A mixture having the following composition was prepared and subjected to ligation reaction.

TABLE 15
Composition information for cloning

| Component | Volume | |
|---|---|---|
| 6X All in one buffer | 1 [symbol] | Reaction at |
| All in one vector | 1 [symbol] | room temperature |
| PCR Product or Cassette | 4 [symbol] | for 30 minutes |
| Total | 6 [symbol] | |

After allowing reaction to occur for 30 minutes, transformation was performed on E. coli C.P. cells. After completion of incubation on a LB Plate, positive colonies were identified through colony PCR and incubation was performed in 3 ml of LB medium. After Miniprep, sequencing was performed to finally identify sequence matching.

### 9. Vector Construction

The following procedure was performed using the Cas14a1 Ver. 4.0 Dual gRNA vector (Korean Patent Application Nos. 10-2021-0050093 and 10-2021-0051552). Restriction enzyme ends of the vector to be cloned were checked, and a dual gRNA oligo was designed and ordered for custom fabrication. The custom-fabricated oligos were diluted to become 100 pmol. 4.5 μl each of forward and reverse primers was taken and put into a PCR tube, and then 1 μl of 10× annealing buffer was added to adjust the volume to 10 μl. Then, annealing was performed (annealing was performed under the conditions of 95° C., 5 minutes and −1° C./min from 95° C. to 4° C.). The Cas14a1 Ver. 4.0 Dual gRNA vector was prepared and digested with the following composition and conditions.

TABLE 16
Composition information for digestion

| Reagent | Volume | |
|---|---|---|
| NEB 10X 2.1 buffer | 5 [symbol] | Reaction at |
| Vector | 10 [symbol] | 37° C., 500 rpm |
| Bbs I | 10 [symbol] | for 2 hours. |
| DW | Up to 50 [symbol] | |
| Total | 50 [symbol] | |

After the digestion, the digested vector was obtained through electrophoresis and gel elution. Ligation was performed using the digested vector and the annealed oligo.

TABLE 17
Composition information for ligation

| Reagent | Volume | |
|---|---|---|
| DNA ligation mix | 2 [symbol] | Reaction at |
| (TAKARA) | | room temperature |
| Annealed Oligo | 1.5 [symbol] | for 5 minutes |
| Bbsl cut vector | 0.5 [symbol] | |
| Total | 4 [symbol] | |

After the ligation, DH5a transformation was performed. After completion of incubation on a LB Plate, positive colonies were identified through colony PCR and incubation was performed in 3 ml of LB medium. After Miniprep, sequencing was performed to finally identify sequence matching.

### 10. DH5α Transformation

The previously produced vector was transformed into E. coli to produce the vectors. DH5a competent cells were taken out and thawed on ice. The ligated vector was added up to 1/10 of the amount of DH5a and then reaction was allowed to occur on ice for 30 minutes. The resultant was subjected to heat shock at 42° C. for 30 seconds, and then cooled on ice for 2 minutes. Incubation was performed at 37° C. for 1 hour using 100 μl of LB medium or SOC medium. The resultant was smeared on an LB plate warmed to room temperature (which contains ampicillin or kanamycin depending on the vector), and incubation was performed at 37° C. for 14 to 16 hours.

### 11. Production of ARPE19/HPV16-LCA10 Cell Line

ARPE19/HPV16 cells were seeded in a 100 mm dish on the day before transfection (in which seeding was performed so that the confluency reached 70% on the day of transfection). The next day, confluency was checked, and all cells were removed from the dish using trypsin. Washing was performed twice with PBS, and the supernatant was discarded. Mixing of 100 μl of NEON Transfection R buffer with DNA (vector 6 μg, donor 6 μg) was performed, and then the mixture was mixed well with the previously obtained cell pellet. Transfection was carried out under the conditions of 1350V, 30 ms, 2 pulses. Then, the resultant was placed in a 100 mm dish containing the prepared cell media, shaken well, and incubated for 24 hours. After performing the incubation for 24 hours, selection was carried out with puromycin (8 μg/ml). After carrying out the selection with puromycin for 2 weeks, the clones were transferred to a 96-well plate and genotyping was performed. After the genotyping, the selected cells were sub-cultured in a 24-well plate and analyzed by RT-PCR when the cells grew beyond a certain amount.

### 12. Screening of ARPE19/HPV16-LCA10 Cells

The clones, which were selected for 2 weeks by puromycin (8 μg/ml), were checked for gene insertion through direct PCR and Sanger sequencing. The PCR process was carried out as follows. Direct PCR was performed twice, and then the PCR product was purified. A request for sequence analysis thereof was made to Macrogen. After the analysis, cells containing c.2991+1655A>G were checked, and the cells containing the mutation were named according to the following nomenclature: A to Z, A to H, numbers of 1 to 12 (A to Z: number of experiments, A being for first experiment, B for second experiment, C for third experiment . . . in such an order up to Z; A to H: characters corresponding to rows of a 96-well plate (rows A to H from top); numbers of 1 to 12: numbers corresponding to columns in respective rows, for example, AC7: clone at row C, seventh column in a 96-well plate for first experiment). As a result of the sequence analysis, c.2991+1655A>G was identified in the LCA10-AC7 clone.

TABLE 18
First PCR-related information

| 1st PCR | | | PCR Condition | |
|---|---|---|---|---|
| 2X KOD One | 5 [symbol] | 1 X | 95° C. | 5 min |
| F (10 pmol) | 0.5 [symbol] | 0.5 μM | 98° C. | 10 sec |
| R (10 pmol) | 0.5 [symbol] | 0.5 μM | 59° C. | 5 sec |
| Tween20 | 0.2 | | 68° C. | 10 sec |
| Cell lysate | 3.8 [symbol] | | 68° C. | 2 min |
| Total | 10 [symbol] | | Cycle | 33 |

TABLE 19
Second PCR-related information

| 2nd PCR | | | PCR Condition | |
|---|---|---|---|---|
| 2X KOD One | 5 [symbol] | 1 X | 95° C. | 5 min |
| F (10 pmol) | 0.5 [symbol] | 1 μM | 98° C. | 10 sec |
| R (10 pmol) | 0.5 [symbol] | 1 μM | 59° C. | 5 sec |
| DW | 2 [symbol] | | 68° C. | 10 sec |
| 1st PCR product | 2 [symbol] | | 68° C. | 2 min |
| Total | 10 [symbol] | | Cycle | 33 |

TABLE 20
Primer information

| | Primer | | Product |
|---|---|---|---|
| Target | set | | size |
| c.2991 + | Forward | CTTGGCTCACTGCAAGCTCCAC | 397 bp |
| 1655A > G | primer | (SEQ ID NO: 193) | |
| site | Reverse | ACAGGGTAGGATTCATGTTTAG | |
| | primer | AATGATCA | |
| | | (SEQ ID NO: 194) | |

### Example 1. Selection of Candidates for Protospacer Sequence and Guide Sequence

In order to decrease expression of the cryptic exon X caused by the c.2991+1655A>G gene mutation in the CEP290 gene, the target region was divided into a forward region (F region) and a reverse region (R region), in which the F region and the R region correspond to the upstream 1654 bp portion and the downstream 2000 bp portion, respectively, based on the c.2991+1655A>G gene mutation, and protospacer sequences corresponding to the respective regions were selected. Due to the nature of Cas14a1 in which TTTR is used as a PAM, among the existing Cas12a1 target sequences, protospacer sequences whose PAM is TTTR excluding TTTC were preferentially selected. The selected protospacer sequences are summarized in Tables 21 and 22. In order to easily distinguish the respective sequences, the sequences present in the F region were classified as F and numbered, and the sequences present in the R region were classified as R and numbered. These sequences are summarized in Tables 21 and 22.

TABLE 21
Candidates for protospacer sequence

| | | | | SEQ |
|---|---|---|---|---|
| Target | | PAM | Protospacer sequence | ID |
| region | Name | (TTTR) | (20 nt) | NO |
| F region | PS-F01 | TTTA | TAGAAAATTATGCCTATTTA | 195 |
| | PS-F02 | TTTG | GCAAAAGCAGCAGAAAGCAA | 196 |
| | PS-F03 | TTTA | GTAGGAATCCTGAAAGCTAG | 197 |
| | PS-F04 | TTTA | CTGAGGACAGAACAAGCTCC | 198 |
| | PS-F05 | TTTG | GGAAAATGATAACGTTAAAC | 199 |
| | PS-F06 | TTTA | CTGAATGTGTCTCTATGAGC | 200 |
| | PS-F07 | TTTG | TTCATCTTCCTCATCAGAAA | 201 |
| | PS-F08 | TTTG | GATTATTCTTATCTAAGATC | 202 |
| | PS-F09 | TTTA | TCAATATTATTTAGAAGTAA | 203 |
| | PS-F10 | TTTA | CAGAGTGCATCCATGGTCCA | 204 |
| | PS-F11 | TTTG | TTCCTTGGAATGGATGTAGC | 205 |
| | PS-F12 | TTTA | ACCAGACATCTAAGAGAAAA | 206 |
| | PS-F13 | TTTA | TTCTACATTAAAATTCTACA | 207 |
| | PS-F14 | TTTG | TTCTGGGTACAGGGGTAAGA | 208 |
| | PS-F15 | TTTG | ATTCAGCTAAATCATGCAAG | 209 |
| | PS-F16 | TTTA | TAGTGGCTGAATGACTTCTG | 210 |
| | PS-F17 | TTTG | TTCCCACATAACACACACAC | 211 |
| | PS-F18 | TTTA | TAACTAAACTGAATGAATAA | 212 |
| | PS-F19 | TTTA | ATTAAAATCTGCCAAATCCC | 213 |
| | PS-F20 | TTTG | TGTTTGGGGGGATTTGGCAG | 214 |
| | PS-F21 | TTTG | TGTGATTCTTGAACCTTGTG | 215 |
| | PS-F22 | TTTA | TTCTACAATAAAAAATGATG | 216 |
| | PS-F23 | TTTG | CTGGCTCATAGAGACACATTC | 217 |
| | PS-F24 | TTTG | AGCTAATAGAATTAGATCTTA | 218 |

TABLE 22
Candidates for protospacer sequence

| | | | | SEQ |
|---|---|---|---|---|
| Target | | PAM | Protospacer sequence | ID |
| region | Name | (TTTR) | (20 nt) | NO |
| R region | PS-R01 | TTTG | AGAGGTAAAGGTTCATGAGA | 219 |
| | PS-R02 | TTTG | GCACAGAGTTCAAGCTAATA | 220 |
| | PS-R03 | TTTA | CATATCTGTCTTCCTTAATG | 221 |
| | PS-R04 | TTTA | ATGGAACAAAGTCACTOCTG | 222 |
| | PS-R05 | TTTA | CCCTTCAGGTAACCGGTAGC | 223 |
| | PS-R06 | TTTA | GAATGATCATTCTTGTGGCA | 224 |
| | PS-R07 | TTTA | AGGCGGGGAGTCACATGGGA | 225 |
| | PS-R08 | TTTA | CCTCTCAAAAAGCAACAATA | 226 |
| | PS-R09 | TTTG | AAACAGGAATAGAAATTCAC | 227 |
| | PS-R10 | TTTG | TTCCATTAAAAAAAGTATGC | 228 |
| | PS-R11 | TTTA | ATTTGTTTCTGTGTGTTGAG | 229 |
| | PS-R12 | TTTG | TATTTCCTTATTATCTATTC | 230 |
| | PS-R13 | TTTG | ATAGTAGAAGAAAAAAGAAA | 231 |
| | PS-R14 | TTTG | AGATCCCCTTGAAACACAGG | 232 |
| | PS-R15 | TTTA | CAATTCCTGTGTTTCAAGGG | 233 |
| | PS-R16 | TTTA | TCACTGAAATCCATTGCTTA | 234 |
| | PS-R17 | TTTA | TTTATCACTGAAATCCATTG | 235 |
| | PS-R18 | TTTG | AGTGGAGTACCTGGAAAATA | 236 |
| | PS-R19 | TTTA | GTCTCATTACCCTGTTTGTA | 237 |
| | PS-R20 | TTTA | TATACTATGAATACACAGGA | 238 |
| | PS-R21 | TTTG | TAACAAACAATAGGATTATC | 239 |
| | PS-R22 | TTTA | GAGTTACTAGGGAGAGAGTG | 240 |
| | PS-R23 | TTTA | AAGGAGACCAGTGAAGTTTC | 241 |
| | PS-R24 | TTTG | GATCTCAGGTAATCCGCCAG | 242 |
| | PS-R25 | TTTA | AGTAAAGTACATAATTATAG | 243 |
| | PS-R26 | TTTG | AGACCAGCCCGGCCAACATG | 244 |

Guide RNAs were designed based on the selected protospacer sequences. The guide RNA has a guide sequence that binds complementarily to a target sequence. Such a guide sequence may be designed using a protospacer sequence. The protospacer sequence is a sequence that binds complementarily to a target sequence, and correlation between the protospacer sequence and the target sequence is similar to correlation between the target sequence and the guide sequence. Due to this feature, when designing a guide sequence, the guide sequence may be generally designed using a protospacer sequence. That is, when designing a guide sequence that binds complementarily to a target sequence, the guide sequence may be designed as a nucleotide sequence having the same nucleotide sequence as the protospacer sequence. Here, the guide sequence is designed by replacing T with U in the nucleotide sequence of the protospacer sequence. Therefore, guide sequences were designed using the selected protospacer sequences, and these guide sequences are summarized in Tables 3 and 4.

### Example 2. Selection of Guide Sequence

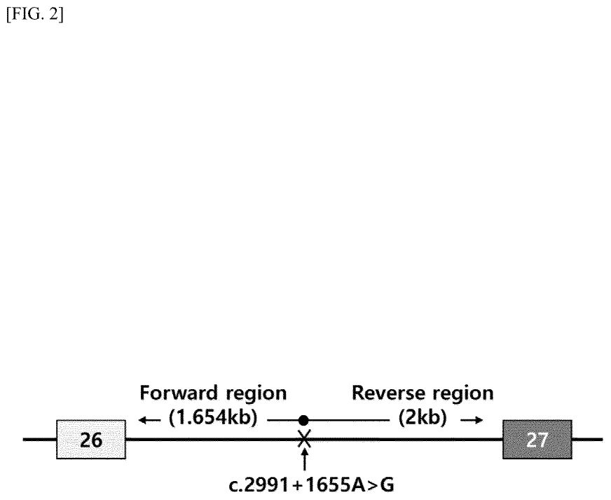

A nucleic acid sequence encoding each of guide RNAs designed based on the selected protospacer sequences was prepared as a cassette and transfected into HEK-293T cells using FuGENE HD along with a Cas14a1 vector. Here, the guide sequences of the guide RNAs are the same as in Tables 3 and 4, and 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUUCGGAA AGUAACCCUCGAAACAAAUUCAGUGCUCCUCUCCAAUUCUGCACAAGAA AGUUGCAGAACCCGAAUAGAGCAAUGAAGGAAUGCAAC-3′ (SEQ ID NO: 126) was used as a scaffold sequence of the guide RNAs. In addition, the guide RNA was designed to contain the sequence 5′-U₄AU₄-3′, which is a U-rich tail sequence, at the 3′ end. Using gDNA from the cells that were 60 to 72 hours after the transfection, indel efficiency was checked by NGS. As a result of the analysis, guide RNAs having the guide sequences F14, F16, F17, and R22, respectively, showed high indel efficiency (FIG. 2).

TABLE 23
Guide RNAs having respective guide sequences and indel efficiency
(%) thereof caused by Cas14a1

| Target | | | | | % |
|---|---|---|---|---|---|
| region | Name | Guide sequence (20 nt) | SEQ ID NO | NGS | Indel |
| F region | Guide-F14 | UUCUGGGUACAGGGGUAAGA | 64 | O | 34.4 |
| | Guide-F16 | UAGUGGCUGAAUGACUUCUG | 66 | O | 17.07 |
| | Guide-F17 | UUCCCACAUAACACACACAC | 67 | O | 26.6 |
| R region | Guide-R22 | GAGUUACUAGGGAGAGAGUG | 96 | O | 26.73 |
| | Guide-R23 | AAGGAGACCAGUGAAGUUUC | 97 | O | 0.16 |
| | Guide-R24 | GAUCUCAGGUAAUCCGCCAG | 98 | O | 0.21 |

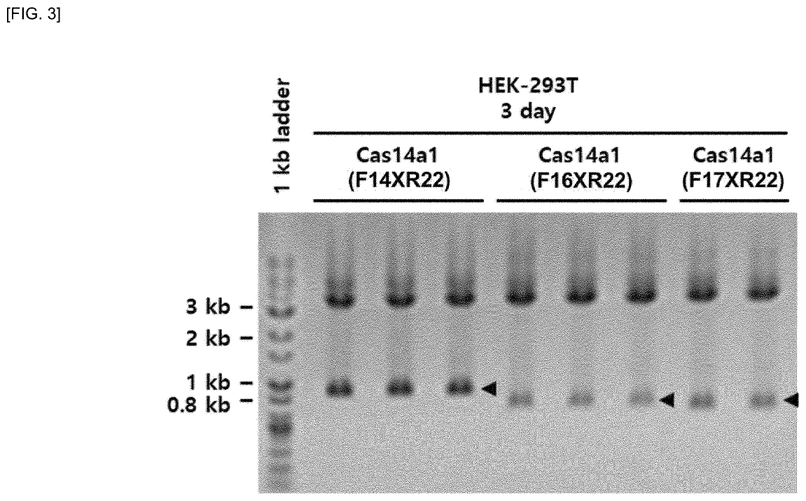

Based on these results, sets were constructed by combining a guide RNA having the guide sequence F14, F16, or F17 that targets the F region, and a guide RNA having the guide sequence R22 that targets the R region. The respective sets were compared for deletion efficiency of a partial region in the CEP290 gene. As a result, NT, which is a control with no treatment, showed a band of 3672 bp, the F14× R22 set (that is, a guide RNA having F14 as a guide sequence and a guide RNA having R22 as a guide sequence) resulted in deletion of about 2500 bp and showed band of about 1170 bp, and the F16× R22 set (that is, a guide RNA having F16 as a guide sequence and a guide RNA having R22 as a guide sequence) resulted in deletion of about 2700 bp and showed band of about 1000 bp. In addition, the F17× R22 set (that is, a guide RNA having F17 as a guide sequence and a guide RNA having R22 as a guide sequence) resulted in deletion of about 2750 bp and showed a band of 950 bp. As a result of comparing intensity of the deletion bands, it was identified that the F14× R22 set exhibited the best efficiency as in the NGS results (FIG. 3).

### Example 2-1. Identification of Deletion of Partial Region in CEP290 Gene

Nucleic acids encoding the guide RNA set, which previously showed high indel efficiency, that is, the F14× R22 set (that is, a guide RNA having F14 as a guide sequence and a guide RNA having R22 as a guide sequence), were vectorized into a cassette through T-blunt end cloning, and deletion of a partial region in the CEP290 gene was additionally identified. EDIT101 (Morgan L. Maeder et al., Nature Medicine, 25, 229-233 (2019)) was used as a control for comparison of efficiency.

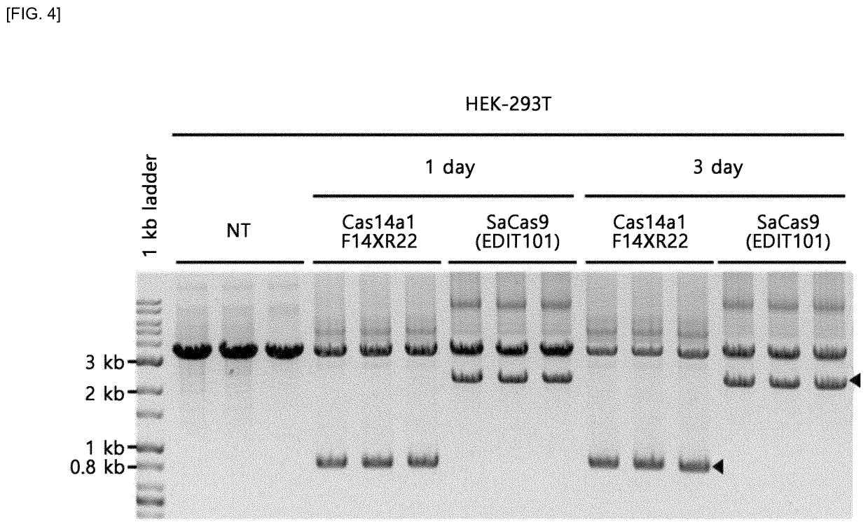

As a result of identifying deletion by transfection into HEK-293T, it was difficult to determine efficiency by intensity due to a large difference in size of deletion band between the experimental group and the control; however, as a result of comparing intensity between the band of the negative control NT with no treatment and a band of the same size (that is, a portion not cleaved by the CRISPR/Cas system), it was identified that Cas14a1 and F14× R22 showed lower band intensity than EDIT101. As a result, it was predictable that Cas14a1 and F14× R22 showed higher deletion efficiency (FIG. 4).

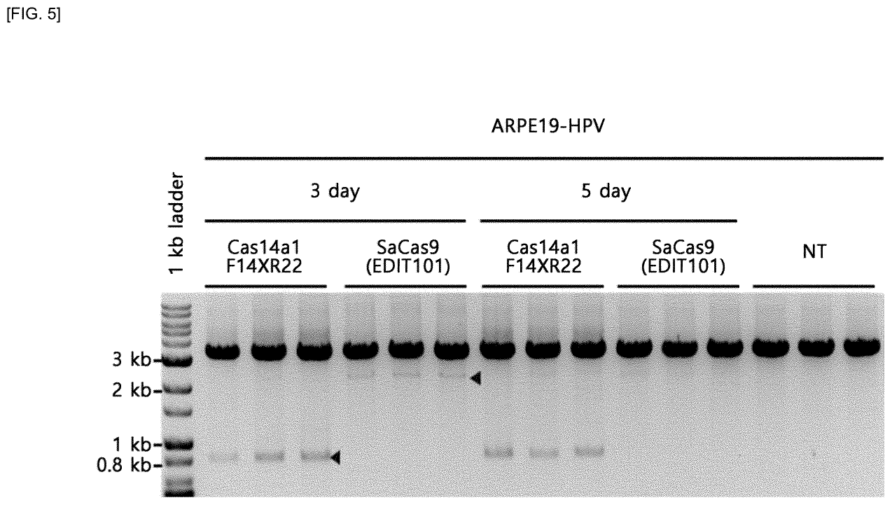

In addition, as a result of identifying deletion by transfection into ARPE19-HPV, although poor transfection efficiency was observed, from the viewpoint that when determined by intensity of deletion band, Cas14a1 and F14× R22 showed higher intensity than EDIT101 despite its smaller size of deletion band, it was identified that Cas14a1 and F14× R22 showed higher efficiency than EDIT101 (FIG. 5).

### Example 3. Selection of Guide Sequence

A nucleic acid sequence encoding each of the guide RNAs designed based on the selected protospacer sequences was prepared as a cassette (PCR amplicon), and transfected into HEK-293T cells using FuGENE HD along with a Cas14a1 vector. Here, the guide sequences of the guide RNAs are the same as in Tables 3 and 4, and 5′-ACCGCUUCACCAAAAGCUGUCCCUUAGGGGAUUAGAACUUGAGUGAA GGUGGGCUGCUUGCAUCAGCCUAAUGUCGAGAAGUGCUUUCUGGAAAG UAACCCUCGAAACAAAGAAAGGAAUGCAAC-3′ (SEQ ID NO: 125) was used as a scaffold sequence of the guide RNAs. In addition, the guide RNA was designed to contain the sequence 5′-U₄AU₄-3′, which is a U-rich tail sequence, at the 3′ end. Using gDNA from the cells that were 60 to 72 hours after the transfection, indel efficiency was checked by NGS. As a result of the analysis, guide RNAs having the guide sequences F10, F14, F16, F17, R05, R07, R18, R20, R22, and R26, respectively, showed high indel efficiency.

TABLE 24
Indel efficiency (%) caused by guide RNAs having respective guide
sequences and Cas14a1

| Target | | | | | % |
|---|---|---|---|---|---|
| region | Name | Guide sequence (20 nt) | SEQ ID NO | NGS | Indel |
| F region | Guide-F01 | UAGAAAAUUAUGCCUAUUUA | 51 | O | 0.01 |
| | Guide-F02 | GCAAAAGCAGCAGAAAGCAA | 52 | O | 0 |
| | Guide-F03 | GUAGGAAUCCUGAAAGCUAC | 53 | O | 0.01 |
| | Guide-F04 | CUGAGGACAGAACAAGCUCC | 54 | O | 0.14 |
| | Guide-F05 | GGAAAAUGAUAACGUUAAAC | 55 | O | 0.01 |
| | Guide-F06 | CUGAAUGUGUCUCUAUGAGC | 56 | O | 0.05 |
| | Guide-F07 | UUCAUCUUCCUCAUCAGAAA | 57 | O | 0 |
| | Guide-F08 | GAUUAUUCUUAUCUAAGAUC | 58 | O | 0.01 |
| | Guide-F09 | UCAAUAUUAUUUAGAAGUAA | 59 | O | 0 |
| | Guide-F10 | CAGAGUGCAUCCAUGGUCCA | 60 | O | 34.1 |
| | Guide-F11 | UUCCUUGGAAUGGAUGUAGC | 61 | O | 1.44 |
| | Guide-F12 | ACCAGACAUCUAAGAGAAAA | 62 | O | 0.42 |
| | Guide-F13 | UUCUACAUUAAAAUUCUACA | 63 | O | 0.01 |
| | Guide-F14 | UUCUGGGUACAGGGGUAAGA | 64 | O | 43.12 |
| | Guide-F15 | AUUCAGCUAAAUCAUGCAAG | 65 | O | 2.82 |
| | Guide-F16 | UAGUGGCUGAAUGACUUCUG | 66 | O | 26.74 |
| | Guide-F17 | UUCCCACAUAACACACACAC | 67 | O | 26.33 |
| | Guide-F18 | UAACUAAACUGAAUGAAUAA | 68 | O | 0.42 |
| | Guide-F19 | AUUAAAAUCUGCCAAAUCCC | 69 | O | 0.31 |
| | Guide-F20 | UGUUUGGGGGGAUUUGGCAG | 70 | O | 0.25 |
| | Guide-F21 | UGUGAUUCUUGAACCUUGUG | 71 | O | 0.02 |
| | Guide-F22 | UUCUACAAUAAAAAAUGAUG | 72 | O | 0 |
| | Guide-F23 | CUGGCUCAUAGAGACACAUUC | 73 | O | 0.97 |
| | Guide-F24 | AGCUAAUAGAAUUAGAUCUUA | 74 | O | 0.75 |

TABLE 25
Indel efficiency (%) caused by guide RNAs having respective guide
sequences and Cas14a1

| Target | | | | | % |
|---|---|---|---|---|---|
| region | Name | Guide sequence (20 nt) | SEQ ID NO | NGS | Indel |
| R region | Guide-R01 | AGAGGUAAAGGUUCAUGAGA | 75 | O | 1.39 |
| | Guide-R02 | GCACAGAGUUCAAGCUAAUA | 76 | O | 0.04 |
| | Guide-R03 | CAUAUCUGUCUUCCUUAAUG | 77 | O | 1.47 |
| | Guide-R04 | AUGGAACAAAGUCACUCCUG | 78 | O | 1.53 |
| | Guide-R05 | CCCUUCAGGUAACCGGUAGC | 79 | O | 10.12 |
| | Guide-R06 | GAAUGAUCAUUCUUGUGGCA | 80 | O | 0.01 |
| | Guide-R07 | AGGCGGGGAGUCACAUGGGA | 81 | O | 15.85 |
| | Guide-R08 | CCUCUCAAAAAGCAACAAUA | 82 | O | 0.13 |
| | Guide-R09 | AAACAGGAAUAGAAAUUCAC | 83 | O | 0.01 |
| | Guide-R10 | UUCCAUUAAAAAAAGUAUGC | 84 | O | 0.03 |
| | Guide-R11 | AUUUGUUUCUGUGUGUUGAG | 85 | O | 0 |
| | Guide-R12 | UAUUUCCUUAUUAUCUAUUC | 86 | O | 0 |
| | Guide-R13 | AUAGUAGAAGAAAAAAGAAA | 87 | O | 0 |
| | Guide-R14 | AGAUCCCCUUGAAACACAGG | 88 | O | 0 |
| | Guide-R15 | CAAUUCCUGUGUUUCAAGGG | 89 | O | 0.02 |
| | Guide-R16 | UCACUGAAAUCCAUUGCUUA | 90 | O | 0.04 |
| | Guide-R17 | UUUAUCACUGAAAUCCAUUG | 91 | O | 0.02 |
| | Guide-R18 | AGUGGAGUACCUGGAAAAUA | 92 | O | 26.76 |
| | Guide-R19 | GUCUCAUUACCCUGUUUGUA | 93 | X | — |
| | Guide-R20 | UAUACUAUGAAUACACAGGA | 94 | O | 17.61 |
| | Guide-R21 | UAACAAACAAUAGGAUUAUC | 95 | O | 0.02 |
| | Guide-R22 | GAGUUACUAGGGAGAGAGUG | 96 | O | 32.09 |
| | Guide-R23 | AAGGAGACCAGUGAAGUUUC | 97 | O | 0.24 |
| | Guide-R24 | GAUCUCAGGUAAUCCGCCAG | 98 | O | 0 |
| | Guide-R25 | AGUAAAGUACAUAAUUAUAG | 99 | O | 0.51 |
| | Guide-R26 | AGACCAGCCCGGCCAACAUG | 100 | O | 7.9 |

Nucleic acids encoding the selected 10 guide RNAs were vectorized into cassettes through T-blunt end cloning, and indel efficiency thereof was checked by NGS. After vectorization, it was identified that the indel efficiency was increased. Among them, guide RNAs having the guide sequences F10, F14, R05, R07, R18, and R22, respectively, were selected in consideration of location from the c.2991+1655A>G gene mutation site and indel efficiency.

TABLE 26
Indel efficiency (%) caused by guide RNAs having respective guide
sequences and Cas14a1

| Target | | | | | % |
|---|---|---|---|---|---|
| region | Name | Guide sequence (20 nt) | SEQ ID NO | NGS | Indel |
| F region | Guide-F10 | CAGAGUGCAUCCAUGGUCCA | 60 | O | 62.12 |
| | Guide-F14 | UUCUGGGUACAGGGGUAAGA | 64 | O | 60.66 |
| | Guide-F16 | UAGUGGCUGAAUGACUUCUG | 66 | O | 53.9 |
| | Guide-F17 | UUCCCACAUAACACACACAC | 67 | O | 51.6 |
| R region | Guide-R05 | CCCUUCAGGUAACCGGUAGC | 79 | O | 10.87 |
| | Guide-R07 | AGGCGGGGAGUCACAUGGGA | 81 | O | 19.88 |
| | Guide-R18 | AGUGGAGUACCUGGAAAAUA | 92 | O | 61.41 |
| | Guide-R20 | UAUACUAUGAAUACACAGGA | 94 | O | 49.58 |
| | Guide-R22 | GAGUUACUAGGGAGAGAGUG | 96 | O | 58.81 |
| | Guide-R26 | AGACCAGCCCGGCCAACAUG | 100 | O | 18.44 |

### Example 3-1. Optimization of Guide Sequence

The length of the guide sequences was adjusted using 19- to 25-mer protospacer sequences based on the PAM of each target. Cas14a1 ver.4.0 dual vectors containing nucleic acids encoding guide sequences of various lengths were constructed, and indel efficiency depending on the length of the guide sequence was checked (Tables 27 and 28).

TABLE 27
Indel efficiency (%) caused by guide RNAs having guide sequences
of various lengths and Cas14a1

| Target | | | SEQ ID | % |
|---|---|---|---|---|
| region | Name | Guide sequence | NO | Indel |
| F region | Guide-F10-19 nt | CAGAGUGCAUCCAUGGUCC | 245 | 29.89 |
| | Guide-F10-20 nt | CAGAGUGCAUCCAUGGUCCA | 60 | 25.98 |
| | Guide-F10-21 nt | CAGAGUGCAUCCAUGGUCCAG | 246 | 30.23 |
| | Guide-F10-22 nt | CAGAGUGCAUCCAUGGUCCAGG | 247 | 34.55 |
| | Guide-F10-23 nt | CAGAGUGCAUCCAUGGUCCAGGA | 248 | 29.69 |
| | Guide-F10-24 nt | CAGAGUGCAUCCAUGGUCCAGGAA | 249 | 28.23 |
| | Guide-F10-25 nt | CAGAGUGCAUCCAUGGUCCAGGAAG | 250 | 17.8 |
| | Guide-F14-19 nt | UUCUGGGUACAGGGGUAAG | 251 | 30.98 |
| | Guide-F14-20 nt | UUCUGGGUACAGGGGUAAGA | 64 | 45.89 |
| | Guide-F14-21 nt | UUCUGGGUACAGGGGUAAGAG | 252 | 33.98 |
| | Guide-F14-22 nt | UUCUGGGUACAGGGGUAAGAGA | 253 | 36.91 |
| | Guide-F14-23 nt | UUCUGGGUACAGGGGUAAGAGAA | 254 | 30.22 |
| | Guide-F14-24 nt | UUCUGGGUACAGGGGUAAGAGAAA | 255 | 35.21 |
| | Guide-F14-25 nt | UUCUGGGUACAGGGGUAAGAGAAAG | 256 | 25.35 |

TABLE 28
Indel efficiency (%) caused by guide RNAs having guide sequences
of various lengths and Cas14a1

| Target | | | SEQ ID | % |
|---|---|---|---|---|
| region | Name | Guide sequence | NO | Indel |
| Rregion | Guide-R05-19 nt | CCCUUCAGGUAACCGGUAG | 257 | 3.86 |
| | Guide-R05-20 nt | CCCUUCAGGUAACCGGUAGC | 79 | 8.51 |
| | Guide-R05-25 nt | CCCUUCAGGUAACCGGUAGCUUUUG | 258 | 5.19 |
| | Guide-R07-19 nt | AGGCGGGGAGUCACAUGGG | 259 | 13.47 |
| | Guide-R07-20 nt | AGGCGGGGAGUCACAUGGGA | 81 | 15.18 |
| | Guide-R07-21 nt | AGGCGGGGAGUCACAUGGGAG | 260 | 16.17 |
| | Guide-R07-23 nt | AGGCGGGGAGUCACAUGGGAGUC | 261 | 16.27 |
| | Guide-R07-24 nt | AGGCGGGAGUCACAUGGGAGUCA | 262 | 14.55 |
| | Guide-R07-25 nt | AGGCGGGGAGUCACAUGGGAGUCAC | 263 | 14.02 |
| | Guide-R18-20 nt | AGUGGAGUACCUGGAAAAUA | 92 | 33.95 |
| | Guide-R18-21 nt | AGUGGAGUACCUGGAAAAUAA | 264 | 37.51 |
| | Guide-R18-22 nt | AGUGGAGUACCUGGAAAAUAAG | 265 | 35.78 |
| | Guide-R18-23 nt | AGUGGAGUACCUGGAAAAUAAGC | 266 | 34.25 |
| | Guide-R18-24 nt | AGUGGAGUACCUGGAAAAUAAGCC | 267 | 27.9 |
| | Guide-R18-25 nt | AGUGGAGUACCUGGAAAAUAAGCCA | 268 | 21.53 |
| | Guide-R22-20 nt | GAGUUACUAGGGAGAGAGUG | 96 | 37.03 |
| | Guide-R22-21 nt | GAGUUACUAGGGAGAGAGUGA | 269 | 33.96 |
| | Guide-R22-23 nt | GAGUUACUAGGGAGAGAGUGAUG | 270 | 34.37 |
| | Guide-R22-24 nt | GAGUUACUAGGGAGAGAGUGAUGA | 271 | 35.54 |
| | Guide-R22-25 nt | GAGUUACUAGGGAGAGAGUGAUGAA | 272 | 38.9 |

### Example 4. Production of ARPE19/HPV16-LCA10 Cell Line

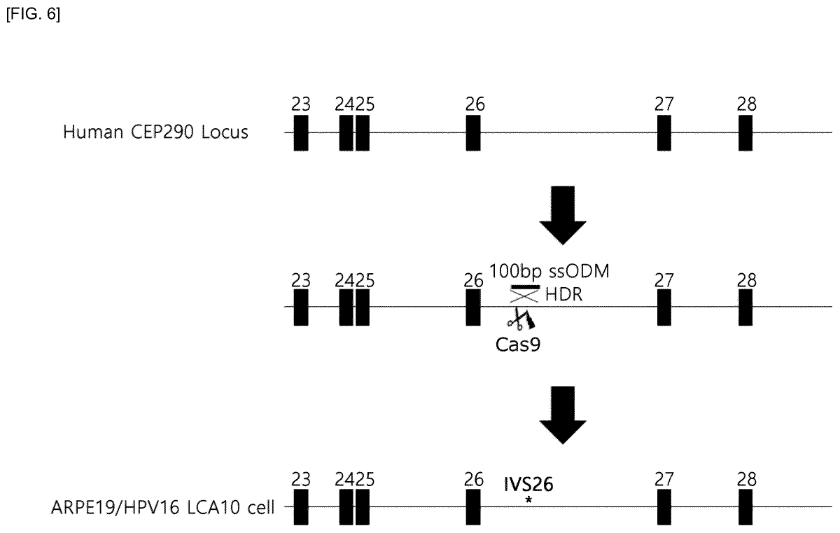

To identify effects obtained by artificially manipulating the CEP290 gene having the IVS26 mutation using the guide RNAs selected through the above-described screening, an ARPE19/HPV16-LCA10 cell line containing the CEP290 gene having the IVS26 mutation was produced. To briefly describe a strategy for producing the ARPE19/HPV16-LCA10 cell line, an experiment was designed in such a way that a CRIPSR/Cas9 system and a single-strand oligonucleotide (ssODN) were used to introduce a point mutation (c.2991+1655A>G) into intron 26 of the CEP290 locus (FIG. 6). To this end, 5′-ATCCCTATTGGCAGTGTCTT-3′ (SEQ ID NO: 273) was used as a target sequence for the CRIPSR/Cas9 system, and GAGCCACCGCACCTGGCCCCAGTTGTAATTGTGAGTATCTCATAGATATCCC TATTGGCAGTGTCTTAGTTTTATTTTTTATTATCTTTATTGTGGCAGC-3′ (SEQ ID NO: 274) was used as a donor sequence, that is, ssODN. Here, the underlined sequence in the donor sequence is a point mutation. To facilitate selection, a puromycin resistance gene was inserted together into a vector for expression of the CRISPR/Cas9 system used in the experiment. By transfecting the expression vector for the CRISPR/Cas9 system and ssODN together into ARPE19 cells, the target sequence was cleaved by the CRISPR/Cas9 system and repaired by HDR using ssODN, which caused a point mutation (c.2991+1655A) to be introduced into intron 26 of the CEP290 locus of interest.

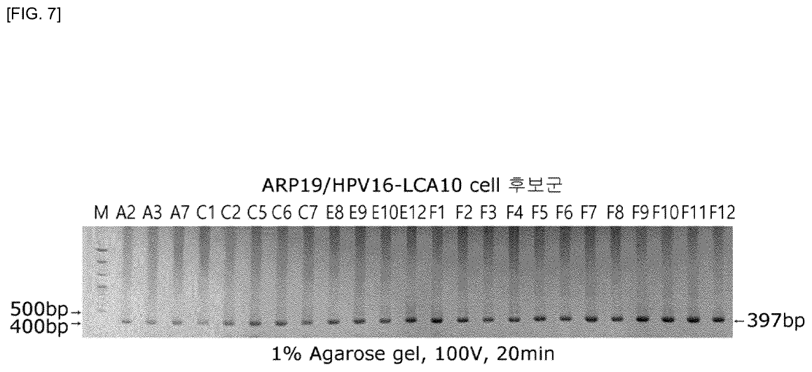

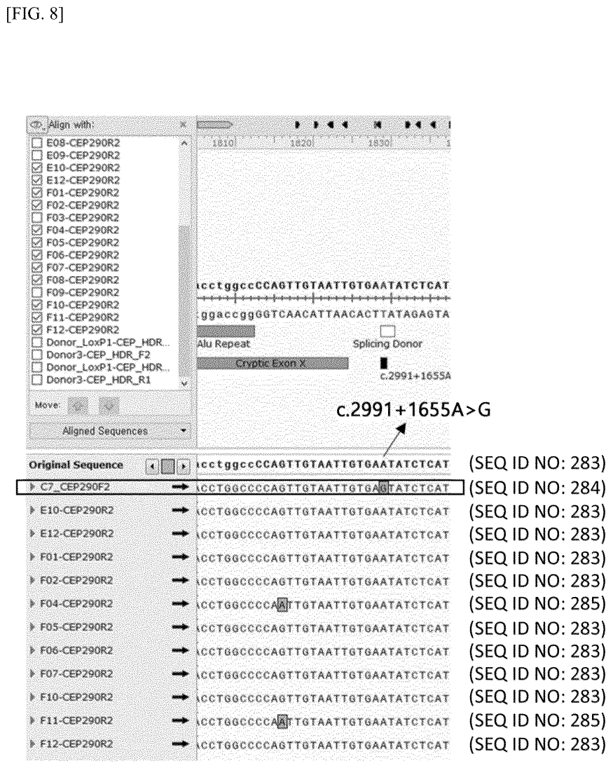

The thus produced ARPE19/HPV16-LCA10 cells were first checked by PCR using the primer set as shown in Table 20. As a result, a band of 397 bp was identified in the candidate cells A2, A3, A7, C1, C2, C5 to C7, E8 to E12, and F1 to F12 (FIG. 7). The PCR products of the corresponding candidate cells were purified and a request for sequencing analysis thereof was made to Macrogen. As a result of the sequencing analysis, the c.2991+1655A>G mutation was identified in C7 cells (AC7 cells). For the other cells, deletion and insertion were identified in a site located after CCT that is a target portion of Cas9 (FIG. 8).

### Example 4-1. LCA10-AC7 Clone Expression Analysis

RNA was extracted from a clone of the previously obtained AC7 cells (hereinafter referred to as LCA10-AC7 cells) and the ARPE19/HPV16 (WT) cells, and cDNA was synthesized therefrom (RNA extraction: Rneasy kit, Qiagen; cDNA synthesis: TOPscript RT DryMIX, enzynomics). RNA extraction and cDNA synthesis were performed according to the manufacturer's manual. The obtained cDNA was subjected to qRT-PCR and RT-PCR (1.5% agarose gel, 100 v, electrophoresis for 20 minutes) using the following primers in an equal amount of 10 ng.

TABLE 29
Primer information for qRT-PCR

| | Primer | | Product |
|---|---|---|---|
| Target | set | | size |
| Wt cDNA | CEP290 | CCATTTTCAAGATTGCAGCTCTC | 166 bp |
| (exon | WT F#01 | CAA (SEQ ID NO: 275) | |
| 26) | CEP290 | TCCAGGTGTTCCAAGTTACTTGT | |
| | WT R#01 | TCT (SEQ ID NO: 276) | |
| Mutation | CEP290 | TGACTGCTAAGTACAGGGACATC | 173 bp |
| cDNA | Mu F#02 | TTG (SEQ ID NO: 277) | |
| (exon | CEP290 | Atctgtaatcccagcactttagg | |
| 26~28) | Mu R#02 | agg (SEQ ID NO: 278) | |

TABLE 30
Primer information for RT-PCR

| | Primer | | Product |
|---|---|---|---|
| Target | set | | size |
| 26-X-27 | CEP290 | AGGAAATGGCCATTTTCAAGAT | 340 bp |
| (26-27) | WT F#02 | TGCA (SEQ ID NO: 279) | |
| | CEP290 | AGACTCCACTTGTTCTTTTAAG | (212 bp) |
| | CDNA | GAG (SEQ ID NO: 280) | |
| | Screening | | |

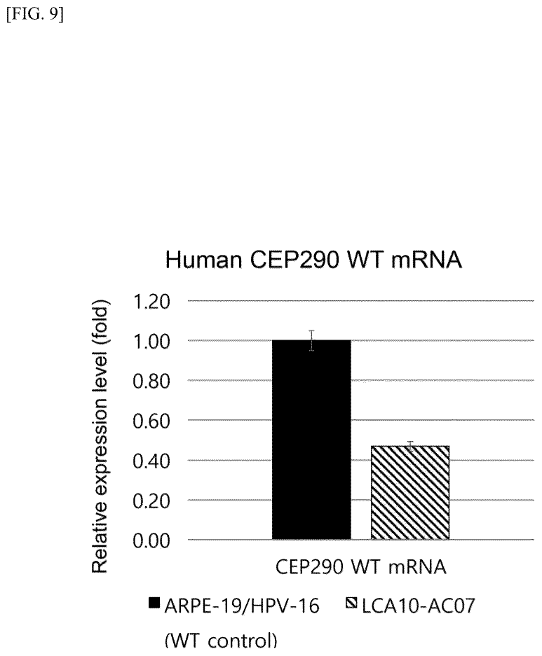

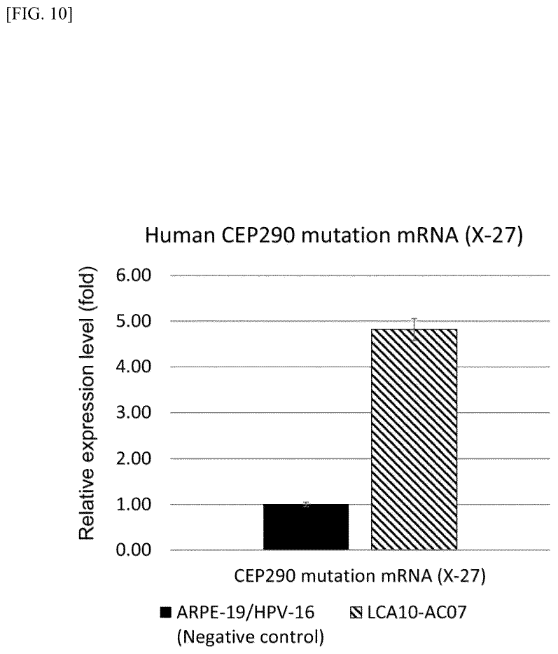

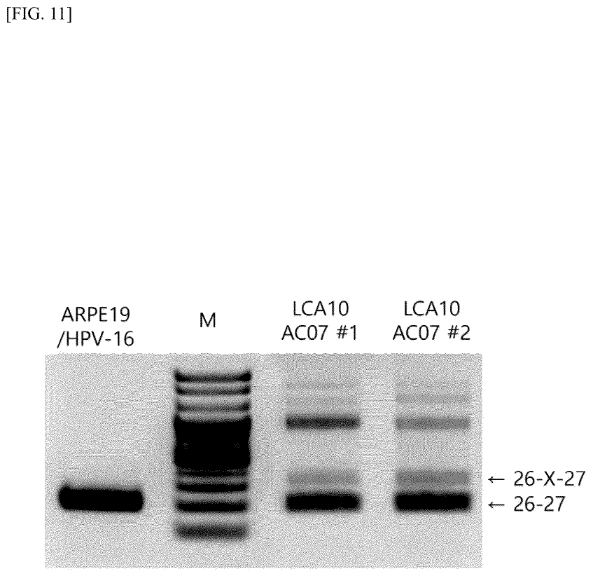

The qRT-PCR results were subjected to relative quantification with WT through the 2\hat{ }-ddCt method. ARPE-19/HPV16 CEP290 WT mRNA and LCA10-AC07 CEP290 WT mRNA were compared (FIG. 9), and ARPE-19/HPV16 CEP290 mutation mRNA (X-27) and LCA10-AC07 CEP290 mutation mRNA (X-27) were compared (FIG. 10). As a result, due to generation of the C.2991+1655A>G mutation, it was identified that expression of the WT mRNA and the mutantion mRNA showed a different tendency from ARPE-19/HPV16 WT. In a case where cryptic exon X was expressed due to generation of the C.2991+1655A>G mutation, a ratio of WT decreased and a ratio of mutation mRNA increased among the total CEP290 mRNA. Therefore, in a case where the corresponding portion is corrected with a genetic scissor, a ratio of WT increases and a ratio of mutation mRNA decreases, again (Morgan L. Maeder et al., Nature Medicine, 25, 229-233 (2019)). In addition, as a result of RT-PCR, the band 26-27 (212 bp) was detected in both WT and the LCA10-AC7 cells, and the band 26-X-27 (340 bp) was detected only in the LCA10-AC7 cells, indicating that the cryptic exon X was generated by a point mutation (c.2991+1655A>G) in intron 26 of the CEP290 locus in the LCA10-AC7 cells (FIG. 11).

### Example 4-2. Additional Production of ARPE19/HPV16-LCA10 Cell Line

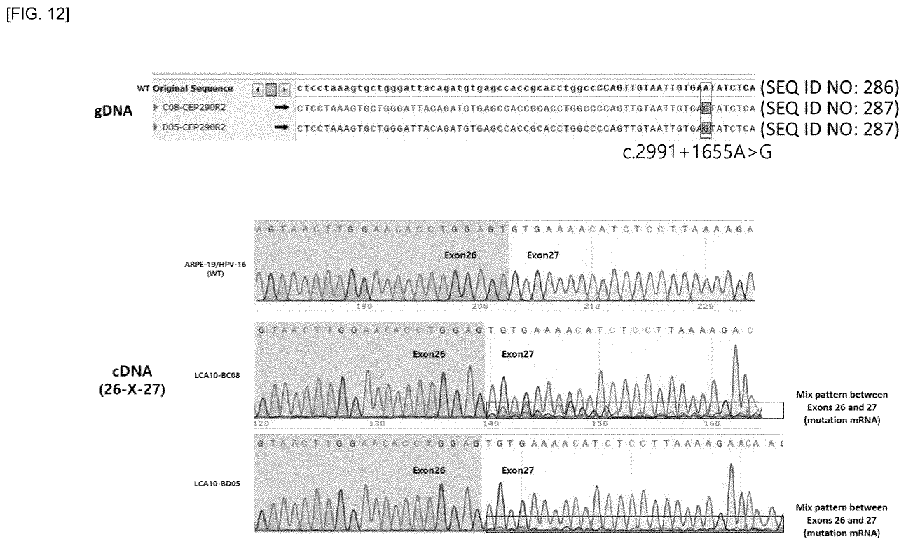

Since it is difficult to identify accuracy of the in vitro assay using only the previously obtained LCA10-AC7 cells, additional production was performed to obtain additional ARPE19/HPV16-LCA10 cells. The production method is the same as the previously described production method. As a result of the additional production, a point mutation (c.2991+1655A>G) was identified in intron 26 of the CEP290 locus in LCA10-BC8 cells and LCA10-BD5 cells (FIG. 12).

RNA was extracted from each clone of the obtained LCA10-AC7 cells, LCA10-BC8 cells, and LCA10-BD5 cells and from the ARPE19/HPV16 (WT) cells, and cDNA was synthesized therefrom (RNA extraction: Rneasy kit, Qiagen; cDNA synthesis: TOPscript RT DryMIX, enzynomics). RNA extraction and cDNA synthesis were performed according to the manufacturer's manual. The obtained cDNA was subjected to qRT-PCR and RT-PCR (1.5% agarose gel, 100 v, electrophoresis for 20 minutes) using the following primers in an equal amount of 10 ng. The RT-PCR-related primer information is the same as the primer information in Table 29.

TABLE 31
qRT-PCR-related primer information

| | Primer | | Product |
|---|---|---|---|
| Target | set | | size |
| Mutation | CEP290 | tagagatggggtttcaccttg | 164 bp |
| cDNA | Mu F#03 | ttagc (SEQ ID NO: 281) | |
| (exon | CEP290 | AGACTCCACTTGTTCTTTTAA | |
| 26~28) | CDNA | GGAG (SEQ ID NO: 282) | |
| | Screening | | |

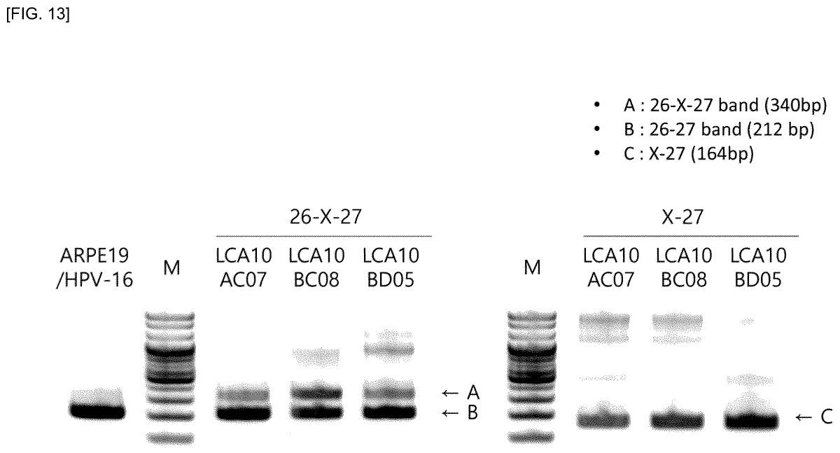

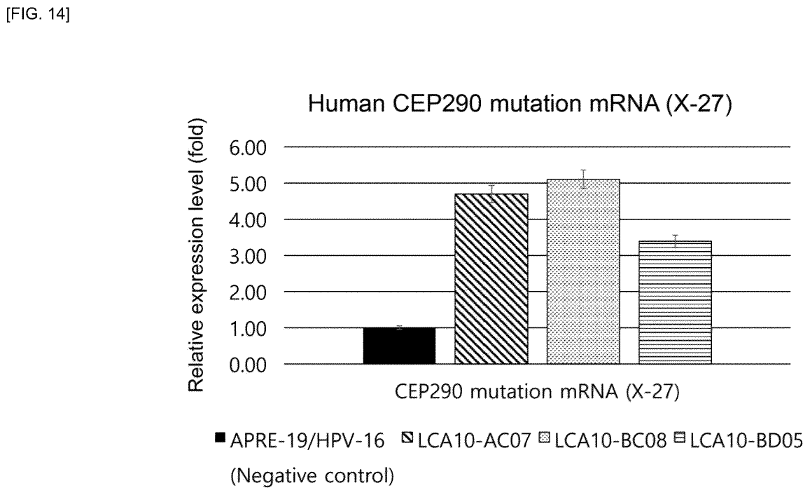

As a result of RT-PCR, the band 26-27 (212 bp) was detected in all of WT, the LCA10-AC7 cells, the LCA10-BC8 cells, and the LCA10-BD5 cells; the band 26-X-27 (340 bp) was detected only in the LCA10-AC7 cells, the LCA10-BC8 cells, and the LCA10-BD5 cells; and the band X-27 (164 bp) was also detected in the LCA10-AC7 cells, the LCA10-BC8 cells, and the LCA10-BD5 cells. From the results, it was identified that cryptic exon X was generated by a point mutation (c.2991+1655A>G) in intron 26 of the CEP290 locus in the LCA10-AC7 cells, the LCA10-BC8 cells, and the LCA10-BD5 cells (FIG. 13). In addition, an expression level of cryptic exon X was checked in the LCA10-BC8 cells and the LCA10-BD5 cells. Here, each of the expression levels was compared relative to the expression of the cryptic exon X mRNA of the LCA10-AC7 cells that were produced first. As a result, it was identified that the LCA10-BC8 cells showed slightly increased expression of cryptic exon X as compared with the LCA10-AC7 cells, whereas the LCA10-BD5 cells showed approximately 30% lower expression of cryptic exon X than the LCA10-AC7 cells (FIG. 14).

### Example 5. Identification of Efficiency of LCA10 Cas12f1 Therapeutic Agent

As a result of performing comparative analysis of efficiency of the dual gRNA vector and the one vector using the previously produced LCA10-AC7 cells, LCA10-BC8 cells, and LCA10-BD5 cells, the most significant result was obtained in the BC8 clone. Based on these results, efficiency was evaluated using the LCA10-BC8 cells to identify efficiency of a Cas12f1-based LCA10 therapeutic agent. Here, for the guide RNAs used in the experiment, a guide RNA having F14 as a guide sequence and a guide RNA having R22 as a guide sequence, which were previously selected through screening, were used. The two guide RNAs were intended to be used to remove the point mutation (c.2991+1655A>G) present in intron 26 of the CEP290 locus, thereby inhibiting generation of cryptic exon X.

To identify efficiency of the LCA10 Cas12f1 therapeutic agent, LCA10-BC8 cells were seeded one day before transfection so that the confluency reached 70% the next day. The next day, Cas12f1_LCA10_F14_vector (1 μg)+Cas12f1_LCA10_R22_vector (1 μg)+FuGENE HD 6 μg were mixed, and reaction was allowed to occur at room temperature for 20 minutes. Then, transfection was performed. After 72 hours, RNA was extracted from the transfected LCA10-BC8 cells and the ARPE19/HPV16 (WT) cells, and cDNA was synthesized therefrom (RNA extraction: Rneasy kit, Qiagen; cDNA synthesis: TOPscript RT DryMIX, enzynomics). RNA extraction and cDNA synthesis were performed according to the manufacturer's manual. The obtained cDNA was subjected to qRT-PCR and RT-PCR using the following primers in an equal amount of 10 ng. The RT-PCR-related primer information is the same as the primer information in Table 29, and the qRT-PCR-related primer information is the same as the primer information in Table 30. The qRT-PCR results were subjected to relative comparative quantification with WT through the 2\hat{ }-ddCt method. The RT-PCR results were subjected to electrophoresis on 2% agarose gel (Etbr) at 135 V for 15 minutes. The cDNA was sequenced to identify a signal of the cryptic exon.

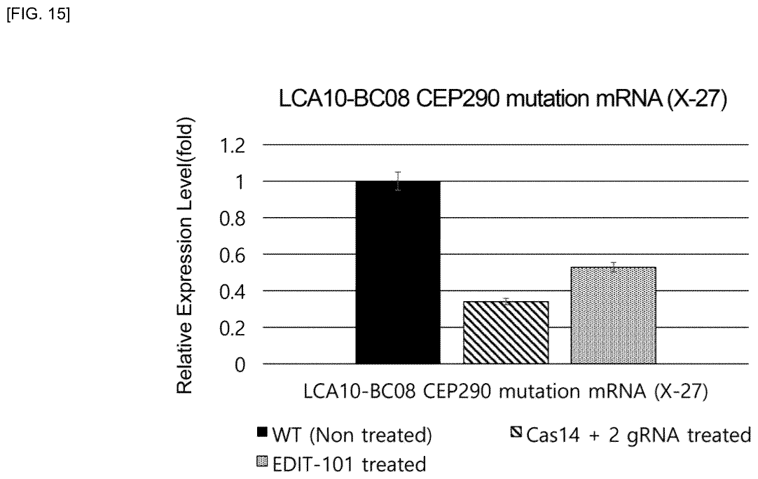

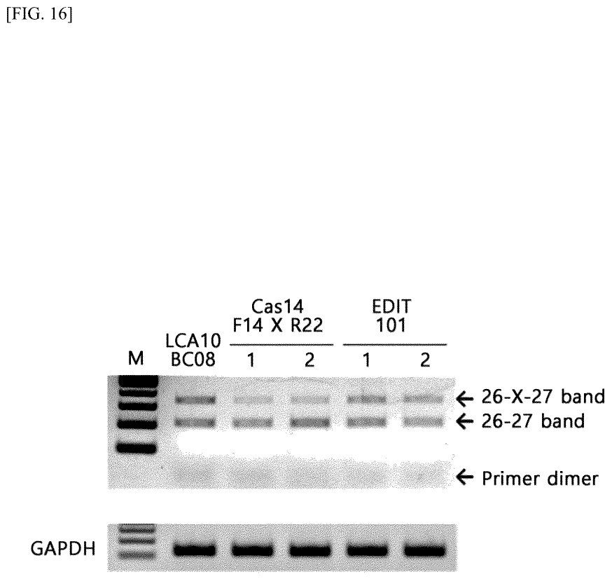

As a result, it was identified that the guide RNA having F14 as a guide sequence and the guide RNA having R22 as a guide sequence resulted in decreased expression of X-27 of the CEP290 gene (FIG. 15). In addition, it was identified that the two guide RNAs resulted in a decreased amount of the band 26-X-27 even in RT-PCR (FIG. 16). In addition, it was also identified that the two guide RNAs resulted in deletion of the target region. Here, EDIT101 was used as a comparator group.

### Example 6. Production and Screening of 661W Mouse LCA10 Disease Model Cell Line

### Example 6-1. Production of 661W Mouse LCA10 Disease Model Cell Line

A 661W mouse LCA10 disease model cell line was produced. The procedure was performed similarly to the method described in the section <Production of ARPE19/HPV16-LCA10 cell line>, except that 661W mouse cells were used instead of ARPE19/HPV16 cells. In addition, unlike the ARPE19 cells for which introduction of a point mutation was performed, in a case of the 661W mouse cells, insertion of the entire sequence of human exon 26 to exon 27, which contains the IVS26 mutation, was performed using a homology-directed repair (HDR) method.

Specifically, exon 25 and exon 26 of the mouse CEP290 gene were targeted to delete the corresponding sequences, and insertion of the entire sequence of human exon 26 to exon 27, which contains the IVS26 mutation, was performed with a donor sequence of SEQ ID NO: 476 through the repair (HDR) method. In addition, instead of the puromycin resistance gene used in Example 4, a neomycin resistance gene was inserted together into the right homology arm of the donor sequence to allow for selection.

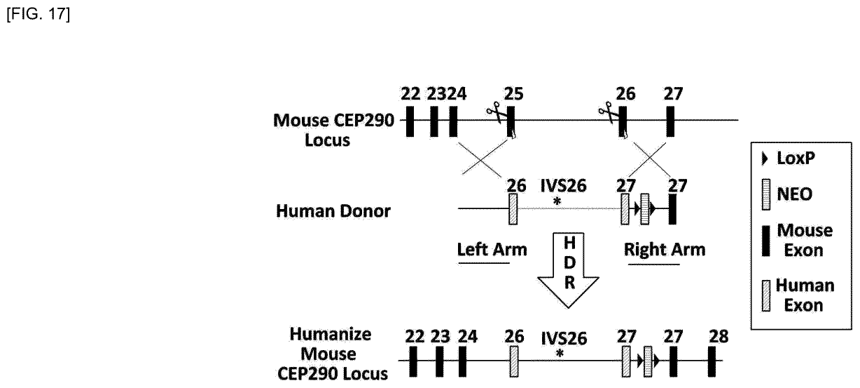

A schematic diagram for production of the 661W/LCA10 cells is shown in FIG. 17.

### Example 6-2. Screening of 661W/LCA10 Cells

Screening of the 661W/LCA10 cells was performed similarly to the method described in the section <12. Screening of ARPE19/HPV16-LCA10 cells>.

Here, instead of puromycin, neomycin (600 μg/ml) was used to perform selection.

DNA of the selected clone was extracted, and it was identified whether the sequence insertion was properly performed through PCR using 1) Inner Primer F (5′-TGTGGCAGCCATTATTCCTGTCTCTA-3′ (SEQ ID NO: 487)) and Inner Primer R (5′-GCAACACAGTGAGACTCTGTTTCAAA-3′ (SEQ ID NO: 488)), 2) Left Arm Primer F (5′-TCTTCTGTGGCTATCATGAGAGCGTC-3′ (SEQ ID NO: 491)) and Left Arm Primer R (5′-TGGGTACAGGGGTAAGAGAAAGGGAT-3′ (SEQ ID NO: 492)), and 3) Right Arm Primer F (5′-GGAACTATGATTCAAGATGAGATTTGGGTA-3′ (SEQ ID NO: 489)) and Right Arm Primer R (5′-TTGGACACTTCAACTTTGAGCTCC-3′ (SEQ ID NO: 490)), respectively.

Then, Sanger sequencing was performed to identify whether the entire sequence was inserted correctly.

The cells containing the mutation were named according to the following nomenclature: A to H, numbers of 1 to 12 (A to H: characters corresponding to rows of a 96-well plate; numbers of 1 to 12: numbers corresponding to columns in respective rows, for example, E08: clone at row E, eighth column in a 96-well plate).

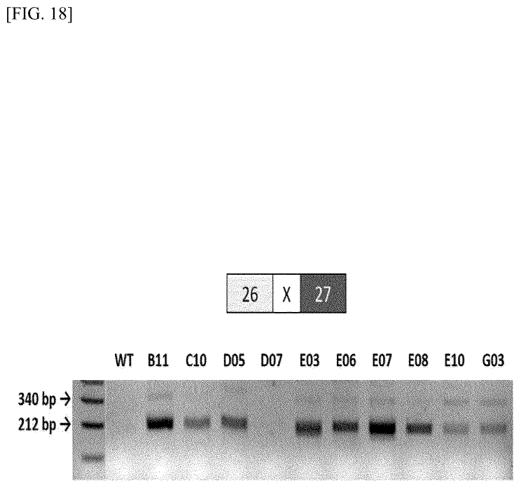

Then, for each clone, expression of 26-X-27 (X means cryptic exon X) was checked through RT-PCR (FIG. 18). The primer set used is as shown in Table 32.

TABLE 32
Primer set

| | PCR | Primer | | Product |
|---|---|---|---|---|
| Target | product | name | Sequence | size |
| Human | 26-X-27 | CEP290 | AGGAAATGGCCATTTTCAAGATTGCA | 212 bp |
| CEP290 | | WT F#02 | (SEQ ID NO: 279) | |
| exon 26-27 | | Screening | AGACTCCACTTGTTCTTTTAAGGAG | (340 bp) |
| | | R#02 | (SEQ ID NO: 280) | |

Then, from the viewpoint that mouse cell lines show expression of Y and Z in addition to cryptic exon X, in order to select a cell line with high expression of cryptic exon X, expression of X-27 was checked through RT-PCR and the optimal clone was selected. The primer set used is as shown in Table 33.

TABLE 33
Primer set

| | PCR | Primer | | Product |
|---|---|---|---|---|
| Target | product | name | Sequence | size |
| Human | X-27 | CEP290 | tagagatggggtttcaccttgttagc | 164 bp |
| CEP290 | | Mu F#3 | (SEQ ID NO: 281) | |
| cryptic exon | | CEP290 | AGACTCCACTTGTTCTTTTAAGGAG | |
| X | | CDNA | (SEQ ID NO: 280) | |
| | | Screening | | |
| | | R#02 | | |

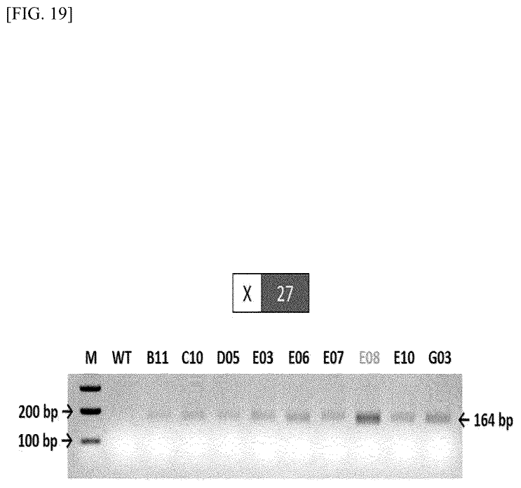

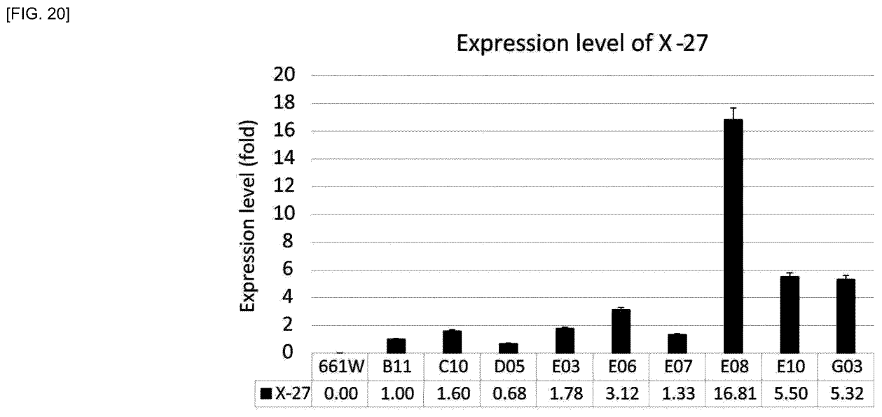

As a result of the experiment, the clone 661W/LCA10-E08 having the highest expression level of X-27 was selected and used for the experiments (FIGS. 19 and 20).

### Example 7. Identification of Deletion of Partial Region of CEP290 Gene for 661W/LCA10 Cell Line

For the 661W/LCA10-E08 cell line produced in Example 6, deletion of a partial region of the CEP290 gene caused by the LCA10 Cas12f1 therapeutic agent was identified.

Specifically, the procedure was performed similarly to the method described in the section <Example 2-1. Identification of deletion of partial region in CEP290 gene>, except that 1) a one-vector system was used which is capable of expressing all of a Cas12f1 (Cas14a1) protein, a guide RNA targeting the F region, and a guide RNA targeting the R region in a form of a single vector, 2) the 661W/LCA10-E08 cells produced in Example 6 were used instead of the HEK-293T cells, and 3) the guide sequences F14× R18 and F14× R22 were used to identify the deletion.

The primer sets used for RT-PCR are as shown in Table 34.

TABLE 34
Primer set

| | Primer | | Product |
|---|---|---|---|
| Target | name | Sequence | size |
| Human | CEP290 | TGGACCAGACCCTTTGTAGTTATC | 197 bp |
| CEP290 | qPCR F#1 | TT (SEQ ID NO: 483) | |
| | CEP290 | TGAGCCAGCAAAAGCTTTTGAGCT | |
| | qPCR R#1 | (SEQ ID NO: 484) | |
| | CEP290 | CTTACATCCTCCTTACTGCCACAA | 195 bp |
| | 64 qPCR | GA (SEQ ID NO: 485) | |
| | F#01 | | |
| | CEP290 | actgtggtcagaaaactcagctag | |
| | 64 qPCR | tt (SEQ ID NO: 486) | |
| | R#01 | | |

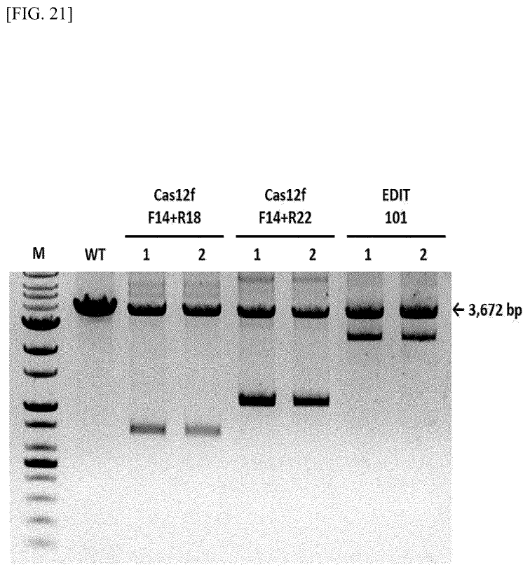

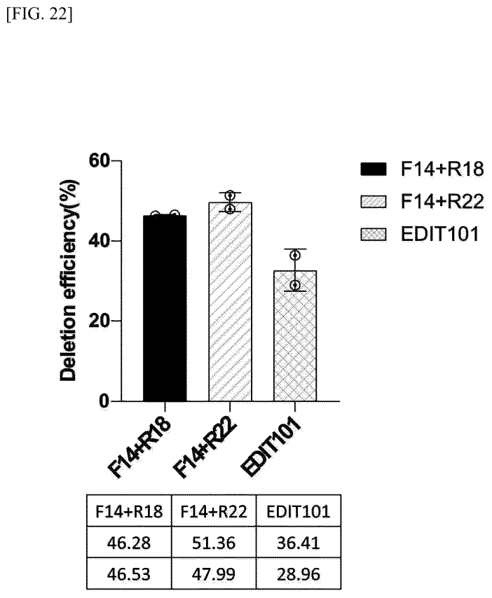

As a result of the experiment, upon comparison of signal intensity between the band of WT, which is a negative control with no treatment, and the bands of the same size, it was identified that the signal intensity of the bands of Cas12f1 F14+R18 and Cas12f1 F14+R22 was lower than EDIT101 (FIG. 21). In addition, as a result of identifying deletion through q-PCR, it was found that deletion efficiency of Cas12f1 F14+R18 and Cas12f1 F14+R22 was higher than EDIT101 (FIG. 22).

### Example 8. Target Screening for Cas12f1 Variants

### Example 8-1. Target Screening 1 for Cas12f1 Variants

In order to screen an efficient target sequence for the Cas12f1 variant of SEQ ID NO: 288 (transposon-associated transposase B derived from Candidatus Woesearchaeota archaeon; also known as TnpB), an experiment was conducted similarly to that described in the section <Example 2. Selection of guide sequence>.

Here, the experiment was conducted in such a way that 1) 5′-ACCGCTTCACCAAAAGCTGTCCCTTAGGGGATTAGAACTTGAGTGAAGG TGGGCTGCTTGCATCAGCCTAATGTCGAGAAGTGCTTTCTTCGGAAAGTAA CCCTCGAAACAAAGAAAGGAATGCAAC-3′ (SEQ ID NO: 477) was used as a scaffold sequence of the guide RNAs, 2) 5′-UUUUAUUUU-3′ was used as a U-rich tail sequence, and 3) F10, F11, F12, F14, F15, F16, F17, F18, F23, F24, R01, R03, R04, R05, R07, R18, R20, R22, R23, and R26 in Tables 3 and 4 were used as guide sequences, respectively.

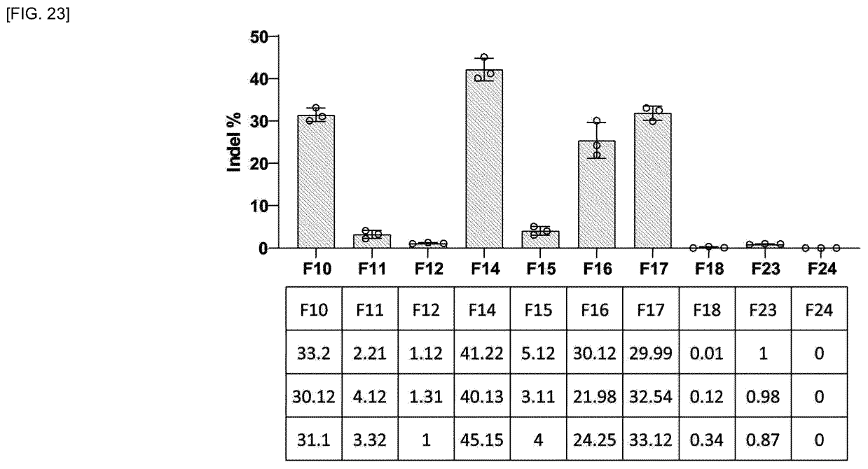

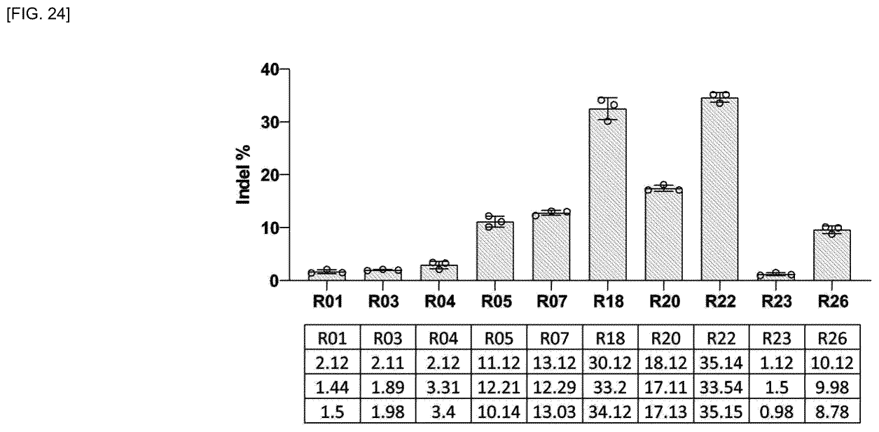

As a result of the experiment, indel efficiency was observed in most of the targets except F24, and in particular, high indel efficiency was observed in F10, F14, F16, F17, R18, and R22 (FIGS. 23 and 24).

### Example 8-2. Target Screening 2 for Cas12f1 Variants

For the targets F14, R18, and R22, which showed high indel efficiency in Example 8-1, different guide sequences were compared in terms of efficiency.

Specifically, an experiment was conducted similarly to that described in the section <Example 2. Selection of guide sequence>, except that 1) each of 5′-ACCGCTTCACCAAAAGCTGTCCCTTAGGGGATTAGAACTTGAGTGAAGG TGGGCTGCTTGCATCAGCCTAATGTCGAGAAGTGCTTTCTTCGGAAAGTAA CCCTCGAAACAAAGAAAGGAATGCAAC-3′ (SEQ ID NO: 477) (denoted as ver. 4.0), and 5′-ACCGCUUCACCGAGUGAAGGUGGGCUGCUUGCAUCAGCCUAAUGUCG AGAAGUGCUUUCUUCGGAAAGUAACCCUCGAAACAAAgaaaGGAAUGCAA C-3′ (SEQ ID NO: 478) (denoted as ver 4.1) was used as a scaffold sequence of the guide RNAs, 2) 5′-UUUUAUUUU-3′ was used as a U-rich tail sequence, and 3) F14, F18, and R22 in Tables 3 and 4 were used as guide sequences, respectively.

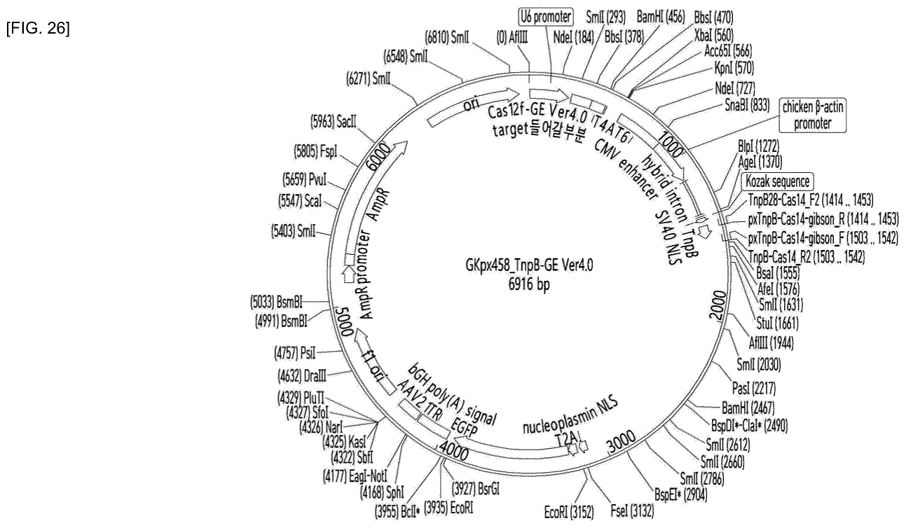

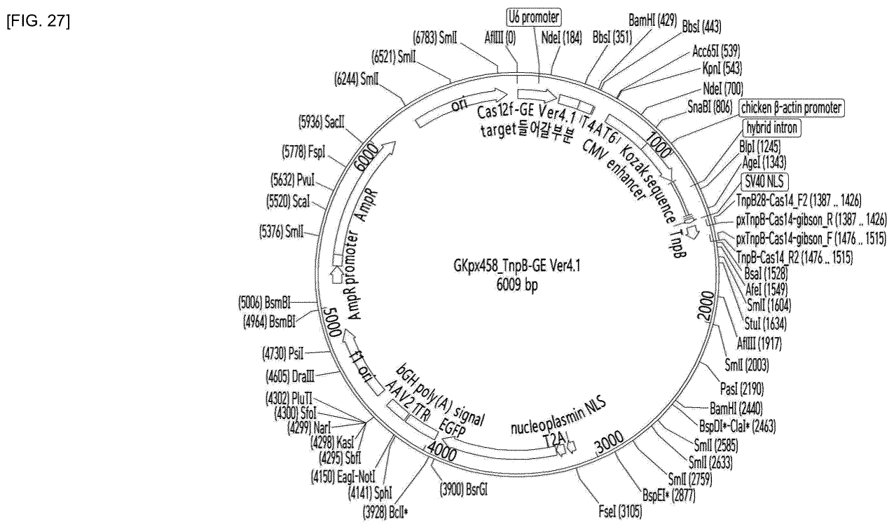

In addition, a one-vector system expressing the Cas12f1 variant of SEQ ID NO: 288 and a guide RNA was constructed and used for transfection. Specifically, the guide sequences were cloned using the px458-TnpB-GE vector (TnpB, gRNA scaffold Bbsl cloning) and the Bbsl enzyme, and used (FIGS. 26 and 27).

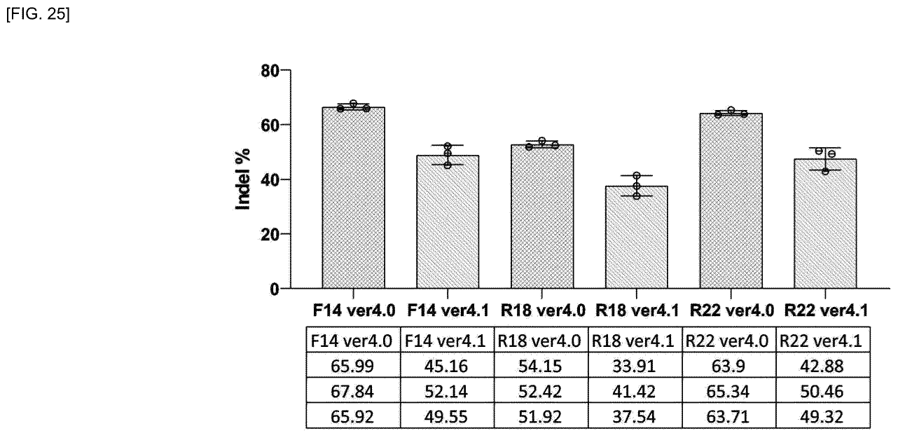

As a result of the experiment, significant indel efficiency was observed for both the guide RNA scaffolds of SEQ ID NO: 477 (ver 4.0) and SEQ ID NO: 478 (ver. 4.1); and in general, it was found that slightly higher indel efficiency was observed in a case where the guide RNA scaffold of SEQ ID NO: 477 was used (FIG. 25).

### Example 8-3. Target Screening 3 for Cas12f1 Variants

In order to optimize the length of the guide sequence, an experiment was conducted similarly to that described in the section <Example 3-1. Optimization of guide sequence>.

Specifically, the experiment was conducted in such a way that 1) nucleic acids encoding the Cas12f1 variant of SEQ ID NO: 288, the guide RNA scaffold sequence of SEQ ID NO: 477, and the U-rich tail of 5′-UUUUAUUUU-3′ were used, 2) guide sequences were cloned using the px458-TnpB-GE vectors (TnpB, gRNA scaffold Bbsl cloning), which contain nucleic acids encoding guide sequences of various lengths, and the Bbsl enzyme, and 3) among the guide sequences shown in Tables 27 and 28, each of the guide sequences of Guide-F14-19nt to Guide-F14-25nt, Guide-RI8-20nt to Guide-RI8-25nt, and Guide-R22-20nt to Guide-R22-25nt was used.

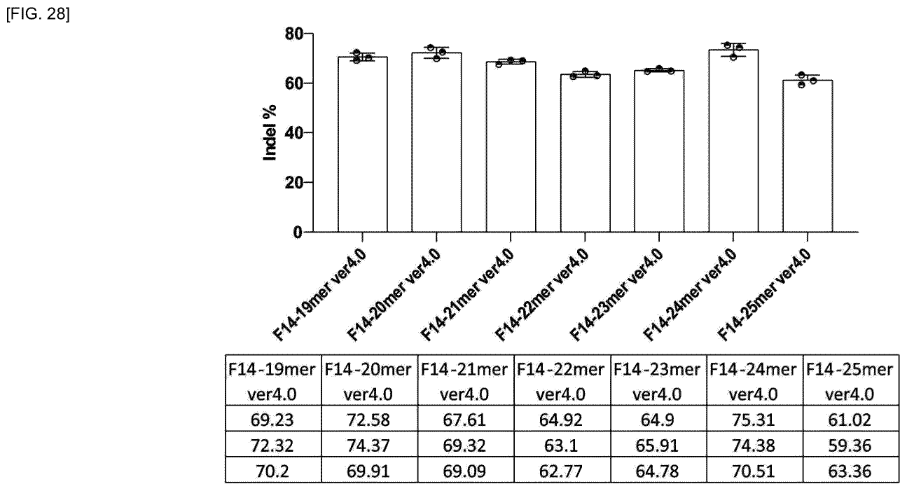

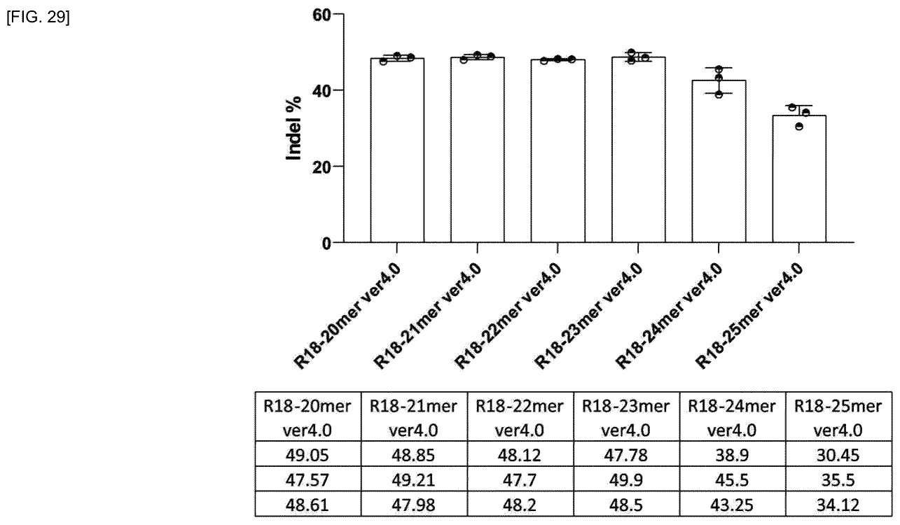

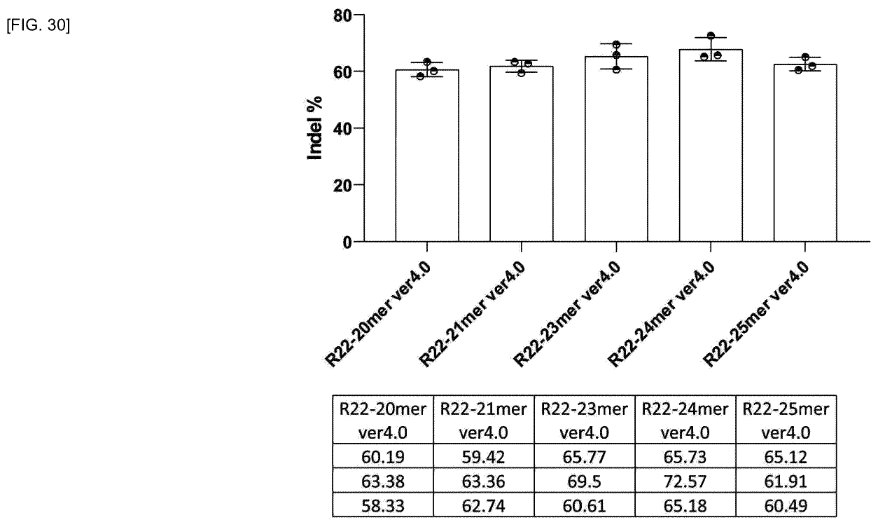

As a result of the experiment, all showed high activity for various lengths of spacer (FIGS. 28 to 30).

### Example 9. Identification of Efficiency of LCA10 Cas12f1 Variant Therapeutic Agent

In order to identify efficiency of the Cas12f1 variant-based LCA10 therapeutic agent based on the results of Example 8, evaluation of efficiency was performed using the ARPE19/LCA10-BC08 cell line and the 661W/LCA10-E08 cell line.

Specifically, an experiment was performed similarly to that described in the section <Example 5. Identification of efficiency of LCA10 Cas12f1 therapeutic agent>, except that 1) the ARPE19/LCA10-BC08 cells and the 661W/LCA10-E08 cells were used, 2) the px458-TnpB-GE vector (TnpB, gRNA scaffold Bbsl cloning), which contains a nucleic acid encoding the Cas12f1 variant of SEQ ID NO: 288 and a nucleic acid encoding two guide RNAs, was used, 3) each guide RNA had, as components, the guide RNA scaffold sequence of SEQ ID NO: 477 and the U-rich tail of 5′-UUUUAUUUU-3′, and 4) (F14 and F18) and (F14 and R22) were used as combinations of the two guide RNAs.

Here, the method of identifying an expression level of X-27 for the ARPE19/LCA10-BC08 cell line was the same as that described in the section <Example 5. Identification of efficiency of LCA10 Cas12f1 therapeutic agent>, and the method of identifying an expression level of X-27 for 661W/LCA10-E08 was the same as that described in the section <Example 6-2. Screening of 661W/LCA10 cells>.

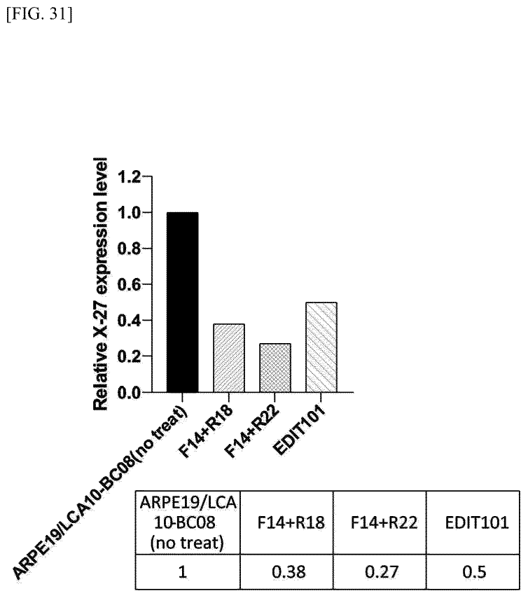

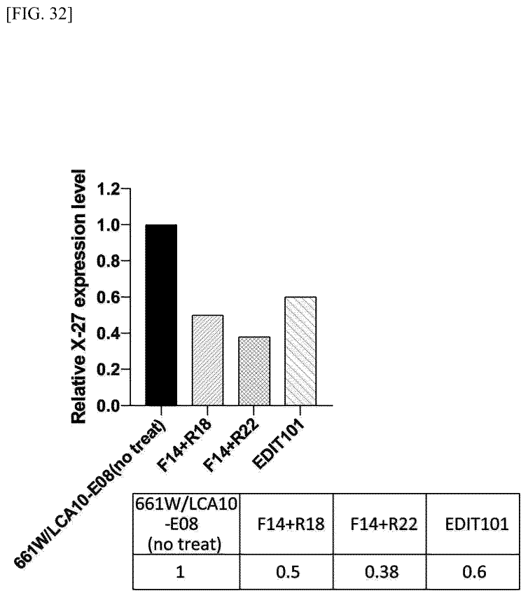

As a result of the experiment, it was found that the LCA10 Cas12f1 variant therapeutic agent of the present disclosure significantly decreased an expression level of X-27 in both the ARPE19/LCA10-BC08 cell line and the 661W/LCA10-E08 cell line, and showed a superior effect to that of the comparator group EDIT101 (FIGS. 31 and 32).

### Example 10. Identification of Effect of Improving Deletion Efficiency, Caused by Factor for Decreasing NHEJ Activity

This example was intended to identify an effect of improving deletion efficiency in a case of additionally using a factor for decreasing non-homologous end joining DNA repair pathway (NHEJ) activity together with a LCA10 Cas12f1 therapeutic agent and a LCA10 Cas12f1 variant therapeutic agent.

An experiment was conducted in HEK293T cells similarly to that described in the section <Example 2-1. Identification of deletion of partial region in CEP290 gene>.

Here, each of the following one-vector systems was used: 1) a one-vector system that contains a Cas12f1 protein, a guide RNA targeting F14, a guide RNA targeting R22, and a nucleic acid encoding a factor for decreasing NHEJ activity, and 2) a one-vector system that contains the Cas12f1 protein variant of SEQ ID NO: 288, a guide RNA targeting F14, a guide RNA targeting R22, and a nucleic acid encoding a factor for decreasing NHEJ activity.

Each of the guide RNAs contained the guide RNA scaffold region of SEQ ID NO: 477 and the U-rich tail of 5′-UUUUAUAUUUU-3′.

In addition, the experiment was conducted in such a way that for the factor for decreasing NHEJ activity used, one or two units of nucleic acid encoding shDCLRE1C-3 of SEQ ID NO: 473 were included in the vector, and shscramble was used as a comparator group.

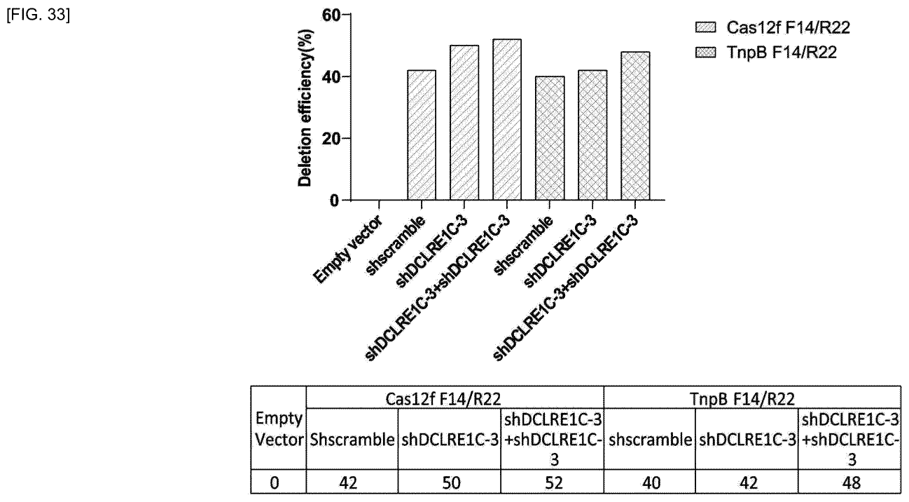

As a result of the experiment, it was found that higher deletion efficiency was observed in a case where the factor for decreasing NHEJ activity was used together with the LCA10 Cas12f1 therapeutic agent or the LCA10 Cas12f1 variant therapeutic agent (FIG. 33).

### INDUSTRIAL APPLICABILITY

The present disclosure relates to treatment of LCA10 disease using the CRISPR/Cas12f1 (Cas14a1) system. Through the technology disclosed by the present specification, it is possible to provide a composition comprising the CRISPR/Cas12f1 (Cas14a1) system for treatment of LCA10 disease and a treatment method using the same. In addition, it is possible to provide a composition for artificially manipulating or modifying the CEP290 gene, comprising the CRISPR/Cas12f1 (Cas14a1) system, and a manipulation or modification method using the same.

1. A guide RNA for a CRISPR/Cas12f1 system, comprising:
a scaffold region represented by a nucleotide sequence selected from SEQ ID NO: 125, SEQ ID NO: 126, SEQ ID NO: 477, and SEQ ID NO: 478;
a spacer region comprising, as a guide sequence, a nucleotide sequence selected from SEQ ID NOS: 51 to 101 and SEQ ID NOS: 245 to 272; and
a U-rich tail,
wherein the scaffold region, the guide region, and the U-rich tail are sequentially linked to each other in a 5′ to 3′ direction,
the scaffold region is a region capable of interacting with a Cas12f1 protein, the guide region is capable of forming a complementary bond with a target sequence present in CEP290 gene, and
the U-rich tail is represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10.

2. A DNA encoding the guide RNA of claim 1.

3. A guide RNA-Cas12f1 protein complex, comprising:
a first Cas12f1 protein represented by an amino acid sequence selected from SEQ ID NO: 129 or SEQ ID NO: 288;
a second Cas12f1 protein having the same amino acid sequence as the first Cas12f1 protein; and
a guide RNA in which a scaffold region represented by a nucleotide sequence selected from SEQ ID NO: 125, SEQ ID NO: 126, SEQ ID NO: 477, and SEQ ID NO: 478, a spacer region comprising, as a guide sequence, a nucleotide sequence selected from SEQ ID NOS: 51 to 101 and SEQ ID NOS: 245 to 272, and a U-rich tail are sequentially linked to each other in a 5′ to 3′ direction,
wherein the first Cas12f1 protein and the second Cas12f1 protein form a dimer, the scaffold region of the guide RNA and the dimer formed by the Cas12f1 proteins interact with each other to form a complex,
the U-rich tail is represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10, and
the guide region is capable of forming a complementary bond with a target sequence present in CEP290 gene.

4. A vector comprising:
a nucleic acid that encodes a Cas12f1 protein represented by an amino acid sequence selected from SEQ ID NO: 129 or SEQ ID NO: 288; and
a nucleic acid that encodes a guide RNA, in which a scaffold region represented by a nucleotide sequence selected from SEQ ID NO: 125, SEQ ID NO: 126, SEQ ID NO: 477, and SEQ ID NO: 478, a spacer region comprising, as a guide sequence, a nucleotide sequence selected from SEQ ID NOS: 51 to 101 and SEQ ID NOS: 245 to 272, and a U-rich tail are sequentially linked to each other, wherein the U-rich tail is represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10.

5. A vector comprising:
a nucleic acid that encodes a Cas12f1 protein represented by an amino acid sequence selected from SEQ ID NO: 129 or SEQ ID NO: 288; and
a nucleic acid that encodes a first guide RNA in which a first scaffold region represented by a nucleotide sequence selected from SEQ ID NO: 125, SEQ ID NO: 126, SEQ ID NO: 477, and SEQ ID NO: 478, a first spacer region comprising, as a guide sequence, a nucleotide sequence selected from SEQ ID NOS: 51 to 74 and SEQ ID NOS: 245 to 256, and a first U-rich tail are sequentially linked to each other; and
a nucleic acid that encodes a second guide RNA in which a second scaffold region represented by a nucleotide sequence selected from SEQ ID NO: 125, SEQ ID NO: 126, SEQ ID NO: 477, and SEQ ID NO: 478, a second spacer region comprising, as a guide sequence, a nucleotide sequence selected from SEQ ID NOS: 75 to 101 and SEQ ID NOS: 257 to 272, and a second U-rich tail are sequentially linked to each other, wherein the first U-rich tail and the second U-rich tail are each independently represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10.

6. The vector of claim 4, further comprising at least one unit of a shRNA, which decreases activity of one or more genes selected from ATM1, XRCC4, XLF-1, XRCC6, LIG4, and DCLRE1C, or a nucleic acid encoding the shRNA.

7. The vector of claim 4, wherein the vector is one or more selected from:
plasmid; mRNA (transcript); PCR amplicon; retroviral (retrovirus) vector;
lentiviral (lentivirus) vector; adenoviral (adenovirus) vector; adeno-associated viral (adeno-associated virus; AAV) vector; vaccinia viral (vaccinia virus) vector; poxviral (poxvirus) vector; and herpes simplex viral (herpes simplex virus) vector.

8. The vector of claim 4, wherein the vector is a single vector.

9. The vector of claim 6, wherein the first spacer region of the first guide RNA comprises, as a guide sequence, the nucleotide sequence of SEQ ID NO: 64, the second spacer region of the second guide RNA comprises, as a guide sequence, the nucleotide sequence of SEQ ID NO: 92 or SEQ ID NO: 96, the vector comprises two units of the nucleic acid encoding the shRNA, and the vector is an adeno-associated viral vector and is a single vector.

10. A CRISPR/Cas12f1 composition comprising:
a Cas12f1 protein represented by an amino acid sequence selected from SEQ ID NO: 129 or SEQ ID NO: 288, or a nucleic acid encoding the Cas12f1 protein; and
a guide RNA in which a scaffold region represented by a nucleotide sequence selected from SEQ ID NO: 125, SEQ ID NO: 126, SEQ ID NO: 477, and SEQ ID NO: 478, a spacer region comprising, as a guide sequence, a nucleotide sequence selected from SEQ ID NOS: 51 to 101 and SEQ ID NOS: 245 to 272, and a U-rich tail are sequentially linked to each other, or a nucleic acid encoding the guide RNA, wherein the Cas12f1 protein and the scaffold region of the guide RNA are capable of interacting with each other to form a complex,
the spacer region of the guide RNA is capable of forming a complementary bond with a target sequence present in CEP290 gene, and
the U-rich tail is represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10.

11. A CRISPR/Cas12f1 composition comprising:
a Cas12f1 protein represented by an amino acid sequence selected from SEQ ID NO: 129 or SEQ ID NO: 288, or a nucleic acid encoding the Cas12f1 protein; and
a first guide RNA in which a first scaffold region represented by a nucleotide sequence selected from SEQ ID NO: 125, SEQ ID NO: 126, SEQ ID NO: 477, and SEQ ID NO: 478, a first spacer region comprising, as a guide sequence, a nucleotide sequence selected from SEQ ID NOS: 51 to 74 and SEQ ID NOS: 245 to 256, and a first U-rich tail are sequentially linked to each other, or a nucleic acid encoding the first guide RNA; and
a second guide RNA in which a second scaffold region represented by a nucleotide sequence selected from SEQ ID NO: 125, SEQ ID NO: 126, SEQ ID NO: 477, and SEQ ID NO: 478, a second spacer region comprising, as a guide sequence, a nucleotide sequence selected from SEQ ID NOS: 75 to 101 and SEQ ID NOS: 257 to 272, and a second U-rich tail are sequentially linked to each other, or a nucleic acid encoding the second guide RNA,
wherein the Cas12f1 protein and the first scaffold region are capable of interacting with each other to form a complex,
the Cas12f1 protein and the second scaffold region are capable of interacting with each other to form a complex,
the first spacer region and the second spacer region are each independently capable of forming a complementary bond with a target sequence present in CEP290 gene, and
the first U-rich tail and the second U-rich tail are each independently represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10.

12. The CRISPR/Cas12f1 composition of claim 10, further comprising at least one unit of a shRNA, which decreases activity of one or more genes selected from ATM1, XRCC4, XLF-1, XRCC6, LIG4, and DCLRE1C, or a nucleic acid encoding the shRNA.

13. The CRISPR/Cas12f1 composition of claim 12, wherein the CRISPR/Cas12f1 composition comprises the first guide RNA and the second guide RNA,
the first spacer region of the first guide RNA comprises, as a guide sequence, the nucleotide sequence of SEQ ID NO: 64,
the second spacer region of the second guide RNA comprises, as a guide sequence, the nucleotide sequence of SEQ ID NO: 92 or SEQ ID NO: 96, and
the CRISPR/Cas12f1 composition comprises two units of the shRNA or the nucleic acid encoding the shRNA.

14. A pharmaceutical composition for treatment of LCA10, comprising:
the CRISPR/Cas12f1 composition of claim 11; and
a pharmaceutically acceptable carrier.

15. The pharmaceutical composition of claim 14, further comprising a shRNA, which decreases activity of one or more genes selected from ATM1, XRCC4, XLF-1, XRCC6, LIG4, and DCLRE1C, or a nucleic acid encoding the shRNA.

16. A method for editing CEP290 gene that comprises IVS26 mutation (c.2991+1655A>G), comprising:
introducing a CRISPR/Cas12f1 composition into a subject,
wherein the CRISPR/Cas12f1 composition comprises:
a Cas12f1 protein represented by an amino acid sequence selected from SEQ ID NO: 129 or SEQ ID NO: 288, or a nucleic acid encoding the Cas12f1 protein; and
a first guide RNA in which a first scaffold region represented by a nucleotide sequence selected from SEQ ID NO: 125, SEQ ID NO: 126, SEQ ID NO: 477, and SEQ ID NO: 478, a first spacer region comprising, as a guide sequence, a nucleotide sequence selected from SEQ ID NOS: 51 to 74 and SEQ ID NOS: 245 to 256, and a first U-rich tail are sequentially linked to each other, or a nucleic acid encoding the first guide RNA; and
a second guide RNA in which a second scaffold region represented by a nucleotide sequence selected from SEQ ID NO: 125, SEQ ID NO: 126, SEQ ID NO: 477, and SEQ ID NO: 478, a second spacer region comprising, as a guide sequence, a nucleotide sequence selected from SEQ ID NOS: 75 to 101 and SEQ ID NOS: 257 to 272, and a second U-rich tail are sequentially linked to each other, or a nucleic acid encoding the second guide RNA,
wherein the Cas12f1 protein and the first scaffold region are capable of interacting with each other to form a complex,
the Cas12f1 protein and the second scaffold region are capable of interacting with each other to form a complex,
the first spacer region of the first guide RNA of the CRISPR/Cas12f1 composition targets a region between the 3′ end of exon 26 of the CEP290 gene and the IVS26 mutation (c.2991+1655A>G) of the CEP290 gene,
the second spacer region of the second guide RNA of the CRISPR/Cas12f1 composition targets a region between the IVS26 mutation (c.2991+1655A>G) of the CEP290 gene and the 5′ end of exon 27 of the CEP290 gene,
the first U-rich tail and the second U-rich tail are each independently represented by a sequence selected from 5′-(UaN)dUe-3′, 5′-UaVUaVUe-3′, and 5′-UaVUaVUaVUe-3′ wherein N is A, C, G or U, each V is independently A, C or G, a is an integer from 0 to 4, d is an integer from 0 to 3, and e is an integer from 1 to 10, and
introduction of the CRISPR/Cas12f1 composition to the subject induces removal of a partial nucleic acid sequence, which comprises the IVS26 mutation (c.2991+1655A>G), in a region of intron 26 of the CEP290 gene.

17. The method of claim 16, wherein the introduction of the CRISPR/Cas12f1 composition into the subject is done by administering the CRISPR/Cas12f1 composition to a human, or a non-human animal having the CEP290 gene.

18. The method of claim 16, wherein the introduction of the CRISPR/Cas12f1 composition into the subject is done by introducing the CRISPR/Cas12f1 composition into a human cell, a non-human animal cell having the CEP290 gene, a cell obtained from a human with LCA10 disease, or a cell obtained from a non-human animal with LCA10 disease.

19. The method of claim 16, wherein the CRISPR/Cas12f1 composition further comprises at least one unit of a shRNA, which decreases activity of one or more genes selected from ATM1, XRCC4, XLF-1, XRCC6, LIG4, and DCLRE1C, or a nucleic acid encoding the shRNA.

20. The method of claim 19, wherein the first spacer region of the first guide RNA of the CRISPR/Cas12f1 composition comprises, as a guide sequence, the nucleotide sequence of SEQ ID NO: 64,
the second spacer region of the second guide RNA of the CRISPR/Cas12f1 composition comprises, as a guide sequence, the nucleotide sequence of SEQ ID NO: 92 or SEQ ID NO: 96, and
the CRISPR/Cas12f1 composition further comprises two units of the shRNA or the nucleic acid encoding the shRNA.

21. A method for treating LCA10 disease, comprising:
administering the composition of claim 12 to a subject with LCA10 disease.

22. The method of claim 21, wherein the subject is a human with LCA10 disease or a non-human animal with LCA10 disease.

23. The method of claim 21, wherein the first spacer region of the first guide RNA of the CRISPR/Cas12f1 composition comprises, as a guide sequence, the nucleotide sequence of SEQ ID NO: 64,
the second spacer region of the second guide RNA of the CRISPR/Cas12f1 composition comprises, as a guide sequence, the nucleotide sequence of SEQ ID NO: 92 or SEQ ID NO: 96, and
the composition further comprises two units of the shRNA or the nucleic acid encoding the shRNA.

24-26. (canceled)
